(19) United States
(12) Patent Application Publication
(10) Pub. No.: US 2020/0362058 A1
(43) Pub. Date: Nov. 19, 2020

(54) ANTIBODY-CYTOKINE ENGRAFTED PROTEINS AND METHODS OF USE

(71) Applicant: NOVARTIS AG, Basel (CH)

(72) Inventors: Michael DiDonato, San Diego, CA (US); Glen Spraggon, San Diego, CA (US)

(21) Appl. No.: 16/616,131

(22) PCT Filed: May 22, 2018
(86) PCT No.: PCT/IB2018/053625

§ 371 Date: Nov. 22, 2019

Related U.S. Application Data
(60) Provisional application No. 62/510,573, filed on May 24, 2017.

(51) Int. Cl. C07K 16/46; C07K 14/52; A61P 35/00

(52) U.S. Cl. CPC C07K 16/46; A61K 45/06; A61P 35/00; C07K 14/52

(57) ABSTRACT

The present invention provides antibody cytokine engrafted (ACE) proteins, including those that stimulate intracellular signaling, and are useful in the treatment of cancer, immunotherapy and metabolic disorders. In particular, the provided ACE protein compositions provide preferred biological effects over wild type cytokine proteins. For example, the provided ACE proteins can convey improved half-life, stability and produceability over the corresponding recombinant cytokine formulations.

### CROSS-REFERENCE TO RELATED APPLICATIONS

This application claims the benefit of U.S. Provisional Application No. 62/510,573 filed May 24, 2017, the content of which is hereby incorporated by reference in its entirety.

### FIELD

The present invention relates to Antibody Cytokine Engrafted (ACE) proteins, compositions and methods of treatment.

### SEQUENCE LISTING

The instant application contains a Sequence Listing which has been submitted electronically in ASCII format and is hereby incorporated by reference in its entirety. Said ASCII copy, created on May 14, 2018, is named PAT057624-WO-PCT_SL.txt and is 4,389,055 bytes in size.

### BACKGROUND

Helical cytokines are compact molecules made up of four to seven alpha helices, with a total helical content of 70-90%. A signature element of all helical cytokines is a four-helix bundle, the amphipathic helices of which are arranged in an almost antiparallel manner so that the majority of the hydrophobic amino acids are involved in the formation of an internal hydrophobic core inside the helical bundle.

Four alpha helix bundles display some common characteristics. The first to examine the four helix bundle proteins was Weber and Salemme (Weber and Salemme, Nature 1980; 287:82-84). In this work, the four-helix bundles considered were antiparallel helices arranged in an up-down-up-down topology. Using a larger data set, this type of protein topology was further defined in the work by Presnell, including the topology of helical cytokines with helices arranged in an up-up-down-down conformation (Presnell and Cohen, PNAS USA 1989; 86:6592-6596).

Several four helix bundle proteins, including IL-6, leukemia inhibitory factor (LIF), oncostatin M (OSM), ciliary neurotrophic factor (CNTF), cardiotrophin-1 (CT-1), cardiotrophin-like cytokine (CLC), IL-11 and IL-31, belong to a family of cytokines wherein signaling is mediated via the receptor subunit GP130 (Barton et al., J. Biol. Chem 1999; 274:5755-5761). Therefore, these cytokines partly show functional overlaps, despite some unique biological activities (Negandaripour et al., Cytokine and Growth Factor Rev. 2016; 32:41-61). In addition to GP130, several other receptor subunits may be involved in the signaling transduction of this family

### DESCRIPTION

The present disclosure provides for a cytokine engrafted into a CDR sequence of an antibody, and henceforth, these Antibody Cytokine Engrafted proteins will be known as ACE proteins. In particular, the provided ACE protein compositions provide preferred biological effects over wild type cytokine proteins. For example, the provided ACE proteins can convey improved half-life, stability and produceability over the corresponding recombinant cytokine formulations. The disclosure provides for an ACE protein comprising: (a) a heavy chain variable region (VH), comprising Complementarity Determining Regions (CDR) HCDR1, HCDR2, HCDR3; and (b) a light chain variable region (VL), comprising LCDR1, LCDR2, LCDR3; and (c) a cytokine molecule engrafted into a CDR of the VH or the VL. In some embodiments, the cytokine molecule is directly engrafted into the CDR. In some embodiments, the cytokine molecule is not interleukin-10 (IL-10).

In some embodiments, the cytokine molecule is engrafted into a heavy chain CDR.

In some embodiments, the heavy chain CDR is selected from complementarity determining region 1 (HCDR1), complementarity determining region 2 (HCDR2) or complementarity determining region 3 (HCDR3).

In some embodiments, the cytokine molecule is engrafted into the HCDR1.

In some embodiments, the cytokine molecule is engrafted into the HCDR2.

In some embodiments, the cytokine molecule is engrafted into the HCDR3.

In some embodiments, the cytokine molecule is engrafted into a light chain CDR.

In some embodiments, the light chain CDR is selected from complementarity determining region 1 (LCDR1), complementarity determining region 2 (LCDR2) or complementarity determining region 3 (LCDR3).

In some embodiments, the cytokine molecule is engrafted into the LCDR1.

In some embodiments, the cytokine molecule is engrafted into the LCDR2.

In some embodiments, the cytokine molecule is engrafted into the LCDR3.

In some embodiments, the cytokine molecule is directly engrafted into the CDR without a peptide linker.

In some embodiments, the cytoline molecule is a molecule selected from Table 1.

In some embodiments, the ACE protein further comprises an IgG class antibody heavy chain.

In some embodiments, the IgG class heavy chain is selected from IgG1, IgG2, or IgG4.

In some embodiments, the binding specificity of the CDRs to the target protein is reduced by the engrafted cytokine molecule.

In some embodiments, the binding wherein the binding specificity of the CDRs to the target protein is reduced by 10%, 20%, 30%, 40%, 50%, 60%, 70%, 80%, 90%, 95%, 98%, 99%, or 100%, by the engrafted cytokine molecule.

In some embodiments, the binding specificity of the CDRs to the target protein is retained in the presence of the engrafted cytokine molecule.

In some embodiments, the binding specificity of the CDRs to the target protein is retained by 10%, 20%, 30%, 40%, 50%, 60%, 70%, 80%, 90%, 95%, 98%, 99%, or 100%, in the presence of the engrafted cytokine molecule.

In some embodiments, the binding specificity of the CDRs is distinct from the binding specificity of the cytokine molecule.

In some embodiments, the binding specificity of the CDRs is to a non-human antigen.

In some embodiments, the non-human antigen is a virus.

In some embodiments, the virus is respiratory syncytial virus (RSV).

In some embodiments, the RSV is selected from RSV subgroup A or RSV subgroup B.

In some embodiments, the antibody scaffold of the ACE protein is humanized or human

In some embodiments, the antibody scaffold of the ACE protein is palivizumab.

In some embodiments, the binding affinity of the engrafted cytokine molecule to a receptor is increased in comparison to a free cytokine molecule.

In some embodiments, the binding affinity of the engrafted cytokine molecule to a receptor is decreased in comparison to a free cytokine molecule.

In some embodiments, the binding avidity of the engrafted cytokine molecule to a receptor is increased in comparison to a free cytokine molecule.

In some embodiments, the binding avidity of the engrafted cytokine molecule to a receptor is decreased in comparison to a free cytokine molecule.

In some embodiments, the the differential binding affinity or avidity of the engrafted cytokine molecule to two or more receptors is changed in comparison to a free cytokine molecule.

In some embodiments, an activity of the engrafted cytokine molecule is increased in comparison to a free cytokine molecule.

In some embodiments, an activity of the engrafted cytokine molecule is decreased in comparison to a free cytokine molecule.

Some embodiments disclosed herein provide ACE proteins comprising: a heavy chain variable region that comprises: (a) a HCDR1, (b) a HCDR2, and (c) a HCDR3, wherein each of the HCDR sequences are set forth in TABLE 2, and a light chain variable region that comprises: (d) a LCDR1, (e) a LCDR2, and (f) a LCDR3, wherein each of the LCDR sequences are set forth in TABLE 2, wherein a cytokine molecule is engrafted into a CDR.

Some embodiments disclosed herein provide ACE proteins with the proviso that ACE proteins comprising an IL10 cytokine are excluded.

Some embodiments disclosed herein provide ACE proteins with the proviso that ACE proteins set forth in TABLE 3 are excluded.

Some embodiments disclosed herein provide ACE proteins comprising: a heavy chain variable region (VH) that comprises a VH set forth in TABLE 2, and a light chain variable region (VL) that comprises a VL set forth in TABLE 2, wherein a cytokine molecule is engrafted into a VH or VL.

In some embodiments, the ACE protein further comprises a modified Fc region corresponding with reduced effector function.

In some embodiments, the modified Fc region comprises a mutation selected from one or more of D265A, P329A, P329G, N297A, L234A, and L235A.

In some embodiments, the modified Fc region comprises a combination of mutations selected from one or more of D265A/P329A, D265A/N297A, L234/L235A, P329A/L234A/L235A, and P329G/L234A/L235A.

In some embodiments, the Fc region mutation is D265A/P329A.

Some embodiments disclosed herein provide isolated nucleic acids encoding an ACE protein comprising: a heavy chain variable region as set forth in TABLE 2 and/or a light chain variable region as set forth in TABLE 2, wherein a cytokine molecule is engrafted into the heavy chain variable region or the light chain variable region.

Some embodiments disclosed herein provide recombinant host cells suitable for the production of an ACE protein comprising the nucleic acids disclosed herein, and optionally, a secretion signal.

In some embodiments, the recombinant host cell is a mammalian cell line.

In some embodiments, the mammalian cell line is a CHO cell line.

Some embodiments disclosed herein provide pharmaceutical compositions comprising the ACE proteins disclosed herein and a pharmaceutically acceptable carrier.

Some embodiments disclosed herein provide methods of treating a disease in an individual in need thereof, comprising administering to the individual a therapeutically effective amount of the pharmaceutical compositions disclosed herein.

In some embodiments, the disease is a cancer.

In some embodiments, the cancer is selected from the group consisting of: melanoma, lung cancer, colorectal cancer, prostate cancer, breast cancer and lymphoma.

In some embodiments, the pharmaceutical composition is administered in combination with another therapeutic agent.

In some embodiments, the therapeutic agent is an immune checkpoint inhibitor.

In some embodiments, the antagonist to the immune checkpoint is selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4 and TGFR.

In some embodiments, the immune checkpoint inhibitor is an anti-PD-L1 antibody.

In some embodiments, the immune checkpoint inhibitor is an anti-TIM3 antibody.

In some embodiments, the disease is an immune related disorder.

In some embodiments, the immune related disorder is selected from the group consisting of: inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

In some embodiments, the pharmaceutical composition is administered in combination with another therapeutic agent.

In some embodiments, the therapeutic agent is an anti-TNF agent selected from the group consisting of: infliximab, adalimumab, certolizumab, golimumab, natalizumab, and vedolizumab.

In some embodiments, the therapeutic agent is an aminosalicylate agent selected from the group consisting of: sulfasalazine, mesalamine, balsalazide, olsalazine and other derivatives of 5-aminosalicylic acid.

In some embodiments, the therapeutic agent is a corticosteroid selected from the group consisting of: methylprednisolone, hydrocortisone, prednisone, budenisonide, mesalamine, and dexamethasone.

In some embodiments, the therapeutic agent is an antibacterial agent.

Some embodiments disclosed herein provide uses of an ACE protein comprising: a heavy chain variable region that comprises (a) a HCDR1 (b) a HCDR2 (c) a HCDR3 wherein each of the HCDR sequences are set forth in TABLE 2, and a light chain variable region that comprises: (d) a LCDR1, (e) a LCDR2, and (f) a LCDR3, wherein each of the LCDR sequences are set forth in TABLE 2, in the treatment of of a disease, wherein a cytokine molecule is engrafted into a CDR.

In some embodiments, the disease is a cancer.

In some embodiments, the cancer is selected from the group consisting of: melanoma, lung cancer, colorectal cancer, prostate cancer, breast cancer and lymphoma.

In some embodiments, the pharmaceutical composition is administered in combination with another therapeutic agent.

In some embodiments, the therapeutic agent is an immune checkpoint inhibitor.

In some embodiments, the antagonist to the immune checkpoint is selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4 and TGFR.

In some embodiments, the immune checkpoint inhibitor is an anti-PD-L1 antibody.

In some embodiments, the immune checkpoint inhibitor is an anti-TIM3 antibody.

In some embodiments, the disease is an immune related disorder.

In some embodiments, the immune related disorder is selected from the group consisting of: inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

In some embodiments, the pharmaceutical composition is administered in combination with another therapeutic agent.

In some embodiments, the therapeutic agent is an anti-TNF agent selected from the group consisting of: infliximab, adalimumab, certolizumab, golimumab, natalizumab, and vedolizumab.

In some embodiments, the therapeutic agent is an aminosalicylate agent selected from the group consisting of: sulfasalazine, mesalamine, balsalazide, olsalazine and other derivatives of 5-aminosalicylic acid.

In some embodiments, the therapeutic agent is a corticosteroid selected from the group consisting of: methylprednisolone, hydrocortisone, prednisone, budenisonide, mesalamine, and dexamethasone.

In some embodiments, the therapeutic agent is an antibacterial agent.

In certain embodiments, the ACE protein comprises an IgG class antibody Fc region. In particular embodiments, the antibody Fc region is selected from IgG1, IgG2, or IgG4 subclass Fc region. In some embodiments, the antibody optionally contains at least one modification that modulates (i.e., increases or decreases) binding of the antibody to an Fc receptor. The antibody Fc region may optionally comprise a modification conferring modified effector function. In particular embodiments the antibody Fc region may comprise a mutation conferring reduced effector function selected from any of D265A, P329A, P329G, N297A, D265A/P329A, D265A/N297A, L234/L235A, P329A/L234A/L235A, and P329G/L234A/L235A. In some embodiments, the Fc mutation is D265A/P329A.

In some embodiments, the ACE protein also comprises a wild type cytokine or a variant thereof. The variations can be single amino acid changes, single amino acid deletions, multiple amino acid changes and multiple amino acid deletions. For example, a variation in the cytokine portion of the molecule can decrease or increase the affinity of the ACE protein for the cytokine receptor.

In some embodiments, an IL10 wild type or variant cytokine is excluded. In other embodiments, the IL10 ACE proteins as disclosed in TABLE 3 are excluded. In some embodiments, the IL10 ACE protein from Example 39, Example 40, Example 41, Example 42, Example 43, Example 44, Example 45, Example 46, Example 47, Example 48, Example 49, Example 50, or Example 41 are excluded.

Furthermore, the disclosure provides polynucleotides encoding at least a heavy chain and/or a light chain protein of an ACE protein as described herein. In another related aspect, host cells are provided that are suitable for the production of an ACE protein as described herein. In particular embodiments, host cells comprise nucleic acids encoding an ACE protein as described herein. In still another aspect, methods for producing ACE proteins are provided, comprising culturing provided host cells as described herein under conditions suitable for expression, formation, and secretion of the ACE protein and recovering the ACE protein from the culture. In a further aspect, the disclosure further provides kits comprising an ACE protein, as described herein.

In another related aspect, the disclosure further provides compositions comprising an ACE protein, as described herein, and a pharmaceutically acceptable carrier. In some embodiments, the disclosure provides pharmaceutical compositions comprising an ACE protein for administering to an individual.

### Definitions

An “antibody” refers to a molecule of the immunoglobulin family comprising a tetrameric structural unit. Each tetramer is composed of two identical pairs of polypeptide chains, each pair having one “light” chain (about 25 kD) and one “heavy” chain (about 50-70 kD), connected through a disulfide bond. Recognized immunoglobulin genes include the κ, λ, α, γ, δ, ε, and μ constant region genes, as well as the myriad immunoglobulin variable region genes. Light chains are classified as either κ or Heavy chains are classified as γ, μ, α, δ, or ε, which in turn define the immunoglobulin classes, IgG, IgM, IgA, IgD, and IgE, respectively. Antibodies can be of any isotype/class (e.g., IgG, IgM, IgA, IgD, and IgE), or any subclass (e.g., IgG1, IgG2, IgG3, IgG4, IgA1, IgA2).

Both the light and heavy chains are divided into regions of structural and functional homology. The terms “constant” and “variable” are used structurally and functionally. The N-terminus of each chain defines a variable (V) region or domain of about 100 to 110 or more amino acids primarily responsible for antigen recognition. The terms variable light chain (V_{L}) and variable heavy chain (V_{H}) refer to these regions of light and heavy chains respectively. The pairing of a VH and VL together forms a single antigen-binding site. In addition to V regions, both heavy chains and light chains contain a constant (C) region or domain A secreted form of a immunoglobulin C region is made up of three C domains, CH1, CH2, CH3, optionally CH4 (CO, and a hinge region. A membrane-bound form of an immunoglobulin C region also has membrane and intracellular domains. Each light chain has a V_{L} at the N-terminus followed by a constant domain (C) at its other end. The constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively. The VL is aligned with the VH and the CL is aligned with the first constant domain of the heavy chain. As used herein, an “antibody” encompasses conventional antibody structures and variations of antibodies. Thus, within the scope of this concept are ACE proteins, full length antibodies, chimeric antibodies, humanized antibodies, human antibodies, and antibody fragments thereof.

Antibodies exist as intact immunoglobulin chains or as a number of well-characterized antibody fragments produced by digestion with various peptidases. The term “antibody fragment,” as used herein, refers to one or more portions of an antibody that retains six CDRs. Thus, for example, pepsin digests an antibody below the disulfide linkages in the hinge region to produce F(ab)′₂, a dimer of Fab′ which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with a portion of the hinge region (Paul, Fundamental Immunology 3d ed. (1993)). While various antibody fragments are defined in terms of the digestion of an intact antibody, one of skill will appreciate that such fragments may be synthesized de novo either chemically or by using recombinant DNA methodology. As used herein, an “antibody fragment” refers to one or more portions of an antibody, either produced by the modification of whole antibodies, or those synthesized de novo using recombinant DNA methodologies, that retain binding specificity and functional activity. Examples of antibody fragments include Fv fragments, single chain antibodies (ScFv), Fab, Fab′, Fd (Vh and CH1 domains), dAb (Vh and an isolated CDR); and multimeric versions of these fragments (e.g., F(ab′)₂,) with the same binding specificity. ACE proteins can also comprise antibody fragments necessary to achieve the desired binding specificity and activity.

A “Fab” domain as used in the context comprises a heavy chain variable domain, a constant region CH1 domain, a light chain variable domain, and a light chain constant region CL domain. The interaction of the domains is stabilized by a disulfide bond between the CH1 and CL domains. In some embodiments, the heavy chain domains of the Fab are in the order, from N-terminus to C-terminus, VH-CH and the light chain domains of a Fab are in the order, from N-terminus to C-terminus, VL-CL. In some embodiments, the heavy chain domains of the Fab are in the order, from N-terminus to C-terminus, CH-VH and the light chain domains of the Fab are in the order CL-VL. Although the Fab fragment was historically identified by papain digestion of an intact immunoglobulin, in the context of this disclosure, a “Fab” is typically produced recombinantly by any method. Each Fab fragment is monovalent with respect to antigen binding, i.e., it has a single antigen-binding site.

“Complementarity-determining domains” or “complementary-determining regions” (“CDRs”) interchangeably refer to the hypervariable regions of VL and VH. CDRs are the target protein-binding site of antibody chains that harbors specificity for such target protein. There are three CDRs (CDR1-3, numbered sequentially from the N-terminus) in each human V_{L} or V_{H}, constituting about 15-20% of the variable domains CDRs are structurally complementary to the epitope of the target protein and are thus directly responsible for the binding specificity. The remaining stretches of the V_{L} or V_{H}, the so-called framework regions (FR), exhibit less variation in amino acid sequence (Kuby, Immunology, 4th ed., Chapter 4. W.H. Freeman & Co., New York, 2000).

Positions of CDRs and framework regions can be determined using various well known definitions in the art, e.g., Kabat, Chothia, and AbM (see, e.g., Kabat et al. 1991 Sequences of Proteins of Immunological Interest, Fifth Edition, U.S. Department of Health and Human Services, NIH Publication No. 91-3242, Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); (ImMunoGenTics (IMGT) numbering) Lefranc, M.-P., The Immunologist, 7, 132-136 (1999); Lefranc, M.-P. et al., Dev. Comp. Immunol., 27, 55-77 (2003); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

Under Kabat, CDR amino acid residues in the V_{H} are numbered 31-35 (HCDR1), 50-65 (HCDR2), and 95-102 (HCDR3); and the CDR amino acid residues in the V_{L} are numbered 24-34 (LCDR1), 50-56 (LCDR2), and 89-97 (LCDR3). Under Chothia, CDR amino acids in the V_{H} are numbered 26-32 (HCDR1), 52-56 (HCDR2), and 95-102 (HCDR3); and the amino acid residues in V_{L} are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

An “antibody variable light chain” or an “antibody variable heavy chain” as used herein refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions (FR). The term “variable region” or “V-region” interchangeably refer to a heavy or light chain comprising FR1-CDR1-FR2-CDR2-FR3-CDR3-FR4. A V-region can be naturally occurring, recombinant or synthetic. In this application, antibody light chains and/or antibody heavy chains may, from time to time, be collectively referred to as “antibody chains.” As provided and further described herein, an “antibody variable light chain” or an “antibody variable heavy chain” and/or a “variable region” and/or an “antibody chain” optionally comprises a cytokine polypeptide sequence incorporated into a CDR.

The C-terminal portion of an immunoglobulin heavy chain herein, comprising, e.g., CH2 and CH3 domains, is the “Fc” domain. An “Fc region” as used herein refers to the constant region of an antibody excluding the first constant region (CH1) immunoglobulin domain. Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that boundaries of the Fc region may vary, however, the human IgG heavy chain Fc region is usually defined to comprise residues C226 or P230 to its carboxyl-terminus, using the numbering is according to the EU index as in Kabat et al. (1991, NIH Publication 91-3242, National Technical Information Service, Springfield, Va.). “Fc region” may refer to this region in isolation or this region in the context of an antibody or antibody fragment. “Fc region” includes naturally occurring allelic variants of the Fc region, e.g., in the CH2 and CH3 region, including, e.g., modifications that modulate effector function. Fc regions also include variants that don't result in alterations to biological function. For example, one or more amino acids are deleted from the N-terminus or C-terminus of the Fc region of an immunoglobulin without substantial loss of biological function. For example, in certain embodiments a C-terminal lysine is modified replaced or removed. In particular embodiments one or more C-terminal residues in the Fc region is altered or removed. In certain embodiments one or more C-terminal residues in the Fc (e.g., a terminal lysine) is deleted. In certain other embodiments one or more C-terminal residues in the Fc is substituted with an alternate amino acid (e.g., a terminal lysine is replaced). Such variants are selected according to general rules known in the art so as to have minimal effect on activity (see, e.g., Bowie, et al., Science 247:306-1310, 1990). The Fc domain is the portion of the immunoglobulin (Ig) recognized by cell receptors, such as the FcR, and to which the complement-activating protein, C1 q, binds. The lower hinge region, which is encoded in the 5′ portion of the CH2 exon, provides flexibility within the antibody for binding to FcR receptors.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, and drug; or (b) the variable region, or a portion thereof, is altered, replaced or exchanged with a variable region having a different or altered antigen specificity.

A “humanized” antibody is an antibody that retains the reactivity (e.g., binding specificity, activity) of a non-human antibody while being less immunogenic in humans. This can be achieved, for instance, by retaining non-human CDR regions and replacing remaining parts of an antibody with human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

A “human antibody” includes antibodies having variable regions in which both the framework and CDR regions are derived from sequences of human origin. Furthermore, if an antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000). Human antibodies may include amino acid residues not encoded by human sequences (e.g., mutations introduced by random or site-specific mutagenesis in vitro or by somatic mutation in vivo, or a conservative substitution to promote stability or manufacturing).

The term “corresponding human germline sequence” refers to a nucleic acid sequence encoding a human variable region amino acid sequence or subsequence that shares the highest determined amino acid sequence identity with a reference variable region amino acid sequence or subsequence in comparison to all other all other known variable region amino acid sequences encoded by human germline immunoglobulin variable region sequences. A corresponding human germline sequence can also refer to the human variable region amino acid sequence or subsequence with the highest amino acid sequence identity with a reference variable region amino acid sequence or subsequence in comparison to all other evaluated variable region amino acid sequences. A corresponding human germline sequence can be framework regions only, complementary determining regions only, framework and complementary determining regions, a variable segment (as defined above), or other combinations of sequences or sub-sequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence.

The term “valency” as used herein refers to the number of potential target binding sites in a polypeptide. Each target binding site specifically binds one target molecule or a specific site on a target molecule. When a polypeptide comprises more than one target binding site, each target binding site may specifically bind the same or different molecules (e.g., may bind to different molecules, e.g., different antigens, or different epitopes on the same molecule). A conventional antibody, for example, has two binding sites and is bivalent; “trivalent” and “tetravalent” refer to the presence of three binding sites and four binding sites, respectively, in an antibody molecule. The ACE proteins can be monovalent (i.e., bind one target molecule), bivalent, or multivalent (i.e., bind more than one target molecule).

The phrase “specifically binds” when used in the context of describing the interaction between a target (e.g., a protein) and an ACE protein, refers to a binding reaction that is determinative of the presence of the target in a heterogeneous population of proteins and other biologics, e.g., in a biological sample, e.g., a blood, serum, plasma or tissue sample. Thus, under certain designated conditions, an ACE protein with a particular binding specificity binds to a particular target at least two times the background and do not substantially bind in a significant amount to other targets present in the sample. In one embodiment, under designated conditions, an ACE protein with a particular binding specificity bind to a particular antigen at least ten (10) times the background and do not substantially bind in a significant amount to other targets present in the sample. Specific binding to an ACE protein under such conditions can require an ACE protein to have been selected for its specificity for a particular target protein. As used herein, specific binding includes ACE proteins that selectively bind to a human cytokine receptor and do not include ACE proteins that cross-react with, e.g., other cytokine receptor superfamily members. In some embodiments, ACE proteins are selected that selectively bind to the human cytokine receptor and cross-react with non-human primate cytokine receptors (e.g., cynomolgus). In some embodiments, antibody engrafted proteins are selected that selectively bind to human cytokine receptors and react with an additional target. A variety of formats may be used to select ACE proteins that are specifically reactive with a particular target protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time divided by the association rate constant (kₐ, time⁻¹, M⁻¹). Equilibrium dissociation constants can be measured using any known method in the art. The ACE proteins generally will have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M or 10⁻¹³ M.

As used herein, the term “epitope” or “binding region” refers to a domain in the antigen protein that is responsible for the specific binding between the antibody CDRs and the antigen protein.

As used herein, the term “receptor-cytokine binding region” refers to a domain in the engrafted cytokine portion of the ACE protein that is responsible for the specific binding between the engrafted cytokine and its receptor. There is at least one such receptor-cytokine binding region present in each ACE protein, and each of the binding regions may be identical or different from the others.

The term “agonist” refers to an antibody capable of activating a receptor to induce a full or partial receptor-mediated response. For example, an agonist of the cytokine receptor binds to the receptor and induces cytokine-mediated intracellular signaling, cell activation and/or proliferation of T cells. The ACE protein agonist stimulates signaling through its receptor similarly in some respects to the native cytokine. For example, the binding of cytokine to its receptor induces downstream signaling, for example, Jak1 and Jak3 activation which results in STAT5 phosphorylation. In some embodiments, an ACE protein agonist can be identified by its ability to bind its receptor and induce a biological effect such as cell proliferation or STAT phosphorylation.

The term “ACE protein” or “antibody cytokine engrafted molecule” or “engrafted” means that at least one cytokine is incorporated directly within a CDR of the antibody, interrupting the sequence of the CDR. The cytokine can be incorporated within HCDR1, HCDR2, HCDR3, LCDR1, LCDR2 or LCDR3. The cytokine can be incorporated within HCDR1, HCDR2, HCDR3, LCDR1, LCDR2 or LCDR3 and incorporated toward the N-terminal sequence of the CDR or toward the C-terminal sequence of the CDR. The cytokine incorporated within a CDR can disrupt the specific binding of the antibody portion to the original target protein or the ACE protein can retain its specific binding to its target protein.

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 91%, 92%, 93%, 94%, 95%, 96%, 97%, 98%, 99% or 100% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides or polynucleotides that are substantially identical to the polypeptides or polynucleotides, respectively, exemplified herein (e.g., the variable regions exemplified in any one of the sequences in TABLE 2. The identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “link,” or “linked” when used in the context of describing how the binding regions are connected within an ACE protein of this invention, encompasses all possible means for physically joining the regions. The multitude of binding regions are frequently joined by chemical bonds such as a covalent bond (e.g., a peptide bond or a disulfide bond) or a non-covalent bond, which can be either a direct bond (i.e., without a linker between two binding regions) or indirect bond (i.e., with the aid of at least one linker molecule between two or more binding regions).

The terms “subject,” “patient,” and “individual” interchangeably refer to a mammal, for example, a human or a non-human primate mammal. The mammal can also be a laboratory mammal, e.g., mouse, rat, rabbit, hamster. In some embodiments, the mammal can be an agricultural mammal (e.g., equine, ovine, bovine, porcine, camelid) or domestic mammal (e.g., canine, feline).

As used herein, the terms “treat,” “treating,” or “treatment” of any disease or disorder refer in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of a disease or disorder.

The term “therapeutically acceptable amount” or “therapeutically effective dose” interchangeably refer to an amount sufficient to effect the desired result (i.e., reduction in tumor volume). In some embodiments, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of an ACE protein can prevent the onset of, or result in a decrease in severity of, respectively, disease symptoms, including symptoms associated with cancer and cancer treatment.

The term “co-administer” refers to the simultaneous presence of two (or more) active agents in an individual. Active agents that are co-administered can be concurrently or sequentially delivered.

As used herein, the phrase “consisting essentially of” refers to the genera or species of active pharmaceutical agents included in a method or composition, as well as any inactive carrier or excipients for the intended purpose of the methods or compositions. In some embodiments, the phrase “consisting essentially of” expressly excludes the inclusion of one or more additional active agents other than an ACE protein. In some embodiments, the phrase “consisting essentially of” expressly excludes the inclusion of more additional active agents other than an ACE protein and a second co-administered agent.

The terms “a,” “an,” and “the” include plural referents, unless the context clearly indicates otherwise.

### BRIEF DESCRIPTION OF THE DRAWINGS

FIG. 1A/C is a schematic of four helix bundle cytokine topology a) the left handed arrangement of helices numbered A-D as seen from above adapted from (Presnell and Cohen, 1989). b) Two dimensional connection schematic of helices for the short and long chain cytokine family. c) Two dimensional connection schematic of the IL10, interferon family showing inserted helices relative to b) in the A/B and C/D overhand loops.

FIG. 2A/D demonstrates examples of the diversity of structure of the different four helix bundle cytokine families, helices are numbered sequentially by letter from N-terminus to C-terminus a) Short chain cytokine IL4, b) Long chain cytokine, IL6, c) IL10 family showing the monomer IL10 arrangement of the E F helices motif utilized to interdigitate with another IL monomer to generate a functional dimer, c) IL22, another member of the IL10 family this time forming a monomer six helix bundle.

FIG. 3A/B shows the activity of IL7 ACE proteins on CD8 and CD4 cells.

FIG. 4A compares pSTAT5 activity of IL7 ACE proteins on CD4 T cells, CD8 T cells, B cells and NK cells. FIG. 4B shows the effects of increasing concentrations of IL7 ACE protein on CD8 T cells as measured by pSTAT5. FIG. 4C shows the effects of increasing concentrations of IL7 ACE protein on CD4 T cells as measured by pSTAT5.

FIGS. 5A-5D is show the pharmacodynamics of IL7 ACE proteins, demonstrating increased CD8 T cell proliferation.

FIGS. 6A-6B demonstrates that IL7 ACE proteins reduce tumor growth as a single agent. FIG. 6C shows the increase in CD8 T cells in the blood. FIG. 6D shows the increase of CD8 tumor infiltrating lymphocytes upon administration of IL7 ACE protein. FIG. 6E shows the increase of CD4 tumor infiltrating lymphocytes upon administration of IL7 ACE protein.

FIG. 7 is a graphical representation of the synergistic combination of IL7 ACE proteins with an anti-PD-L1 antibody.

FIG. 8 is a structural diagram of IL7 inserted into HCDR2 or HCDR3 respectively.

FIG. 9 is a graph of the binding of various IL7 antibody cytokine engrafted proteins to RSV.

FIG. 10 is a Gyros assay showing that IL7 antibody cytokine engrafted proteins have a longer half-life than recombinant IL7.

FIG. 11 is a FACS plot and graphs showing the expansion of CD8+ cells in the blood upon administration of IL7 antibody cytokine engrafted proteins as a single agent and upon administration of IL7 antibody cytokine engrafted proteins in combination with an anti-PD-L1 antibody.

FIG. 12 shows that IL7 antibody cytokine engrafted proteins induces the reduction of Tim-3, either alone or in combination with anti-PD-L1.

FIG. 13 shows the increase in total numbers of naïve, central memory and effector memory CD8+ T cells in the blood upon dosing with IL7 antibody cytokine engrafted proteins.

FIG. 14 demonstrates that administration of IL7 antibody cytokine engrafted protein also induces an increase in CD8+PD-1+ cells.

FIG. 15 shows that administration of IL7 antibody cytokine engrafted proteins were able to reduce viral load as a single agent, or in combination with an anti-PD-L1 antibody.

FIG. 16 demonstrates that the administration of IL7 antibody cytokine engrafted proteins, in combination with anti-PD-L1, resulted in the increase of IFN-gamma.

FIG. 17 is a table of antibody cytokine engrafted constructs, showing that IgG.IL2D49A.H1 preferentially expands Tregs. This figure also shows a number of ACE proteins made. Wild type IL2 was cloned into all six CDRs and the N-terminus of HCDR1 (nH1), the C-terminus of HCDR1(cH1), the N-terminus of HCDR2(nH2), and the C-terminus of HCDR2(cH2). Engrafting wild type into LCDR2 resulted in an ACE protein that did not express.

FIG. 18 is a table comparing antibody cytokine engrafted proteins with recombinant IL2 (Proleukin®). Note that the IgG.IL2D49.H1 molecule stimulates the IL2 receptor on Treg cells, but not on T effector cells (Teff) or NK cells as measured by STAT5 phosphorylation. This molecule also has a longer half-life than Proleukin® and causes greater expansion of Treg cells in vivo.

FIG. 19 is a table of the fold changes in a panel of different immunomodulatory cell types when equimolar doses of antibody cytokine engrafted proteins, for example IgG.IL2D49.H1, are compared to Proleukin®.

FIG. 20 represents the differential activation of the IL2 low affinity or high affinity receptor by antibody cytokine engrafted protein as compared to Proleukin® and as measured by STAT5 phosphorylation. Note that the IgG.IL2D49A.H1 stimulates the high affinity IL2 receptors expressed on Treg cells but not on CD4+ or CD8+ Tcon cells.

FIG. 21 shows in graphical form that Tregs expanded with antibody cytokine engrafted proteins (e.g. IgG.IL2D49A.H1) are better suppressors of Teffector cells (Teff) (see upper panel). The lower panel shows that Treg cells expanded by antibody cytokine engrafted proteins are stable by Foxp3 protein expression and by Foxp3 methylation.

FIG. 22 demonstrates that antibody cytokine engrafted proteins have little to no effect on NK cells which express the IL2 low affinity receptor. In contrast, Proleukin® stimulates NK cells as measured by pSTAT5 activation.

FIG. 23 is a pharmacokinetic (PK), pharmacodynamic (PD) and toxicity profile of antibody cytokine engrafted protein compared to Proleukin® in cynomolgous monkeys. For example, IgG.IL2D49A.H1 has a much reduced eosinophilia toxicity profile than Proleukin®.

FIG. 24 is a graph depicting the extended half-life of IgG.IL2D49.H1.

FIG. 25 is a graphic representation of antibody cytokine engrafted protein molecules in a mouse GvHD model. This shows that treatment with antibody cytokine engrafted proteins in this model expand Tregs better than Proleukin®, while having little to no effect on CD4+/CD8+ Teff cells or NK cells.

FIG. 26 shows graphically the loss of body weight associated with Proleukin® treatment in a GvHD mouse model, while there is little body weight loss associated with administration of IgG.IL2D49.H1.

FIG. 27 compares antibody cytokine engrafted proteins to Proleukin® in a prediabetic (NOD) mouse model, and demonstrates that IgG.IL2D49A.H1 prevents Type 1 diabetes in this model.

FIG. 28 compares the ratio of Treg to CD8 Teffector cells in a pre-diabetic NOD mouse model.

FIG. 29 shows the pharmacokinetics of IgG.IL2D49A.H1 in the NOD mouse model at a 1.3 mg/kg dose.

FIG. 30 shows the pharmacokinetics of IgG.IL2D49A.H1 in the NOD mouse model at a 0.43 mg/kg dose.

FIG. 31 is a table of dose ranges used in the pre-diabetic NOD mouse model, and compares equimolar amounts of Proleukin®.

FIGS. 32-33 are a series of graphs depicting amount of pSTAT5 activation on human cells treated with IgG.IL2D49.H1. Cells were taken from a normal donor, a donor with vitiligo (FIG. 32) and type 1 diabetes (T1D) (FIG. 33).

FIG. 34 is a graph of the binding of various IL2 antibody cytokine engrafted proteins to RSV.

FIG. 35 shows Treg expansion in cynomolgus monkey after a single dose of IgG.IL2D49A.H1.

FIG. 36 is a table summarizing exemplary the IL2 antibody cytokine engrafted proteins and their activities on CD8 T effector cells.

FIG. 37 shows that IgG.IL2R67A.H1 has a greater half-life than that of Proleukin®. IgG.IL2R67A.H1 has a half-life of 12-14 hours as shown in the graph, while Proleukin® has a T1/2 of less than 4 hours and cannot be shown on the graph.

FIG. 38A-38C demonstrates that IgG.IL2R67A.H1 expands CD8+T effector cells more effectively and with less toxicity than Proleukin® or an IL2-Fc fusion molecule in C57BL/6 mice at a 100 μg equivalent dose, at day 4, day 8 and day 11 time points.

FIG. 38D-38F demonstrates that IgG.IL2R67A.H1 expands CD8+T effector cells more effectively and with less toxicity than Proleukin® or an IL2-Fc fusion molecule in C57BL/6 mice at a 500 μg equivalent dose at day 4, day 8 and day 11 time points.

FIG. 39A shows that IgG.IL2R67A.H1 selectively expands CD8 T effectors and is better tolerated than Proleukin® in NOD mice.

FIG. 39B is a table depicting the increased activity of IgG.IL2R67A.H1 and IgG.IL2F71A.H1 on CD8 T effectors in NOD mice.

FIG. 40 is a graph of single agent efficacy of IgG.IL2R67A.H1 in a CT26 tumor model.

FIG. 41 presents the data of IgG.IL2R67A.H1 either as a single agent or in combination with an antibody in a B16 melanoma mouse model. The graph shows that IgG.IL2R67A.H1 in combination with TA99, an anti-TRP1 antibody, is more efficacious than TA99 alone, an IL2-Fc fusion molecule alone or TA99 plus an IL2-Fc fusion. Synergy was seen with TA99 and IgG.IL2R67A.H1 at the 100 and 500 μg doses.

FIG. 42 is a graph with values monitoring pSTAT5 in a panel of human cells comparing IgG.IL2R67A.H1 with Proleukin® and a native IL-2 (no muteins) grafted into HCDR1 and HCDR2.

FIG. 43 is a graph of the binding of various IL2 antibody cytokine engrafted proteins to RSV.

FIG. 44 depicts results of CyTOF analysis of IL-6 dependent pSTAT1, pSTAT3, pSTAT4, and pSTAT5 signaling in human whole blood stimulated with equal molar amounts native human IL-6 or IL-6 antibody cytokine engrafted proteins.

FIG. 45 depicts results of CyTOF data of pSTAT1, pSTAT3, and pSTAT5 activity of various IL-6 antibody cytokine engrafted proteins on CD4 T cells, CD8 T cells, B cells, NK cells, monocytes, dendritic cells, etc.

FIGS. 46A and 46B show line graphs illustrating the half-life of the IL-6 antibody cytokine engrafted proteins IgG.IL-6.H2 and IgG.IL-6.H3 in an IL-6Fc Gyros assay in C57Bl/6 DIO mice.

FIG. 47 shows a dot plot illustrating in vivo activity of IL-6 antibody cytokine engrafted protein in fat and muscle tissues in C57Bl/6 DIO mice measured by phospho-Stat3 (pSTAT3) after subcutaneous dosing.

FIGS. 48A, 48B and 48C show line graphs illustrating in vivo activity of IL-6 antibody cytokine engrafted protein in C57Bl/6 DIO mice measured by changes in body weight (A), fat tissue (B) and lean tissue (C) after subcutaneous dosing.

FIGS. 49A, 49B and 49C show line graphs illustrating in vivo activity of IL-6 antibody cytokine engrafted protein in C57Bl/6 DIO mice measured by respiratory exchange ratio (RER) pre-dosing (A), at days 3-5 (B) and at days 7-9 (C) after subcutaneous dosing.

FIGS. 50A, 50B, 50C, 50D and 50E show graphs illustrating in vivo activity of IL-6 antibody cytokine engrafted protein on food intake in pair fed C57Bl/6 DIO mice measured by changes in body weight (A), food intake (B), over-all fat mass (C), lean mass (D) and tibialis anterior muscle weight (7E) after subcutaneous dosing.

FIG. 51A-51B depicts results of in vitro biological assays of recombinant human IL10 (rhIL10, gray square) and the IgGIL10M13 antibody cytokine engrafted protein (black triangle). FIG. 51A illustrates that IgGIL10M13 demonstrated decreased pro-inflammatory activity as compared to rhIL10 as measured by IFN gamma induction in CD8 T cell assays. Similar differential activity was found on human primary NK cells, B cells, and mast cells, as well as using granzyme-B as a readout measurement. FIG. 51B illustrates that rhIL10 and IgGIL10M13 demonstrate similar anti-inflammatory activity as measured by inhibition of TNFα in whole blood assays.

FIG. 52 depicts results of CyTOF analysis of IL10 dependent pSTAT3 signaling in human whole blood stimulated with equal molar amounts recombinant human IL10 rhIL10 (left panel) or IgGIL10M13 (right panel). IL10 induces anti-inflammatory activities in monocytes; and activation of T, B or NK cells induces pro-inflammatory cytokines. Results of fold change in activity of cells over unstimulated are depicted by heat map (changes in shading). Left panel indicates rhIL10 confers stimulation across all IL10 sensitive cell types (with outline); however, as seen in the right panel IgGIL10M13 confers less potent stimulation on T, B, and NK cells, with levels similar or slightly above unstimulated cells; while a similar potency of stimulation of monocytes (outlined) and mDC cells was demonstrated with IgG-IL10M and rhIL10. These relevant cell types (monocytes, mDC) are key cells for maintenance of gut homeostatis in inflammatory bowel disease.

FIG. 53A-53D illustrates improved characteristics of antibody cytokine engrafted protein IgGIL10M13 in in vivo assays. FIG. 53A-53B depicts results of pharmacokinetic studies of rhIL10 and IgGIL10M13. Following intravenous administration, IgGIL10M13 demonstrates prolonged pharmacokinetics (half-life) as antibody cytokine engrafted protein is still detectable after 4.4 days (FIG. 53B), while rhIL10 had a half-life of approximately 1 hour (FIG. 53A). FIGS. 53C and 53D depict results of pharmacodynamic assays of in vivo activity of antibody cytokine engrafted proteins. FIG. 53C depicts in vivo activity in colon tissue as measured by pSTAT3 signaling seventy-two (72) hours post dosing. FIG. 53D depicts improved duration of in vivo response of IgGIL10M13 as compared to rhIL10 as measured by inhibition of TNFα in response to LPS challenge following administration of IgGIL10M13.

FIG. 54 is the results of an LPS challenge model, demonstrating IgGIL10M13 reduces TNFα induction 48 hours after LPS challenge.

FIG. 55 is a graph representing the improved % CMAX of IL10 antibody cytokine engrafted proteins.

FIG. 56 depicts CyTOF data of pSTAT3 activity in various immune cells from healthy subjects and patients when stimulated with rhIL10 or with IgGIL10M13.

FIGS. 57-61 are graphical representations demonstrating IgGIL10M13 has reduced pro-inflammatory activity in PHA stimulated human whole blood compared to rhIL10.

FIG. 62 shows the graphs of a titration experiment with rhIL10 and IgGIL10M13.

FIGS. 63-64 depict the aggregation properties of IL10 wild type or monomeric when conjugated via a linker to an Fc, compared to the aggregation properties of an antibody cytokine engrafted protein.

FIG. 65 is ELISA data showing that the IL10 antibody cytokine engrafted protein still binds to RSV.

FIG. 66 is a representation of the mechanism of action of an IL10 antibody cytokine engrafted protein. The left panel shows how a normal rhIL10 dimer binds IL-10R1, and initiates strong pSTAT3 signaling. The right panel depicts how an IL10 monomer engrafted into a CDR of an antibody is constrained to have less efficient binding to IL-10R1 and thus produces a weaker pSTAT3 signal.

FIG. 67A-C is the crystal structure resolution of IL10 monomer engrafted into LCDR1 of palivizumab.

FIG. 68 is a graph and a table showing IC50 values for IL10 ACE proteins engrafted into a different antibody scaffold.

FIG. 69 is a graph and a table showing IC50 values for IL10 ACE proteins engrafted into a different antibody scaffold wherein the IL10 cytokine is engrafted into different CDRs.

FIG. 70A shows the expansion of CD8+ Teffector cells in a mouse model after treatment with an IL2 ACE protein engrafted into a different antibody scaffold.

FIG. 70B shows the expansion of CD4+ Treg cells in a mouse model after treatment with an IL2 ACE protein engrafted into a different antibody scaffold.

FIG. 70C shows the expansion of NK cells in a mouse model after treatment with an IL2 ACE protein engrafted into a different antibody scaffold.

FIGS. 71-100 is Cytof data showing the pSTAT activity for its respective ACE protein.

FIG. 101 shows that H1, H3 and L3 Flt3L grafts are capable of inducing B220+CD11c+ plasmacytoid DC differentiation (top panels) and CD370+DC1 differentiation (bottom panels) comparable to what is observed with recombinant human Flt3L. Top plots are gated on live, singlet cells. Bottom plots are gated on live, singlet cells that are CD11c+.

FIG. 102 shows that GM-CSF cytokine grafts are capable of inducing monocyte DC differentiation as evidenced by upregulation of DC-SIGN on the cells and downregulation of CD14. The event was specific to cells cultured with GM-CSF or GM-CSF containing grafts, as a Palivizumab graft control did not induce these cellular changes.

FIG. 103 shows that monocyte DCs generated with GM-CSF grafts are capable of responding to TLR7/8 activation. Cells were incubated with R848, a well characterized TLR7/8 agonist overnight and cell surface CD86 upregulation was measured as a marker of cellular activation. Monocyte DCs generated with wild type human GM-CSF or the GM-CSF grafts were equally capable of upregulating CD86 after R848 simulation, indicating functionality of the monocyte DCs generated with GM-CSF grafts.

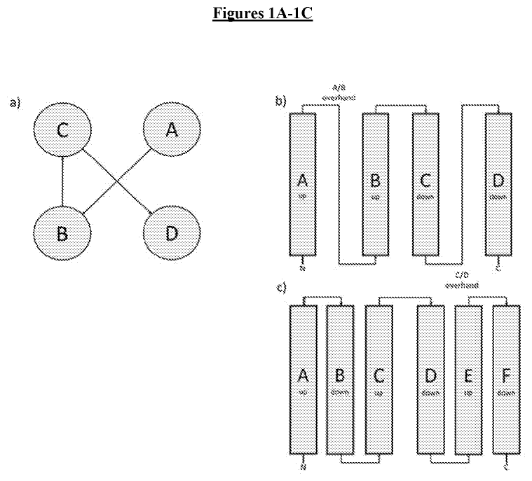

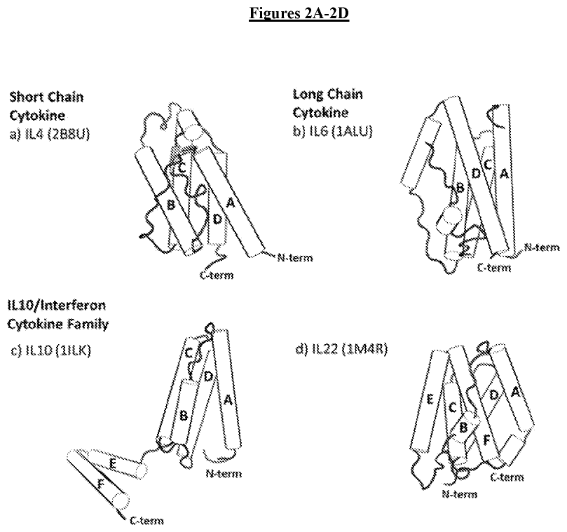

### ACE PROTEINS

Embodiments disclosed herein provide ACE proteins comprising: (a) a heavy chain variable region (VH), comprising Complementarity Determining Regions (CDR) HCDR1, HCDR2, HCDR3; and (b) a light chain variable region (VL), comprising LCDR1, LCDR2, LCDR3; and (c) a cytokine molecule engrafted into a CDR of the VH or the VL.

In some embodiments, the cytokine molecule is directly engrafted into the CDR. In some embodiments, the cytokine molecule is directly engrafted into the CDR without a peptide linker, with no additional amino acids between the CDR sequence and the cytokine sequence.

In some embodiments, the cytokine molecules engrafted into the CDR belong to the 4-helix bundle family of cytokines. For example, the cytokine molecules may be chosen from those listed in Table 1. In some embodiments, the cytokine molecule is not interleukin-10 (IL-10). In some embodiments, the full-length cytokine molecule is engrafted into the CDR. In some embodiments, the cytokine molecule without the signal peptide is engrafted into the CDR.

Without being bound by theory, it is contemplated that by engrafting a cytokine molecule directly into the CDR sequence of an antibody scaffold, the natural conformation of the cytokine may or may not be modified by the CDR sequence or other part of the antibody scaffold, which may result in a change to the characteristics of the engrafted cytokine molecule. For example, depending on the length of the CDR sequence the cytokine molecule is engrafted into, its binding to a receptor(s) may be negatively or positively affected, as well as its signalling through the receptor(s).

Therefore, in some embodiments, the binding affinity of the engrafted cytokine molecule of the ACE protein to a receptor is increased in comparison to a free cytokine molecule. For example, the binding affinity of the engrafted cytokine molecule of the ACE protein to a receptor is increased by 10%, by 20%, by 30%, by 40%, by 50%, by 60%, by 70%, by 80%, by 90%, by 100%, by 2 fold, by 3 fold, by 4 fold, by 5 fold, by 10 fold, by 100 fold, by 1,000 fold, or more, in comparison to a free cytokine molecule.

In some embodiments, the binding affinity of the engrafted cytokine molecule of the ACE protein to a receptor is decreased in comparison to a free cytokine molecule. For example, the binding affinity of the engrafted cytokine molecule of the ACE protein to a receptor is decreased by 10%, by 20%, by 30%, by 40%, by 50%, by 60%, by 70%, by 80%, by 90%, by 95%, by 98%, by 99%, by 100%, in comparison to a free cytokine molecule.

In some embodiments, the binding avidity of the engrafted cytokine molecule of the ACE protein to a receptor is increased in comparison to a free cytokine molecule. For example, the binding avidity of the engrafted cytokine molecule of the ACE protein to a receptor is increased by 10%, by 20%, by 30%, by 40%, by 50%, by 60%, by 70%, by 80%, by 90%, by 100%, by 2 fold, by 3 fold, by 4 fold, by 5 fold, by 10 fold, by 100 fold, by 1,000 fold, or more, in comparison to a free cytokine molecule.

In some embodiments, the binding avidity of the engrafted cytokine molecule of the ACE protein to a receptor is decreased in comparison to a free cytokine molecule. For example, the binding avidity of the engrafted cytokine molecule of the ACE protein to a receptor is decreased by 10%, by 20%, by 30%, by 40%, by 50%, by 60%, by 70%, by 80%, by 90%, by 95%, by 98%, by 99%, by 100%, in comparison to a free cytokine molecule.

In some embodiments, the the differential binding affinity or avidity of the engrafted cytokine molecule of the ACE protein to two or more receptors is changed in comparison to a free cytokine molecule.

In some embodiments, an activity of the engrafted cytokine molecule of the ACE protein is increased in comparison to a free cytokine molecule. For example, the activity of the engrafted cytokine molecule of the ACE protein, e.g., cell-proliferation activity, anti-cell-proliferation activity, apoptotic activity, pro-inflammatory activity, anti-inflammatory activity, etc., is increased by 10%, by 20%, by 30%, by 40%, by 50%, by 60%, by 70%, by 80%, by 90%, by 100%, by 2 fold, by 3 fold, by 4 fold, by 5 fold, by 10 fold, by 100 fold, by 1,000 fold, or more, in comparison to a free cytokine molecule.

In some embodiments, an activity of the engrafted cytokine molecule of the ACE protein is decreased in comparison to a free cytokine molecule. For example, the activity of the engrafted cytokine molecule of the ACE protein, e.g., cell-proliferation activity, anti-cell-proliferation activity, apoptotic activity, pro-inflammatory activity, anti-inflammatory activity, etc., is decreased by 10%, by 20%, by 30%, by 40%, by 50%, by 60%, by 70%, by 80%, by 90%, by 95%, by 98%, by 99%, by 100%, in comparison to a free cytokine molecule.

In some embodiments, the antibody cytokine engrafted confers anti-inflammatory properties superior to a free cytokine molecule. In some embodiments, the antibody cytokine engrafted proteins disclosed herein confer increased activity on Treg cells while providing reduced proportional pro-inflammatory activity as compared to the free cytokine molecule. In some embodiments, the antibody cytokine engrafted proteins disclosed herein provide preferential activation of Treg cells over Teff cells, Tcon cells, and/or NK cells. In some embodiments, the antibody cytokine engrafted proteins disclosed herein provide preferential expansion of Treg cells over Teff cells, Tcon cells, and/or NK cells. In some embodiments, the antibody cytokine engrafted proteins disclosed herein provide increased expansion of Treg cells without expansion of CD8 T effector cells or NK cells. In some embodiments, the antibody cytokine engrafted proteins disclosed herein provide a ratio of expansion of Treg cells:NK cells that is, is about, is greater than, 1, 2, 3, 4, 5, 6, 7, 8, 9, 10. In some embodiments, the antibody cytokine engrafted proteins disclosed herein provide a ratio of expansion of Treg cells:CD8 T effector cells that is, is about, is greater than, 1, 2, 3, 4, 5, 6, 7, 8, 9, 10. In some embodiments, the antibody cytokine engrafted proteins disclosed herein provide a ratio of expansion of Treg cells:CD4 Tcon cells that is, is about, is greater than, 1, 2, 3, 4, 5, 6, 7, 8, 9, 10.

In some embodiments, the antibody cytokine engrafted proteins disclosed herein provide receptor signalling potency that is reduced in CD4 Tcon cells in comparison to the free cytokine molecule. In some embodiments, the antibody cytokine engrafted proteins disclosed herein provide receptor signalling potency that is reduced in CD8 Teff cells in comparison to the free cytokine molecule. In some embodiments, the antibody cytokine engrafted proteins disclosed herein provide receptor signalling potency that is reduced in NK cells in comparison to the free cytokine molecule. In some embodiments, the antibody cytokine engrafted proteins disclosed herein provide specific activation of Treg cells over CD4 T effector cells that is about 1,000 fold, about 2,000 fold, about 3,000 fold, about 4,000 fold, about 5,000 fold, about 6,000 fold, about 7,000 fold, about 8,000 fold, about 9,000 fold, about 10,000 fold, or more, higher than the free cytokine molecule. In some embodiments, the antibody cytokine engrafted proteins disclosed herein provide specific activation of Treg cells over CD8 T effector cells that is about 100 fold, about 200 fold, about 300 fold, about 400 fold, about 500 fold, about 600 fold, about 700 fold, about 800 fold, about 900 fold, about 1,000 fold, or more, higher than the free cytokine molecule. In some embodiments, the antibody cytokine engrafted proteins disclosed herein provide specific activation of Treg cells over CD8 T effector/memory cells that is about 100 fold, about 200 fold, about 300 fold, about 400 fold, about 500 fold, about 600 fold, about 700 fold, about 800 fold, about 900 fold, about 1,000 fold, or more, higher than the free cytokine molecule.

In some embodiments, the antibody cytokine engrafted proteins disclosed herein provide reduced toxicity the free cytokine. In some embodiments, the antibody cytokine engrafted proteins disclosed herein provide increased half life, such as more than 4 hours, more than 6 hours, more than 8 hours, more than 12 hours, more than 24 hours, more than 48 hours, more than 3 days, more than 4 days, more than 7 days, more than 14 days, or longer.

In some embodiments antibody cytokine engrafted proteins comprise heavy and light chain immunoglobulin sequences having binding specificity of the immunoglobulin variable domains to a target distinct from the binding specificity of the cytokine molecule. In some embodiments the binding specificity of the immunoglobulin variable domain to its target is retained by 10%, 20%, 30%, 40%, 50%, 60%, 70%, 80%, 90%, 95%, 98%, 99%, or 100%, in the presence of the engrafted cytokine. In certain embodiments the retained binding specificity is to a non-human target. In certain embodiments the retained binding specificity it to a virus, for example, RSV. In other embodiments the binding specificity is to a human target having therapeutic utility in conjunction with the cytokine molecule. In certain embodiments, targeting the binding specificity of the immunoglobulin conveys additional therapeutic benefit to the cytokine. In certain embodiments the binding specificity of the immunoglobulin to its target conveys synergistic activity with the cytokine.

In still other embodiments, the binding specificity of the immunoglobulin to its target is reduced 10%, 20%, 30%, 40%, 50%, 60%, 70%, 80%, 90%, 95%, 98%, 99%, or 100% by the engrafting of the cytokine molecule.

### ACE Proteins Targeting the IL7Ra

Provided herein are ACE proteins comprising an IL7 molecule engrafted into the complementarity determining region (CDR) of an antibody. The ACE proteins of the present disclosure show suitable properties to be used in human patients, for example, they retain immunostimulatory activity similar to that of native or recombinant human IL7. Other activities and characteristics are also demonstrated throughout the specification. Thus, provided are ACE proteins having an improved therapeutic profile over previously known IL7 and modified IL7 therapeutic agents, and methods of use of the provided ACE proteins in cancer treatment.

Accordingly, the present disclosure provides ACE proteins that are agonists of the IL7Ra, with selective activity profiles. Provided ACE proteins comprise an immunoglobulin heavy chain sequence and an immunoglobulin light chain sequence. Each immunoglobulin heavy chain sequence comprises a heavy chain variable region (VH) and a heavy chain constant region (CH), wherein the heavy chain constant region consists of CH1, CH2, and CH3 constant regions. Each immunoglobulin light chain sequence comprises a light chain variable region (VL) and a light chain constant region (CL). In each ACE protein an IL7 molecule is incorporated into a complementarity determining region (CDR) of the VH or VL.

In some embodiments, the ACE protein comprises an IL7 molecule incorporated into a heavy chain CDR. In certain embodiments IL7 is incorporated into heavy chain complementarity determining region 1 (HCDR1). In certain embodiments IL7 is incorporated into heavy chain complementarity determining region 2 (HCDR2). In certain embodiments IL7 is incorporated into heavy chain complementarity determining region 3 (HCDR3).

In some embodiments, the ACE protein comprises IL7 incorporated into a light chain CDR. In certain embodiments IL7 is incorporated into light chain complementarity determining region 1 (LCDR1). In certain embodiments IL7 is incorporated into light chain complementarity determining region 2 (LCDR2). In certain embodiments IL7 is incorporated into light chain complementarity determining region 3 (LCDR3).

In some embodiments, the ACE comprises an IL7 sequence incorporated into a CDR, whereby the IL7 sequence is inserted into the CDR sequence. The insertion may be at or near the N-terminal region of the CDR, in the middle region of the CDR or at or near the C-terminal region of the CDR. In other embodiments, the ACE comprises IL7 incorporated into a CDR, whereby the IL7 sequence does not frameshift the CDR sequence.

In some embodiments IL7 is engrafted directly into a CDR without a peptide linker, with no additional amino acids between the CDR sequence and the IL7 sequence.

In some embodiments ACE proteins comprise immunoglobulin heavy chains of an IgG class antibody heavy chain. In certain embodiments an IgG heavy chain is any one of an IgG1, an IgG2 or an IgG4 subclass.

### ACE Proteins Targeting the IL2 High Affinity Receptor

Provided herein are protein constructs comprising IL2 engrafted to into the complementarity determining region (CDR) of an antibody. The antibody cytokine engrafted proteins show suitable properties to be used in human patients, for example, they retain immunostimulatory activity on Treg cells similar to that of native or recombinant human IL2. However, the negative effects are diminished, for example stimulation of NK cells. Other activities and characteristics are also demonstrated throughout the specification. Thus, provided are antibody cytokine engrafted proteins having an improved therapeutic profile over previously known IL2 and modified IL2 therapeutic agents, and methods of use of the provided antibody cytokine engrafted proteins in therapy.

Accordingly, the present disclosure provides antibody cytokine engrafted proteins that are agonists of the IL2 high affinity receptor, with selective activity profiles. Provided antibody cytokine engrafted proteins comprising an immunoglobulin heavy chain sequence and an immunoglobulin light chain sequence. Each immunoglobulin heavy chain sequence comprises a heavy chain variable region (VH) and a heavy chain constant region (CH), wherein the heavy chain constant region consists of CH1, CH2, and CH3 constant regions. Each immunoglobulin light chain sequence comprises a light chain variable region (VL) and a light chain constant region (CL). In each antibody cytokine engrafted protein an IL2 molecule is incorporated into a complementarity determining region (CDR) of the VH or VL of the antibody.

In some embodiments, the antibody cytokine engrafted protein comprises IL2 incorporated into a heavy chain CDR. In certain embodiments IL2 is incorporated into heavy chain complementarity determining region 1 (HCDR1). In certain embodiments IL2 is incorporated into heavy chain complementarity determining region 2 (HCDR2). In certain embodiments monomeric IL2 is incorporated into heavy chain complementarity determining region 3 (HCDR3).

In some embodiments, the antibody cytokine engrafted protein comprises an IL2 incorporated into a light chain CDR. In certain embodiments IL2 is incorporated into light chain complementarity determining region 1 (LCDR1). In certain embodiments IL2 is incorporated into light chain complementarity determining region 2 (LCDR2). In certain embodiments IL2 is incorporated into light chain complementarity determining region 3 (LCDR3).

In some embodiments, the antibody cytokine engrafted comprises an IL2 sequence incorporated into a CDR, whereby the IL2 sequence is inserted into the CDR sequence. The insertion may be at or near the beginning of the CDR, in the middle region of the CDR or at or near the end of the CDR. In other embodiments, the antibody cytokine engrafted comprises IL2 incorporated into a CDR, whereby the IL2 sequence replaces all or part of a CDR sequence. A replacement may be at or near the beginning of the CDR, in the middle region of the CDR or at or near the end of the CDR. A replacement may be as few as one or two amino acids of a CDR sequence, or as many as an entire CDR sequence.

In some embodiments IL2 is incorporated directly into a CDR without a peptide linker, with no additional amino acids between the CDR sequence and the IL2 sequence.

In some embodiments antibody cytokine engrafted proteins comprise immunoglobulin heavy chains of an IgG class antibody heavy chain. In certain embodiments an IgG heavy chain is any one of an IgG1, an IgG2 or an IgG4 subclass.

In some embodiments antibody cytokine engrafted proteins comprise heavy and light chain immunoglobulin sequences selected from a known, clinically utilized immunoglobulin sequence. In certain embodiments antibody cytokine engrafted proteins comprise heavy and light chain immunoglobulin sequences which are humanized sequences. In other certain embodiments antibody cytokine engrafted proteins comprise heavy and light chain immunoglobulin sequences which are human sequences.

In some embodiments antibody cytokine engrafted proteins comprise heavy and light chain immunoglobulin sequences selected from germline immunoglobulin sequences.

In some embodiments antibody cytokine engrafted proteins comprise heavy and light chain immunoglobulin sequences having binding specificity of the immunoglobulin variable domains to a target distinct from the binding specificity of the IL2 molecule. In some embodiments the binding specificity of the immunoglobulin variable domain to its target is retained in the presence of the engrafted. In certain embodiments the retained binding specificity is to a non-human target. In other embodiments the binding specificity is to a human target having therapeutic utility in conjunction with IL2 therapy. In certain embodiments, targeting the binding specificity of the immunoglobulin conveys additional therapeutic benefit to the IL2 component. In certain embodiments the binding specificity of the immunoglobulin to its target conveys synergistic activity with IL2.

In still other embodiments, the binding specificity of the immunoglobulin to its target is reduced by the engrafting of the IL2 molecule.

### ACE Proteins Targeting the IL2 Low Affinity Receptor

Provided herein are antibody cytokine engrafted proteins comprising an IL2 molecule engrafted to into the complementarity determining region (CDR) of an antibody. The antibody cytokine engrafted proteins of the present disclosure show suitable properties to be used in human patients, for example, they retain immunostimulatory activity similar to that of native or recombinant human IL2. However, the negative effects are diminished. For example, there is less stimulation of Treg cells and an improved response of CD8 T effector cells. Other activities and characteristics are also demonstrated throughout the specification. Thus, provided are antibody cytokine engrafted proteins having an improved therapeutic profile over previously known IL2 and modified IL2 therapeutic agents, and methods of use of the provided antibody cytokine engrafted proteins in cancer treatment.

Accordingly, the present disclosure provides antibody cytokine engrafted proteins that are agonists of the IL2 low affinity receptor, with selective activity profiles. Provided antibody cytokine engrafted proteins comprise an immunoglobulin heavy chain sequence and an immunoglobulin light chain sequence. Each immunoglobulin heavy chain sequence comprises a heavy chain variable region (VH) and a heavy chain constant region (CH), wherein the heavy chain constant region consists of CH1, CH2, and CH3 constant regions. Each immunoglobulin light chain sequence comprises a light chain variable region (VL) and a light chain constant region (CL). In each antibody cytokine engrafted protein an IL2 molecule is incorporated into a complementarity determining region (CDR) of the VH or VL.

In some embodiments, the antibody cytokine engrafted protein comprises IL2 molecule incorporated into a heavy chain CDR. In certain embodiments IL2 is incorporated into heavy chain complementarity determining region 1 (HCDR1). In certain embodiments IL2 is incorporated into heavy chain complementarity determining region 2 (HCDR2). In certain embodiments IL2 is incorporated into heavy chain complementarity determining region 3 (HCDR3).

In some embodiments, the antibody cytokine engrafted protein comprises IL2 incorporated into a light chain CDR. In certain embodiments IL2 is incorporated into light chain complementarity determining region 1 (LCDR1). In certain embodiments IL2 is incorporated into light chain complementarity determining region 2 (LCDR2). In certain embodiments IL2 is incorporated into light chain complementarity determining region 3 (LCDR3).

In some embodiments, the antibody cytokine engrafted comprises an IL2 sequence incorporated into a CDR, whereby the IL2 sequence is inserted into the CDR sequence. The insertion may be at or near the N-terminal region of the CDR, in the middle region of the CDR or at or near the C-terminal region of the CDR. In other embodiments, the antibody cytokine engrafted comprises IL2 incorporated into a CDR, whereby the IL2 sequence does not frameshift the CDR sequence.

In some embodiments IL2 is engrafted directly into a CDR without a peptide linker, with no additional amino acids between the CDR sequence and the IL2 sequence.

In some embodiments antibody cytokine engrafted proteins comprise immunoglobulin heavy chains of an IgG class antibody heavy chain. In certain embodiments an IgG heavy chain is any one of an IgG1, an IgG2 or an IgG4 subclass.

In some embodiments antibody cytokine engrafted proteins comprise heavy and light chain immunoglobulin sequences selected from a known, clinically utilized immunoglobulin sequence. In certain embodiments antibody cytokine engrafted proteins comprise heavy and light chain immunoglobulin sequences which are humanized sequences. In other certain embodiments antibody cytokine engrafted proteins comprise heavy and light chain immunoglobulin sequences which are human sequences.

In some embodiments antibody cytokine engrafted proteins comprise heavy and light chain immunoglobulin sequences selected from germline immunoglobulin sequences.

In some embodiments antibody cytokine engrafted proteins comprise heavy and light chain immunoglobulin sequences having binding specificity of the immunoglobulin variable domains to a target distinct from the binding specificity of the IL2 molecule. In some embodiments the binding specificity of the immunoglobulin variable domain to its target is retained in the presence of the engrafted. In certain embodiments the retained binding specificity is to a non-human target. In other embodiments the binding specificity is to a human target having therapeutic utility in conjunction with IL2 therapy. In certain embodiments, targeting the binding specificity of the immunoglobulin conveys additional therapeutic benefit to the IL2 component. In certain embodiments the binding specificity of the immunoglobulin to its target conveys synergistic activity with IL2.

In still other embodiments, the binding specificity of the immunoglobulin is reduced by the engrafting of the IL2 molecule.

### ACE Proteins Targeting the IL6 Receptor

Provided herein are ACE proteins comprising an IL6 molecule engrafted into the complementarity determining region (CDR) of an antibody. The ACE proteins of the present disclosure show suitable properties to be used in human patients, for example, they retain activity similar to that of native or recombinant human IL6. Other activities and characteristics are also demonstrated throughout the specification. Thus, provided are ACE proteins having an improved therapeutic profile over previously known IL6 and modified IL6 therapeutic agents, and methods of use of the provided ACE proteins in cancer treatment.

Accordingly, the present disclosure provides ACE proteins that are agonists of the IL6 receptor, with selective activity profiles. Provided ACE proteins comprise an immunoglobulin heavy chain sequence and an immunoglobulin light chain sequence. Each immunoglobulin heavy chain sequence comprises a heavy chain variable region (VH) and a heavy chain constant region (CH), wherein the heavy chain constant region consists of CH1, CH2, and CH3 constant regions. Each immunoglobulin light chain sequence comprises a light chain variable region (VL) and a light chain constant region (CL). In each ACE protein an IL6 molecule is incorporated into a complementarity determining region (CDR) of the VH or VL.

In some embodiments, the ACE protein comprises IL6 molecule incorporated into a heavy chain CDR. In certain embodiments IL6 is incorporated into heavy chain complementarity determining region 1 (HCDR1). In certain embodiments IL6 is incorporated into heavy chain complementarity determining region 2 (HCDR2). In certain embodiments IL6 is incorporated into heavy chain complementarity determining region 3 (HCDR3).

In some embodiments, the ACE protein comprises IL6 incorporated into a light chain CDR. In certain embodiments IL6 is incorporated into light chain complementarity determining region 1 (LCDR1). In certain embodiments IL6 is incorporated into light chain complementarity determining region 2 (LCDR2). In certain embodiments IL6 is incorporated into light chain complementarity determining region 3 (LCDR3).

In some embodiments, the ACE comprises an IL6 sequence incorporated into a CDR, whereby the IL6 sequence is inserted into the CDR sequence. The insertion may be at or near the N-terminal region of the CDR, in the middle region of the CDR or at or near the C-terminal region of the CDR. In other embodiments, the ACE comprises IL6 incorporated into a CDR, whereby the IL6 sequence does not frameshift the CDR sequence.

In some embodiments IL6 is engrafted directly into a CDR without a peptide linker, with no additional amino acids between the CDR sequence and the IL6 sequence.

In some embodiments ACE proteins comprise immunoglobulin heavy chains of an IgG class antibody heavy chain. In certain embodiments an IgG heavy chain is any one of an IgG1, an IgG2 or an IgG4 subclass.

In some embodiments ACE proteins comprise heavy and light chain immunoglobulin sequences selected from a known, clinically utilized immunoglobulin sequence. In certain embodiments ACE proteins comprise heavy and light chain immunoglobulin sequences which are humanized sequences. In other certain embodiments ACE proteins comprise heavy and light chain immunoglobulin sequences which are human sequences.

In some embodiments ACE proteins comprise heavy and light chain immunoglobulin sequences selected from germline immunoglobulin sequences.

In some embodiments ACE proteins comprise heavy and light chain immunoglobulin sequences having binding specificity of the immunoglobulin variable domains to a target distinct from the binding specificity of the cytokine molecule. In some embodiments the binding specificity of the immunoglobulin variable domain to its target is retained by 10%, 20%, 30%, 40%, 50%, 60%, 70%, 80%, 90%, 95%, 98%, 99%, or 100%, in the presence of the engrafted cytokine. In certain embodiments the retained binding specificity is to a non-human target. In other embodiments the binding specificity is to a human target having therapeutic utility in conjunction with therapy. In certain embodiments, targeting the binding specificity of the immunoglobulin conveys additional therapeutic benefit to the cytokine component. In certain embodiments the binding specificity of the immunoglobulin to its target conveys synergistic activity with cytokine.

In still other embodiments, the binding specificity of the immunoglobulin is reduced 10%, 20%, 30%, 40%, 50%, 60%, 70%, 80%, 90%, 95%, 98%, 99%, or 100%, by the engrafting of the cytokine molecule.

In some embodiments, the ACE proteins comprise a modified immunoglobulin IgG having a modified Fc conferring modified effector function. In certain embodiments the modified Fc region comprises a mutation selected from one or more of D265A, P329A, P329G, N297A, L234A, and L235A. In particular embodiments the immunoglobulin heavy chain may comprise a mutation or combination of mutations conferring reduced effector function selected from any of D265A, P329A, P329G, N297A, D265A/P329A, D265A/N297A, L234/L235A, P329A/L234A/L235A, and P329G/L234A/L235A. In some embodiments, the Fc mutation is D265A/P329A.

In some embodiments, the ACE proteins comprise (i) a heavy chain variable region having at least 85%, 89%, 90%, 91%, 92%, 93%, 94%, 95%, 96%, 97%, 98%, 99%, or 100% amino acid sequence identity to a heavy chain variable region set forth in TABLE 2 and (ii) a light chain variable region having at least 85%, 89%, 90%, 91%, 92%, 93%, 94%, 95%, 96%, 97%, 98%, 99%, or 100% amino acid sequence identity to a light chain variable region set forth in TABLE 2. The immunoglobulin chain is an IgG class selected from IgG1, IgG2, or IgG4. In certain embodiments the immunoglobulin optionally comprises a mutation or combination of mutations conferring reduced effector function selected from any of D265A, P329A, P329G, N297A, D265A/P329A, D265A/N297A, L234/L235A, P329A/L234A/L235A, and P329G/L234A/L235A. In some embodiments, the Fc mutation is D265A/P329A.

Engineered and/or Modified ACE Proteins

In certain aspects, ACE proteins are generated by engineering a cytokine sequence into a CDR region of an immunoglobulin scaffold. Both heavy and light chain immunoglobulin chains are produced to generate final antibody engrafted proteins. ACE proteins confer preferred therapeutic activity on T cells, and the ACE proteins as compared with native or recombinant human cytokine or a cytokine fused to an Fc.

To engineer ACE proteins, cytokine sequences are inserted into a CDR loop of an immunoglobulin chain scaffold protein. Engrafted ACE proteins can be prepared using any of a variety of known immunoglobulin sequences which have been utilized in clinical settings, known immunoglobulin sequences which are in current discovery and/or clinical development, human germline antibody sequences, as well as sequences of novel antibody immunoglobulin chains. Constructs are produced using standard molecular biology methodology utilizing recombinant DNA encoding relevant sequences. Sequences of cytokines in exemplary scaffolds, referred to as GFTX3b, and GFTX are depicted in TABLE 2. Insertion points were selected to be the mid-point of the loop based on available structural or homology model data, however, insertion points can be adjusted toward one or another end of the CDR loop. In some embodiments, engrafted constructs can be prepared using an immunoglobulin scaffold that does not have binding specificity to any antigen. In some embodiments, engrafted constructs can be prepared using an immunoglobulin scaffold that does not have binding specificity to a human antigen. In some embodiments, engrafted constructs can be prepared using an immunoglobulin scoffold that has binding specificity to a human antigen, such as a tumor antigen

Thus the present disclosure provides antibodies or fragments thereof that specifically bind to cytokine receptors comprising a cytokine protein recombinantly inserted into a heterologous antibody protein or polypeptide to generate engrafted proteins. In particular, the disclosure provides engrafted proteins comprising an antibody or antigen-binding fragment of an antibody described herein or any other relevant scaffold antibody polypeptide (e.g., a full antibody immunoglobulin protein, a Fab fragment, Fc fragment, Fv fragment, F(ab)2 fragment, a VH domain, a VH CDR, a VL domain, a VL CDR, etc.) and a heterologous cytokine protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341. Additionally, ACE proteins may be generated through the techniques of gene-shuffling, motif-shuffling, exon-shuffling, and/or codon-shuffling (collectively referred to as “DNA shuffling”). DNA shuffling may be employed to prepare engrafted protein constructs and/or to alter the activities of antibodies or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313. Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an antigen protein of interest may be recombined with one or more components, motifs, sections, parts, domains, fragments, etc. of one or more heterologous cytokine molecules, for preparation of ACE proteins as provided herein.

An antibody Fab contains six CDR loops, 3 in the light chain (CDRL1, CDRL2, CDRL3) and 3 in the heavy chain (CDRH1, CDRH2, CDRH3) which can serve as potential insertion sites for a cytokine protein. Structural and functional considerations are taken into account in order to determine which CDR loop(s) to insert the cytokine. As a CDR loop size and conformation vary greatly across different antibodies, the optimal CDR for insertion can be determined empirically for each particular antibody/protein combination. Additionally, since a cytokine protein will be inserted into a CDR loop, this can put additional constraints on the structure of the cytokine protein.

CDRs of immunoglobulin chains are determined by well-known numbering systems known in the art, including those described herein. For example, CDRs have been identified and defined by (1) using the numbering system described in Kabat et al. (1991), “Sequences of Proteins of Immunological Interest,” 5th Ed. Public Health Service, National Institutes of Health, Bethesda, Md. (“Kabat” numbering scheme), NIH publication No. 91-3242; and (2) Chothia, see Al-Lazikani et al., (1997) “Standard conformations for the canonical structures of immunoglobulins,” J. Mol. Biol. 273:927-948. For identified CDR amino acid sequences less than 20 amino acids in length, one or two conservative amino acid residue substitutions can be tolerated while still retaining the desired specific binding and/or agonist activity.

An ACE protein further can be prepared using an antibody having one or more of the CDRs and/or V_{H} and/or V_{L} sequences shown herein (e.g., TABLE 2) as starting material to engineer a modified ACE protein, which may have altered properties from the starting antibody engrafted protein. Alternatively any known antibody sequences may be utilized as a scaffold to engineer modified ACE protein. For example, any known, clinically utilized antibody may be utilized as a starting materials scaffold for preparation of antibody engrafted protein. Known antibodies and corresponding immunoglobulin sequences include, e.g., palivizumab, alirocumab, mepolizumab, necitumumab, nivolumab, dinutuximab, secukinumab, evolocumab, blinatumomab, pembrolizumab, ramucirumab vedolizumab, siltuximab, obinutuzumab, trastuzumab, raxibacumab, pertuzumab, belimumab, ipilimumab. denosumab, tocilizumab, ofatumumab, canakinumab, golimumab, ustekinumab, certolizumab, catumaxomab, eculizumab, ranibizumab, panitumumab, natalizumab, bevacizumab, cetuximab, efalizumab, omalizumab, tositumomab, ibritumomab tiuxetan, adalimumab, alemtuzumab, gemtuzumab, infliximab, basiliximab, daclizumab, rituximab, abciximab, muromonab, or modifications thereof. Known antibodies and immunoglobulin sequences also include germline antibody sequences. Framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836. In still other examples, antibody and corresponding immunoglobulin sequences from other known entities which can be in early discovery and/or drug development can be similarly adapted as starting material to engineer a modified ACE protein.

A wide variety of antibody/immunoglobulin frameworks or scaffolds can be employed so long as the resulting polypeptide includes at least one binding region which accommodates incorporation of a cytokine. Such frameworks or scaffolds include the 5 main idiotypes of human immunoglobulins, or fragments thereof, and include immunoglobulins of other animal species, preferably having humanized and/or human aspects. Novel antibodies, frameworks, scaffolds and fragments continue to be discovered and developed by those skilled in the art.

Antibodies can be generated using methods that are known in the art. For preparation of monoclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies for use in ACE proteins. Also, transgenic mice, or other organisms such as other mammals, may be used to express and identify primatized or humanized or human antibodies. Alternatively, phage display technology can be used to identify antibodies and heteromeric Fab fragments that specifically bind to selected antigens for use in ACE proteins (see, e.g., McCafferty et al., supra; Marks et al., Biotechnology, 10:779-783, (1992)).

Methods for primatizing or humanizing non-human antibodies are well known in the art. Generally, a primatized or humanized antibody has one or more amino acid residues introduced into it from a source which is non-primate or non-human Such non-primate or non-human amino acid residues are often referred to as import residues, which are typically taken from an import variable domain Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, primatized or humanized antibodies are typically primate or human antibodies in which some complementary determining region (“CDR”) residues and possibly some framework (“FR”) residues are substituted by residues from analogous sites in an originating species (e.g., rodent antibodies) to confer binding specificity.

Alternatively or additionally, an in vivo method for replacing a nonhuman antibody variable region with a human variable region in an antibody while maintaining the same or providing better binding characteristics relative to that of the nonhuman antibody may be utilized to convert non-human antibodies into engineered human antibodies. See, e.g., U.S. Patent Publication No. 20050008625, U.S. Patent Publication No. 2005/0255552. Alternatively, human V segment libraries can be generated by sequential cassette replacement in which only part of the reference antibody V segment is initially replaced by a library of human sequences; and identified human “cassettes” supporting binding in the context of residual reference antibody amino acid sequences are then recombined in a second library screen to generate completely human V segments (see, U.S. Patent Publication No. 2006/0134098).

Various antibodies or antigen-binding fragments for use in preparation of ACE proteins can be produced by enzymatic or chemical modification of the intact antibodies, or synthesized de novo using recombinant DNA methodologies (e.g., single chain Fv), or identified using phage display libraries (see, e.g., McCafferty et al., Nature 348:552-554, 1990). For example, minibodies can be generated using methods described in the art, e.g., Vaughan and Sollazzo, Comb. Chem. High Throughput Screen 4:417-30 2001. Bispecific antibodies can be produced by a variety of methods including engrafted of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries or ribosome display libraries, gene shuffled libraries. Such libraries can be constructed from synthetic, semi-synthetic or native and immunocompetent sources. Selected immunoglobulin sequences may thus be utilized in preparation of ACE protein constructs as provided herein.

Antibodies, antigen-binding molecules or ACE molecules of use in the present disclosure further include bispecific antibodies. A bispecific or bifunctional antibody is an artificial hybrid antibody having two different heavy/light chain pairs and two different binding sites. Other antigen-binding fragments or antibody portions include bivalent scFv (diabody), bispecific scFv antibodies where the antibody molecule recognizes two different epitopes, single binding domains (dAbs), and minibodies. Selected immunoglobulin sequences may thus be utilized in preparation of ACE protein constructs as provided herein.

Antigen-binding fragments of antibodies e.g., a Fab fragment, scFv, can be used as building blocks to construct ACE proteins, and may optionally include multivalent formats. In some embodiments, such multivalent molecules comprise a constant region of an antibody (e.g., Fc).

ACE proteins can be engineered by modifying one or more residues within one or both variable regions (i.e., VH and/or VL) of an antibody, for example, within one or more CDR regions, and such adapted VH and/or VL region sequences are utilized for engrafting a cytokine or for preparation of cytokine engrafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. CDR sequences are responsible for most antibody-antigen interactions, it is possible to express recombinant antibodies that mimic the properties of a specific antibody by constructing expression vectors that include CDR sequences from a specific antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.). In certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

In some aspects mutation of amino acid residues within the VH and/or VL CDR1, CDR2, and/or CDR3 regions to thereby improve one or more binding properties (e.g., affinity) of the antibody of interest, known as “affinity maturation,” may be beneficial, e.g., to optimize antigen binding of an antibody in conjunction with the context of the cytokine engrafted protein. Site-directed mutagenesis or PCR-mediated mutagenesis can be performed to introduce the mutation(s) and the effect on antibody binding, or other functional property of interest, can be evaluated in in vitro or in vivo assays as described herein and/or alternative or additional assays known in the art. Conservative modifications can be introduced. The mutations may be amino acid substitutions, additions or deletions. Moreover, typically no more than one, two, three, four or five residues within a CDR region are altered.

Engineered antibodies or antibody fragments include those in which modifications have been made to framework residues within VH and/or VL, e.g. to improve the properties of the antibody. In some embodiments such framework modifications are made to decrease immunogenicity of the antibody. For example, one approach is to change one or more framework residues to the corresponding germline sequence. More specifically, an antibody that has undergone somatic mutation may contain framework residues that differ from germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Additional framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Constant regions of the antibodies or antibody fragments utilized for preparation of the ACE protein can be any type or subtype, as appropriate, and can be selected to be from the species of the subject to be treated by the present methods (e.g., human, non-human primate or other mammal, for example, agricultural mammal (e.g., equine, ovine, bovine, porcine, camelid), domestic mammal (e.g., canine, feline) or rodent (e.g., rat, mouse, hamster, rabbit). In some embodiments antibodies utilized in ACE proteins are engineered to generate humanized or Humaneered® antibodies. In some embodiments antibodies utilized in ACE proteins are human antibodies. In some embodiments, antibody constant region isotype is IgG, for example, IgG1, IgG2, IgG3, IgG4. In certain embodiments the constant region isotype is IgG1. In some embodiments, ACE proteins comprise an IgG. In some embodiments, ACE proteins comprise an IgG1 Fc. In some embodiments, ACE proteins comprise an IgG2 Fc.

In addition or alternative to modifications made within framework or CDR regions, antibodies or antibody fragments utilized in preparation of ACE proteins may be engineered to include modifications within an Fc region, typically to alter one or more functional properties of the antibody, such as, e.g., serum half-life, complement fixation, Fc receptor binding, and/or antigen-dependent cellular cytotoxicity. Furthermore, an antibody, antibody fragment thereof, or ACE protein can be chemically modified (e.g., one or more chemical moieties can be attached to the antibody) or be modified to alter its glycosylation, again to alter one or more functional properties of the ACE protein.

In one embodiment, a hinge region of CH1 is modified such that the number of cysteine residues in the hinge region is altered, e.g., increased or decreased. For example, by the approach is described further in U.S. Pat. No. 5,677,425 by Bodmer et al. wherein the number of cysteine residues in the hinge region of CH1 is altered to, for example, facilitate assembly of the light and heavy chains or to increase or decrease the stability of the ACE protein. In another embodiment, an Fc hinge region of an antibody is mutated to alter the biological half-life of the ACE protein. More specifically, one or more amino acid mutations are introduced into the CH2-CH3 domain interface region of the Fc-hinge fragment such that the ACE protein has impaired Staphylococcyl protein A (SpA) binding relative to native Fc-hinge domain SpA binding. This approach is described in further detail in U.S. Pat. No. 6,165,745 by Ward et al.

The present disclosure provides for ACE proteins that specifically bind to a cytokine receptor which have an extended half-life in vivo. In another embodiment, an ACE protein is modified to increase its biological half-life. Various approaches are possible. ACE proteins having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). For example, one or more of the following mutations can be introduced: T252L, T254S, T256F, as described in U.S. Pat. No. 6,277,375 to Ward. See, e.g., International Publication No. WO 98/23289; International Publication No. WO 97/34631; and U.S. Pat. No. 6,277,375. Alternatively, to increase the biological half-life, the ACE protein is altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. In yet other embodiments, the Fc region is altered by replacing at least one amino acid residue with a different amino acid residue to alter the effector functions of the ACE protein. For example, one or more amino acids can be replaced with a different amino acid residue such that the ACE protein has an altered affinity for an effector ligand but retains antigen-binding ability of the parent antibody. The effector ligand to which affinity is altered can be, for example, an Fc receptor (FcR) or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the ACE protein has altered C1q binding and/or reduced or abolished complement dependent cytotoxicity (CDC). This approach is described in further detail in U.S. Pat. No. 6,194,551 by Idusogie et al.

ACE proteins containing such mutations mediate reduced or no antibody-dependent cellular cytotoxicity (ADCC) or complement-dependent cytotoxicity (CDC). In some embodiments, amino acid residues L234 and L235 of the IgG1 constant region are substituted to Ala234 and Ala235. In some embodiments, amino acid residue N267 of the IgG1 constant region is substituted to Ala267.

In another embodiment, one or more amino acid residues are altered to thereby alter the ability of the ACE protein to fix complement. This approach is described further in PCT Publication WO 94/29351 by Bodmer et al.

In yet another embodiment, an Fc region is modified to increase the ability of the antibody to mediate antibody dependent cellular cytotoxicity (ADCC) and/or to increase the affinity of the ACE protein for an Fcγ receptor by modifying one or more amino acids. This approach is described further in PCT Publication WO 00/42072 by Presta. Moreover, binding sites on human IgG1 for FcγR1, FcγRII, FcγRIII and FcRn have been mapped and variants with improved binding have been described (see Shields, R. L. et al., 2001 J. Biol. Chem. 276:6591-6604).

In still another embodiment, glycosylation of an ACE protein is modified. For example, an aglycoslated ACE protein can be made (i.e., the ACE protein lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an ACE protein can be made that has an altered type of glycosylation, such as a hypofucosylated ACE protein having reduced amounts of fucosyl residues or an antibody having increased bisecting GlcNac structures. Such altered glycosylation patterns have been demonstrated to increase the antibody dependent cellular cytotoxicity (ADCC) ability of antibodies. Such carbohydrate modifications can be accomplished by, for example, expressing the ACE protein in a host cell with altered glycosylation machinery. Cells with altered glycosylation machinery have been described in the art and can be used as host cells in which to express recombinant ACE proteins to thereby produce an ACE protein with altered glycosylation. For example, EP 1,176,195 by Hang et al. describes a cell line with a functionally disrupted FUT8 gene, which encodes a fucosyl transferase, such that ACE proteins expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of ACE proteins expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that ACE proteins expressed in the engineered cell lines exhibit increased bisecting GlcNac structures which results in increased ADCC activity of the antibodies (see also Umana et al., 1999 Nat. Biotech. 17:176-180).

In some embodiments, one or more domains, or regions, of an ACE protein are connected via a linker, for example, a peptide linker, such as those that are well known in the art (see e.g., Holliger, P., et al. (1993) Proc. Natl. Acad. Sci. USA 90:6444-6448; Poljak, R J., et al. (1994) Structure 2:1121-1123). A peptide linker may vary in length, e.g., a linker can be 1-100 amino acids in length, typically a linker is from five to 50 amino acids in length, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments the cytokine is engrafted into the CDR sequence optionally with one or more peptide linker sequences. In certain embodiments one or more peptide linkers is independently selected from a (Glyₙ-Ser)ₘ sequence (SEQ ID NO: 3974), a (Glyₙ-Ala)ₘ sequence (SEQ ID NO: 3975), or any combination of a (Glyₙ-Ser)ₘ/(Glyₙ-Ala)ₘ sequence (SEQ ID NOS 3974-3975), wherein each n is independently an integer from 1 to 5 and each m is independently an integer from 0 to 10. Examples of linkers include, but are not limited to, glycine-based linkers or gly/ser linkers G/S such as (GₘS)ₙ wherein n is a positive integer equal to 1, 2, 3, 4, 5, 6, 7, 8, 9, or 10 and m is an integer equal to 0, 1, 2, 3, 4, 5, 6, 7, 8, 9, or 10 (SEQ ID NO: 3976). In certain embodiments one or more linkers include G₄S (SEQ ID NO: 3972) repeats, e.g., the Gly-Ser linker (G₄S)ₙ wherein n is a positive integer equal to or greater than 1 (SEQ ID NO: 3972). For example, n=1, n=2, n=3. n=4, n=5 and n=6, n=7, n=8, n=9 and n=10. In some embodiments, Ser can be replaced with Ala e.g., linkers G/A such as (GₘA)ₙ wherein n is a positive integer equal to 1, 2, 3, 4, 5, 6, 7, 8, 9, or 10 and m is an integer equal to 0, 1, 2, 3, 4, 5, 6, 7, 8, 9, or 10 (SEQ ID NO: 3977). In certain embodiments one or more linkers include G₄A (SEQ ID NO: 3973) repeats, (G₄A)ₙ wherein n is a positive integer equal to or greater than 1 (SEQ ID NO: 3973). For example, n=1, n=2, n=3. n=4, n=5 and n=6, n=7, n=8, n=9 and n=10. In some embodiments, the linker includes multiple repeats of linkers. In other embodiments, a linker includes combinations and multiples of G₄S (SEQ ID NO: 3972) and G₄A (SEQ ID NO: 3973).

Other examples of linkers include those based on flexible linker sequences that occur naturally in antibodies to minimize immunogenicity arising from linkers and junctions. For example, there is a natural flexible linkage between the variable domain and a CH1 constant domain in antibody molecular structure. This natural linkage comprises approximately 10-12 amino acid residues, contributed by 4-6 residues from C-terminus of V domain and 4-6 residues from the N-terminus of the CH1 domain. ACE proteins can, e.g., employ linkers incorporating terminal 5-6 amino acid residues, or 11-12 amino acid residues, of CH1 as a linker. The N-terminal residues of the CH1 domain, particularly the first 5-6 amino acid residues, adopt a loop conformation without strong secondary structure, and, therefore, can act as a flexible linker. The N-terminal residues of the CH1 domain are a natural extension of the variable domains, as they are part of the Ig sequences, and, therefore, minimize to a large extent any immunogenicity potentially arising from the linkers and junctions. In some embodiments a linker sequence includes a modified peptide sequence based on a hinge sequence.

Moreover, the ACE proteins can include marker sequences, such as a peptide to facilitate purification of ACE proteins. In preferred embodiments, a marker amino acid sequence is a hexa-histidine (SEQ ID NO: 3978) peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine (SEQ ID NO: 3978) provides for convenient purification of the engrafted protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Assays for ACE Protein Activity

Assays for identifying ACE proteins are known in the art and described herein. Agonist ACE proteins bind to their cognate cytokine receptor and promote, induce, stimulate intracellular signaling resulting in intracellular signaling as well as other biological effects.

Binding of the ACE proteins to their receptor can be determined using any method known in the art. For example, binding to the receptor can be determined using known techniques, including without limitation ELISA, Western blots, surface plasmon resonance (SPR) (e.g., BIAcore), and flow cytometry.

Intracellular signaling through the cytokine receptor can be measured using any method known in the art. For example, activation of the IL7Ra by IL7 promotes STAT5 activation and signaling. Methods for measuring STAT5 activation are standard in the art (e.g., phosphorylation status of STAT5 protein, reporter gene assays, downstream signaling assays, etc.). As another example, activation through the IL7Ra expands T cells, so the absolute numbers of T cells can be assayed for. In addition, either CD8+ or CD4+ T cells can be assayed for independently. Methods for measuring proliferation of cells are standard in the art (e.g., ³H-thymidine incorporation assays, CFSE labelling). Methods for measuring cytokine production are well known in the art (e.g., ELISA assays, ELISpot assays). In performing in vitro assays, test cells or culture supernatant from test cells contacted with ACE proteins can be compared to control cells or culture supernatants from control cells that have not been contacted with an ACE protein and/or those that have been contacted with recombinant human cytokine or an cytokine-Fc fusion molecule.

The activity of the ACE proteins can also be measured ex vivo and/or in vivo. In some aspects, methods for measuring receptor activation across various cell types ex vivo from animals treated with ACE proteins as compared to untreated control animals and/or animals similarly treated with native cytokine may be used to show differential activity of the ACE proteins across cell types. Preferred agonist ACE proteins have the ability to induce intracellular signaling. The efficacy of the ACE proteins can be determined by administering a therapeutically effective amount of the ACE protein to a subject and comparing the subject before and after administration of the ACE protein. Efficacy of the ACE proteins can also be determined by administering a therapeutically effective amount of an ACE protein to a test subject and comparing the test subject to a control subject who has not been administered the antibody and/or comparison to a subject similarly treated with the native cytokine.

### Polynucleotides Encoding ACE Proteins

In another aspect, isolated nucleic acids encoding heavy and light chain proteins of the ACE proteins are provided. ACE proteins can be produced by any means known in the art, including but not limited to, recombinant expression, chemical synthesis, and enzymatic digestion of antibody tetramers. Recombinant expression can be from any appropriate host cells known in the art, for example, mammalian host cells, bacterial host cells, yeast host cells, insect host cells, etc.

Provided herein are polynucleotides that encode the variable regions exemplified in TABLE 2. In some embodiments, the polynucleotide encoding the heavy chain variable regions comprises a sequence having at least 85%, 89%, 90%, 91%, 92%, 93%, 94%, 95%, 96%, 97%, 98%, 99%, or 100% nucleic acid sequence identity with a polynucleotide encoding a variable heavy chain or a variable light chain as set forth in TABLE 2.

Polynucleotides can encode only the variable region sequence of an ACE protein. They can also encode both a variable region and a constant region of the ACE protein. Some of the polynucleotide sequences encode a polypeptide that comprises variable regions of both the heavy chain and the light chain of one of the ACE proteins. Some other polynucleotides encode two polypeptide segments that respectively are substantially identical to the variable regions of the heavy chain and the light chain of one of the ACE proteins.

In certain embodiments polynucleotides or nucleic acids comprise DNA. In other embodiments polynucleotides or nucleic acids comprise RNA, which may be single stranded or double stranded.

In some embodiments a recombinant host cell comprising the nucleic acids encoding one or more immunoglobulin protein chain of an ACE protein, and optionally, secretion signals are provided. In certain embodiments a recombinant host cell comprises a vector encoding one immunoglobulin protein chain and secretion signals. In other certain embodiments a recombinant host cell comprises one or more vectors encoding two immunoglobulin protein chains of the ACE protein and secretion signals. In some embodiments a recombinant host cell comprises a single vector encoding two immunoglobulin protein chains of the ACE protein and secretion signals. In some embodiments a recombinant host cell comprises two vectors, one encoding a heavy chain immunoglobulin protein chain, and another encoding a light chain immunoglobulin protein chain of the ACE protein, with each including secretion signals. A recombinant host cell may be a prokaryotic or eukaryotic cell. In some embodiments a host cell is a eukaryotic cell line. In some embodiments a host cell is a mammalian cell line.

Additionally provided are methods for producing the ACE proteins. In some embodiments the method comprises the steps of (i) culturing a host cell comprising one or more vectors encoding immunoglobulin protein chains of an ACE protein under conditions suitable for expression, formation, and secretion of the ACE protein and (ii) recovering the ACE protein.

The polynucleotide sequences can be produced by de novo solid-phase DNA synthesis or by PCR mutagenesis of an existing sequence (e.g., sequences as described herein) encoding a polypeptide chain of an ACE protein. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the disclosure are expression vectors and host cells for producing the ACE proteins described above. Various expression vectors can be employed to express polynucleotides encoding the immunoglobulin polypeptide chains, or fragments, of the ACE proteins. Both viral-based and nonviral expression vectors can be used to produce the immunoglobulin proteins in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the ACE protein polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an immunoglobulin protein of the ACE protein. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an immunoglobulin chain or fragment of the ACE proteins. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

Expression vectors may also provide a secretion signal sequence position to form an ACE protein that exported out of the cell and into the culture medium. In certain aspects, the inserted immunoglobulin sequences of the ACE proteins are linked to a signal sequences before inclusion in the vector. Vectors to be used to receive sequences encoding immunoglobulin light and heavy chain variable domains sometimes also encode constant regions or parts thereof. Such vectors allow expression of the variable regions as engrafted proteins with the constant regions thereby leading to production of intact ACE proteins or fragments thereof. Typically, such constant regions are human.

Host cells for harboring and expressing the ACE protein chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express ACE protein polypeptides. Insect cells in combination with baculovirus vectors can also be used.

In some preferred embodiments, mammalian host cells are used to express and produce the ACE protein polypeptides. For example, they can be either a mammalian cell line containing an exogenous expression vector. These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, engrafted to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express ACE protein immunoglobulin chains can be prepared using expression vectors which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Pharmaceutical Compositions Comprising ACE Proteins

Provided are pharmaceutical compositions comprising an ACE protein formulated together with a pharmaceutically acceptable carrier. Optionally, pharmaceutical compositions additionally contain other therapeutic agents that are suitable for treating or preventing a given disorder. Pharmaceutically acceptable carriers enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present disclosure can be administered by a variety of methods known in the art. Route and/or mode of administration vary depending upon the desired results. It is preferred that administration be by parenteral administration (e.g., selected from any of intravenous, intramuscular, intraperitoneal, intrathecal, intraarterial, or subcutaneous), or administered proximal to the site of the target. A pharmaceutically acceptable carrier is suitable for administration by any one or more of intravenous, intramuscular, intraperitoneal, intrathecal, intraarterial, subcutaneous, intranasal, inhalational, spinal or epidermal administration (e.g., by injection). Depending on the route of administration, active compound, e.g., ACE protein, may be coated in a material to protect the compound from the action of acids and other natural conditions that may inactivate the compound. In some embodiments the pharmaceutical composition is formulated for intravenous administration. In some embodiments the pharmaceutical composition is formulation for subcutaneous administration.

An ACE protein, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In some embodiments, a pharmaceutical composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin. In certain embodiments compositions can be prepared for storage in a lyophilized form using appropriate excipients (e.g., sucrose).

Pharmaceutical compositions can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions. Applicable methods for formulating an ACE protein and determining appropriate dosing and scheduling can be found, for example, in Remington: The Science and Practice of Pharmacy, 21st Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of an ACE protein is employed in the pharmaceutical compositions. An ACE protein is formulated into pharmaceutically acceptable dosage form by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### Articles of Manufacture/Kits

In some aspects an ACE protein is provided in an article of manufacture (i.e., a kit). A provided ACE protein is generally in a vial or a container. Thus, an article of manufacture comprises a container and a label or package insert, on or associated with the container. Suitable containers include, for example, a bottle, vial, syringe, solution bag, etc. As appropriate, the ACE protein can be in liquid or dried (e.g., lyophilized) form. The container holds a composition which, by itself or combined with another composition, is effective for preparing a composition for treating, preventing and/or ameliorating cancer. The label or package insert indicates the composition is used for treating, preventing and/or ameliorating cancer. Articles of manufacture (kits) comprising an ACE protein, as described herein, optionally contain one or more additional agent. In some embodiments, an article of manufacture (kit) contains ACE protein and a pharmaceutically acceptable diluent. In some embodiments an ACE protein is provided in an article of manufacture (kit) with one or more additional active agent in the same formulation (e.g., as mixtures). In some embodiments an ACE protein is provided in an article of manufacture (kit) with a second or third agent in separate formulations (e.g., in separate containers). In certain embodiments an article of manufacture (kit) contains aliquots of the ACE protein wherein the aliquot provides for one or more doses. In some embodiments aliquots for multiple administrations are provided, wherein doses are uniform or varied. In particular embodiments varied dosing regimens are escalating or decreasing, as appropriate. In some embodiments dosages of an ACE protein and a second agent are independently uniform or independently varying. In certain embodiments, an article of manufacture (kit) comprises an additional agent such as an anti-cancer agent or immune checkpoint molecule. Selection of one or more additional agent will depend on the dosage, delivery, and disease condition to be treated.

### Methods of Treatment and Use of Pharmaceutical Compositions for Treatment

### Treatment of Cancer

ACE proteins find use in treatment, amelioration or prophylaxis of cancer. In one aspect, the disclosure provides methods of treatment of cancer in an individual in need thereof, comprising administering to the individual a therapeutically effective amount of an ACE protein, as described herein. In some embodiment an ACE protein is provided for use as a therapeutic agent in the treatment or prophylaxis of cancer in an individual. In a further aspect, the disclosure provides a composition comprising such an ACE protein for use in treating or ameliorating cancer in an individual in need thereof.

Conditions subject to treatment include various cancer indications. For therapeutic purposes, an individual was diagnosed with cancer. For preventative or prophylactic purposes, an individual may be in remission from cancer or may anticipate future onset. In some embodiments, the patient has cancer, is suspected of having cancer, or is in remission from cancer. Cancers subject to treatment with an ACE protein usually derive benefit from activation of cytokine signaling, as described herein. Cancer indications subject to treatment include without limitation: melanoma, lung cancer, colorectal cancer, prostate cancer, breast cancer and lymphoma.

### Treatment of Immune Related Disorder

ACE proteins find use in treatment, amelioration or prophylaxis of immune related disorder. In one aspect, the disclosure provides methods of treatment of immune related disorder in an individual in need thereof, comprising administering to the individual a therapeutically effective amount of an ACE protein, as described herein. In some embodiment an ACE protein is provided for use as a therapeutic agent in the treatment or prophylaxis of immune related disorder in an individual. In a further aspect, the disclosure provides a composition comprising such an ACE protein for use in treating or ameliorating immune related disorder in an individual in need thereof.

Conditions subject to treatment include various immune related disorders. For therapeutic purposes, an individual was diagnosed with an immune related disorder. For preventative or prophylactic purposes, an individual may be in remission from an immune related disorder or may anticipate future onset. In some embodiments, the patient has immune related disorder, is suspected of having immune related disorder, or is in remission from immune related disorder Immune related disorders subject to treatment with an ACE protein usually derive benefit from activation of cytokine signaling, as described herein. Immune related disorders subject to treatment include without limitation: inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

### Treatment of Obesity

ACE proteins find uses in treatment, amelioration or prophylaxis of obesity. In one aspect, the disclosure provides methods of treating obesity in an individual in need thereof, comprising administering to the individual a therapeutically effective amount of an ACE protein as described herein. In some embodiments, an ACE protein is provided for use as a therapeutic agent in the treatment or prophylaxis of obesity in an individual. In a further aspect, the disclosure provides a composition comprising such an ACE protein for use in treating or ameliorating obesity in an individual in need thereof.

Conditions subject to treatment include various obesity indications. For therapeutic purposes, an individual was diagnosed with obesity. For preventative or prophylactic purposes, an individual may anticipate future onset of obesity. In some embodiments, the patient has obesity, is suspected of having obesity, or is recovering from obesity. Obesity subject to treatment with an antibody cytokine engrafted protein may derive benefits from activation of cytokine signaling, as described herein.

### Administration of ACE Proteins

A physician or veterinarian can start doses of an ACE protein employed in the pharmaceutical composition at levels lower than that required to achieve the desired therapeutic effect and gradually increase the dosage until the desired effect is achieved. In general, effective doses of the compositions vary depending upon many different factors, including the specific disease or condition to be treated, means of administration, target site, physiological state of the patient, whether a patient is human or an animal, other medications administered, and whether treatment is prophylactic or therapeutic. Treatment dosages typically require titration to optimize safety and efficacy. For administration with an ACE protein, dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. Dosing can be daily, weekly, bi-weekly, monthly, or more or less often, as needed or desired. An exemplary treatment regime entails administration once weekly, once per every two weeks or once a month or once every 3 to 6 months.

The ACE protein can be administered in single or divided doses. An ACE protein is usually administered on multiple occasions. Intervals between single dosages can be weekly, bi-weekly, monthly or yearly, as needed or desired. Intervals can also be irregular as indicated by measuring blood levels of ACE protein in the patient. In some methods, dosage is adjusted to achieve a plasma ACE protein concentration of 1-1000 μg/ml and in some methods 25-300 μg/ml. Alternatively, ACE proteins can be administered as a sustained release formulation, in which case less frequent administration is required. Dosage and frequency vary depending on the half-life of the ACE protein in the patient. In general, antibody engrafted proteins comprising humanized antibodies show longer half-life than that of native cytokines. Dosage and frequency of administration can vary depending on whether treatment is prophylactic or therapeutic. In general for prophylactic applications, a relatively low dosage is administered at relatively infrequent intervals over a long period of time. Some patients continue to receive treatment for the duration of their lives. In general for therapeutic applications, a relatively high dosage in relatively short intervals is sometimes required until progression of the disease is reduced or terminated, and preferably until the patient shows partial or complete amelioration of symptoms of disease. Thereafter, a patient may be administered a prophylactic regime.

Co-Administration with a Second Agent

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one aspect, the present disclosure provides a method of treating cancer by administering to a subject in need thereof an ACE protein in combination with one or more tyrosine kinase inhibitors, including but not limited to, EGFR inhibitors, Her2 inhibitors, Her3 inhibitors, IGFR inhibitors, and Met inhibitors.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); nilotinib (Tasigna®); Regorafenib (Stivarga®) and Imatinib or Imatinib mesylate (Gilvec® and Gleevec®).

Epidermal growth factor receptor (EGFR) inhibitors include but are not limited to, Erlotinib hydrochloride (Tarceva®), Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PM166, CAS 187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Human Epidermal Growth Factor Receptor 2 (HER2 receptor) (also known as Neu, ErbB-2, CD340, or p185) inhibitors include but are not limited to, Trastuzumab (Herceptin®); Pertuzumab (Omnitarg®); Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin-3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGF1R inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another aspect, the present disclosure provides a method of treating cancer by administering to a subject in need thereof an ACE protein in combination with one or more FGF downstream signaling pathway inhibitors, including but not limited to, MEK inhibitors, Braf inhibitors, PI3K/Akt inhibitors, SHP2 inhibitors, and also mTor inhibitors.

For example, mitogen-activated protein kinase (MEK) inhibitors include but are not limited to, XL-518 (also known as GDC-0973, Cas No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

Phosphoinositide 3-kinase (PI3K) inhibitors include but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6).

mTor inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(“L-arginylglycyl-L-α-aspartylL-serine” disclosed as SEQ ID NO: 3979), inner salt (SF1126, CAS 936487-67-1).

In yet another aspect, the present disclosure provides a method of treating cancer by administering to a subject in need thereof an ACE protein in combination with one or more pro-apoptotics, including but not limited to, IAP inhibitors, Bcl2 inhibitors, MCL1 inhibitors, Trail agents, Chk inhibitors.

For examples, IAP inhibitors include but are not limited to, NVP-LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, 052006/0014700, 052006/0025347, WO 06/069063, WO 06/010118, WO 06/017295, and WO08/134679.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinyl-pyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (Sha et al., Mol. Cancer. Ther 2007; 6(1):147-153), and CBP501.

In one aspect, the present disclosure provides a method of treating cancer by administering to a subject in need thereof an ACE protein in combination with one or more FGFR inhibitors. For example, FGFR inhibitors include but are not limited to, Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Vargatef (BIBF1120, CAS 928326-83-4); Dovitinib dilactic acid (TKI258, CAS 852433-84-2); 3-(2,6-Dichloro-3,5-dimethoxy-phenyl)-1-{6-[4-(4-ethyl-piperazin-1-yl)-phenylamino]-pyrimidin-4-yl}-1-methyl-urea (BGJ398, CAS 872511-34-7); Danusertib (PHA-739358); and (PD173074, CAS 219580-11-7). In a specific aspect, the present disclosure provides a method of treating cancer by administering to a subject in need thereof an antibody drug conjugate in combination with an FGFR2 inhibitor, such as 3-(2,6-dichloro-3,5-dimethoxyphenyl)-1-(6((4-(4-ethylpiperazin-1-yl)phenyl)amino)pyrimidin-4-yl)-1-methylurea (also known as BGJ-398); or 4-amino-5-fluoro-3-(5-(4-methylpiperazin1-yl)-1H-benzo[d]imidazole-2-yl)quinolin-2(1H)-one (also known as dovitinib or TKI-258). AZD4547 (Gavine et al., 2012, Cancer Research 72, 2045-56, N-[5-[2-(3,5-Dimethoxyphenyl)ethyl]-2H-pyrazol-3-yl]-4-(3R,5S)-diemthylpiperazin-1-yl)benzamide), Ponatinib (AP24534; Gozgit et al., 2012, Mol Cancer Ther., 11; 690-99; 3-[2-(imidazo[1,2-b]pyridazin-3-yl)ethynyl]-4-methyl-N-{4-[(4-methylpiperazin-1-yl)methyl]-3-(trifluoromethyl)phenyl}benzamide, CAS 943319-70-8).

The ACE proteins can also be administered in combination with another cytokine, or ACE protein. In some embodiments, the cytokine is IL15, IL15-Fc, IL15 linked to a sushi domain of IL15 receptor or IL15/soluble IL15Ra. In some embodiments, the cytokine is interleukin-10 (IL-10), interleukin-11 (IL-11), Ciliary neurotrophic factor (CNTF), Oncostatin M (OSM) or leukemia inhibitory factor (LIF).

The ACE proteins can also be administered in combination with an immune checkpoint inhibitor. In one embodiment, the ACE proteins can be administered in combination with an inhibitor of an immune checkpoint molecule chosen from one or more of PD-1, PD-L1, PD-L2, TIM3, CTLA-4, LAG-3, CEACAM-1, CEACAM-5, VISTA, BTLA, TIGIT, LAIR1, CD160, 2B4 or TGFR In one embodiment, the immune checkpoint inhibitor is an anti-PD-1 antibody, wherein the anti-PD-1 antibody is selected from Nivolumab, Pembrolizumab or Pidilizumab. In some embodiments, the anti-PD-1 antibody molecule is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168.

In some embodiments, the anti-PD-1 antibody is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611.

Other anti-PD1 antibodies include AMP 514 (Amplimmune) and, e.g., anti-PD1 antibodies disclosed in U.S. Pat. No. 8,609,089, US 2010/028330, and/or US 2012/0114649 and US2016/0108123.

In some embodiments, the ACE proteins can be administered with the anti-Tim3 antibody disclosed in US2015/0218274. In other embodiments, the ACE proteins can be administered with the anti-PD-L1 antibody disclosed in US2016/0108123, Durvalumab® (MEDI4736), Atezolizumab® (MPDL3280A) or Avelumab®, or the anti-PD-L1 antibody disclosed in WO2016/061142.

In some embodiments, the pharmacological compositions comprise a mixture of an antibody cytokine engrafted protein and one or more additional pharmacological agent(s). Exemplary second agents for inclusion in mixtures with the present antibody cytokine engrafted protein include without limitation anti-inflammatory agents, immunomodulatory agents, aminosalicylates, and antibiotics. Appropriate selection may depend on preferred formulation, dosage and/or delivery method.

In some embodiments an antibody cytokine engrafted protein is co-formulated (i.e., provided as a mixture or prepared in a mixture) with an anti-inflammatory agent. In particular embodiments, corticosteroid anti-inflammatory agents can be used in conjunction with the antibody cytokine engrafted protein. Corticosteroids for use can be selected from any of methylprednisolone, hydrocortisone, prednisone, budenisonide, mesalamine, and dexamethasone. Appropriate selection will depend on formulation and delivery preferences.

In some embodiments, an antibody cytokine engrafted protein is co-formulated with an immunomodulatory agent. In particular embodiments, the immunomodulatory agent is selected from any of 6-mercaptopurine, azathioprine, cyclosporine A, tacrolimus, and methotrexate. In a particular embodiment, the immunomodulatory agent is selected from an anti-TNF agent (e.g., infliximab, adalimumab, certolizumab, golimumab), natalizumab, and vedolizumab.

In some embodiments an antibody cytokine engrafted protein is co-formulated with an aminosalicylate agent. In particular embodiments, an aminosalicylate is selected from sulfasalazine, mesalamine, balsalazide, olsalazine or other derivatives of 5-aminosalicylic acid.

In some embodiments an antibody cytokine engrafted protein is co-formulated with an antibacterial agent. Exemplary antibacterial agents include without limitation sulfonamides (e.g., sulfanilamide, sulfadiazine, sulfamethoxazole, sulfisoxazole, sulfacetamide), trimethoprim, quinolones (e.g., nalidixic acid, cinoxacin, norfloxacin, ciprofloxacin, ofloxacin, sparfloxacin, fleroxacin, perloxacin, levofloxacin, garenoxacin and gemifloxacin), methenamine, nitrofurantoin, penicillins (e.g., penicillin G, penicillin V, methicilin oxacillin, cloxacillin, dicloxacillin, nafcilin, ampicillin, amoxicillin, carbenicillin, ticarcillin, mezlocillin, and piperacillin), cephalosporins (e.g., cefazolin, cephalexin, cefadroxil, cefoxitin, cefaclor, cefprozil, cefuroxime, cefuroxime acetil, loracarbef, cefotetan, ceforanide, cefotaxime, cefpodoxime proxetil, cefibuten, cefdinir, cefditoren pivorxil, ceftizoxime, ceftriaxone, cefoperazone, ceftazidime, and cefepine), carbapenems (e.g., imipenem, aztreonam), and aminoglycosides (e.g., neomycin, kanamycin, streptomycin, gentamicin, toramycin, netilmicin, and amikacin).

### EXAMPLES

### Example 1: Creation of ACE Protein Constructs

ACE proteins were generated by engineering a cytokine sequence into CDR regions of various immunoglobulin scaffolds, then both heavy and light chain immunoglobulin chains were used to generate final ACE proteins. ACE proteins confer preferred therapeutic properties of the cytokine; and have additional beneficial effects, such as increased half-life, and ease of manufacture.

To create ACE proteins, a mature form of a cytokine sequence was inserted into CDR loops of an immunoglobulin chain scaffold. Cytokines chosen for ACE proteins are listed in TABLE 1, with the addition of IL2 ACE molecules. ACE proteins were prepared using a variety of known immunoglobulin sequences which have been utilized in clinical settings as well as germline antibody sequences. Sequences of cytokines in exemplary scaffolds, referred to as GFTX3b and GFTX are depicted in TABLE 2. Insertion points were selected to be the mid-point of the CDR loop based on available structural or homology model data. ACE proteins were produced using standard molecular biology methodology utilizing recombinant DNA encoding the relevant sequences.

The selection of which CDR was chosen for cytokine engraftment was on the parameters of: the required biology, biophysical properties and a favorable development profile. Modeling software was only partially useful in predicting which CDR and which location within the CDR will provide the desired parameters, so therefore all six possible antibody cytokine grafts are made and then evaluated in biological assays. If the required biological activity is achieved, then the nature of the interactions of the ACE molecule with the respective cytokine receptor is resolved.

For the ACE proteins, the structure of the antibody candidate considered for cytokine engrafting was initially solved. Because of the engrafting technology, each ACE protein is constrained by a CDR loop of different length, sequence and structural environments. As such, each cytokine was engrafted into all six CDRs, corresponding to HCDR1, HCDR2, HCDR3 and LCDR1, LCDR2, LCDR3.

For the selection of the insertion point, the structural center of the CDR loop was chosen as this would provide the most space on either side (of linear size 3.8 Å×the number of residues on an adjacent side) and without being bound by any one theory, this provided a stable molecule by allowing the cytokine to more readily fold independently. As the structures of the grafting scaffolds GFTX3b and GFTX were already known, the structural center of each CDR was also known. This usually coincides with the center of the CDR loop sequence as defined using the Chothia numbering format.

In summary, the insertion point in each CDR was chosen on a structural basis, and which CDR graft was best for the cytokine was based on desired biology and biophysical properties. The nature of the cytokine receptor, the cytokine/receptor interactions and the mechanism of signaling also played a role and this was investigated by comparing each individual antibody cytokine molecule for their respective properties.

Lengthy table referenced here
US20200362058A1-20201119-T00001
Please refer to the end of the specification for access instructions.

Lengthy table referenced here
US20200362058A1-20201119-T00002
Please refer to the end of the specification for access instructions.

### Example 2: In Vitro Activity of ACE Proteins in Mouse Splenocytes

Cells were isolated from mouse spleens and single cell suspensions were added to each well. Each IL7 ACE protein, recombinant human IL7, or IL7-Fc molecule was added to the wells, and incubated for 30 minutes at 37° C. After 20 minutes, cells were fixed with Cytofix buffer (BD #554655), washed and stained with surface markers. After 30 minutes at room temperature, samples were washed and re-suspended cell pellets were permeabilized with −20° C. Perm Buffer III (BD #558050), washed and stained with pSTAT5 Ab (BD #612567). Cells were acquired on LSR Fortessa and data analyzed with FlowJo® software. Data was graphed with Prism® software.

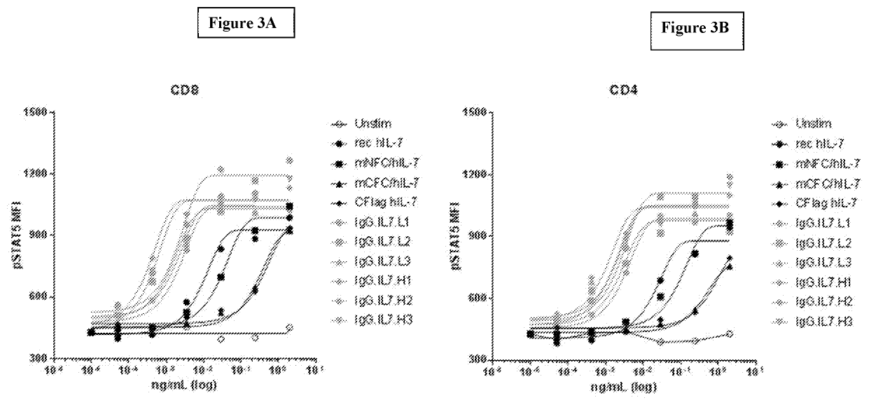

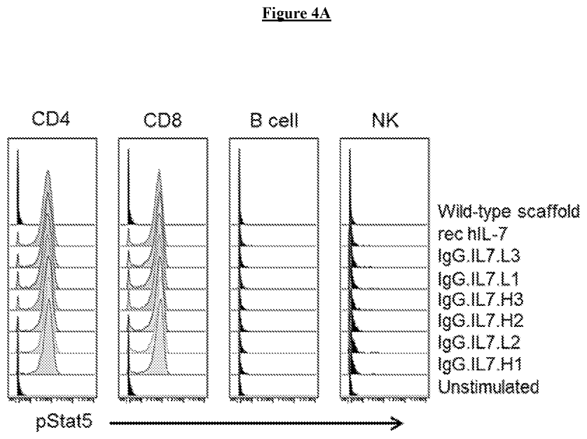

IL7 ACE proteins were assessed for stimulation on the IL7Ra on mouse splenocytes. All of the IL7 ACE proteins displayed increased activation of the IL7Ra pathway on both CD8 (FIG. 3A) and CD4 (FIG. 3B) T cells when compared to equimolar amounts of recombinant human IL7 (rec hIL7), as well as human IL-7 combined to an Fc portion. Thus, grafting of IL7 increases the potency of the cytokine.

### Example 3: In Vitro Activity of IL7ACE Proteins in Human PBMCs

PBMC cells were placed in serum-free test media, and IL7 ACE protein or recombinant human IL7 was added to the cells and incubated for 20 minutes at 37° C. After 20 minutes, cells were fixed with 1.6% formaldehyde, washed and stained with surface markers. After 30 minutes at room temperature, samples were washed and re-suspended cell pellets were permeabilized with −20° C. methanol, washed and stained with pSTAT5 Ab (BD #612567) and DNA intercalators. Cells were run on Cytof and data analyzed with FlowJo® software.

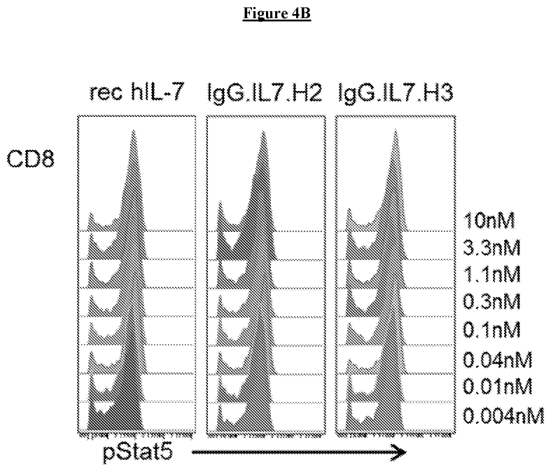

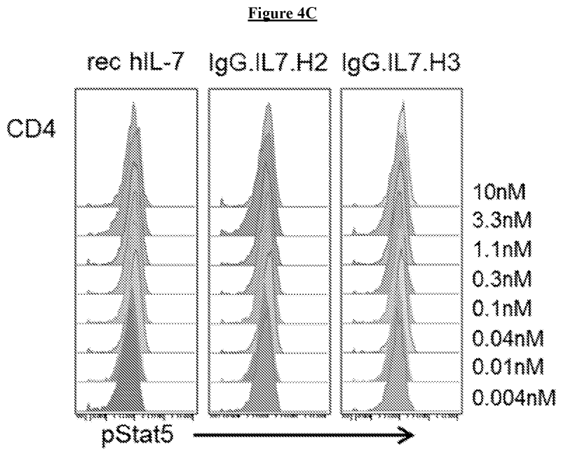

All the molecules tested, independent of its format, induced activation of the IL7Ra pathway on both CD8 and CD4 T cells, but not B cells or NK cells, when compared to wild-type scaffold or unstimulated cells (FIG. 4A). In addition, both CD8 (FIG. 4B) and CD4 (FIG. 4C) T cells were strongly activated, by either recombinant hIL7, or the IL7 ACE proteins IgG.IL7.H3 and IgG.IL7.H2, and independent of the concentration used. Thus, hIL7 ACE proteins strongly stimulate both CD8 and CD4 human T cells, without stimulating B cells or NK cells.

### Example 4: In Vivo Activity of hIL7 ACE Proteins in C57B16 Mice

B6 female mice were administered hIL7, hIL7-Fc and IL7 ACE proteins once a day for 4 days at different concentrations. One day after last treatment (day 5), spleens were processed to obtain a single cell suspension and washed in RPMI (10% FBS). Red blood cells were lysed with Red Blood Cell Lysis Buffer (Sigma #R7757) and cells counted for cell number and viability. FACS staining was performed under standard protocols using FACS buffer (1×PBS+0.5% BSA+0.05% sodium azide). Cells were stained with surface antibodies: Rat anti-mouse CD8-BUV737 (BD Biosciences #564297), Rat anti-mouse CD19-PeCF594/TR (BD Biosciences #562291), Rat anti-mouse CD3-PerCP (Biolegend #100218), Rat anti-mouse CD127-e450 (ebioscience #48-1273-82), Rat anti-mouse CD4-BV510 (BD Biosciences #563106), Rat anti-mouse CD44-BV711 (BD Biosciences #563971), Rat anti-mouse CD62L-APC-Cy7 (BD Biosciences #560514), and subsequently fixed/permeabilized and stained for both Rat anti-mouse Ki-67-e660 (ebioscience #50-5698-82) and FoxP3 according to the Anti-Mouse/Rat FoxP3 FITC Staining Set (ebioscience #71-5775-40). Cells were analyzed on the BD LSR Fortessa or BD FACS LSR II, and data analyzed with FlowJo® software. Data was graphed with Prism software.

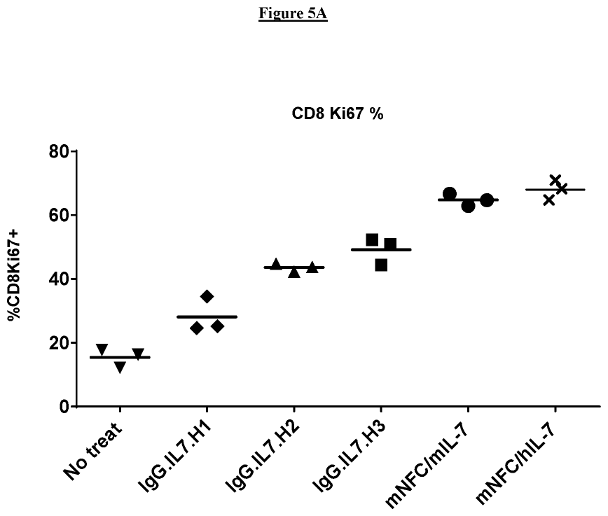

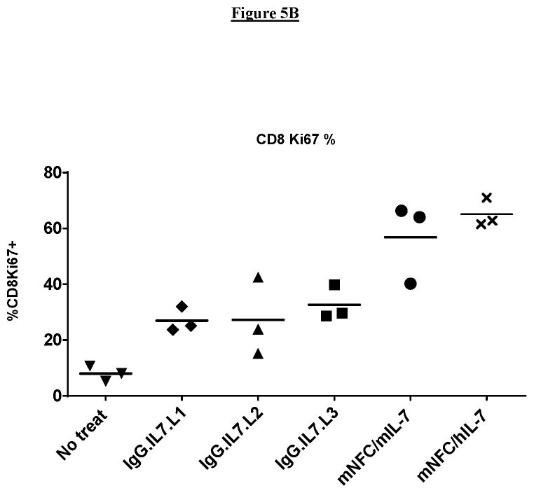

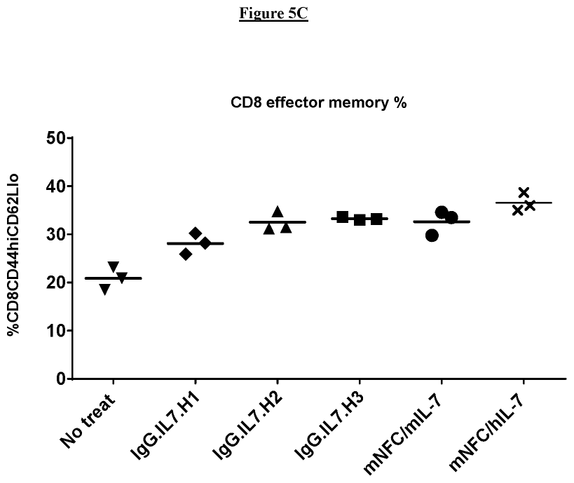

From the six different IL7 ACE proteins tested, IgG.IL7.H3 and IgG.IL7.H2 consistently increased CD8 Ki67+ T cells (FIG. 5A-B), as well as the frequency of effector memory (CD44_{high} CD62L_{low}) T cells (FIG. 5C-D) after daily IP administration for 4 consecutive days. IgG.IL7.H3 and IgG.IL7.H2 consistently increased CD4+ T cells as well (data not shown). Of note, the molar amount of IL7 ACE proteins was 5 times lower than the amount used for Fc fusion IL7 to achieve the same relative expansion of CD8+ and CD4+ T cells. All of the IL7 ACE proteins were well tolerated by the mice, and no ill effects were seen.

### Example 5: In Vivo Activity of IL7ACE Proteins in a CT26 Syngeneic Mouse Tumor Model

CT26 (ATCC) cells are an aggressive, undifferentiated human colorectal cancer line and frequently used to test anti-cancer activity of molecules in syngeneic mouse models. CT26 cells were grown in sterile conditions in a 37° C. incubator with 5% CO². The cells were cultured in RPMI 1640 media supplemented with 10% FBS. Cells were passaged every 3-4 days. For the day of injection, cells were harvested at passage 11 and re-suspended in HBSS at a concentration of 2.5×10⁶/ml. Cells were Radil tested for mycoplasma and murine viruses. For each mouse, 0.25×10⁶ cells were implanted with subcutaneously injection into right flank using a 28g needle (100 μl injection volume). After implantation, animals were calipered and weighed 3 times per week once tumors were palpable. Caliper measurements were calculated using (L×W×W)/2. Mice were fed with normal diet and housed in SPF animal facility in accordance with the Guide for Care and Use of Laboratory Animals and regulations of the Institutional Animal Care and Use Committee.

When tumors reached about 100 mm³, mice were administered 20-100 μg of IL7-flag, IL7-Fc-fusion, or the IL7 ACE proteins IgG.IL7.H3 and IgG.IL7.H2, intraperitoneally twice per week for a total of 4 doses. Tumors were measured twice a week. Average tumor volumes were plotted using Prism 5 (GraphPad) software. An endpoint for efficacy studies was achieved when tumor size reached a volume of 1000 mm3. Following injection, mice were also closely monitored for signs of clinical deterioration. The mice were monitored for any signs of morbidity, including respiratory distress, hunched posture, decreased activity, hind leg paralysis, tachypnea as a sign for pleural effusions, weight loss approaching 20% or 15% plus other signs, or if their ability to carry on normal activities (feeding, mobility).

One day after last treatment (day 13), spleens and tumors were collected. Spleens were processed to obtain a single cell suspension and washed in RPMI (10% FBS). Red blood cells were lysed with Red Blood Cell Lysis Buffer (Sigma #R7757) and cells counted for cell number and viability. FACS staining was performed under standard protocols using FACS buffer (1×PBS+0.5% BSA+0.05% sodium azide). Cells were stained with the following surface antibodies: Rat anti-mouse CD8-BUV737 (BD Biosciences #564297), Rat anti-mouse CD19-PeCF594/TR (BD Biosciences #562291), Rat anti-mouse CD3-PerCP (Biolegend #100218), Rat anti-mouse CD127-e450 (ebioscience #48-1273-82), Rat anti-mouse CD4-BV510 (BD Biosciences #563106), Rat anti-mouse CD44-BV711 (BD Biosciences #563971), Rat anti-mouse CD62L-APC-Cy7 (BD Biosciences #560514), and subsequently fixed/permeabilized and stained for both Rat anti-mouse Ki-67-e660 (ebioscience #50-5698-82) and FoxP3 according to the Anti-Mouse/Rat FoxP3 FITC Staining Set (ebioscience #71-5775-40). Cells were analyzed on the BD LSR Fortessa® or BD FACS LSR II, and data analyzed with FlowJo® software. Tumors were fixed in formalin and paraffin embedded for Immunohistochemistry staining against mouse CD8 (eBioscience #14-0808-82) and mouse CD4 (Abcam #ab183685). The numbers of positive cells were quantified using Matlab software (MathWorks), Data was graphed with Prism software.

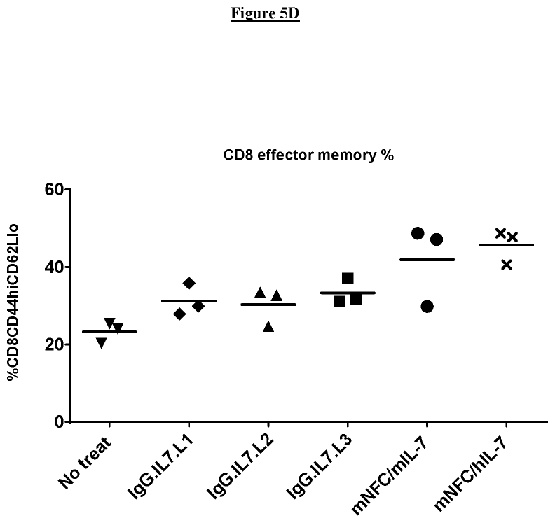

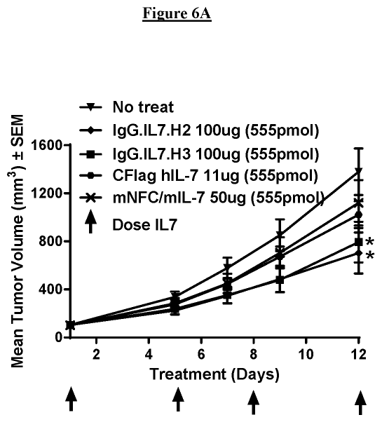

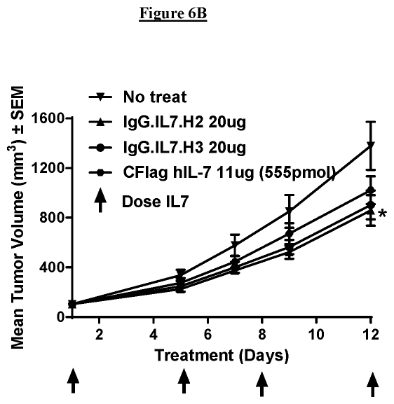

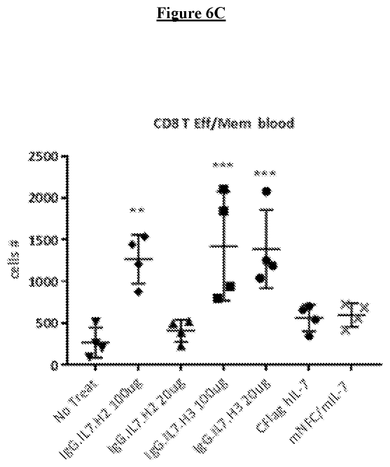

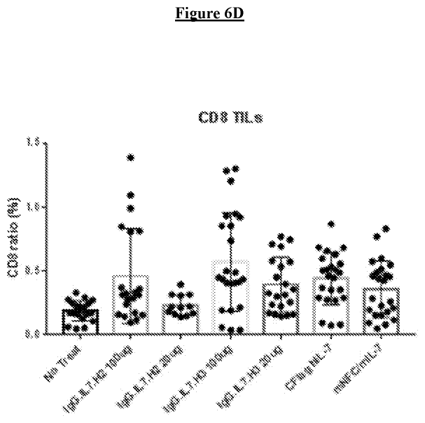

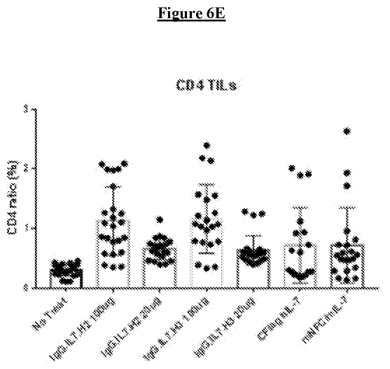

The ACE proteins IgG.IL7.H3 and IgG.IL7.H2 were tested in vivo for their efficacy against CT26 tumors. Administration of IgG.IL7.H2 or IgG.IL7.H2 significantly decreased tumor growth when compared to an IL7 flag protein or an IL7-Fc fusion at equimolar doses (FIG. 6A). The same trend was observed at lower doses (FIG. 6B). Also, the frequency of effector memory (CD44_{high}/CD62L_{low}) CD8+ T cells was significantly increased in mice treated with IgG.IL7.H3 and IgG.IL7.H2 when compared to control groups (FIG. 6C), one day after the last of 4 doses. In addition, the frequencies of both CD8 and CD4+ Tumor Infiltrating Lymphocytes (TILs) were increased at high doses of IgG.IL7.H3 and IgG.IL7.H2 when compared to control groups (FIGS. 6D, and 6E respectively). Therefore, the ACE proteins IgG.IL7.H3 and IgG.IL7.H2 showed enhanced IL7 activity, reduced tumor volume as a single agent, and increased the number of TILs when compared to recombinant IL7.

### Example 6: Activity of IL7 ACE Proteins in an Ex Vivo Model of Exhaustion

B6 female mice were intravenously (iv) infected with 2×10⁶ PFU of Lymphocytic Choriomeningitis Virus (LCMV) clone 13. Three weeks after infection, spleens were collected and processed to obtain a single cell suspension. After B cell depletion using the EasySep™ StemCell B cell depletion kit (Stemcell, Cambridge Mass.) cells were added to wells in RPMI (10% FBS) with a cocktail of three MHC-I and one MHC-II LCMV-specific peptides, with (Media+anti-PD-L1) or without (Media) anti-PD-L1 antibody. INF gamma was measured after 24 hours by ELISA and data was graphed with Prism software.

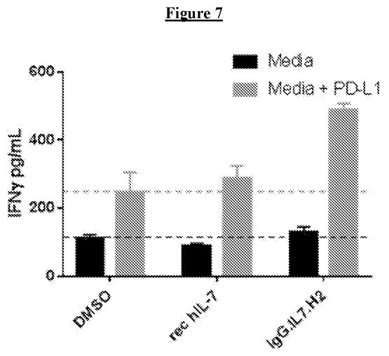

The ACE proteins IgG.IL7.H3 increased IFN-gamma production in synergy with anti-PD-L1 antibody. While the addition of recombinant human IL7 did not result in any further increase in IFN-gamma production respect to anti-PD-L1 treatment (DMSO), addition of IgG.IL7.H3 resulted in a significant increase of IFN-gamma (FIG. 7). Thus, IL7 ACE proteins were able to revert the exhaustion phenotype of CD8+ T cells in an ex vivo model.

### Example 7: Structural Resolution of IgG.IL7.H3 and IgG.IL7.H2

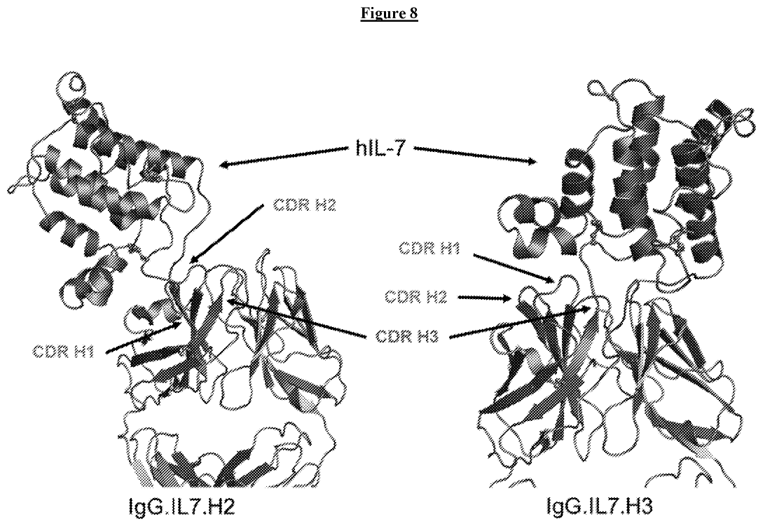

FIG. 8 is a structural diagram of IgG.IL7.H2 and IgG.IL7.H3, respectively as inserted into a Fab fragment. For IgG.IL7.H3, FIG. 8 demonstrates that by engrafting IL7 into either HCDR2 or HCDR3, the IL7 molecule is exposed and available for binding to the IL7Ra, and that the IgG sequences do not interfere.

### Example 8: Binding of Antibody Cytokine Engrafted Proteins

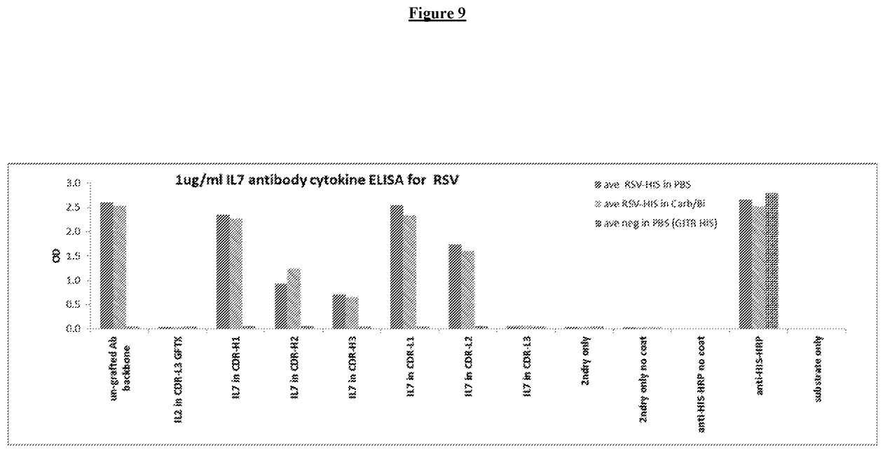

IL7 sequences were inserted into CDR loops of an immunoglobulin chain scaffold. Antibody cytokine engrafted proteins were prepared using a variety of known immunoglobulin sequences which have been utilized in clinical settings as well as germline antibody sequences. One of the antibodies used has RSV as its target antigen. To determine if engrafting IL7 into the CDRs of this antibody reduced binding to RSV, an ELISA assay was run on RSV proteins either in PBS or a carbonate buffer. As shown in FIG. 9, this appears to be influenced by which CDR was chosen for IL7 engrafting. For example, IL7 engrafted into heavy chain CDR1 (CDR-H1) has RSV binding similar to the ungrafted (un-modified) original antibody. In contrast, engrafting IL7 into heavy chain CDR2 (CDR-H2) and into CDR-H3 reduces binding to RSV. IL7 engrafted into light chain CDR3 (CDR-L3) has almost no RSV binding. As expected, IL2 engrafted into a GFTX antibody scaffold which targets IgE produces no binding. This demonstrates that antibody cytokine engrafted proteins can retain binding to the original target of the antibody scaffold, or this binding can be reduced.

### Example 9: In Vivo Pharmacokinetics of IL7 Antibody Cytokine Engrafted Proteins in CD1 Mice

CD1 female mice were administered a single dose of equimolar amounts of IL7, IL7-Fc and IL7 cytokine engrafted proteins, and IL7 protein was measured by Gyros assay in serum samples at different time points (FIG. 10). Data was analysed and graphed with Prism software.

From the six different IL7 proteins tested, IgG.IL7.H2 showed the best exposure when compared to the other formats (FIG. 10). Of note, the amount of recombinant human IL7 was below the Limit of Quantification (LOQ) after just 6 hours, while the same was true for the Fc fusion IL7 after 24 hours. All of the IL7 antibody cytokine engrafted proteins were measurable up to 72 hours.

### Example 10: Activity of IgG.IL7.H2 Cytokine Engrafted Protein in an In Vivo Model of T Cell Exhaustion

B6 female mice were intravenously (iv) infected with 2×10⁶ PFU of Lymphocytic Choriomeningitis Virus (LCMV) clone 13. Three weeks after infection, mice were administered with 200 μg of an Isotype control antibody alone, 100 μg of IgG.IL7.H2 alone, 200 μg of anti-PD-L1 alone, or co-dose with 100 μg of IgG.IL7.H2 plus 200 μg of anti-PD-L1 twice a week for 2 weeks. Three days after last dose (day 35), blood, spleen cells and liver were analysed.

Spleen and blood were processed to obtain a single cell suspension and washed in RPMI (10% FBS). Red blood cells were lysed with Red Blood Cell Lysis Buffer (Sigma #R7757) and cells counted for cell number and viability. FACS staining was performed under standard protocols using FACS buffer (1×PBS+0.5% BSA+0.05% sodium azide). Cells were stained with the following surface antibodies and tetramer molecules: Rat anti-mouse CD8-PerCP (BD Biosciences #553036), Rat anti-mouse CD19-APC-Cγ7 (BD Biosciences #560143), Rat anti-mouse KLRG1-BV421 (BD Biosciences #560733), Rat anti-mouse CD127-PE-Cγ7 (BD Biosciences #560733), Rat anti-mouse CD4-BUV395 (BD Biosciences #563790), Rat anti-mouse CD44-BUV737 (BD Biosciences #564392), Rat anti-mouse CD62L-FITC (Tonbo #35-0621-U100), Rat anti-mouse CD366-APC (Biolegend #119706), Rat anti-mouse CD279-BV605 (Biolegend #135219), T-Select H-2Db LCMV gp33 (C9M) Tetramer-PE (MBL #TS-M512-1) and T-Select H-2Db LCMV gp276-286 Tetramer-BV421 (MBL #TB-5009-4). Cells were analyzed on the BD LSR Fortessa® or BD FACS LSR II, and data analyzed with FlowJo® software. Data was graphed with Prism® software.

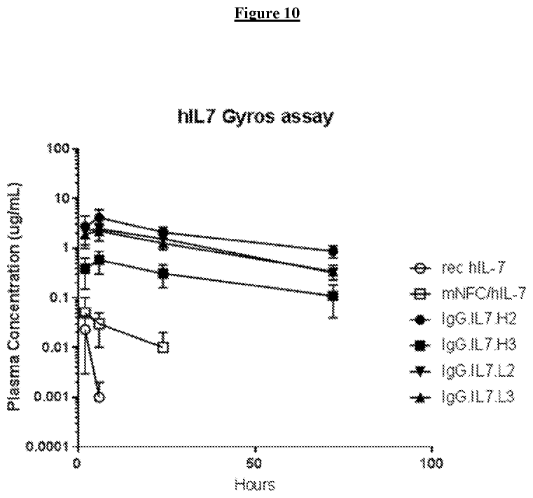

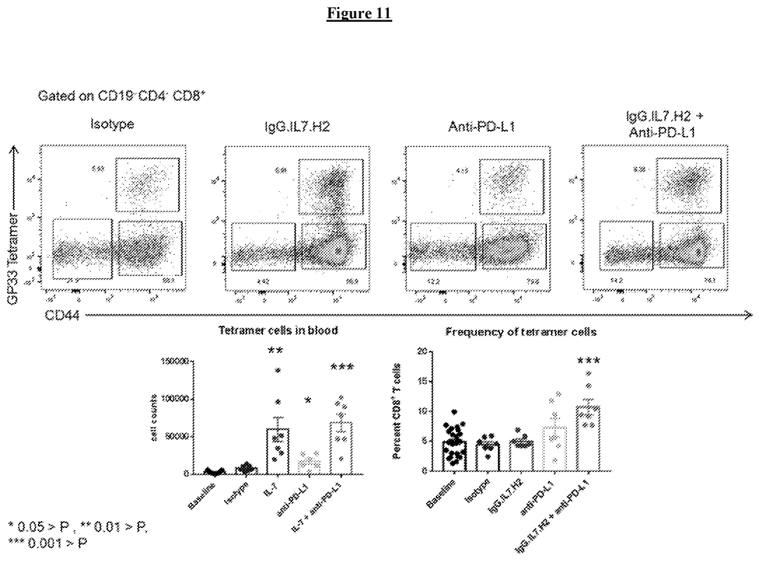

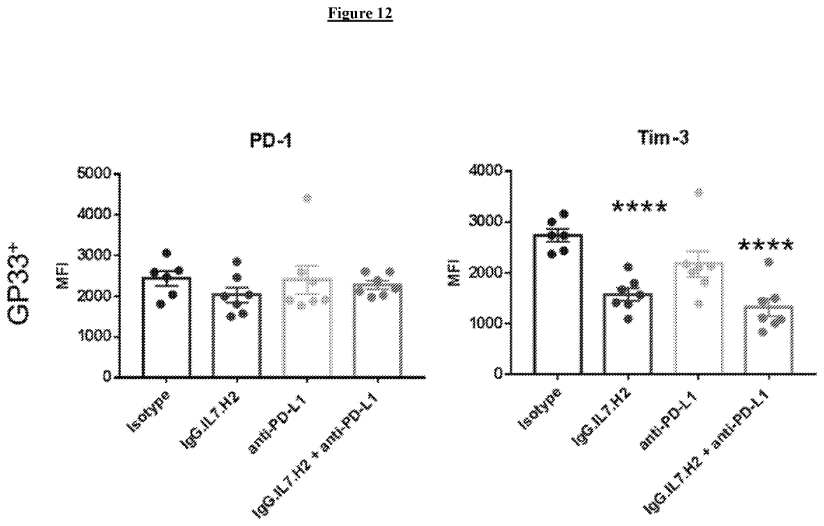

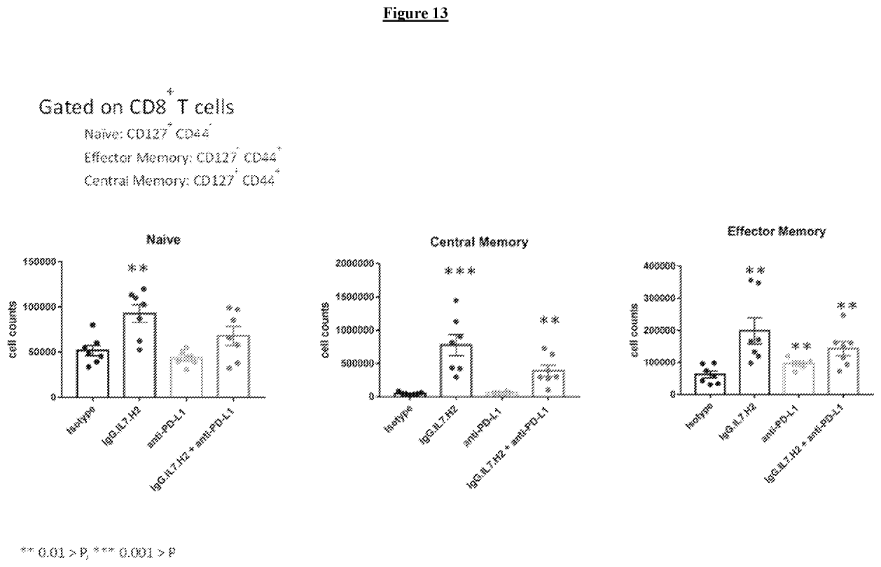

An increase in virus-specific CD8+ T cells was observed in the blood upon dosing with IgG.IL7.H2 antibody cytokine engrafted protein, independent of the presence of anti-PD-L1 antibody (FIG. 11). An increase in total numbers of naïve, central memory and effector memory CD8+ T cells was also observed in the blood upon dosing with IgG.IL7.H2 cytokine engrafted protein, but not with anti-PD-L1 alone (FIG. 13). Analysis of spleen cells also showed that IgG.IL7.H2 antibody cytokine engrafted protein induces the reduction of another checkpoint molecule Tim-3, either alone or in combination with anti-PD-L1 (FIG. 12). In addition, dosing with IgG.IL7.H2 cytokine engrafted protein also induces an increase in CD8+PD-1+ cells, known to be the best responder upon PD-1 blocking therapies (FIG. 14).

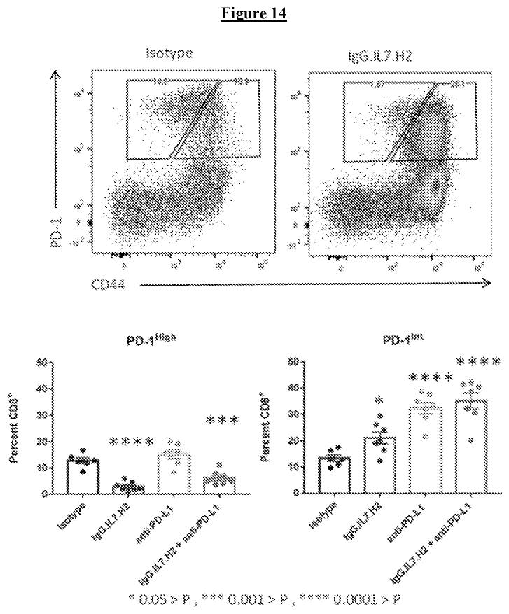

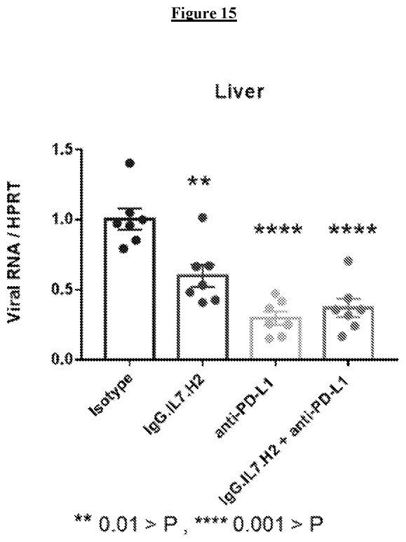

Addition of the IgG.IL7.H2 cytokine engrafted protein, in combination with anti-PD-L1, resulted in a significant increase of IFN-gamma (FIG. 16). Taken together these data indicated that the IgG.IL7.H2 antibody cytokine engrafted protein reverted the exhaustion phenotype of CD8+ T cells in an in vivo model. Analysis of viral RNA in the liver indicates that administration of IgG.IL7.H2 was able to reduce viral load as a single agent (FIG. 15). Anti-PD-L1 antibody and the combination of IgG.IL7.H2 and an anti-PD-L1 antibody further reduced viral load (FIG. 15).

### Example 11: Antibody Cytokine Engrafted Proteins Show Greater Activity on Treg Cells and Increased Half Life

IgG.IL2D49A.H1 and IgG.IL2.L3 were selected as they achieved the desired biological effects over Proleukin® (FIG. 17 summarizes relative changes). These effects include; selectivity for the IL-2R on Tregs vs. Tcon and NK cells, greater half-life expansion of Tregs vs. Tcon and NK cells in mice.

In assessing for high affinity IL-2 receptor stimulation, both Proleukin® and IgG.IL2D49A.H1 graft showed comparable signal potency on Treg cells, but IgG.IL2D49A.H1 showed decreased to no activity on both CD8 Teffector cells and NK cells, unlike Proleukin®. IL2 engrafted into CDRL3 (IgG.IL2.L3) showed less signal potency on Tregs than Proleukin®, but no activity on NK cells. Human Peripheral blood mononuclear cells (hPBMC) were purchased from HemaCare Corp. and tested in vitro with either Proleukin®, IgG.IL2D49A.H1 or IgG.IL2.L3 to assess selective activity on the IL-2 high affinity receptor. Cells were rested in serum free test media, and added to each well. Either antibody cytokine engrafted protein or native human IL-2 were added to the wells, and incubated for 20 min at 37° C. After 20 min, cells were fixed, stained with surface markers, permeabilized and stained with STAT5 antibody (BD Biosciences) following manufacturer's instructions.

Pharmacokinetics of IgG.IL2D49A.H1 or IgG.IL2.L3 in plasma showed an extended half-life over Proleukin® after only 1 dose. Cellular expansion was assessed in the spleen of pre-diabetic NOD mice 8 days after one treatment with either Proleukin® or the grafts. IgG.IL2D49A.H1 achieved superior Treg expansion over Teffector cells and NK cells and was better tolerated than Proleukin® in pre-diabetic mice. The summary of the STAT5 stimulation, the PK/PD of IgG.IL2D49A.H1 and IgG.IL2.L3 is shown in FIG. 18. This shows that antibody cytokine engrafted proteins can not only have greater half-life than Proleukin®, but stimulation of the targeted Treg cells, without unwanted stimulation of Teffector and NK cells.

### Example 12: Antibody Cytokine Engrafted Protein Shows Greater Activity on Treg Cells

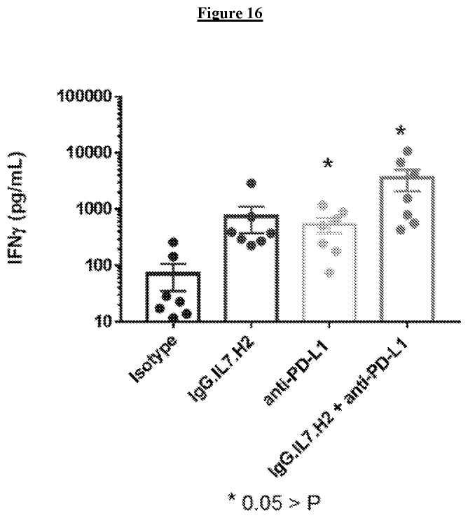

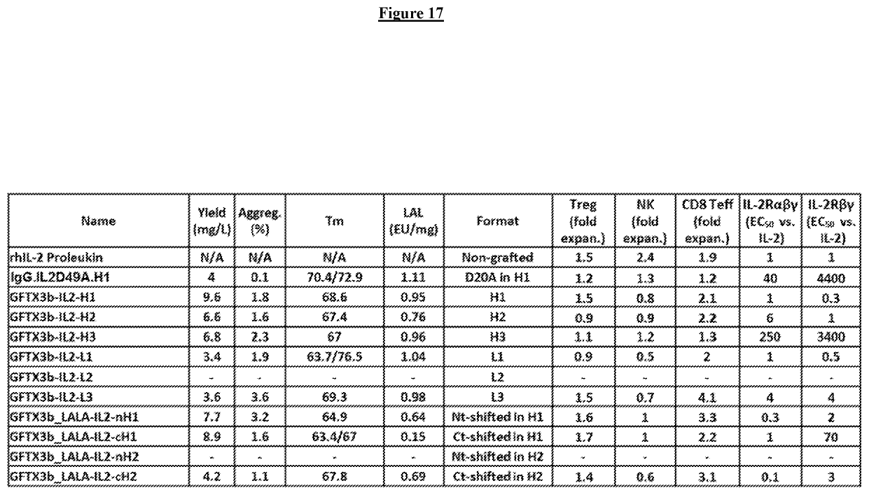

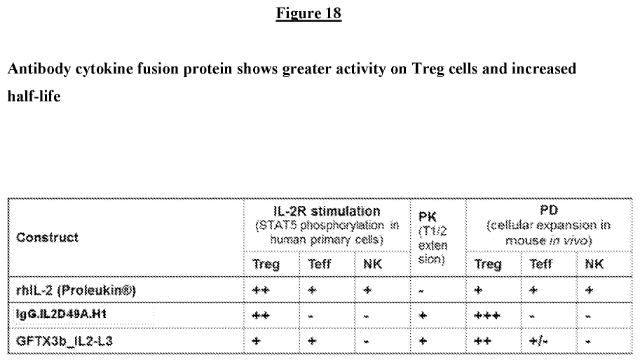

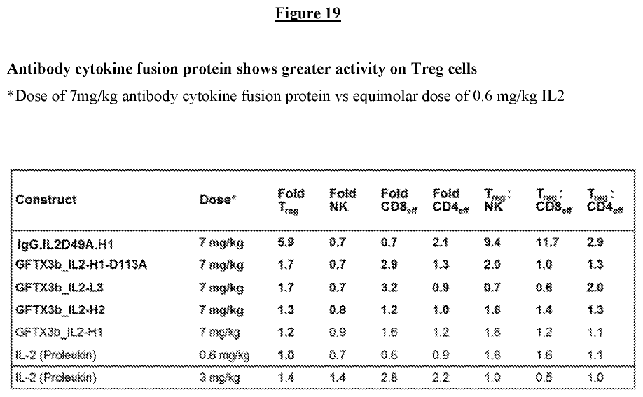

Pre-diabetic NOD mice were administered equimolar Proleukin® (3× weekly) and different antibody cytokine engrafted proteins (1×/week). Eight days after first treatment, spleens were processed to obtain a single cell suspension and washed in RPMI (10% FBS). Red blood cells were lysed with Red Blood Cell Lysis Buffer (Sigma #R7757) and cells counted for cell number and viability. FACS staining was performed under standard protocols using FACS buffer (1×PBS+0.5% BSA+0.05% sodium azide). Cells were stained with surface antibodies: Rat anti-mouse CD3-BV605 (BD Pharmingen #563004), Rat anti-mouse CD4-Pacific Blue (BD Pharmingen #558107), Rat antimouse CD8-PerCp (BD Pharmingen #553036), CD44 FITC (Pharmingen #553133) Rat anti-mouse CD25-APC (Ebioscience #17-0251), and subsequently fixed/permeabilized and stained for FoxP3 according to the Anti-Mouse/Rat FoxP3 Staining Set PE (Ebioscience #72-5775). Cells were analyzed on the BD LSR Fortessa® or BD FACS LSR II®, and data analyzed with FlowJo® software. FIG. 19 shows the fold values and ratios calculated from each spleen as an absolute number, comparing IgG.IL2D49A.H1 and IgG.IL2D113A.H1 with Proleukin®. The increased expansion of Treg cells without expansion of CD 8 T effector cells or NK cells with IgG.IL2D49A.H1 is shown in the top row. This is in contrast to low dose and higher dose Proleukin®, which leads to expansion of all cell types.

### Example 13: IL-2R Signaling Potency is Reduced in CD4 Tcon and CD8 Teff but not in Tregs In Vitro

Both Proleukin® and IgG.IL2D49A.H1 were tested in vitro for signal potency on the IL-2R, on both human and cynomologus monkey PBMC. Both IgG.IL2D49A.H1 and Proleukin® at equimolar IL2 concentrations showed similar signal potency on the Treg cells which express high affinity IL-2R, but only IgG.IL2D49A.H1 showed reduced potency on conventional CD4 and CD8 T effector cells which express the low affinity IL-2 receptor. These results were observed in both human and cynomolgus PBMC. For the assay, PBMC cells were rested in serum-free test media, and added to each well. Either IgG.IL2D49A.H1 or Proleukin® were added to the wells, and incubated for 20 minutes at 37° C. After 20 minutes, cells were fixed, stained with surface markers, permeabilized and stained with STAT5 antibody (BD Biosciences) following manufacturer's instructions. Cells were analyzed on the BD LSR Fortessa® and data analyzed with FlowJo® software.

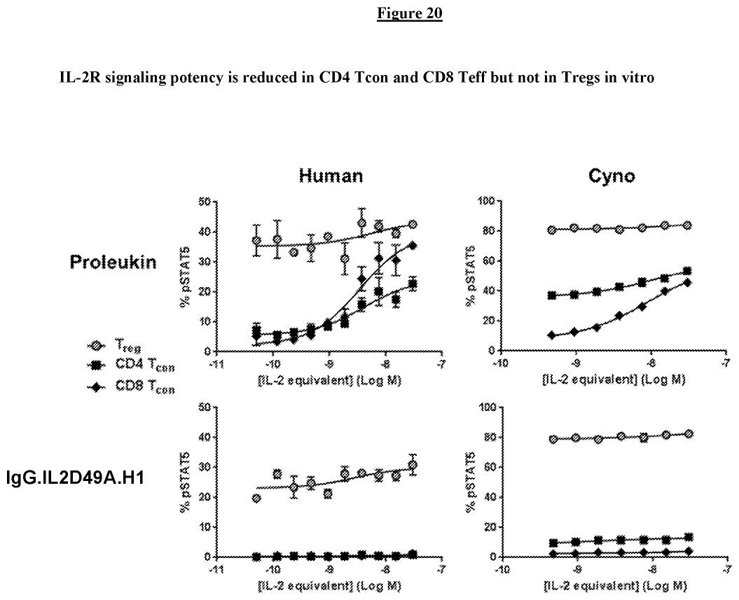

This result as shown in FIG. 20 was especially apparent. Both in human and cynomolgus PBMC, pSTAT5 activation by IgG.IL2D49A.H1 was found on Tregs, with very little on CD8 T effectors.

### Example 14: IgG.IL2D49A.H1 expands functional and stable Tregs in vitro

Improved selectivity for Tregs is accompanied by a functional effect. Tregs expanded with IgG.IL2D49A.H1 are equivalent or better suppressors of Teffectors than Proleukin® expanded Tregs. For this assay, human PBMC were purified from whole blood by centrifugation over Ficoll-Hypaque gradients (GE HealthCare cat #17-1440-03). PBMCs were RBC Lysed (Amimed cat #3-13F00-H). CD4+ Tcells were enriched using EasySep CD4+ T-cell enrichment kit (StemCell Technologies cat #19052). Enriched CD4+ were stained with V500 anti-CD4 (clone RPAT4), PerCP-Cy5.5 anti-CD127 (and APC anti-CD25 and sorted to isolate CD4+CD127-CD25+ natural regulatory T cells (nTregs) and CD4+CD127+CD25− T responder (Tresp). Sorted Tregs were plated (1×10⁵/100 μl/well) in replicates in 96-well round-bottom microplates filled with medium and stimulated with microbeads at 3:1 bead-to-cell ratios in the presence of 1 or 0.3 nM Proleukin® or IgG.IL2D49A.H1 at equimolar IL2 concentrations. After 24 hour incubation at 37° C., wells were refilled with 100 μl medium containing the same IL2 concentration. On day 3, cultures were suspended, split in half and refilled with 100 μl medium containing the same IL2 concentration. On day 6, cultures were processed as on day 3. On day 8, cells were harvested, pooled in tubes and the beads removed by placing tubes on a multistand magnet for 1-2 minutes. Supernatants containing cells were collected and centrifuged at 200 g for 5 minutes at room temperature. Cells were then counted, and plated again at about 5×10⁵/ml in 48-well flat-bottom microplates filled with medium containing 1/5 of the original IL2 concentration. After 2 days rest, cells were harvested, counted and analyzed or used in suppression assay. Expanded Tregs and freshly thawed CD4+CD127+CD25− T responder (Tresp) cells were labeled as described in manufacturer's instructions with 0.8 μM CTViolet (Life Technologies cat #C34557) and 1 μM CFSE (Life Technologies cat #C34554), respectively. To assess the suppressive properties of expanded Tregs, 3×10⁴ CFSE-labeled Tresp were plated in triplicates alone or with CTViolet-labeled Tregs (different Tresp:Treg ratio) and stimulated with Dynabeads at 1:8 bead-to-cell ratio (final volume 200 μl/well). After 4-5 days, cells were collected and the proliferation of responder cells evaluated by flow cytometry.

The methylation status was evaluated in fresh and expanded Tregs compared with Tresp cells. Genomic DNA (gDNA) was isolated from >5.00E+05 cells using Allprep® DNA/RNA Mini from Qiagen (cat #80204). Then, 200 ng of gDNA was processed using Imprint® DNA modification kit from Sigma (cat #MOD50) to convert unmethylated cytosines to uracil (while the methylatd cytosines remain unchanged). Quantitative methylation was then evaluated on 8 ng of bisulfite converted gDNA using sequence-specific probe-based real-time PCR utilizing EpiTect MethyLight® PCR+ROX (Qiagen cat #59496), Epitect control DNA (Qiagen cat #59695), Standard methylated (Life Technologies, cat #12AAZ7FP) and unmethylated (Life Technologies, cat #12AAZ7FP) plasmids, Treg-specific demethylated region (TSDR) methylated and unmethylated forward and reverse primers, and probes (MicroSynth). % of methylation was calculated as described in the EpiTect MethyLight® PCR Handbook.

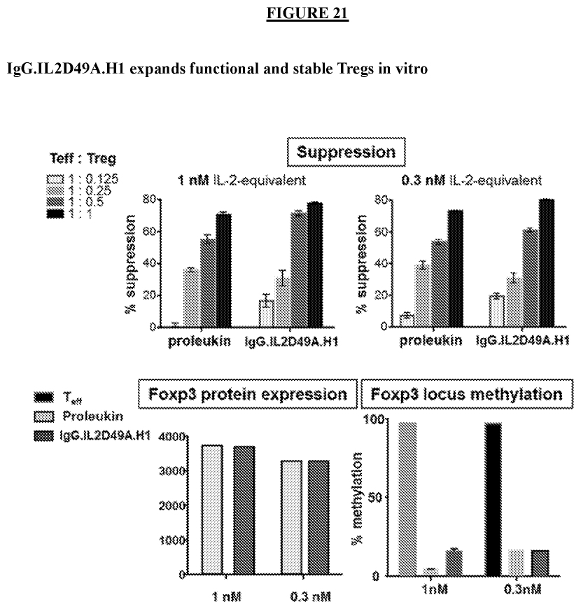

FIG. 21 shows graphically the stable demethylation of the Foxp3 locus with Proleukin® and IgG.IL2D49A.H1 expanded Tregs. Human Tregs expanded with IgG.IL2D49A.H1 in vitro are stable by Foxp3 expression and demethylation, which leads to stable Treg cells.

### Example 15: Potency on IL-2R Signaling Reduced in Human NKs In Vitro with IgG.IL2D49.H1

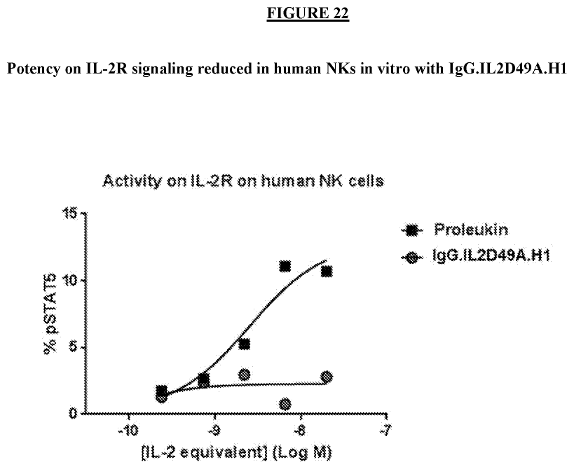

IgG.IL2D49A.H1 showed reduced potency of signaling in NK cells compared to Proleukin® at equimolar concentrations. PBMC cells were rested in serum-free test media, and added to each well. Either IgG.IL2D49A.H1 or Proleukin® were added to the wells, and incubated for 20 minutes at 37° C. After 20 minutes, cells were fixed with 1.6% formaldehyde, washed and stained with surface markers. After 30 minutes at room temperature, samples were washed and re-suspended cell pellets were permeabilized with −20° C. methanol, washed and stained with STAT5 and DNA intercalators. Cells were run on Cytof and data analyzed with FlowJo® software. The results are shown in FIG. 22, wherein IgG.IL2D49A.H1 had little to no effect on NK cells. In contrast, Proleukin® treatement increased pSTAT5 activity on NK cells, as an undesired side effect of the Proleukin® treatment.

### Example 16: Evaluation of the Pharmacokinetic (PK), Pharmacodynamics (PD), and Toxicological Effects of IgG.IL2D49A.H1

IgG.IL2D49A.H1 in cynomolgus monkeys showed extended pharmacokinetics, superior Treg expansion over Teffector cells and less toxicity than low-dose Proleukin®. This nonclinical laboratory study was conducted in accordance with the Novartis Animal Care and Use Committee-approved generic protocol no. TX 4039, with this protocol and with facility Standard Operating Procedures (SOPs).

Animals were dosed subcutaneously with either IgG.IL2D49A.H1 or Proleukin® on the first day of the study. Blood was collected from all animals at each dose level on study. Day 1 at pre-dose, 1 hour, 6 hours and 12 hours post-dose, and then days 2, 3, 4, 5, 6, 7, 8, 10, 12. All blood samples for pharmacokinetics and pharmacodynamics were centrifuged, and plasma samples obtained. Resulting plasma samples were transferred into a single polypropylene tube and frozen at approximately −70° C. or below. All samples were analyzed, and concentrations of IgG.IL2D49A.H1 and Proleukin® in plasma measured using immuno assays. Pharmacokinetic parameters such as half-life were calculated, and cells immunophenotyped by FACS for pharmacodynamics. The IL-2/IL-2 Gyros assay protocol is as follows. Each sample was run in duplicate, with each of the duplicated analyses requiring 5 μL of sample that had been diluted 1:20. Capture antibody is goat anti-human IL-2 biotinylated antibody (R&D Systems BAF202) and detected with Alexa 647 anti-human IL-2, Clone MQ1-17H12 (Biolegend 500315) LOQ: 0.08 ng/ml, all immunoassay were conducted using a Gyrolab Bioaffy200® with Gyros CD-200s®.

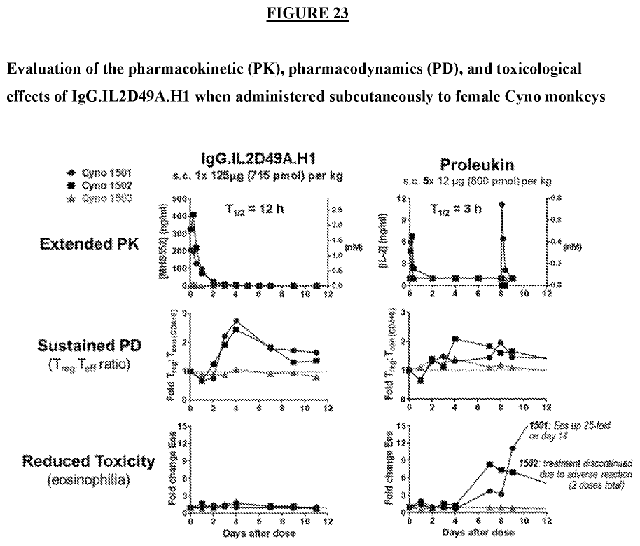

FIG. 23 shows the contrasts between IgG.IL2D49A.H1 and Proleukin®. IgG.IL2D49A.H1 has a half-life of 12 hours, whereas Proleukin® has a half-life of 3 hours. With the extended half-life of IgG.IL2D49A.H1 comes increased Treg activity and much reduced eosinophilia toxicity.

### Example 17: IgG.IL2D49A.H1 Shows an Extended Half-Life Over Proleukin®

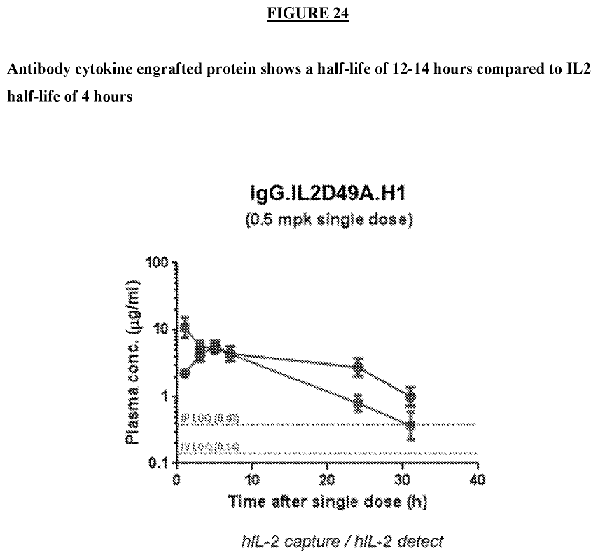

IgG.IL2D49A.H1 showed a half-life of approximately 12 hours compared to the Proleukin® half-life of 4 hours after a single administration. Naïve CD-1 animals were dosed intravenously or subcutaneously and blood collected from all animals at pre-dose, 1 hour, 3, 7, 24, 31, 48, 55 and 72 hours post-dose. Blood samples were centrifuged, and plasma samples obtained. Resulting plasma samples were transferred into a single polypropylene tube and frozen at −80° C. All samples were analysed, and concentrations of IgG.IL2D49A.H1 in plasma was measured using immunoassays. The IL-2/IL-2 Gyros assay protocol is as follows. Each sample was run in duplicate, with each of the duplicated analyses requiring 5 μL of sample that had been diluted 1:20. Capture antibody is goat anti-human IL-2 biotinylated antibody (R&D Systems BAF202) and detected with Alexa 647 anti-human IL-2, Clone MQ1-17H12 (Biolegend 500315) LOQ: 0.08 ng/ml, all immunoassay were conducted using a Gyrolab Bioaffy200® with Gyros CD-200s®. This assay expands upon the half-life determination of Example 16. The results of this assay is shown in FIG. 24, where the half-life of IgG.IL2D49A.H1 is determined to be 12-14 hours, in contrast with Proleukin® which has a half-life of 4 hours.

### Example 18: Human Tregs Expand but not Teffectors or NK Cells in Mice with Xeno-GvHD

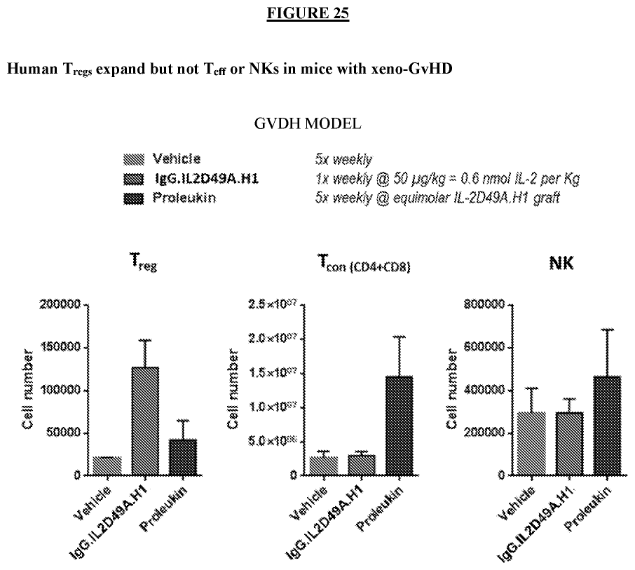

IgG.IL2D49A.H1 selectively expands Tregs over Teffectors or NK cells in the xeno-GvHD model, while Proleukin® does not. NOD-scid IL2R gamma null mice (NSG) were injected with hPBMCs from healthy donors via intraperitoneal injection (HemaCare Corp). 24 hours after injection, the animals were dosed with either IgG.IL2D49A.H1 1×/week or Proleukin® 5×/week every week for the duration of the study. Body weight was monitored twice a week for the duration of the study. Four mice per group were harvested 28 days after the first dose, and spleens were processed to obtain single cell suspensions and washed in RPMI (10% FBS). Red blood cells were lysed with Red Blood Cell Lysis Buffer and cells counted for cell number and viability. FACS staining was performed under standard protocols using FACS buffer (1×PBS+0.5% BSA+0.05% sodium azide). Cells were stained with surface antibodies and subsequently fixed/permeabilized and stained for FoxP3 according to the Anti-Mouse/Rat FoxP3 Staining Set PE (Ebioscience #72-5775). Cells were analyzed on the BD LSR Fortessa® and data analyzed with FlowJo® software. Fold values and ratios are based on the relative number calculated from each spleen absolute number. FIG. 25 shows that IgG.IL2D49A.H1 expands Treg cells much better than Proleukin® in this mouse model and also reduces the undesired expansion of Tcons and NK cells.

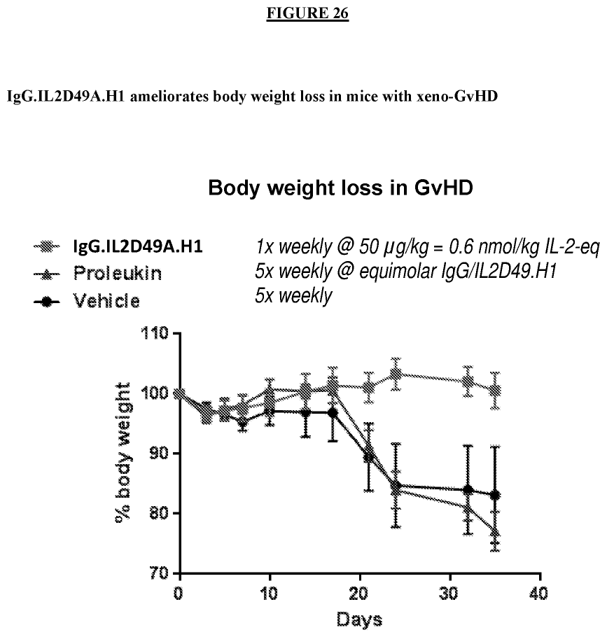

When the xeno-GvHD mice were treated with the IgG.IL2D49.H1, and injected with human PBMCs (the foreign cells), they maintained a normal body weight over the course of the treatment. In contrast, mice treated with Proleukin® had severe body weight loss. Body weight was monitored twice a week for the duration of the study, and percent body weight was calculated taking into consideration the initial weight of the animals at the time of enrollment. This improvement is associated with the effect IgG.IL2D49A.H1 has on Treg enhancement in this model, and the data is shown graphically in FIG. 26. This data indicates that IgG.IL2D49A.H1 and other antibody cytokine engrafted proteins have a greater therapeutic index and margin for safety.

### Example 19: IgG.IL2D49A.H1 Prevents Type 1 Diabetes Development in a NOD Mice Model of Diabetes

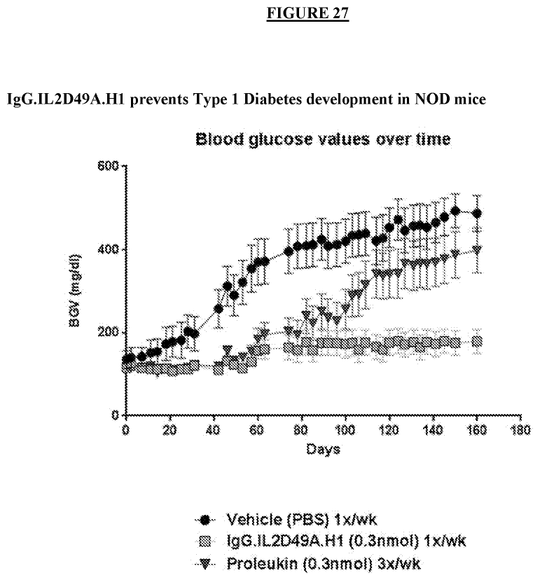

The non-obese diabetic (NOD) mouse develops type 1 diabetes spontaneously and is often used as an animal model for human type 1 diabetes. Pre-diabetic NOD females were administered equimolar Proleukin® (3× weekly) and IgG.IL2D49A.H1 (lx/weekly) by intraperitoneal injection. For the duration of the study (4 months after first dose), the mice were monitored twice a week for blood glucose and body weight. FIG. 27 shows that IgG.IL2D49A.H1 treated mice maintain a low blood glucose value. As such, mice treated with IgG.IL2D49A.H1 did not progress to overt Type 1 diabetes (T1D). In contrast, Proleukin® treated mice began with low blood glucose values, but this increased over time and resulted in type 1 diabetes symptoms.

### Example 20: IgG.IL2D49A.H1 Versus Low Dose Proleukin® in Pre-Diabetic NOD Mice

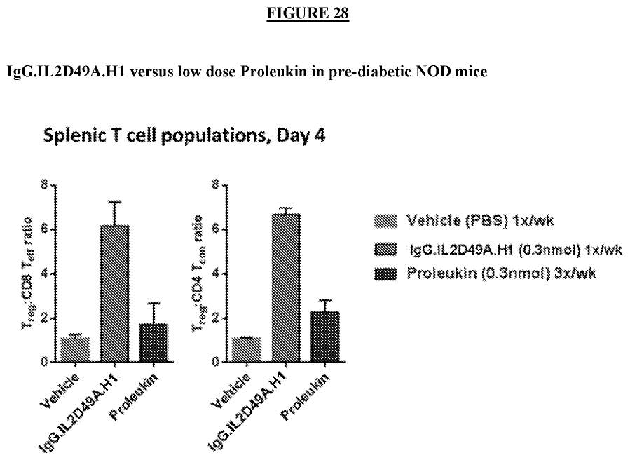

IgG.IL2D49A.H1 showed superior Treg expansion, better tolerability and no adverse events with one dose, compared to 3 doses of Proleukin® in the NOD mouse model. Pre-diabetic NOD females were administered low dose equimolar Proleukin® (3× weekly) and IgG.IL2D49A.H1 (lx/weekly) by intraperitoneal injection. Four mice per group were taken down 4 days after the first dose, and spleens were processed to obtain single cell suspensions and washed in RPMI (10% FBS). Red blood cells were lysed with Red Blood Cell Lysis Buffer and cells counted for cell number and viability. FACS staining was performed under standard protocols using FACS buffer (1×PBS+0.5% BSA+0.05% sodium azide). Cells were stained with surface antibodies: Rat anti-mouse CD3-BV605 (BD Pharmingen #563004), Rat anti-mouse CD4-Pacific Blue (BD Pharmingen #558107), Rat antimouse CD8-PerCp (BD Pharmingen #553036), CD44 FITC (Pharmingen #553133) Rat anti-mouse CD25-APC (Ebioscience #17-0251), and subsequently fixed/permeabilized and stained for FoxP3 according to the Anti-Mouse/Rat FoxP3 Staining Set PE (Ebioscience #72-5775). Cells were analyzed on the BD LSR Fortessa® or BD FACS LSR II®, and data analyzed with FlowJo® software. Fold values and ratios are based on the relative number calculated from each spleen absolute number. Administration of a single dose of IgG.IL2D49A.H1 showed greater expansion of Tregs than repeated administration of Proleukin® in the NOD mouse model as shown in FIG. 28.

### Example 21: Pharmacokinetics of an Efficacious Dose of IgG.IL2D49A.H1 in the NOD Mouse Model

Pharmacokinetics of IgG.IL2D49A.H1 at 1.3 mg/kg and 0.43 mg/kg was assayed in plasma up to 48 hours after 1 dose. Pre-diabetic 10 week old NOD mice were dosed intraperitoneally with IgG.IL2D49A.H1 at two different concentrations and blood collected from all animals at 1 hour, 3, 7, 24 and 48 hours post-dose. Blood samples were centrifuged, and plasma samples obtained. Resulting plasma samples were transferred into a single polypropylene tube and frozen at −80° C. Each sample was analyzed to detect IgG.IL2D49A.H1 plasma concentrations using three different methods adapted to the Gyros platform: 1) IL2-based capture and detect, 2) IL2-based capture and hFc-based detect, and 3) hFc-based capture and detect.

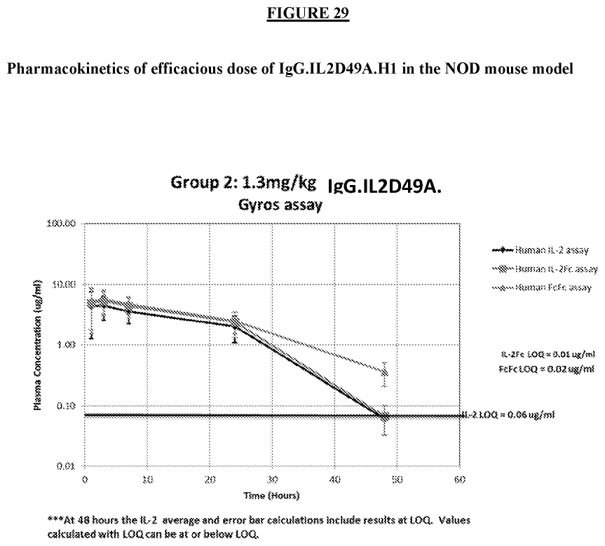

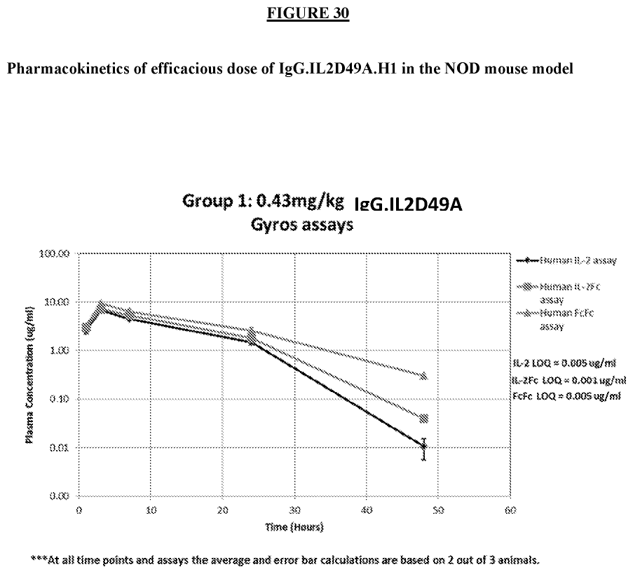

Each sample was run in duplicate, with each of the duplicated analyses requiring 5 μL of sample that had been diluted 1:20. The Gyros IL-2/IL-2 assay uses a capture goat anti-human IL-2 biotinylated antibody (R&D Systems BAF202) and detects with Alexa 647 anti-human IL-2, Clone MQ1-17H12 (Biolegend 500315). For IL-2/Fc detection, a capture goat anti-human IL-2 biotinylated antibody (R&D Systems BAF202) is used, and for detection, an Alexa 647 goat anti-human IgG, Fc specific (Jackson ImmunoResearch 109-605-098) antibody. For the human Fc/Fc assay, a capture Biotinylated goat anti-human IgG, Fc specific (Jackson ImmunoResearch #109-065-098) was used. The detection step used an Alexa 647 goat anti-human IgG, Fcγ specific (Jackson ImmunoResearch #109-605-098). All immunoassays were conducted using a Gyrolab Bioaffy200® with Gyros CD-200s. The limit of quantification (LOQ) in this mouse model is 48 hours as shown in FIG. 29. This is compared with Proleukin® and an IL2-Fc fusion protein in FIG. 30. This graph shows that the LOQ is higher for antibody cytokine engrafted proteins such as IgG.IL2D49.H1.

### Example 22: Dose Range Finding in Pre-Diabetic NOD Mice

IgG.IL2D49A.H1 showed superior Treg expansion over both CD4 Tcon and CD8 Teffectors when compared to Proleukin® at the same equimolar concentrations. Adverse events such as mortality were found in the highest Proleukin® groups, and no mortality was seen in mice treated with any dose of IgG.IL2D49.H1.

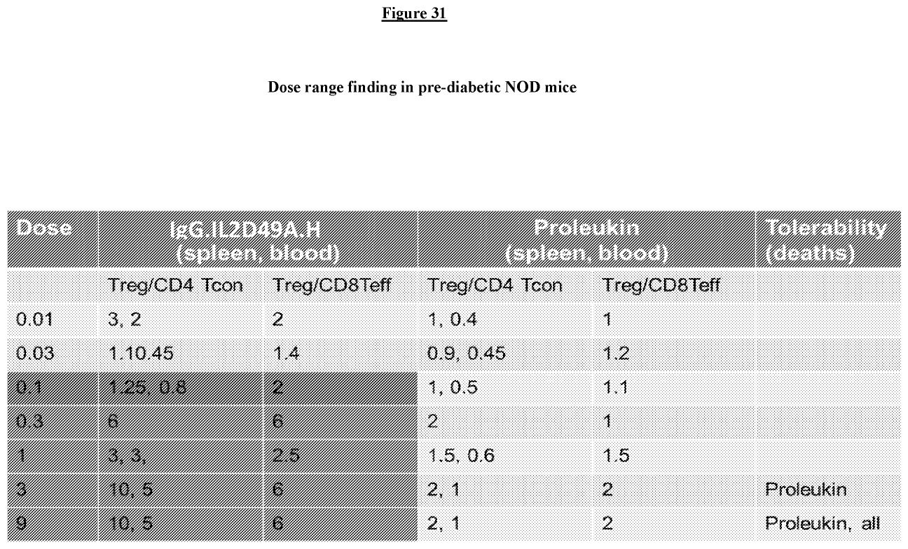

Pre-diabetic NOD females were administered low dose equimolar IL-2 (3× weekly) and IgG.IL2D49A.H1 (lx/weekly) by intraperitoneal injection. Three mice per group were euthanized 8 days days after the first dose and spleens harvested. Spleens were processed to obtain single cell suspensions and washed in RPMI (10% FBS). Blood was collected, red blood cells were lysed with Red Blood Cell Lysis Buffer and cells counted for cell number and viability. FACS staining was performed under standard protocols using FACS buffer (1×PBS+0.5% BSA+0.05% sodium azide). Cells were stained with surface antibodies and subsequently fixed/permeabilized and stained for FoxP3 according to the Anti-Mouse/Rat FoxP3 Staining Set PE (Ebioscience #72-5775). Cells were analyzed on the BD LSR Fortessa® and data analyzed with FlowJo® software. Ratios are based on the relative cell number calculated from each spleen. This data is provided in FIG. 31. The table provides for a dose range format for antibody cytokine engrafted proteins. It also demonstrates that IgG.IL2D49A.H1 had a greater therapeutic index than Proleukin® as dosing was well tolerated over a larger range. In contrast, the administration of Proleukin® at higher doses produced morbidity and mortality in the mice.

### Example 23: STAT5 Signaling on Human PBMC

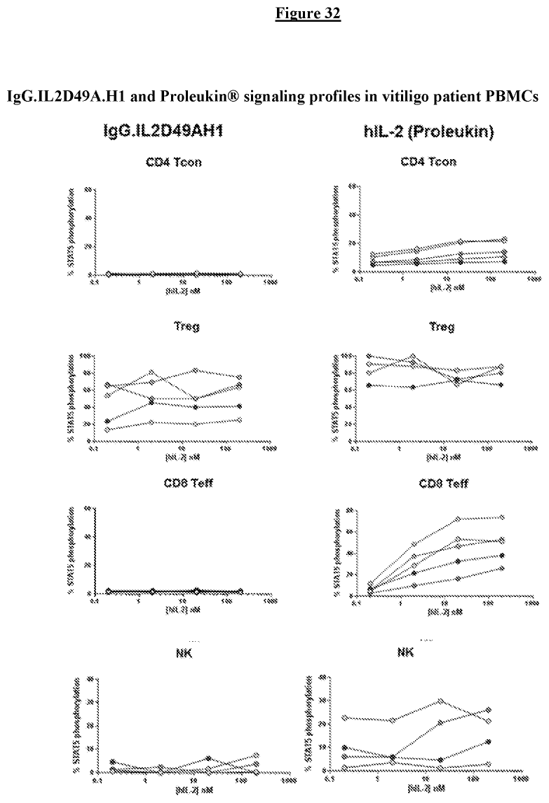

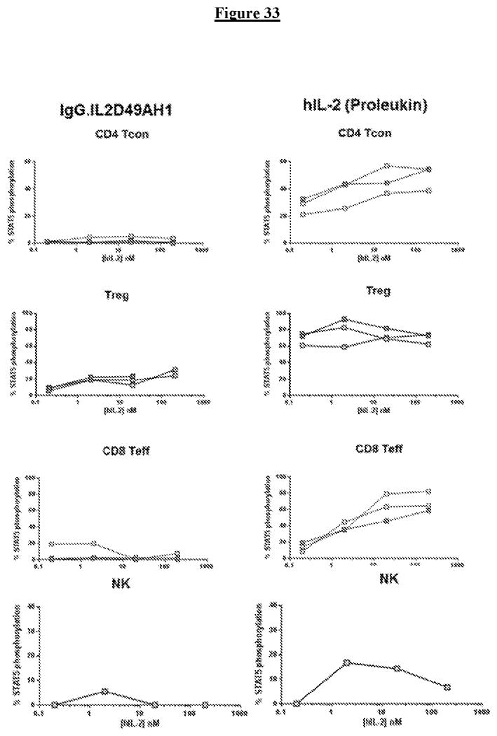

IgG.IL2D49A.H1 was selective for Treg activation over Tcon and NK in healthy donor human PBMC as well as in PBMC from autoimmune donors. Potency of STAT5 signaling was reduced in Tcon but not Tregs after treatment in vitro with IgG.IL2D49.H1. Human PBMC from healthy and autoimmune patients (Hemacare Corp) cells were rested in serum-free test media, and added to each well. IgG.IL2D49A.H1 was added to the wells, and incubated for 20 min at 37° C. After 20 minutes, cells were fixed, stained with surface markers, permeabilized and stained with STAT5 antibody (BD Biosciences) following manufacturer's instructions. Cells were analyzed on the BD LSR Fortessa® and data analyzed with FlowJo® software. The data in FIG. 32 indicates that IgG.IL2D49A.H1 treatment of PBMCs taken from human patients with vitiligo that there was very little activation of NK, CD4 T con, or CD8 T effector cells, while maintaining Treg activity. This result was also observed in PBMCs taken from patients with SLE and Hashimoto's disease (data not shown). FIG. 33 shows that PBMCs taken from human patients with Type 1 Diabetics (T1D) and treated with IgG.IL2D49A.H1 and Proleukin® had much reduced pSTAT5 activity on NK cells, CD8 T effector cells or CD4 Tcon cells. As IgG.IL2D49A.H1 treatment was effective in normal PBMCs and well tolerated in PBMCs taken from T1D patients, this indicates that antibody cytokine proteins would be useful in the treatment of T1D even if the patient is receiving insulin therapy. This indicates that IgG.IL2D49A.H1 would be well tolerated in patients with these immune related disorders, and is effective in dealing with these immune related disorders.

### Example 24: Binding of Antibody Cytokine Engrafted Proteins

Antibody cytokine engrafted proteins were prepared using a variety of known immunoglobulin sequences which have been utilized in clinical settings as well as germline antibody sequences. One of the antibodies used has RSV as its antigen. To determine if engrafting IL2 into the CDRs of this antibody reduced or abrogated binding to RSV, an ELISA assay was run on RSV proteins either in PBS or a carbonate buffer. As shown in FIG. 34, this appears to be influenced by which CDR was chosen for IL2 engrafting. For example, IgG.IL2D49A.H1 has RSV binding similar to the ungrafted (un-modified) original antibody. In contrast, engrafting IL2 into the light chain of CDR3 (CDR-L3) or into CDR-H3 reduces binding. As expected, IL2 engrafted into a GFTX antibody scaffold which targets IgE produces no binding. This demonstrates that antibody cytokine engrafted proteins can retain binding to the original target of the antibody scaffold, or this binding can be reduced.

### Example 25: Treg Expansion in Non-Human Primates

IgG.IL2D49A.H1 was administered to cynomolgus monkeys in two single rising subcutaneous doses given with 4-week dosing free interval alternating between 2 dose groups (3M/group). This was followed by a 2-week multiple dose phase in two groups (3M/group) receiving 6 subcutaneous doses (every other day for two weeks) of buffer or 5 mg/kg IgG.IL2D49A.H1. Changes in lymphocyte populations assessed by flow cytometry (immunophenotyping) from the “single dose phase” (two doses given 29 days apart) are shown in FIG. 35. At the 125 and 375 μg/kg doses, 3-4 fold and up to 5.5 fold increases in absolute numbers of Treg were observed without any apparent effect on Tcon or NK cells. Maximum Treg expansion was seen on day 4 and Treg numbers return to near baseline by day 10. IgG.IL2D49A.H1 was safe and well tolerated and there were no mortalities, clinical signs or changes in body weight, food consumption, cytokine levels or clinical pathology. Furthermore no cardiovascular effects (ECG or blood pressure) were observed in the study after single dose up to 2.4 mg/kg or multiple dosing every other day for two weeks at 5 mg/kg. There was no indication of vascular leak or other CV related findings.

### Example 26: IgG.IL2R67A.H1 Activities and Extended Half-Life

IL2 containing either R67A or F71 A muteins were engrafted into all six CDRs, corresponding to LCDR-1, LCDR-2, LCDR-3 and HCDR-1, HCDR-2 and HCDR-3. From the Table in FIG. 36, it is apparent that the antibody cytokine engrafted proteins differ in their activities, including that IL2 engrafted into the light chain of CDR2 (GFTX3b-IL2-L2) did not express. It was also observed that IL2 when engrafted into HCDR1 with altered Fc function (e.g. Fc silent) had a better biological result on the expansion CD8+T effectors.

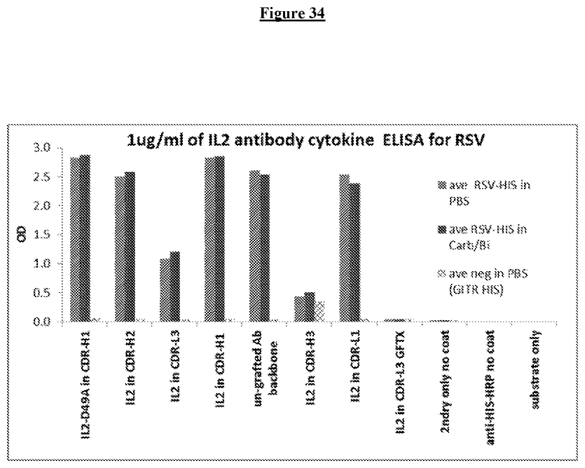

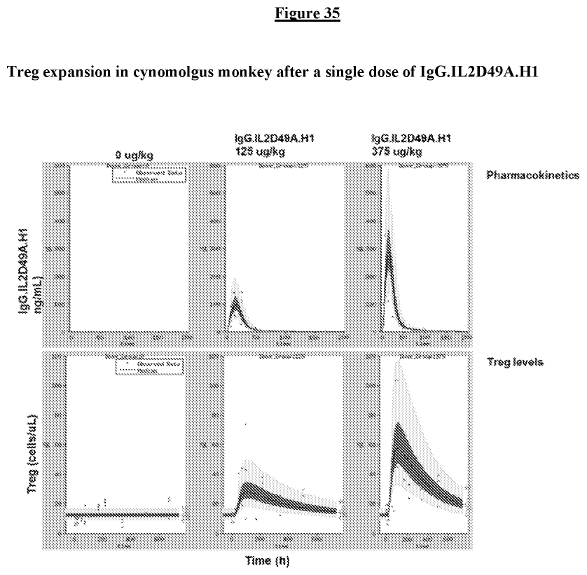

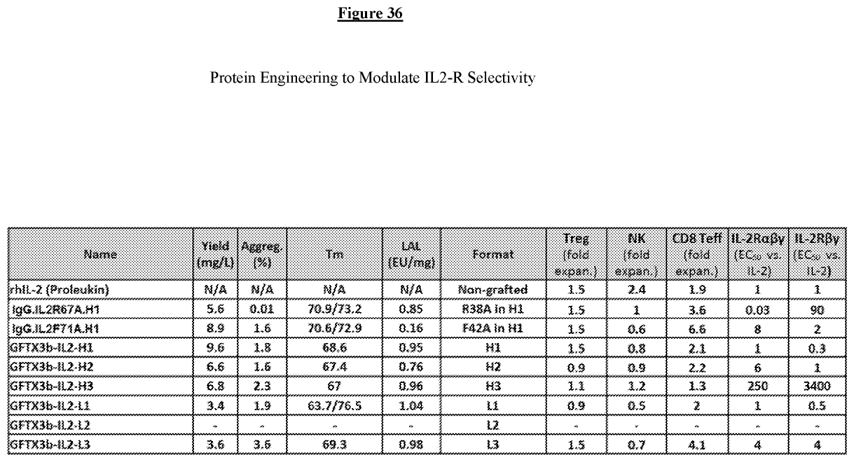

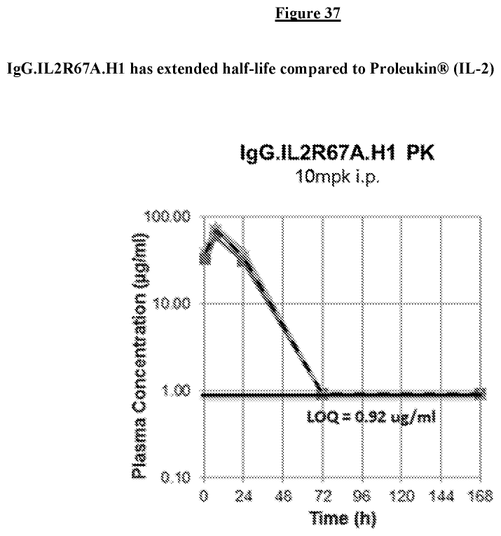

For a half-life determination, naïve CD-1 mice were dosed I.P. and blood collected from all animals at pre-dose, 1 hour, 3, 7, 24, 31, 48, 55 and 72 hours post-dose. Blood samples were centrifuged, and plasma samples obtained. Resulting plasma samples were transferred into a single polypropylene tube and frozen at −80° C. All samples were analyzed, and concentrations of IgG.IL2R67A.H1 in plasma measured using immuno-assays. Pharmacokinetic parameters such as half-life were calculated. Each sample was run in duplicate, with each of the duplicated analyses requiring 5 μL of sample that had been diluted 1:20. Capture: goat anti-human IL-2 biotinylated antibody (R&D Systems BAF202) Detect: Alexa 647 anti-human IL-2, Clone MQ1-17H12 (Biolegend 500315) All immunoassay were conducted using a Gyrolab® Bioaffy200 with Gyros CD-200s. As shown in the graph in FIG. 37, the half-life of IgG.IL2R67A.H1 is approximately 12 hours and then diminishing over the next 48 hours. The Proleukin® half-life could not be shown on this graph as its half-life is approximately 4 hours.

### Example 27: IgG.IL2R67A.H1 Selectively Expands CD8 T Effectors and is Better Tolerated than IL-2 Fc or Proleukin® in Normal B6 Mice

IgG.IL2R67A.H1 augments CD8 T effectors over Tregs without causing the adverse events seen with Proleukin® administration. After dosing mice on day 1, CD8 T effector expansion was monitored at day 4, day 8 and day 11. At each timepoint, the CD8 T effector cell population was greatly expanded, without Treg expansion. This was in contrast to Proleukin® and an IL-2Fc fusion, in which mortality and morbidity were observed at equimolar doses of IL-2.

B6 female mice were administered Proleukin® (5× weekly), IL-2 Fc and IgG.IL2R67A.H1 (1×/week) at equimolar concentrations. Eight days after first treatment, spleens were processed to obtain a single cell suspension and washed in RPMI (10% FBS). Red blood cells were lysed with Red Blood Cell Lysis Buffer (Sigma #R7757) and cells counted for cell number and viability. FACS staining was performed under standard protocols using FACS buffer (1×PBS+0.5% BSA+0.05% sodium azide). Cells were stained with surface antibodies: Rat anti-mouse CD3-efluor 450 (Ebioscience #48-0032), Rat anti-mouse CD4-Pacific Blue (BD Pharmingen #558107), Rat anti-mouse CD8-PerCp (BD Pharmingen #553036), Rat anti-mouse CD44 FITC (Pharmingen #553133), Rat anti-mouse CD25-APC (Ebioscience #17-0251), Rat anti-mouse Nk1.1 (Ebioscience #95-5941) and subsequently fixed/permeabilized and stained for FoxP3 according to the anti-Mouse/Rat FoxP3 Staining Set PE (Ebioscience #72-5775). Cells were analyzed on the Becton-Dickinson LSR Fortessa® or Becton-Dickinson FACS LSR II®, and data analyzed with FlowJo® software.

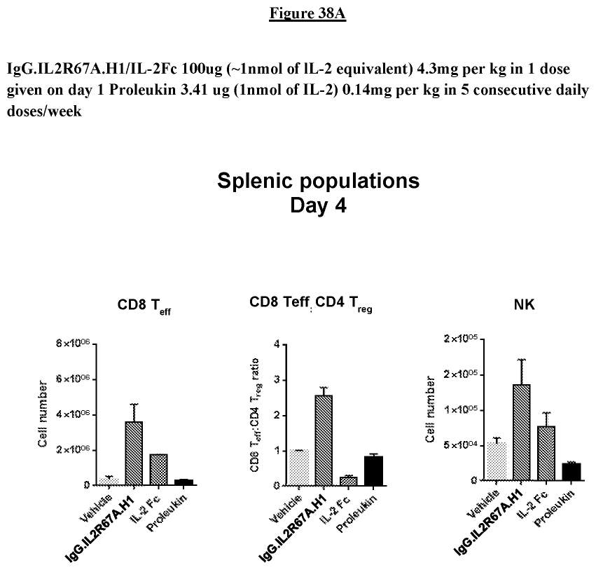

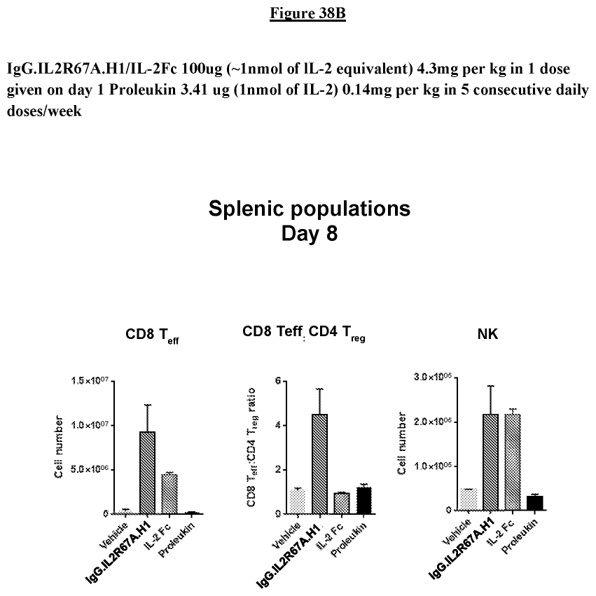

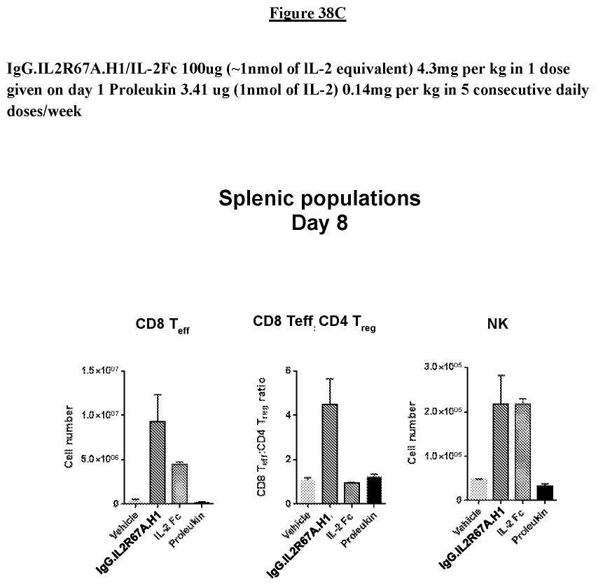

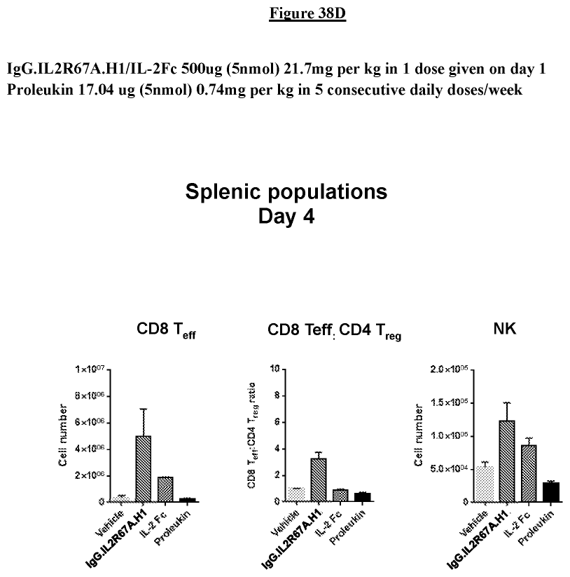

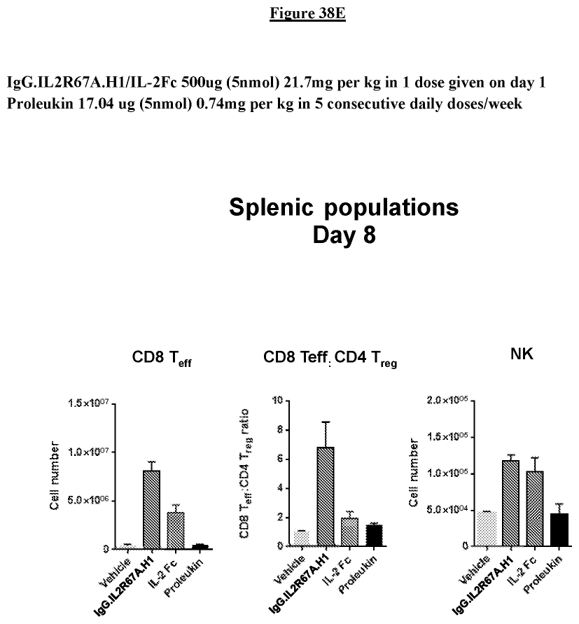

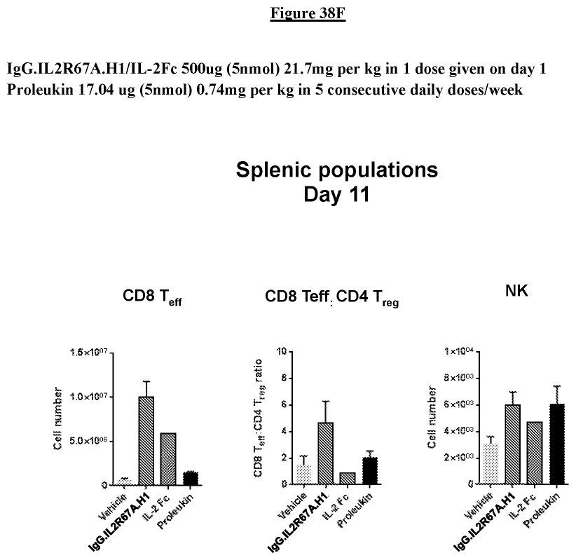

FIGS. 38A-38C show the preferential expansion of CD8 T effector cells in B6 female mice after administration of Proleukin® (5× weekly), IL2-Fc and IgG.IL2R67A.H1 (1×/week) at Proleukin® equimolar concentrations (IgG.IL2R67A.H1/IL2-Fc 100 μg˜1 nmol IL2 equivalent). The data in the graphs demonstrate that CD8 T effector cells proliferate without similar proliferation of Tregs. Contrast this data to Proleukin® which expanded both CD8 T effectors and Tregs. Note that IgG.IL2R67A.H1 was superior in both absolute numbers of CD8 T effector cell expansion and in the ratio CD8 T effector cells:Tregs to an IL2-Fc fusion construct, demonstrating that there is a structural and functional basis for the IgG.IL2R67A.H1 construct. FIGS. 38D-38F show that the beneficial effect of IgG.IL2R67A.H1 is more apparent at higher doses. When 500 μg (5 nmol IL2 equivalent) of IgG.IL2R67A.H1 was administered to B6 mice, the preferential expansion of CD8 T effector cells was seen relative to Treg cells similar to the lower dose. However, in the IL2-Fc treatment group, mice were found dead after only a single dose at the higher level (data not shown). This indicates that IgG.IL2R67A.H1 has a larger therapeutic index than that of IL2-Fc fusion constructs, and can be safely administered in a wider dosage range.

### Example 28: IgG.IL2R67A.H1 Selectively Expands CD8 T Effector Cells, and is Better Tolerated than Proleukin® in NOD Mice

The non-obese diabetic (NOD) mouse develops type 1 diabetes spontaneously and is often used as an animal model for human type 1 diabetes. Using the same protocol for the B6 mice described in Example 27, IgG.IL2R67A.H1, IL2-Fc and Proleukin® were administered to NOD mice at Proleukin® equimolar equivalents. Again, administration of IgG.IL2R67A.H1 at this dose preferentially expanded CD8 T effector cells over Tregs as shown in the graph in FIG. 39A. In addition, administration of IgG.IL2R67A.H1 showed no adverse events in NOD mice, while the Proleukin® treated group had 5 moribund mice and 2 deaths. FIG. 39B is a graph reporting the dosages, fold cellular changes and cell type from the NOD mouse model.

### Example 29: IgG.IL2R67A.H1 Shows Single-Agent Efficacy in a CT26 Colon Tumor Mouse Model

After studying the safety of IgG.IL2R67A.H1, its single-agent efficacy was tested in a CT26 mouse model. The murine CT26 cell line is a rapidly growing grade IV colon carcinoma cell line, used in over 500 published studies and is one of the commonly used models in drug development.

CT26 (ATCC CRL-2638) cells were grown in sterile conditions in a 37° C. incubator with 5% CO₂. The cells were cultured in RPMI 1640 media supplemented with 10% FBS. Cells were passed every 3-4 days. For the day of injection, cells were harvested (Passage 11) and re-suspended in HBSS at a concentration of 2.5×10⁶/ml. Cells were Radil tested on for mycoplasma and murine viruses. Balbc mice were used. For each mouse, 0.25×10⁶ cells were implanted with subcutaneously injection into right flank using a 28g needle (100 μl injection volume). After implantation, animals were calipered and weighed 3 times per week once tumors were palpable. Caliper measurements were calculated using (L×W×W)/2. Mice were fed with normal diet and housed in SPF animal facility in accordance with the Guide for Care and Use of Laboratory Animals and regulations of the Institutional Animal Care and Use Committee.

When tumors reached about 100 mm³, mice were administered by intraperitoneal route 12.5-100 μg of IgG.IL2R67A.H1. Tumors were measured twice a week. Average tumor volumes were plotted using Prism 5 (GraphPad®) software. An endpoint for efficacy studies was achieved when tumor size reached a volume of 1000 mm³. Following injection, mice were also closely monitored for signs of clinical deterioration. If for any reason mice showed any signs of morbidity, including respiratory distress, hunched posture, decreased activity, hind leg paralysis, tachypnea as a sign for pleural effusions, weight loss approaching 20% or 15% plus other signs, or if their ability to carry on normal activities (feeding, mobility), was impaired, mice were euthanized.

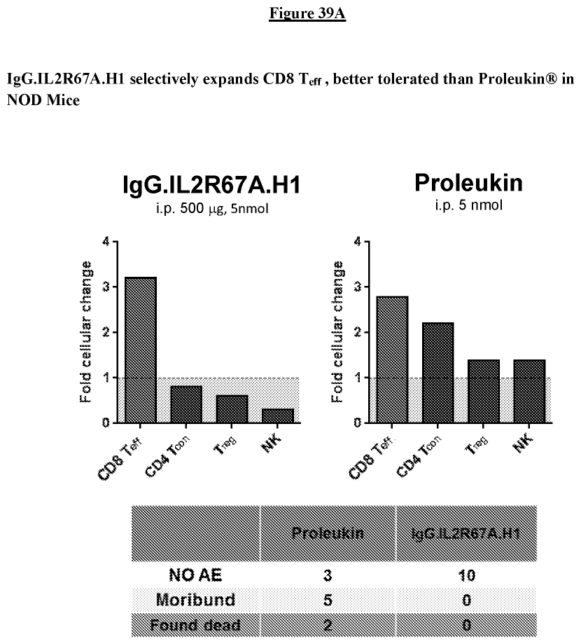

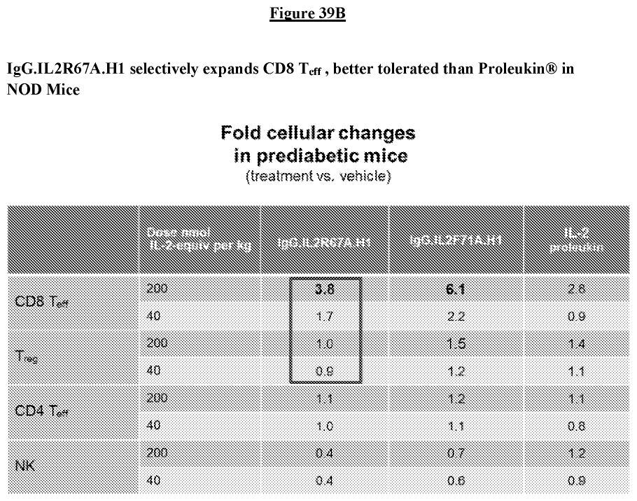

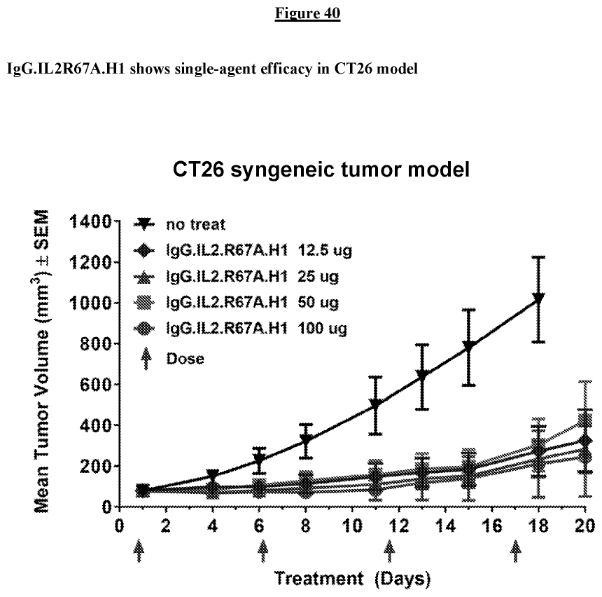

IgG.IL2R67A.H1 was efficacious in the CT26 mouse model at doses ranging from 12.5 μg to 100 μg, with 4 administrations of IgG.IL2R67A.H1 over 17 days in a 20 day study. The tumor volume curves shown in FIG. 40 are indicative of the efficacy of IgG.IL2R67A.H1 in this study, as tumor volumes were kept under 200 mm for 15 days and then under 400 mm for the remaining 5 days.

### Example 30: IgG.IL2R67A.H1 and Additional Cancer Therapeutics Show Efficacy in a B16 Mouse Model

To assess the efficacy of IgG.IL2R67A.H1 in combination with other cancer therapeutics, a B16F10 melanoma mouse model was used. B16F10 cells (ATCC CRL-6475) were grown in sterile conditions in a 37° C. incubator with 5% CO₂ for two weeks. B16F10 cells were cultured in DMEM+10% FBS. Cells were harvested and re-suspended in FBS-free medium DMEM at a concentration of 1×10⁶/100 μl. B16F10 cells were Radil tested for mycoplasma and murine viruses. Cells were implanted into the right flank of B6 mice using a 28 gauge needle (100 μl injection volume). After implant, mice were calipered and weighed 2 times per week once tumors were palpable. Caliper measurements were calculated using (L×W×W)/2.

In this study, IgG.IL2R67A.H1 was used as a single agent or in combination with the TA99 antibody, an anti-Trp1 antibody, with Trp1 expressed at high levels on B16F10 cells. An IL2-Fc fusion was administered as a single agent or in combination with the TA99 antibody. As a control, the TA99 antibody was administered as a single agent.

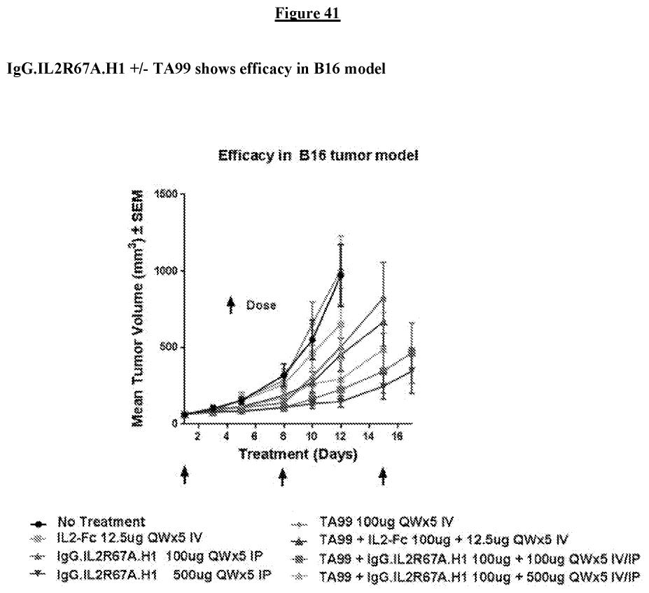

Surprisingly, IgG.IL2R67A.H1 when administered as a single agent at a 500 μg dose was the most efficacious treatment in this model (FIG. 41). The next best treatment was the combination of IgG.IL2R67A.H1 (100 μg) and TA99. This combination was more efficacious than IgG.IL2F71A.H1 as a single agent at 100 μg, TA99 in combination with IgG.IL2F71A.H1 at 500 μg and IL2-Fc as a single agent or as an IL2-Fc/TA99 combination. When TA99 was administered a single agent, it had no effect, and the mean tumor volume was similar to untreated control. This data demonstrates that IgG.IL2R67A.H1 is efficacious as a single agent in melanoma mouse tumor model, but it is also efficacious when paired with another anti-cancer agent.

### Example 31: Activity of IgG.IL2R67A.H1 and IgG.IL2F71A.H1 in Human Cells

In order to test the activity of IgG.IL2R67A.H1 on human CD8 T effectors, human peripheral blood mononuclear cells (PBMC) were assayed for pSTAT5 activity. PBMC cells were rested in serum-free test media, and plated. IgG.IL2R67A.H1, IgG.IL2F71A.H1 or Proleukin® was added to the PBMCs, and incubated for 20 minutes at 37° C. After 20 min, cells were fixed with 1.6% formaldehyde, washed and stained with surface markers. After 30 minutes at room temperature, samples were washed and re-suspended cell pellets were permeabilized with −20° C. methanol, washed and stained for pSTAT5 and DNA intercalators. Cells were run on Cytof® and data analyzed with FlowJo™ software to quantify the level of pSTAT5 activity. The table in FIG. 42 demonstrates the preferential activation IgG.IL2R67A.H1 has for human CD8 T effector cells and minimizes the activation of Treg cells.

### Example 32: Binding of Antibody Cytokine Engrafted Proteins

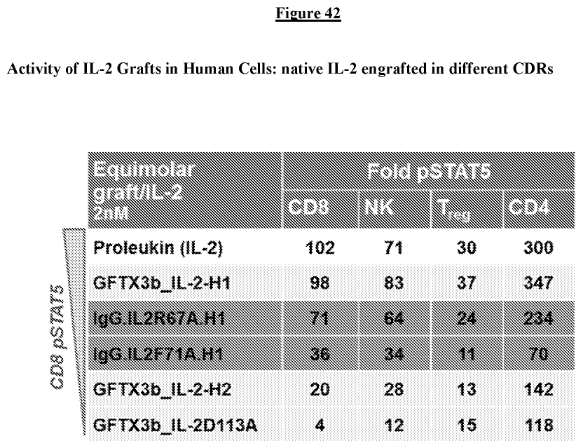

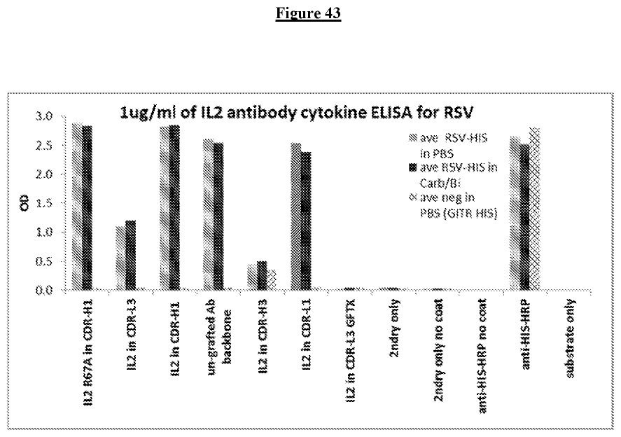

Antibody cytokine engrafted proteins were prepared using a variety of known immunoglobulin sequences which have been utilized in clinical settings as well as germline antibody sequences. One of the antibodies used has RSV as its antigen. To determine if engrafting IL2 into the CDRs of this antibody reduced or abrogated binding to RSV, an ELISA assay was run on RSV proteins either in PBS or a carbonate buffer. As shown in FIG. 43, this appears to be influenced by which CDR was chosen for IL2 engrafting. For example, IgG.IL2R67A.H1 has RSV binding similar to the un-grafted (un-modified) original antibody. In contrast, engrafting IL2 into the light chain of CDR3 (CDR-L3) or into CDR-H3 reduces binding. As expected, IL2 engrafted into a GFTX antibody scaffold which targets IgE produces no binding. This demonstrates that antibody cytokine engrafted proteins can retain binding to the original target of the antibody scaffold, or this binding can be reduced.

### Example 33: In Vitro Activity of IL-6 Antibody Cytokine Engrafted Proteins in Human PBMCs

CyTOF, a FACS based method that combines mass cytometry, incorporates flow cytometry technology with a time-of-flight inductively coupled plasma mass spectrometry (ICP-MS). It allows for the simultaneous detection and quantification of over 40 parameters from a single cell. It utilizes rare-earth metal conjugated monoclonal antibodies to specific cell surface or intracellular molecules. Using CyTOF, in vitro signaling studies were performed on IL-6 antibody cytokine engrafted proteins in human PBMCs assessed by pSTAT1, pSTAT3, pSTAT4, and pSTAT5 detection.

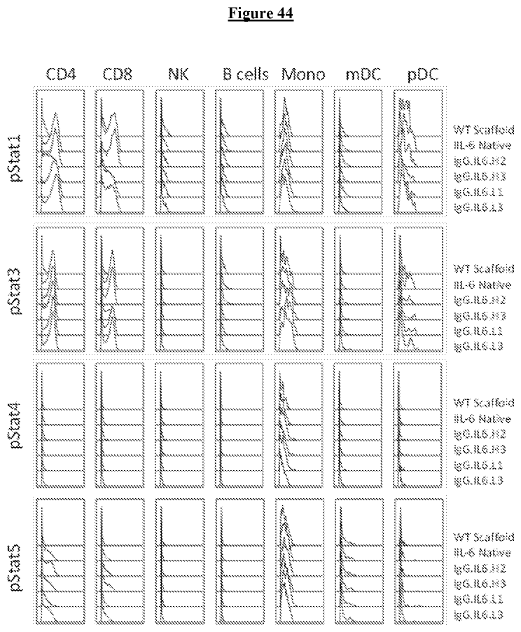

Human PBMCs were treated with an isotype control, IL-6 grafts (IgG.IL-6.L2, IgG.IL-6.L3, IgG.IL-6.H2 and IgG.IL-6.H3), or native IL-6 at molar equivalents of IL-6 for 30 minutes. The cells were fixed with 1.6% PFA to preserve phosphorylation status on signaling molecules. The cells were then stained with a combination of cell surfaces receptors for specific lineages and intracellular signaling molecules of the JAK/Stat pathway. The samples were then acquired and analyzed on the CyTOF. Results indicate that the IL-6 grafts have similar bioactivity as native IL-6 (FIG. 44). They also signal on similar cell populations (CD8 and CD4 T cells) and through the same JAK/Stat pathways.

### Example 34: In Vivo Activity of IL-6 Antibody Cytokine Engrafted Proteins in C57B16 DIO Mice

CyTOF analysis was also run on immune cells in mice. For the mouse in vivo studies, C57/B16 DIO mice were dosed once subcutaneously with 5 mg/kg of IgG.IL-6.L3, IgG.IL-6.H2 and IgG.IL-6.H3 and compared to a naïve mouse. Whole blood was collected at 2 post dose and fixed with 1.6% PFA to preserve phosphorylation status on signaling molecules. The cells were then stained with a combination of cell surfaces receptors for specific lineages and intracellular signaling molecules of the JAK/Stat pathway. The samples where then acquired and analyzed on the CyTOF.

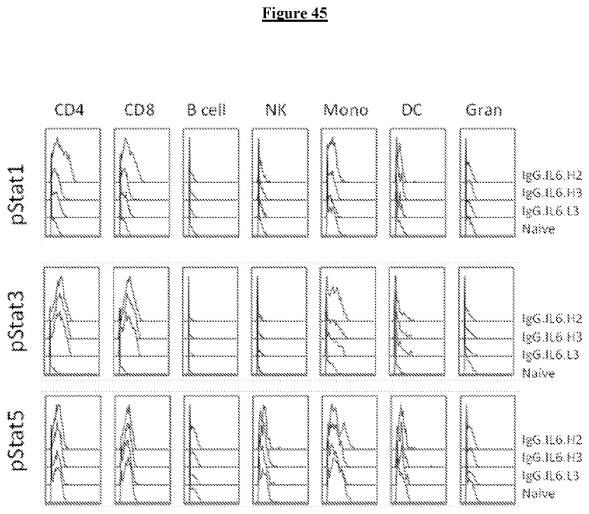

As shown in the graphs in FIG. 45, IL-6 antibody cytokine engrafted proteins stimulated both CD8 and CD4 T cells as measured by pSTAT1, and pSTAT3 levels. Stimulation of monocytes was also observed as measured by pSTAT3 levels.

### Example 35: Pharmacokinetics and Pharmacodynamics Evaluation of IL-6 Antibody Cytokine Engrafted Proteins

Half-life of the antibody cytokine engrafted proteins was assessed in C57Bl/6 DIO mice. Antibody cytokine engrafted proteins were injected at 0.5, 2, 5 and 10 mg/kg (10 ml/kg dose volume) in 0.9% saline subcutaneously and blood was sampled beginning at 2 hours post-injection and up to 240 hours post-injection. Whole blood was collected into heparin-treated tubes at each time point and centrifuged at 12,500 rpm for 10 minutes at 4° C. Plasma supernatant was collected and stored at −80° C. until all time points were collected. Antibody cytokine engrafted proteins levels in plasma were measured using three different immunoassay methods to enable detection of both the IL-6 and antibody domains of the antibody cytokine engrafted protein. The first assay consisted of an in-house biotin labelled goat anti-human IL-6 capture (R&D Systems AF-206-NA) and alexafluor 647 goat anti-human IgG, Fcγ specific detection (Jackson ImmunoResearch #109-605-098). The second assay consisted of a biotinylated goat anti-human IgG, Fcγ specific detection (Jackson ImmunoResearch #109-065-098) and alexafluor 647 goat anti-human IgG, Fcγ specific detection (Jackson ImmunoResearch #109-605-098). And the third assay consisted of an in-house biotin labelled goat anti-human IL-6 capture (R&D Systems AF-206-NA) and in-house alexafluor 647 labelled anti-human IL-6 detection (R&D Duoset DY206-05 Part #840113). All three assays were run on the GyroLab® xP Workstation (Gyros AB Uppsala, Sweden). The assay was run on 200 nL CDs (Gyros #P0004180) using a Gyros-approved wizard method. The buffers used were Rexxip A® (Gyros #P0004820) for standard and sample dilution and Rexxip F® (Gyros #P0004825) for detection preparation. Analysis of results was done using the Gyrolab® data analysis software. As shown in FIG. 46A-B, IgG.IL-6.H2 and IgG.IL-6.H3 shows a half-life of 12-14 hours, both longer than native IL-6.

Consistent with the extended half-life, antibody cytokine engrafted proteins also demonstrated improved pharmacodynamics. Phospho-stat3 (pSTAT3), a marker of IL-6 activation was monitored in target tissues (muscle and fat) after subcutaneous dosing. Antibody cytokine engrafted protein IgG.IL-6.H2 was injected at 0.1 (10 ml/kg dose volume) in 0.9% saline subcutaneously. Terminal quadriceps muscle and gonad fat (1 cm each) were harvested at 4 hours post-injection. Muscle and fat tissue was collected in tubes containing 500 μl MSD Lysis Buffer (Meso Scale Discovery, #K150SVD-2, Lot #Z0055522) and a steel bead (Qiagen, #69989). Tissues were homogenized by tissue-lyser at 30 rps for 5 minutes at room temperature. Lysed tissue was centrifuged for 10 minutes at 14,000×g at 4° C. Supernatant was collected and stored on ice until phospho-STAT3 assay.

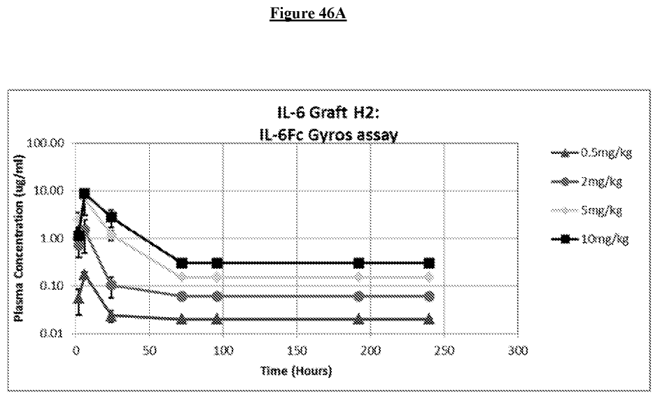

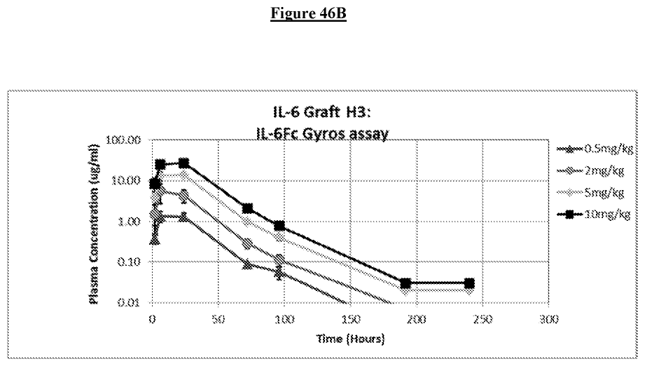

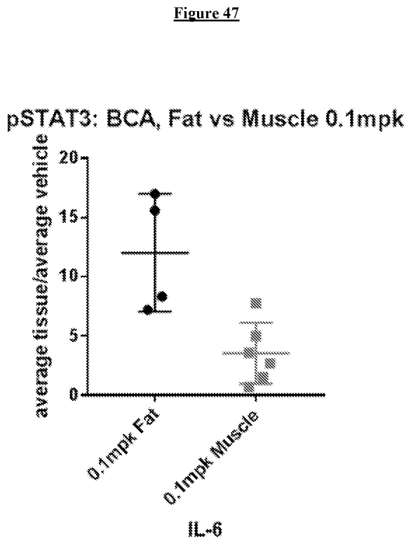

A phospho-Stat3 assay plate (Meso Scale Discovery® pSTAT3(Tyr705) Assay) was run on the same day as tissue collection and processing. Tissue supernatant protein detection was performed using the Bradford Assay (Pierce). Protein was then plated on the phospho-STAT3 assay plate at 50 μl/well. Plates were incubated at room temperature for 2 hours, washed, and treated with phospho-STAT3 or Total STAT3 antibody (Meso Scale Discovery). Plates were analysed for relative fluorescence units (RFU) on the MSD Sector Imager 2400 (Meso Scale Discovery). Protein phospho-STAT3 RFU was normalized to loaded protein concentration. Enhanced pSTAT3 signal is detected in fat tissue, but not muscle at 4 hours post dose (FIG. 47).

### Example 36: In Vivo Activity of IL-6 Antibody Cytokine Engrafted Proteins in C57B16 DIO Mice

A dose response of efficacy for two of the grafts was performed. In the experiment, C57/B16 DIO mice were dosed once per day subcutaneously with vehicle, 5, 10, or 20 mg/kg of both the H2 and H3 versions of the antibody IL-6 grafted protein. Whole blood was collected at 2, 6 and 24 hrs post-dose on day 1 and day 13. Whole blood was collected into heparin-treated tubes at each time point and centrifuged at 12,500 rpm for 10 minutes at 4° C. Plasma supernatant was collected and stored at −80° C. until all time points were collected. Samples were submitted for PK analysis as above. Body weights were taken every other day to monitor weight loss. Once a week NMR analysis was done to assess body mass composition as compared to naive normal diet control mice. On day 20, mice were dosed then fasted overnight. The following morning the received a glucose challenge (20% glucose 1 g/kg bolus). Mice were bled at 20, 40, 60 and 120 minutes after glucose dosing and blood glucose levels were measured on a glucometer.

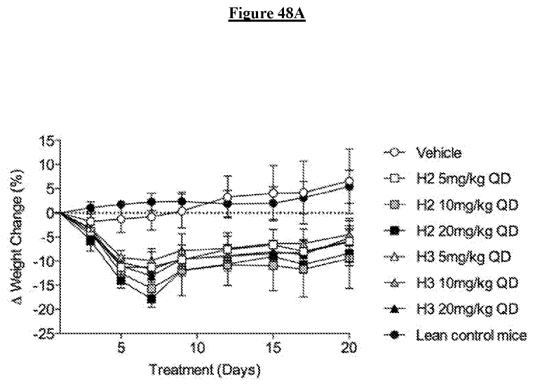

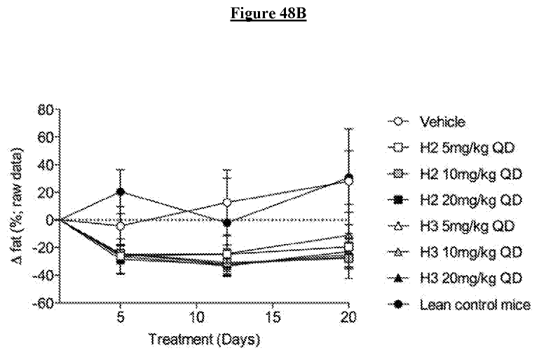

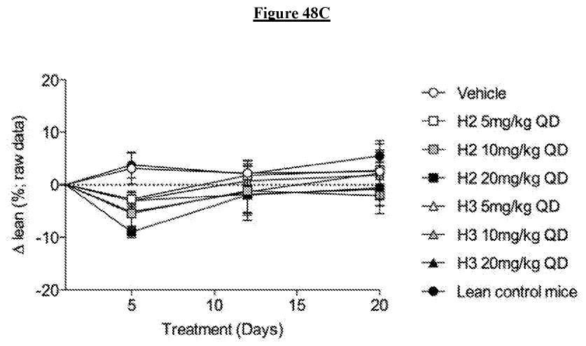

Rapid loss of body weight and fat mass is noted with both grafts and all dose levels (FIGS. 48A and 48B). Less pronounced effect on lean fraction, with possible dose response (FIG. 48C). Effect on lean mass appears to decrease over time, whereas effect on fat loss persists.

### Example 37: In Vivo Activity of IL-6 Antibody Cytokine Engrafted Proteins on Respiratory Exchange Ratio (RER) in C57B16 DIO Mice

A study was designed to test the effect of antibody cytokine engrafted protein IgG.IL-6.H3 on respiratory exchange ratio. C57/B16 DIO mice were dosed once per day subcutaneously with vehicle or 5 mg/kg of the H3 version of the antibody IL-6 grafted protein. Dosing was performed on days 1-3 and 5-7 of the experiment, while 02 consumption, and CO₂ production were assessed in Oxymax indirect calorimetry cages in 48 hours increments on days −1-1, 3-5 and 7-9, during which time mice remained undisturbed. Body weights were taken every other day to monitor weight loss. Respiratory exchange ratio (RER) was calculated from measured 02 consumption and CO₂ production.

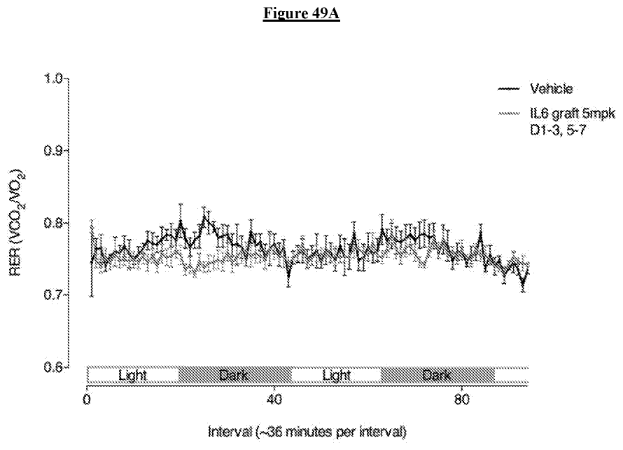

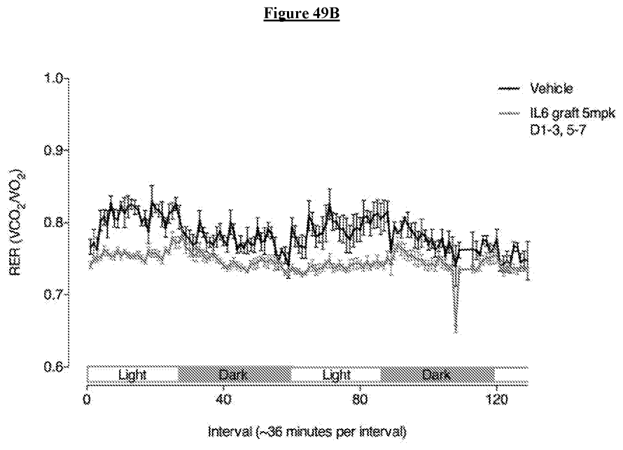

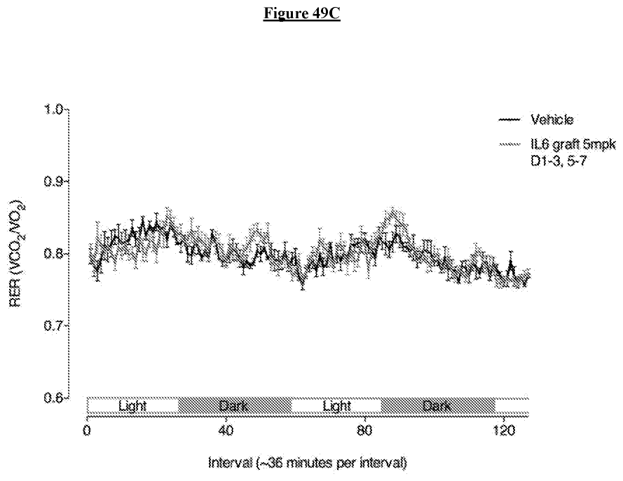

Pre-dosing RER was equivalent between experimental cohorts (FIG. 49A). By contrast, at days 3-5, a clear decrease in RER was noted in the H3 graft dosed animals relative to vehicle controls, indicative of a shift towards fat utilization (FIG. 49B). This difference normalized by 7-9 (FIG. 49C).

### Example 38: In Vivo Activity of IL-6 Antibody Cytokine Engrafted Proteins on Food Intake in Pair Fed C57B16 DIO Mice

A study was designed to test the effect of antibody cytokine engrafted protein IgG.IL-6.H3 on food intake in a pair feeding model. C57/B16 DIO mice were dosed once per day subcutaneously with vehicle or 5 mg/kg of the H3 version of the antibody IL-6 grafted protein. Food intake was assessed by weighing food at beginning of study and twice daily thereafter. The pair fed group received as much food as the dosed group consumed each morning and afternoon, starting on the second day of dosing. NMR analysis was done on days 1, 3, 5, and 7 of dosing to assess body mass composition.

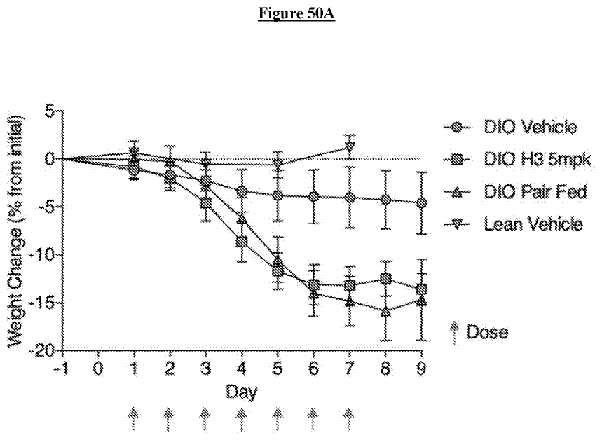

H3 antibody graft dosed animals demonstrated rapid weight reduction, reaching ˜15% body weight loss by day 6 of treatment (FIG. 50A). This effect was accompanied by a significant reduction in food intake, which reached nadir at day 3 of dosing, with subsequent gradual increase to baseline levels of food consumption (FIG. 50B). Pair fed animals demonstrated a degree of weight loss similar to the H3 graft dosed animals, indicating that the weight loss induced by grafted antibody treatment largely reflects a decrease in food intake (FIG. 50A). The loss of body weight in both the H3 antibody graft dosed animals and in pair fed animals was accompanied by ˜30-40% decrease in overall fat mass at day 7 (FIG. 50C); by contrast, lean mass was reduced significantly in pair fed but not H3 antibody graft dosed animals (FIG. 50D). Weight of the isolated tibialis anterior muscle was not significantly decreased in either H3 antibody graft dosed or pair-fed animals (FIG. 50E).

### Example 39: Creation of IL10 Antibody Cytokine Engrafted Proteins

IL10 ACE proteins were generated by engineering a monomeric IL10 sequence into CDR regions of various immunoglobulin scaffolds, then both heavy and light chain immunoglobulin chains were produced to generate final protein constructs. IL10 ACE proteins confer preferred therapeutic anti-inflammatory properties of IL10; however, IgGIL10M engrafted constructs have reduced proportional pro-inflammatory activity as compared with rhIL10.

To create antibody cytokine engrafted proteins, monomeric IL10 (IL10M), comprising residues 19-178 of full length IL10 with a six amino acid linker between residues 134 and 135 was inserted into various CDR loops of immunoglobulin chain scaffold. Engrafted constructs were prepared using a variety of known immunoglobulin sequences which have been utilized in clinical settings as well as germline antibody sequences. Sequences of IL10M in two exemplary scaffolds, referred to as GFTX and GFTX3b, with the GFTX ACE proteins listed in TABLE 2 and the GFTX3b proteins listed in TABLE 3. Insertion points were selected to be the mid-point of the CDR loop based on available structural or homology model data. Antibody cytokine engrafted proteins were produced using standard molecular biology methodology utilizing recombinant DNA encoding the relevant sequences.

For example, a variable region of each antibody containing IL10M inserted into one of the six CDRs was synthesized. DNA encoding variable region was amplified via PCR and the resulting fragment was sub-cloned into vector containing either the light chain constant region or the heavy chain constant and Fc regions. In this manner IL10M antibody cytokine engrafted proteins were made corresponding to insertion of IL10M into each of the 6 CDRs (L1, L2, L3, H1, H2, H3). Resulting constructs are shown in TABLE 2 or TABLE 3. Transfections of the appropriate combination of heavy and light chain vectors results in the expression of a recombinant antibody with two grafted IL10M molecules (one IL10 monomer in each Fab arm).

The selection of which CDR is chosen for cytokine engraftment is chosen on the parameters of: the required biology, the biophysical properties and a favorable development profile. At this time, modeling software is only partially useful in predicting which CDR and which location within the CDR will provide the desired parameters, so therefore all six possible antibody cytokine grafts are made and then evaluated in biological assays. If the required biological activity was achieved, then the biophysical properties such as structural resolution of the antibody cytokine engrafted molecule were resolved.

By virtue of the grafting of IL10 into a CDR, the antibody portion of the antibody cytokine engrafted protein presents the IL10 monomer with a unique structure which influences the binding to the IL10 receptor as discussed below. There are no off-target effects due to the antibody portion. In addition, the Fc portion of the antibody cytokine engrafted protein has been modified to be fully silent regarding ADCC (Antibody Dependent Cell-mediated Cytotoxicity) and CDC (Complement-Dependent Cytotoxicity).

In summary, the insertion point in each CDR was chosen on a structural basis, with the hypothesis that grafting into the CDR would provide some level of steric hindrance to individual subunits of the IL10 receptor. The final selection of which CDR graft is best for a particular cytokine is based on desired biology and biophysical properties. The nature of the cytokine receptor, the cytokine/receptor interactions and the mechanism of signaling also played a role and this was resolved by comparing each individual antibody cytokine engrafted molecule for their respective properties. For example, engrafting of IL10 into the light chain CDR1 (CDRL1) produced the desired biological activity of activating monocytes but not other cells such as NK cells. This was seen in the exemplary antibody cytokine engrafted proteins IgGIL10M7 and IgGIL10M13.

TABLE 3

| SEQ ID NO: | Description | | Comments |
|---|---|---|---|
| 3817 | CDRH1 of | GFSLSTSGM | GFTX3b |
| | IgGIL10M7 | | |
| | (Chothia) | | |
| 3818 | CDRH2 of | WWDDK | GFTX3b |
| | IgGIL10M7 | | |
| | (Chothia) | | |
| 3819 | CDRH3 of | SMITNWYFDV | GFTX3b |
| | IgGIL10M7 | | |
| | (Chothia) | | |
| 3820 | CDRL1 of | QLSSPGQGTQSENSCTHFPGNLPNMLRDL | IL10 |
| | IgGIL10M7 | RDAFSRVKTFFQMKDQLDNLLLKESLLED | grafted |
| | (Chothia) | FKGYLGCQALSEMIQFYLEEVMPQAENQD | into |
| | | PDIKAHVNSLGENLKTLRLRLRRCHRFLP | CDRL1. |
| | | CENGGGSGGKSKAVEQVKNAFNKLQEKGI | IL10 is |
| | | YKAMSEFDIFINYIEAYMTMKIRNVGY | bolded, |
| | | | underlined |
| 3821 | CDRL2 of | DTS | GFTX3b |
| | IgGIL10M7 | | |
| | (Chothia) | | |
| 3822 | CDRL3 of | GSGYPF | GFTX3b |
| | IgGIL10M7 | | |
| | (Chothia) | | |
| 3823 | CDRH1 of | TSGMSVG | GFTX3b |
| | IgGIL10M7 | | |
| | (Kabat) | | |
| 3824 | CDRH2 of | DIWWDDKKDYNPSLKS | GFTX3b |
| | IgGIL10M7 | | |
| | (Kabat) | | |
| 3825 | CDRH3 of | SMITNWYFDV | GFTX3b |
| | IgGIL10M7 | | |
| | (Kabat) | | |
| 3826 | CDRL1 of | KAQLSSPGQGTQSENSCTHFPGNLPNMLR | IL10 |
| | IgGIL10M7 | DLRDAFSRVKTFFQMKDQLDNLLLKESLL | grafted |
| | (Kabat) | EDFKGYLGCQALSEMIQFYLEEVMPQAEN | into CDRL1. |
| | | QDPDIKAHVNSLGENLKTLRLRLRRCHRF | IL10 is |
| | | LPCENGGGSGGKSKAVEQVKNAFNKLQEK | bolded, |
| | | GIYKAMSEFDIFINYIEAYMTMKIRNVGY | underlined |
| | | MH | |
| 3827 | CDRL2 of | DTSKLAS | GFTX3b |
| | IgGIL10M7 | | |
| | (Kabat) | | |
| 3828 | CDRL3 of | FQGSGYPFT | GFTX3b |
| | IgGIL10M7 | | |
| | (Kabat) | | |
| 3829 | VH of | QVTLRESGPALVKPTQTLTLTCTFSGFSL | GFTX3b |
| | IgGIL10M7 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3830 | VL of | DIQMTQSPSTLSASVGDRVTITCKAQLSS | IL10 |
| | IgGIL10M7 | PGQGTQSENSCTHFPGNLPNMLRDLRDAF | grafted |
| | | SRVKTFFQMKDQLDNLLLKESLLEDFKGY | into CDRL1. |
| | | LGCQALSEMIQFYLEEVMPQAENQDPDIK | |
| | | AHVNSLGENLKTLRLRLRRCHRFLPCENG | |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIK | |
| 3831 | Heavy chain | QVTLRESGPALVKPTQTLTLTCTFSGFSL | GFTX3b |
| | of IgGIL10M7 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPELLGGPSVFLFPPKPKDTLMISRTPE | |
| | | VTCVVVDVSHEDPEVKFNWYVDGVEVHNA | |
| | | KTKPREEQYNSTYRVVSVLTVLHQDWLNG | |
| | | KEYKCKVSNKALPAPIEKTISKAKGQPRE | |
| | | PQVYTLPPSREEMTKNQVSLTCLVKGFYP | |
| | | SDIAVEWESNGQPENNYKTTPPVLDSDGS | |
| | | FFLYSKLTVDKSRWQQGNVFSCSVMHEAL | |
| | | HNHYTQKSLSLSPGK | |
| 3832 | Light chain | DIQMTQSPSTLSASVGDRVTITCKAQLSS | IL10 |
| | of IgGIL10M7 | PGQGTQSENSCTHFPGNLPNMLRDLRDAF | grafted |
| | | SRVKTFFQMKDQLDNLLLKESLLEDFKGY | into CDRL1. |
| | | LGCQALSEMIQFYLEEVMPQAENQDPDIK | |
| | | AHVNSLGENLKTLRLRLRRCHRFLPCENG | |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3833 | CDRH1 of | GFSLSTSGM | GFTX3b |
| | IgGIL10M8 | | |
| | (Chothia) | | |
| 3834 | CDRH2 of | WWDDK | GFTX3b |
| | IgGIL10M8 | | |
| | (Chothia) | | |
| 3835 | CDRH3 of | SMITNWYFDV | GFTX3b |
| | IgGIL10M8 | | |
| | (Chothia) | | |
| 3836 | CDRL1 of | QLSVGY | GFTX3b |
| | IgGIL10M8 | | |
| | (Chothia) | | |
| 3837 | CDRL2 of | DTSPGQGTQSENSCTHFPGNLPNMLRDLR | GFTX3b |
| | IgGIL10M8 | DAFSRVKTFFQMKDQLDNLLLKESLLEDF | IL10 |
| | (Chothia) | KGYLGCQALSEMIQFYLEEVMPQAENQDP | grafted |
| | | DIKAHVNSLGENLKTLRLRLRRCHRFLPC | into CDRL2. |
| | | ENGGGSGGKSKAVEQVKNAFNKLQEKGIY | IL10 is |
| | | KAMSEFDIFINYIEAYMTMKIRNS | bolded, |
| | | | underlined |
| 3838 | CDRL3 of | GSGYPF | GFTX3b |
| | IgGIL10M8 | | |
| | (Chothia) | | |
| 3839 | CDRH1 of | TSGMSVG | GFTX3b |
| | IgGIL10M8 | | |
| | (Kabat) | | |
| 3840 | CDRH2 of | DIWWDDKKDYNPSLKS | GFTX3b |
| | IgGIL10M8 | | |
| | (Kabat) | | |
| 3841 | CDRH3 of | SMITNWYFDV | GFTX3b |
| | IgGIL10M8 | | |
| | (Kabat) | | |
| 3842 | CDRL1 of | KAQLSVGYMH | GFTX3b |
| | IgGIL10M8 | | |
| | (Kabat) | | |
| 3843 | CDRL2 of | DTSPGQGTQSENSCTHFPGNLPNMLRDLR | GFTX3b |
| | IgGIL10M8 | DAFSRVKTFFQMKDQLDNLLLKESLLEDF | IL10 |
| | (Kabat) | KGYLGCQALSEMIQFYLEEVMPQAENQDP | grafted |
| | | DIKAHVNSLGENLKTLRLRLRRCHRFLPC | into CDRL2. |
| | | ENGGGSGGKSKAVEQVKNAFNKLQEKGIY | IL10 is |
| | | KAMSEFDIFINYIEAYMTMKIRNSKLAS | bolded, |
| | | | underlined |
| 3844 | CDRL3 of | FQGSGYPFT | GFTX3b |
| | IgGIL10M8 | | |
| | (Kabat) | | |
| 3845 | VH of | QVTLRESGPALVKPTQTLTLTCTFSGFSL | GFTX3b |
| | IgGIL10M8 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3846 | VL of | DIQMTQSPSTLSASVGDRVTITCKAQLSV | GFTX3b |
| | IgGIL10M8 | GYMHWYQQKPGKAPKLLIYDTSPGQGTQS | IL10 |
| | | ENSCTHFPGNLPNMLRDLRDAFSRVKTFF | grafted |
| | | QMKDQLDNLLLKESLLEDFKGYLGCQALS | into CDRL2. |
| | | EMIQFYLEEVMPQAENQDPDIKAHVNSLG | |
| | | ENLKTLRLRLRRCHRFLPCENGGGSGGKS | |
| | | KAVEQVKNAFNKLQEKGIYKAMSEFDIFI | |
| | | NYIEAYMTMKIRNSKLASGVPSRFSGSGS | |
| | | GTAFTLTISSLQPDDFATYYCFQGSGYPF | |
| | | TFGGGTKLEIK | |
| 3847 | Heavy chain | QVTLRESGPALVKPTQTLTLTCTFSGFSL | GFTX3b |
| | of IgGIL10M8 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPELLGGPSVFLFPPKPKDTLMISRTPE | |
| | | VTCVVVDVSHEDPEVKFNWYVDGVEVHNA | |
| | | KTKPREEQYNSTYRVVSVLTVLHQDWLNG | |
| | | KEYKCKVSNKALPAPIEKTISKAKGQPRE | |
| | | PQVYTLPPSREEMTKNQVSLTCLVKGFYP | |
| | | SDIAVEWESNGQPENNYKTTPPVLDSDGS | |
| | | FFLYSKLTVDKSRWQQGNVFSCSVMHEAL | |
| | | HNHYTQKSLSLSPGK | |
| 3848 | Light chain | DIQMTQSPSTLSASVGDRVTITCKAQLSV | GFTX3b |
| | of IgGIL10M8 | GYMHWYQQKPGKAPKLLIYDTSPGQGTQS | IL10 |
| | | ENSCTHFPGNLPNMLRDLRDAFSRVKTFF | grafted |
| | | QMKDQLDNLLLKESLLEDFKGYLGCQALS | into CDRL2. |
| | | EMIQFYLEEVMPQAENQDPDIKAHVNSLG | |
| | | ENLKTLRLRLRRCHRFLPCENGGGSGGKS | |
| | | KAVEQVKNAFNKLQEKGIYKAMSEFDIFI | |
| | | NYIEAYMTMKIRNSKLASGVPSRFSGSGS | |
| | | GTAFTLTISSLQPDDFATYYCFQGSGYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3849 | CDRH1 of | GFSLSTSGM | GFTX3b |
| | IgGIL10M9 | | |
| | (Chothia) | | |
| 3850 | CDRH2 of | WWDDK | GFTX3b |
| | IgGIL10M9 | | |
| | (Chothia) | | |
| 3851 | CDRH3 of | SMITNWYFDV | GFTX3b |
| | IgGIL10M9 | | |
| | (Chothia) | | |
| 3852 | CDRL1 of | QLSVGY | GFTX3b |
| | IgGIL10M9 | | |
| | (Chothia) | | |
| 3853 | CDRL2 of | DTS | GFTX3b |
| | IgGIL10M9 | | |
| | (Chothia) | | |
| 3854 | CDRL3 of | GSGSPGQGTQSENSCTHFPGNLPNMLRDL | GFTX3b |
| | IgGIL10M9 | RDAFSRVKTFFQMKDQLDNLLLKESLLED | IL10 |
| | (Chothia) | FKGYLGCQALSEMIQFYLEEVMPQAENQD | grafted |
| | | PDIKAHVNSLGENLKTLRLRLRRCHRFLP | into CDRL3. |
| | | CENGGGSGGKSKAVEQVKNAFNKLQEKGI | IL10 is |
| | | YKAMSEFDIFINYIEAYMTMKIRNYPF | bolded, |
| | | | underlined |
| 3855 | CDRH1 of | TSGMSVG | GFTX3b |
| | IgGIL10M9 | | |
| | (Kabat) | | |
| 3856 | CDRH2 of | DIWWDDKKDYNPSLKS | GFTX3b |
| | IgGIL10M9 | | |
| | (Kabat) | | |
| 3857 | CDRH3 of | SMITNWYFDV | GFTX3 |
| | IgGIL10M9 | | |
| | (Kabat) | | |
| 3858 | CDRL1 of | KAQLSVGYMH | GFTX3b |
| | IgGIL10M9 | | |
| | (Kabat) | | |
| 3859 | CDRL2 of | DTSKLAS | GFTX3b |
| | IgGIL10M9 | | |
| | (Kabat) | | |
| 3860 | CDRL3 of | FQGSGSPGQGTQSENSCTHFPGNLPNMLR | GFTX3b |
| | IgGIL10M9 | DLRDAFSRVKTFFQMKDQLDNLLLKESLL | IL10 |
| | (Kabat) | EDFKGYLGCQALSEMIQFYLEEVMPQAEN | grafted |
| | | QDPDIKAHVNSLGENLKTLRLRLRRCHRF | into CDRL3. |
| | | LPCENGGGSGGKSKAVEQVKNAFNKLQEK | IL10 is |
| | | GIYKAMSEFDIFINYIEAYMTMKIRNYPF | bolded, |
| | | T | underlined |
| 3861 | VH of | QVTLRESGPALVKPTQTLTLTCTFSGFSL | GFTX3b |
| | IgGIL10M9 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3862 | VL of | DIQMTQSPSTLSASVGDRVTITCKAQLSV | GFTX3b |
| | IgGIL10M9 | GYMHWYQQKPGKAPKLLIYDTSKLASGVP | IL10 |
| | | SRFSGSGSGTAFTLTISSLQPDDFATYYC | grafted |
| | | FQGSGSPGQGTQSENSCTHFPGNLPNMLR | into CDRL3. |
| | | DLRDAFSRVKTFFQMKDQLDNLLLKESLL | |
| | | EDFKGYLGCQALSEMIQFYLEEVMPQAEN | |
| | | QDPDIKAHVNSLGENLKTLRLRLRRCHRF | |
| | | LPCENGGGSGGKSKAVEQVKNAFNKLQEK | |
| | | GIYKAMSEFDIFINYIEAYMTMKIRNYPF | |
| | | TFGGGTKLEIK | |
| 3863 | Heavy chain | QVTLRESGPALVKPTQTLTLTCTFSGFSL | GFTX3b |
| | of IgGIL10M9 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPELLGGPSVFLFPPKPKDTLMISRTPE | |
| | | VTCVVVDVSHEDPEVKFNWYVDGVEVHNA | |
| | | KTKPREEQYNSTYRVVSVLTVLHQDWLNG | |
| | | KEYKCKVSNKALPAPIEKTISKAKGQPRE | |
| | | PQVYTLPPSREEMTKNQVSLTCLVKGFYP | |
| | | SDIAVEWESNGQPENNYKTTPPVLDSDGS | |
| | | FFLYSKLTVDKSRWQQGNVFSCSVMHEAL | |
| | | HNHYTQKSLSLSPGK | |
| 3864 | Light chain | DIQMTQSPSTLSASVGDRVTITCKAQLSV | GFTX3b |
| | of IgGIL10M9 | GYMHWYQQKPGKAPKLLIYDTSKLASGVP | IL10 |
| | | SRFSGSGSGTAFTLTISSLQPDDFATYYC | grafted |
| | | FQGSGSPGQGTQSENSCTHFPGNLPNMLR | into CDRL3. |
| | | DLRDAFSRVKTFFQMKDQLDNLLLKESLL | |
| | | EDFKGYLGCQALSEMIQFYLEEVMPQAEN | |
| | | QDPDIKAHVNSLGENLKTLRLRLRRCHRF | |
| | | LPCENGGGSGGKSKAVEQVKNAFNKLQEK | |
| | | GIYKAMSEFDIFINYIEAYMTMKIRNYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3865 | CDRH1 of | GFSLSPGQGTQSENSCTHFPGNLPNMLRD | GFTX3b |
| | IgGIL10M10 | LRDAFSRVKTFFQMKDQLDNLLLKESLLE | IL10 |
| | (Chothia) | DFKGYLGCQALSEMIQFYLEEVMPQAENQ | grafted |
| | | DPDIKAHVNSLGENLKTLRLRLRRCHRFL | into CDRH1. |
| | | PCENGGGSGGKSKAVEQVKNAFNKLQEKG | IL10 is |
| | | IYKAMSEFDIFINYIEAYMTMKIRNSTSG | bolded, |
| | | M | underlined |
| 3866 | CDRH2 of | WWDDK | GFTX3b |
| | IgGIL10M10 | | |
| | (Chothia) | | |
| 3867 | CDRH3 of | SMITNWYFDV | GFTX3b |
| | IgGIL10M10 | | |
| | (Chothia) | | |
| 3868 | CDRL1 of | QLSVGY | GFTX3b |
| | IgGIL10M10 | | |
| | (Chothia) | | |
| 3869 | CDRL2 of | DTS | GFTX3b |
| | IgGIL10M10 | | |
| | (Chothia) | | |
| 3870 | CDRL3 of | GSGYPF | GFTX3b |
| | IgGIL10M10 | | |
| | (Chothia) | | |
| 3871 | CDRH1 of | SPGQGTQSENSCTHFPGNLPNMLRDLRDA | GFTX3b |
| | IgGIL10M10 | FSRVKTFFQMKDQLDNLLLKESLLEDFKG | IL10 |
| | (Kabat) | YLGCQALSEMIQFYLEEVMPQAENQDPDI | grafted |
| | | KAHVNSLGENLKTLRLRLRRCHRFLPCEN | into CDRH1. |
| | | GGGSGGKSKAVEQVKNAFNKLQEKGIYKA | IL10 is |
| | | MSEFDIFINYIEAYMTMKIRNSTSGMSVG | bolded, |
| | | | underlined |
| 3872 | CDRH2 of | DIWWDDKKDYNPSLKS | GFTX3b |
| | IgGIL10M10 | | |
| | (Kabat) | | |
| 3873 | CDRH3 of | SMITNWYFDV | GFTX3b |
| | IgGIL10M10 | | |
| | (Kabat) | | |
| 3874 | CDRL1 of | KAQLSVGYMH | GFTX3b |
| | IgGIL10M10 | | |
| | (Kabat) | | |
| 3875 | CDRL2 of | DTSKLAS | GFTX3b |
| | IgGIL10M10 | | |
| | (Kabat) | | |
| 3876 | CDRL3 of | FQGSGYPFT | GFTX3b |
| | IgGIL10M10 | | |
| | (Kabat) | | |
| 3877 | VH of | QVTLRESGPALVKPTQTLTLTCTFSGFSL | GFTX3b |
| | IgGIL10M10 | SPGQGTQSENSCTHFPGNLPNMLRDLRDA | IL10 |
| | | FSRVKTFFQMKDQLDNLLLKESLLEDFKG | grafted |
| | | YLGCQALSEMIQFYLEEVMPQAENQDPDI | into CDRH1 |
| | | KAHVNSLGENLKTLRLRLRRCHRFLPCEN | |
| | | GGGSGGKSKAVEQVKNAFNKLQEKGIYKA | |
| | | MSEFDIFINYIEAYMTMKIRNSTSGMSVG | |
| | | WIRQPPGKALEWLADIWWDDKKDYNPSLK | |
| | | SRLTISKDTSANQVVLKVTNMDPADTATY | |
| | | YCARSMITNWYFDVWGAGTTVTVSS | |
| 3878 | VL of | DIQMTQSPSTLSASVGDRVTITCKAQLSV | GFTX3b |
| | IgGIL10M10 | GYMHWYQQKPGKAPKLLIYDTSKLASGVP | |
| | | SRFSGSGSGTAFTLTISSLQPDDFATYYC | |
| | | FQGSGYPFTFGGGTKLEIK | |
| 3879 | Heavy chain | QVTLRESGPALVKPTQTLTLTCTFSGFSL | GFTX3b |
| | of IgGIL10M10 | SPGQGTQSENSCTHFPGNLPNMLRDLRDA | IL10 |
| | | FSRVKTFFQMKDQLDNLLLKESLLEDFKG | grafted |
| | | YLGCQALSEMIQFYLEEVMPQAENQDPDI | into CDRH1 |
| | | KAHVNSLGENLKTLRLRLRRCHRFLPCEN | |
| | | GGGSGGKSKAVEQVKNAFNKLQEKGIYKA | |
| | | MSEFDIFINYIEAYMTMKIRNSTSGMSVG | |
| | | WIRQPPGKALEWLADIWWDDKKDYNPSLK | |
| | | SRLTISKDTSANQVVLKVTNMDPADTATY | |
| | | YCARSMITNWYFDVWGAGTTVTVSSASTK | |
| | | GPSVFPLAPSSKSTSGGTAALGCLVKDYF | |
| | | PEPVTVSWNSGALTSGVHTFPAVLQSSGL | |
| | | YSLSSVVTVPSSSLGTQTYICNVNHKPSN | |
| | | TKVDKRVEPKSCDKTHTCPPCPAPELLGG | |
| | | PSVFLFPPKPKDTLMISRTPEVTCVVVDV | |
| | | SHEDPEVKFNWYVDGVEVHNAKTKPREEQ | |
| | | YNSTYRVVSVLTVLHQDWLNGKEYKCKVS | |
| | | NKALPAPIEKTISKAKGQPREPQVYTLPP | |
| | | SREEMTKNQVSLTCLVKGFYPSDIAVEWE | |
| | | SNGQPENNYKTTPPVLDSDGSFFLYSKLT | |
| | | VDKSRWQQGNVFSCSVMHEALHNHYTQKS | |
| | | LSLSPGK | |
| 3880 | Light chain | DIQMTQSPSTLSASVGDRVTITCKAQLSV | GFTX3b |
| | of IgGIL10M10 | GYMHWYQQKPGKAPKLLIYDTSKLASGVP | |
| | | SRFSGSGSGTAFTLTISSLQPDDFATYYC | |
| | | FQGSGYPFTFGGGTKLEIKRTVAAPSVFI | |
| | | FPPSDEQLKSGTASVVCLLNNFYPREAKV | |
| | | QWKVDNALQSGNSQESVTEQDSKDSTYSL | |
| | | SSTLTLSKADYEKHKVYACEVTHQGLSSP | |
| | | VTKSFNRGEC | |
| 3881 | CDRH1 of | GFSLSTSGM | GFTX3b |
| | IgGIL10M11 | | |
| | (Chothia) | | |
| 3882 | CDRH2 of | WWDSPGQGTQSENSCTHFPGNLPNMLRDL | GFTX3b |
| | IgGIL10M11 | RDAFSRVKTFFQMKDQLDNLLLKESLLED | IL10 |
| | (Chothia) | FKGYLGCQALSEMIQFYLEEVMPQAENQD | grafted |
| | | PDIKAHVNSLGENLKTLRLRLRRCHRFLP | into CDRH2. |
| | | CENGGGSGGKSKAVEQVKNAFNKLQEKGI | IL10 is |
| | | YKAMSEFDIFINYIEAYMTMKIRNDK | bolded, |
| | | | underlined |
| 3883 | CDRH3 of | SMITNWYFDV | GFTX3b |
| | IgGIL10M11 | | |
| | (Chothia) | | |
| 3884 | CDRL1 of | QLSVGY | GFTX3b |
| | IgGIL10M11 | | |
| | (Chothia) | | |
| 3885 | CDRL2 of | DTS | GFTX3b |
| | IgGIL10M11 | | |
| | (Chothia) | | |
| 3886 | CDRL3 of | GSGYPF | GFTX3b |
| | IgGIL10M11 | | |
| | (Chothia) | | |
| 3887 | CDRH1 of | TSGMSVG | GFTX3b |
| | IgGIL10M11 | | |
| | (Kabat) | | |
| 3888 | CDRH2 of | DIWWDSPGQGTQSENSCTHFPGNLPNMLR | GFTX3b |
| | IgGIL10M11 | DLRDAFSRVKTFFQMKDQLDNLLLKESLL | IL10 |
| | (Kabat) | EDFKGYLGCQALSEMIQFYLEEVMPQAEN | grafted |
| | | QDPDIKAHVNSLGENLKTLRLRLRRCHRF | into CDRH2. |
| | | LPCENGGGSGGKSKAVEQVKNAFNKLQEK | IL10 is |
| | | GIYKAMSEFDIFINYIEAYMTMKIRNDKK | bolded, |
| | | DYNPSLKS | underlined |
| 3889 | CDRH3 of | SMITNWYFDV | GFTX3b |
| | IgGIL10M11 | | |
| | (Kabat) | | |
| 3890 | CDRL1 of | KAQLSVGYMH | GFTX3b |
| | IgGIL10M11 | | |
| | (Kabat) | | |
| 3891 | CDRL2 of | DTSKLAS | GFTX3b |
| | IgGIL10M11 | | |
| | (Kabat) | | |
| 3892 | CDRL3 of | FQGSGYPFT | GFTX3b |
| | IgGIL10M11 | | |
| | (Kabat) | | |
| 3893 | VH of | QVTLRESGPALVKPTQTLTLTCTFSGFSL | GFTX3b |
| | IgGIL10M11 | STSGMSVGWIRQPPGKALEWLADIWWDSP | IL10 |
| | | GQGTQSENSCTHFPGNLPNMLRDLRDAFS | grafted |
| | | RVKTFFQMKDQLDNLLLKESLLEDFKGYL | into CDRH2 |
| | | GCQALSEMIQFYLEEVMPQAENQDPDIKA | |
| | | HVNSLGENLKTLRLRLRRCHRFLPCENGG | |
| | | GSGGKSKAVEQVKNAFNKLQEKGIYKAMS | |
| | | EFDIFINYIEAYMTMKIRNDKKDYNPSLK | |
| | | SRLTISKDTSANQVVLKVTNMDPADTATY | |
| | | YCARSMITNWYFDVWGAGTTVTVSS | |
| 3894 | VL of | DIQMTQSPSTLSASVGDRVTITCKAQLSV | GFTX3b |
| | IgGIL10M11 | GYMHWYQQKPGKAPKLLIYDTSKLASGVP | |
| | | SRFSGSGSGTAFTLTISSLQPDDFATYYC | |
| | | FQGSGYPFTFGGGTKLEIK | |
| 3895 | Heavy chain | QVTLRESGPALVKPTQTLTLTCTFSGFSL | GFTX3b |
| | of IgGIL10M11 | STSGMSVGWIRQPPGKALEWLADIWWDSP | IL10 |
| | | GQGTQSENSCTHFPGNLPNMLRDLRDAFS | grafted |
| | | RVKTFFQMKDQLDNLLLKESLLEDFKGYL | into CDRH2 |
| | | GCQALSEMIQFYLEEVMPQAENQDPDIKA | |
| | | HVNSLGENLKTLRLRLRRCHRFLPCENGG | |
| | | GSGGKSKAVEQVKNAFNKLQEKGIYKAMS | |
| | | EFDIFINYIEAYMTMKIRNDKKDYNPSLK | |
| | | SRLTISKDTSANQVVLKVTNMDPADTATY | |
| | | YCARSMITNWYFDVWGAGTTVTVSSASTK | |
| | | GPSVFPLAPSSKSTSGGTAALGCLVKDYF | |
| | | PEPVTVSWNSGALTSGVHTFPAVLQSSGL | |
| | | YSLSSVVTVPSSSLGTQTYICNVNHKPSN | |
| | | TKVDKRVEPKSCDKTHTCPPCPAPELLGG | |
| | | PSVFLFPPKPKDTLMISRTPEVTCVVVDV | |
| | | SHEDPEVKFNWYVDGVEVHNAKTKPREEQ | |
| | | YNSTYRVVSVLTVLHQDWLNGKEYKCKVS | |
| | | NKALPAPIEKTISKAKGQPREPQVYTLPP | |
| | | SREEMTKNQVSLTCLVKGFYPSDIAVEWE | |
| | | SNGQPENNYKTTPPVLDSDGSFFLYSKLT | |
| | | VDKSRWQQGNVFSCSVMHEALHNHYTQKS | |
| | | LSLSPGK | |
| 3896 | Light chain | DIQMTQSPSTLSASVGDRVTITCKAQLSV | GFTX3b |
| | of IgGIL10M11 | GYMHWYQQKPGKAPKLLIYDTSKLASGVP | |
| | | SRFSGSGSGTAFTLTISSLQPDDFATYYC | |
| | | FQGSGYPFTFGGGTKLEIKRTVAAPSVFI | |
| | | FPPSDEQLKSGTASVVCLLNNFYPREAKV | |
| | | QWKVDNALQSGNSQESVTEQDSKDSTYSL | |
| | | SSTLTLSKADYEKHKVYACEVTHQGLSSP | |
| | | VTKSFNRGEC | |
| 3897 | CDRH1 of | GFSLSTSGM | GFTX3b |
| | IgGIL10M12 | | |
| | (Chothia) | | |
| 3898 | CDRH2 of | WWDDK | GFTX3b |
| | IgGIL10M12 | | |
| | (Chothia) | | |
| 3899 | CDRH3 of | SMITSPGQGTQSENSCTHFPGNLPNMLRD | GFTX3b |
| | IgGIL10M12 | LRDAFSRVKTFFQMKDQLDNLLLKESLLE | IL10 |
| | (Chothia) | DFKGYLGCQALSEMIQFYLEEVMPQAENQ | grafted |
| | | DPDIKAHVNSLGENLKTLRLRLRRCHRFL | into CDRH3. |
| | | PCENGGGSGGKSKAVEQVKNAFNKLQEKG | IL10 is |
| | | IYKAMSEFDIFINYIEAYMTMKIRNNWYF | bolded, |
| | | DV | underlined |
| 3900 | CDRL1 of | QLSVGY | GFTX3b |
| | IgGIL10M12 | | |
| | (Chothia) | | |
| 3901 | CDRL2 of | DTS | GFTX3b |
| | IgGIL10M12 | | |
| | (Chothia) | | |
| 3902 | CDRL3 of | GSGYPF | GFTX3b |
| | IgGIL10M12 | | |
| | (Chothia) | | |
| 3903 | CDRH1 of | TSGMSVG | GFTX3b |
| | IgGIL10M12 | | |
| | (Kabat) | | |
| 3904 | CDRH2 of | DIWWDDKKDYNPSLKS | GFTX3b |
| | IgGIL10M12 | | |
| | (Kabat) | | |
| 3905 | CDRH3 of | SMITSPGQGTQSENSCTHFPGNLPNMLRD | GFTX3b |
| | IgGIL10M12 | LRDAFSRVKTFFQMKDQLDNLLLKESLLE | IL10 |
| | (Kabat) | DFKGYLGCQALSEMIQFYLEEVMPQAENQ | grafted |
| | | DPDIKAHVNSLGENLKTLRLRLRRCHRFL | into CDRH3. |
| | | PCENGGGSGGKSKAVEQVKNAFNKLQEKG | IL10 is |
| | | IYKAMSEFDIFINYIEAYMTMKIRNNWYF | bolded, |
| | | DV | underlined |
| 3906 | CDRL1 of | KAQLSVGYMH | GFTX3b |
| | IgGIL10M12 | | |
| | (Kabat) | | |
| 3907 | CDRL2 of | DTSKLAS | GFTX3b |
| | IgGIL10M12 | | |
| | (Kabat) | | |
| 3908 | CDRL3 of | FQGSGYPFT | GFTX3b |
| | IgGIL10M12 | | |
| | (Kabat) | | |
| 3909 | VH of | QVTLRESGPALVKPTQTLTLTCTFSGFSL | GFTX3b |
| | IgGIL10M12 | STSGMSVGWIRQPPGKALEWLADIWWDDK | IL10 |
| | | KDYNPSLKSRLTISKDTSANQVVLKVTNM | grafted |
| | | DPADTATYYCARSMITSPGQGTQSENSCT | into CDRH3 |
| | | HFPGNLPNMLRDLRDAFSRVKTFFQMKOQ | |
| | | LDNLLLKESLLEDFKGYLGCQALSEMIQF | |
| | | YLEEVMPQAENQDPDIKAHVNSLGENLKT | |
| | | LRLRLRRCHRFLPCENGGGSGGKSKAVEQ | |
| | | VKNAFNKLQEKGIYKAMSEFDIFINYIEA | |
| | | YMTMKIRNNWYFDVWGAGTTVTVSS | |
| 3910 | VL of | DIQMTQSPSTLSASVGDRVTITCKAQLSV | GFTX3b |
| | IgGIL10M12 | GYMHWYQQKPGKAPKLLIYDTSKLASGVP | |
| | | SRFSGSGSGTAFTLTISSLQPDDFATYYC | |
| | | FQGSGYPFTFGGGTKLEIK | |
| 3911 | Heavy chain | QVTLRESGPALVKPTQTLTLTCTFSGFSL | GFTX3b |
| | of IgGIL10M12 | STSGMSVGWIRQPPGKALEWLADIWWDDK | IL10 |
| | | KDYNPSLKSRLTISKDTSANQVVLKVTNM | grafted |
| | | DPADTATYYCARSMITSPGQGTQSENSCT | into CDRH3 |
| | | HFPGNLPNMLRDLRDAFSRVKTFFQMKOQ | |
| | | LDNLLLKESLLEDFKGYLGCQALSEMIQF | |
| | | YLEEVMPQAENQDPDIKAHVNSLGENLKT | |
| | | LRLRLRRCHRFLPCENGGGSGGKSKAVEQ | |
| | | VKNAFNKLQEKGIYKAMSEFDIFINYIEA | |
| | | YMTMKIRNNWYFDVWGAGTTVTVSSASTK | |
| | | GPSVFPLAPSSKSTSGGTAALGCLVKDYF | |
| | | PEPVTVSWNSGALTSGVHTFPAVLQSSGL | |
| | | YSLSSVVTVPSSSLGTQTYICNVNHKPSN | |
| | | TKVDKRVEPKSCDKTHTCPPCPAPELLGG | |
| | | PSVFLFPPKPKDTLMISRTPEVTCVVVDV | |
| | | SHEDPEVKFNWYVDGVEVHNAKTKPREEQ | |
| | | YNSTYRVVSVLTVLHQDWLNGKEYKCKVS | |
| | | NKALPAPIEKTISKAKGQPREPQVYTLPP | |
| | | SREEMTKNQVSLTCLVKGFYPSDIAVEWE | |
| | | SNGQPENNYKTTPPVLDSDGSFFLYSKLT | |
| | | VDKSRWQQGNVFSCSVMHEALHNHYTQKS | |
| | | LSLSPGK | |
| 3912 | Light chain | DIQMTQSPSTLSASVGDRVTITCKAQLSV | GFTX3b |
| | of IgGIL10M12 | GYMHWYQQKPGKAPKLLIYDTSKLASGVP | |
| | | SRFSGSGSGTAFTLTISSLQPDDFATYYC | |
| | | FQGSGYPFTFGGGTKLEIKRTVAAPSVFI | |
| | | FPPSDEQLKSGTASVVCLLNNFYPREAKV | |
| | | QWKVDNALQSGNSQESVTEQDSKDSTYSL | |
| | | SSTLTLSKADYEKHKVYACEVTHQGLSSP | |
| | | VTKSFNRGEC | |
| 3913 | CDRH1 of | GFSLSTSGM | GFTX3b |
| | IgGIL10M13 | | |
| | (Chothia) | | |
| 3914 | CDRH2 of | WWDDK | GFTX3b |
| | IgGIL10M13 | | |
| | (Chothia) | | |
| 3915 | CDRH3 of | SMITNWYFDV | GFTX3b |
| | IgGIL10M13 | | |
| | (Chothia) | | |
| 3916 | CDRL1 of | QLSSPGQGTQSENSCTHFPGNLPNMLRDL | IL10 |
| | IgGIL10M13 | RDAFSRVKTFFQMKDQLDNLLLKESLLED | grafted |
| | (Chothia) | FKGYLGCQALSEMIQFYLEEVMPQAENQD | into CDRL1. |
| | | PDIKAHVNSLGENLKTLRLRLRRCHRFLP | IL10 is |
| | | CENGGGSGGKSKAVEQVKNAFNKLQEKGI | bolded, |
| | | YKAMSEFDIFINYIEAYMTMKIRNVGY | underlined |
| 3917 | CDRL2 of | DTS | GFTX3b |
| | IgGIL10M13 | | |
| | (Chothia) | | |
| 3918 | CDRL3 of | GSGYPF | GFTX3b |
| | IgGIL10M13 | | |
| | (Chothia) | | |
| 3919 | CDRH1 of | TSGMSVG | GFTX3b |
| | IgGIL10M13 | | |
| | (Kabat) | | |
| 3920 | CDRH2 of | DIWWDDKKDYNPSLKS | GFTX3b |
| | IgGIL10M13 | | |
| | (Kabat) | | |
| 3921 | CDRH3 of | SMITNWYFDV | GFTX3b |
| | IgGIL10M13 | | |
| | (Kabat) | | |
| 3922 | CDRL1 of | KAQLSSPGQGTQSENSCTHFPGNLPNMLR | IL10 |
| | IgGIL10M13 | DLRDAFSRVKTFFQMKDQLDNLLLKESLL | grafted |
| | (Kabat) | EDFKGYLGCQALSEMIQFYLEEVMPQAEN | into CDRL1 |
| | | QDPDIKAHVNSLGENLKTLRLRLRRCHRF | IL10 is |
| | | LPCENGGGSGGKSKAVEQVKNAFNKLQEK | bolded, |
| | | GIYKAMSEFDIFINYIEAYMTMKIRNVGY | underlined |
| | | MH | GFTX3b |
| 3923 | CDRL2 of | DTSKLAS | GFTX3b |
| | IgGIL10M13 | | |
| | (Kabat) | | |
| 3924 | CDRL3 of | FQGSGYPFT | GFTX3b |
| | IgGIL10M13 | | |
| | (Kabat) | | |
| 3925 | VH of | QVTLRESGPALVKPTQTLTLTCTFSGFSL | GFTX3b |
| | IgGIL10M13 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3926 | VL of | DIQMTQSPSTLSASVGDRVTITCKAQLSS | IL10 |
| | IgGIL10M13 | PGQGTQSENSCTHFPGNLPNMLRDLRDAF | grafted |
| | | SRVKTFFQMKDQLDNLLLKESLLEDFKGY | into CDRL1 |
| | | LGCQALSEMIQFYLEEVMPQAENQDPDIK | GFTX3b |
| | | AHVNSLGENLKTLRLRLRRCHRFLPCENG | |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIK | |
| 3927 | Heavy chain | QVTLRESGPALVKPTQTLTLTCTFSGFSL | GFTX3b |
| | of IgGIL10M13 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPELLGGPSVFLFPPKPKDTLMISRTPE | |
| | | VTCVVVAVSHEDPEVKFNWYVDGVEVHNA | |
| | | KTKPREEQYNSTYRVVSVLTVLHQDWLNG | |
| | | KEYKCKVSNKALAAPIEKTISKAKGQPRE | |
| | | PQVYTLPPSREEMTKNQVSLTCLVKGFYP | |
| | | SDIAVEWESNGQPENNYKTTPPVLDSDGS | |
| | | FFLYSKLTVDKSRWQQGNVFSCSVMHEAL | |
| | | HNHYTQKSLSLSPGK | |
| 3928 | Light chain | DIQMTQSPSTLSASVGDRVTITCKAQLSS | IL10 |
| | of IgGIL10M13 | PGQGTQSENSCTHFPGNLPNMLRDLRDAF | grafted |
| | | SRVKTFFQMKDQLDNLLLKESLLEDFKGY | into CDRL1 |
| | | LGCQALSEMIQFYLEEVMPQAENQDPDIK | GFTX3b |
| | | AHVNSLGENLKTLRLRLRRCHRFLPCENG | |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3929 | Monomeric | SPGQGTQSENSCTHFPGNLPNMLRDLRDA | Mature |
| | IL10 (IL10M) | FSRVKTFFQMKDQLDNLLLKESLLEDFKG | form of |
| | | YLGCQALSEMIQFYLEEVMPQAENQDPDI | IL10, |
| | | KAHVNSLGENLKTLRLRLRRCHRFLPCEN | with an |
| | | GGGSGGKSKAVEQVKNAFNKLQEKGIYKA | internal |
| | | MSEFDIFINYIEAYMTMKIRN | G35G2 |
| | | | spacer |
| | | | (SEQ ID |
| | | | NO: 3971) |
| 3930 | CDRH1 of | GFSLSTSGM | GFTX3b |
| | IgGIL10M14 | | |
| | (Chothia) | | |
| 3931 | CDRH2 of | WWDDK | GFTX3b |
| | IgGIL10M14 | | |
| | (Chothia) | | |
| 3932 | CDRH3 of | SMITNWYFDV | GFTX3b |
| | IgGIL10M14 | | |
| | (Chothia) | | |
| 3933 | CDRL1 of | QLSSPGQGTQSENSCTHFPGNLPNMLRDL | IL10 |
| | IgGIL10M14 | RDAFSRVKTFFQMKDQLDNLLLKESLLED | grafted |
| | (Chothia) | FKGYLGCQALSEMIQFYLEEVMPQAENQD | into CDRL1. |
| | | PDIKAHVNSLGENLKTLRLRLRRCHRFLP | IL10 is |
| | | CENGGGSGGKSKAVEQVKNAFNKLQEKGI | bolded, |
| | | YKAMSEFDIFINYIEAYMTMKIRNVGY | underlined |
| | | | GFTX3b |
| 3934 | CDRL2 of | DTS | GFTX3b |
| | IgGIL10M14 | | |
| | (Chothia) | | |
| 3935 | CDRL3 of | GSGYPF | GFTX3b |
| | IgGIL10M14 | | |
| | (Chothia) | | |
| 3936 | CDRH1 of | TSGMSVG | GFTX3b |
| | IgGIL10M14 | | |
| | (Kabat) | | |
| 3937 | CDRH2 of | DIWWDDKKDYNPSLKS | GFTX3b |
| | IgGIL10M14 | | |
| | (Kabat) | | |
| 3938 | CDRH3 of | SMITNWYFDV | GFTX3b |
| | IgGIL10M14 | | |
| | (Kabat) | | |
| 3939 | CDRL1 of | KAQLSSPGQGTQSENSCTHFPGNLPNMLR | IL10 |
| | IgGIL10M14 | DLRDAFSRVKTFFQMKDQLDNLLLKESLL | grafted |
| | (Kabat) | EDFKGYLGCQALSEMIQFYLEEVMPQAEN | into CDRL1. |
| | | QDPDIKAHVNSLGENLKTLRLRLRRCHRF | IL10 is |
| | | LPCENGGGSGGKSKAVEQVKNAFNKLQEK | bolded, |
| | | GIYKAMSEFDIFINYIEAYMTMKIRNVGY | underlined |
| | | MH | GFTX3b |
| 3940 | CDRL2 of | DTSKLAS | GFTX3b |
| | IgGIL10M14 | | |
| | (Kabat) | | |
| 3941 | CDRL3 of | FQGSGYPFT | GFTX3b |
| | IgGIL10M14 | | |
| | (Kabat) | | |
| 3942 | VH of | QVTLRESGPALVKPTQTLTLTCTFSGFSL | GFTX3b |
| | IgGIL10M14 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3943 | VL of | DIQMTQSPSTLSASVGDRVTITCKAQLSS | IL10 |
| | IgGIL10M14 | PGQGTQSENSCTHFPGNLPNMLRDLRDAF | grafted |
| | | SRVKTFFQMKDQLDNLLLKESLLEDFKGY | into CDRL1 |
| | | LGCQALSEMIQFYLEEVMPQAENQDPDIK | GFTX3b |
| | | AHVNSLGENLKTLRLRLRRCHRFLPCENG | |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIK | |
| 3944 | Heavy chain | QVTLRESGPALVKPTQTLTLTCTFSGFSL | GFTX3b |
| | of IgGIL10M14 | STSGMSVGWIRQPPGKALEWLADIWWDDK | LALA |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPEAAGGPSVFLFPPKPKDTLMISRTPE | |
| | | VTCVVVDVSHEDPEVKFNWYVDGVEVHNA | |
| | | KTKPREEQYNSTYRVVSVLTVLHQDWLNG | |
| | | KEYKCKVSNKALPAPIEKTISKAKGQPRE | |
| | | PQVYTLPPSREEMTKNQVSLTCLVKGFYP | |
| | | SDIAVEWESNGQPENNYKTTPPVLDSDGS | |
| | | FFLYSKLTVDKSRWQQGNVFSCSVMHEAL | |
| | | HNHYTQKSLSLSPGK | |
| 3945 | Light chain | DIQMTQSPSTLSASVGDRVTITCKAQLSS | GFTX3b |
| | of IgGIL10M14 | PGQGTQSENSCTHFPGNLPNMLRDLRDAF | LALA |
| | | SRVKTFFQMKDQLDNLLLKESLLEDFKGY | IL10 |
| | | LGCQALSEMIQFYLEEVMPQAENQDPDIK | grafted |
| | | AHVNSLGENLKTLRLRLRRCHRFLPCENG | into CDRL1. |
| | | GGSGGKSKAVEQVKNAFNKLQEKGIYKAM | |
| | | SEFDIFINYIEAYMTMKIRNVGYMHWYQQ | |
| | | KPGKAPKLLIYDTSKLASGVPSRFSGSGS | |
| | | GTAFTLTISSLQPDDFATYYCFQGSGYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3946 | CDRH1 of | GFSLSTSGM | GFTX3b |
| | IgGIL10M15 | | |
| | (Chothia) | | |
| 3947 | CDRH2 of | WWDDK | GFTX3b |
| | IgGIL10M15 | | |
| | (Chothia) | | |
| 3948 | CDRH3 of | SMITNWYFDV | GFTX3b |
| | IgGIL10M15 | | |
| | (Chothia) | | |
| 3949 | CDRL1 of | QLSSPGQGTQSENSCTHFPGNLPNMLRDL | IL10 |
| | IgGIL10M15 | RDAFSRVKTFFQMKDQLDNLLLKESLLED | grafted |
| | (Chothia) | FKGYLGCQALSEMIQFYLEEVMPQAENQD | into CDRL1. |
| | | PDIKAHVNSLGENLKTLRLRLRRCHRFLP | IL10 is |
| | | CENGGGSGGKSKAVEQVKNAFNKLQEKGI | bolded, |
| | | YKAMSEFDIFINYIEAYMTMKIRNVGY | underlined |
| 3950 | CDRL2 of | DTS | GFTX3b |
| | IgGIL10M15 | | |
| | (Chothia) | | |
| 3951 | CDRL3 of | GSGYPF | GFTX3b |
| | IgGIL10M15 | | |
| | (Chothia) | | |
| 3952 | CDRH1 of | TSGMSVG | GFTX3b |
| | IgGIL10M15 | | |
| | (Kabat) | | |
| 3953 | CDRH2 of | DIWWDDKKDYNPSLKS | GFTX3b |
| | IgGIL10M15 | | |
| | (Kabat) | | |
| 3954 | CDRH3 of | SMITNWYFDV | GFTX3b |
| | IgGIL10M15 | | |
| | (Kabat) | | |
| 3955 | CDRL1 of | KAQLSSPGQGTQSENSCTHFPGNLPNMLR | IL10 |
| | IgGIL10M15 | DLRDAFSRVKTFFQMKDQLDNLLLKESLL | grafted |
| | (Kabat) | EDFKGYLGCQALSEMIQFYLEEVMPQAEN | into CDRL1 |
| | | QDPDIKAHVNSLGENLKTLRLRLRRCHRF | IL10 is |
| | | LPCENGGGSGGKSKAVEQVKNAFNKLQEK | bolded, |
| | | GIYKAMSEFDIFINYIEAYMTMKIRNVGY | underlined |
| | | MH | GFTX3b |
| 3956 | CDRL2 of | DTSKLAS | GFTX3b |
| | IgGIL10M15 | | |
| | (Kabat) | | |
| 3957 | CDRL3 of | FQGSGYPFT | GFTX3b |
| | IgGIL10M15 | | |
| | (Kabat) | | |
| 3958 | VH of | QVTLRESGPALVKPTQTLTLTCTFSGFSL | GFTX3b |
| | IgGIL10M15 | STSGMSVGWIRQPPGKALEWLADIWWDDK | |
| | | KDYNPSLKSRLTISKDTSANQVVLKVTNM | |
| | | DPADTATYYCARSMITNWYFDVWGAGTTV | |
| | | TVSS | |
| 3959 | VL of | DIQMTQSPSTLSASVGDRVTITCKAQLSS | IL10 |
| | IgGIL10M15 | PGQGTQSENSCTHFPGNLPNMLRDLRDAF | grafted |
| | | SRVKTFFQMKDQLDNLLLKESLLEDFKGY | into CDRL1 |
| | | LGCQALSEMIQFYLEEVMPQAENQDPDIK | IL10 is |
| | | AHVNSLGENLKTLRLRLRRCHRFLPCENG | bolded, |
| | | GGSGGKSKAVEQVKNAFNKLQEKGIYKAM | underlined |
| | | SEFDIFINYIEAYMTMKIRNVGYMHWYQQ | GFTX3b |
| | | KPGKAPKLLIYDTSKLASGVPSRFSGSGS | |
| | | GTAFTLTISSLQPDDFATYYCFQGSGYPF | |
| | | TFGGGTKLEIK | |
| 3960 | Heavy chain | QVTLRESGPALVKPTQTLTLTCTFSGFSL | GFTX3b |
| | of IgGIL10M15 | STSGMSVGWIRQPPGKALEWLADIWWDDK | NEM |
| | | KDYNPSLKSRLTISKDTSANQVVLKVTNM | |
| | | DPADTATYYCARSMITNWYFDVWGAGTTV | |
| | | TVSSASTKGPSVFPLAPSSKSTSGGTAAL | |
| | | GCLVKDYFPEPVTVSWNSGALTSGVHTFP | |
| | | AVLQSSGLYSLSSVVTVPSSSLGTQTYIC | |
| | | NVNHKPSNTKVDKRVEPKSCDKTHTCPPC | |
| | | PAPELLGGPSVFLFPPKPKDTLMISRTPE | |
| | | VTCVVVDVSHEDPEVKFNWYVDGVEVHNA | |
| | | KTKPREEQYASTYRVVSVLTVLHQDWLNG | |
| | | KEYKCKVSNKALPAPIEKTISKAKGQPRE | |
| | | PQVYTLPPSREEMTKNQVSLTCLVKGFYP | |
| | | SDIAVEWVSNGQPENNYKTTPPVLDSDGS | |
| | | FFLYSKLTVDKSRWQQGNVFSCSVIHEAL | |
| | | HNHYTQKSLSLSPGK | |
| 3961 | Light chain | DIQMTQSPSTLSASVGDRVTITCKAQLSS | GFTX3b |
| | of IgGIL10M15 | PGQGTQSENSCTHFPGNLPNMLRDLRDAF | NEM IL10 |
| | | SRVKTFFQMKDQLDNLLLKESLLEDFKGY | grafted |
| | | LGCQALSEMIQFYLEEVMPQAENQDPDIK | into CDRL1. |
| | | AHVNSLGENLKTLRLRLRRCHRFLPCENG | IL10 is |
| | | GGSGGKSKAVEQVKNAFNKLQEKGIYKAM | bolded, |
| | | SEFDIFINYIEAYMTMKIRNVGYMHWYQQ | underlined |
| | | KPGKAPKLLIYDTSKLASGVPSRFSGSGS | |
| | | GTAFTLTISSLQPDDFATYYCFQGSGYPF | |
| | | TFGGGTKLEIKRTVAAPSVFIFPPSDEQL | |
| | | KSGTASVVCLLNNFYPREAKVQWKVDNAL | |
| | | QSGNSQESVTEQDSKDSTYSLSSTLTLSK | |
| | | ADYEKHKVYACEVTHQGLSSPVTKSFNRG | |
| | | EC | |
| 3962 | VH of | CAGGTCACACTGAGAGAGTCAGGCCCTGC | |
| | IgGIL10M7 | CCTGGTCAAGCCTACTCAGACCCTGACCC | |
| | | TGACCTGCACCTTTAGCGGCTTTAGCCTG | |
| | | AGCACTAGCGGAATGAGCGTGGGCTGGAT | |
| | | TAGACAGCCCCCTGGTAAAGCCCTGGAGT | |
| | | GGCTGGCCGATATTTGGTGGGACGATAAG | |
| | | AAGGACTATAACCCTAGCCTGAAGTCTAG | |
| | | GCTGACTATCTCTAAGGACACTAGCGCTA | |
| | | ATCAGGTGGTGCTGAAAGTGACTAATATG | |
| | | GACCCCGCCGACACCGCTACCTACTACTG | |
| | | CGCTAGATCTATGATCACTAACTGGTACT | |
| | | TCGACGTGTGGGGCGCTGGCACTACCGTG | |
| | | ACCGTGTCTAGC | |
| 3963 | VL of | GATATTCAGATGACTCAGTCACCTAGCAC | |
| | IgGIL10M7 | CCTGAGCGCTAGTGTGGGCGATAGAGTGA | |
| | | CTATCACCTGTAAAGCTCAGCTGTCTAGC | |
| | | CCAGGTCAGGGAACTCAGTCAGAGAATAG | |
| | | CTGCACTCACTTCCCCGGTAACCTGCCTA | |
| | | ATATGCTGAGAGATCTGAGGGACGCCTTC | |
| | | TCTAGGGTCAAGACCTTCTTTCAGATGAA | |
| | | GGATCAGCTGGATAACCTGCTGCTGAAAG | |
| | | AGTCACTGCTGGAGGACTTTAAGGGCTAC | |
| | | CTGGGCTGTCAGGCCCTGAGCGAGATGAT | |
| | | TCAGTTCTACCTGGAAGAAGTGATGCCCC | |
| | | AGGCCGAGAATCAGGACCCCGATATTAAG | |
| | | GCTCACGTGAACTCACTGGGCGAGAACCT | |
| | | GAAAACCCTGAGACTGAGGCTGAGGCGGT | |
| | | GTCACCGGTTTCTGCCCTGCGAGAACGGC | |
| | | GGAGGTAGCGGCGGTAAATCTAAGGCCGT | |
| | | GGAACAGGTCAAAAACGCCTTTAACAAGC | |
| | | TGCAGGAAAAGGGAATCTATAAGGCTATG | |
| | | AGCGAGTTCGACATCTTTATTAACTATAT | |
| | | CGAGGCCTATATGACTATGAAGATTAGGA | |
| | | ACGTGGGCTATATGCACTGGTATCAGCAG | |
| | | AAGCCCGGTAAAGCCCCTAAGCTGCTGAT | |
| | | CTACGACACCTCTAAGCTGGCTAGTGGCG | |
| | | TGCCCTCTAGGTTTAGCGGTAGCGGTAGT | |
| | | GGCACCGCCTTCACCCTGACTATCTCTAG | |
| | | CCTGCAGCCCGACGACTTCGCTACCTACT | |
| | | ACTGTTTTCAGGGTAGCGGCTACCCCTTC | |
| | | ACCTTCGGCGGAGGCACTAAGCTGGAGAT | |
| | | TAAG | |
| 3964 | Heavy Chain | CAGGTCACACTGAGAGAGTCAGGCCCTGC | |
| | of IgGIL10M7 | CCTGGTCAAGCCTACTCAGACCCTGACCC | |
| | | TGACCTGCACCTTTAGCGGCTTTAGCCTG | |
| | | AGCACTAGCGGAATGAGCGTGGGCTGGAT | |
| | | TAGACAGCCCCCTGGTAAAGCCCTGGAGT | |
| | | GGCTGGCCGATATTTGGTGGGACGATAAG | |
| | | AAGGACTATAACCCTAGCCTGAAGTCTAG | |
| | | GCTGACTATCTCTAAGGACACTAGCGCTA | |
| | | ATCAGGTGGTGCTGAAAGTGACTAATATG | |
| | | GACCCCGCCGACACCGCTACCTACTACTG | |
| | | CGCTAGATCTATGATCACTAACTGGTACT | |
| | | TCGACGTGTGGGGCGCTGGCACTACCGTG | |
| | | ACCGTGTCTAGCGCTAGCACTAAGGGCCC | |
| | | AAGTGTGTTTCCCCTGGCCCCCAGCAGCA | |
| | | AGTCTACTTCCGGCGGAACTGCTGCCCTG | |
| | | GGTTGCCTGGTGAAGGACTACTTCCCCGA | |
| | | GCCCGTGACAGTGTCCTGGAACTCTGGGG | |
| | | CTCTGACTTCCGGCGTGCACACCTTCCCC | |
| | | GCCGTGCTGCAGAGCAGCGGCCTGTACAG | |
| | | CCTGAGCAGCGTGGTGACAGTGCCCTCCA | |
| | | GCTCTCTGGGAACCCAGACCTATATCTGC | |
| | | AACGTGAACCACAAGCCCAGCAACACCAA | |
| | | GGTGGACAAGAGAGTGGAGCCCAAGAGCT | |
| | | GCGACAAGACCCACACCTGCCCCCCCTGC | |
| | | CCAGCTCCAGAACTGCTGGGAGGGCCTTC | |
| | | CGTGTTCCTGTTCCCCCCCAAGCCCAAGG | |
| | | ACACCCTGATGATCAGCAGGACCCCCGAG | |
| | | GTGACCTGCGTGGTGGTGGACGTGTCCCA | |
| | | CGAGGACCCAGAGGTGAAGTTCAACTGGT | |
| | | ACGTGGACGGCGTGGAGGTGCACAACGCC | |
| | | AAGACCAAGCCCAGAGAGGAGCAGTACAA | |
| | | CAGCACCTACAGGGTGGTGTCCGTGCTGA | |
| | | CCGTGCTGCACCAGGACTGGCTGAACGGC | |
| | | AAAGAATACAAGTGCAAAGTCTCCAACAA | |
| | | GGCCCTGCCAGCCCCAATCGAAAAGACAA | |
| | | TCAGCAAGGCCAAGGGCCAGCCACGGGAG | |
| | | CCCCAGGTGTACACCCTGCCCCCCAGCCG | |
| | | GGAGGAGATGACCAAGAACCAGGTGTCCC | |
| | | TGACCTGTCTGGTGAAGGGCTTCTACCCC | |
| | | AGCGATATCGCCGTGGAGTGGGAGAGCAA | |
| | | CGGCCAGCCCGAGAACAACTACAAGACCA | |
| | | CCCCCCCAGTGCTGGACAGCGACGGCAGC | |
| | | TTCTTCCTGTACAGCAAGCTGACCGTGGA | |
| | | CAAGTCCAGGTGGCAGCAGGGCAACGTGT | |
| | | TCAGCTGCAGCGTGATGCACGAGGCCCTG | |
| | | CACAACCACTACACCCAGAAGTCCCTGAG | |
| | | CCTGAGCCCCGGCAAG | |
| 3965 | Light Chain | GATATTCAGATGACTCAGTCACCTAGCAC | |
| | of IgGIL10M7 | CCTGAGCGCTAGTGTGGGCGATAGAGTGA | |
| | | CTATCACCTGTAAAGCTCAGCTGTCTAGC | |
| | | CCAGGTCAGGGAACTCAGTCAGAGAATAG | |
| | | CTGCACTCACTTCCCCGGTAACCTGCCTA | |
| | | ATATGCTGAGAGATCTGAGGGACGCCTTC | |
| | | TCTAGGGTCAAGACCTTCTTTCAGATGAA | |
| | | GGATCAGCTGGATAACCTGCTGCTGAAAG | |
| | | AGTCACTGCTGGAGGACTTTAAGGGCTAC | |
| | | CTGGGCTGTCAGGCCCTGAGCGAGATGAT | |
| | | TCAGTTCTACCTGGAAGAAGTGATGCCCC | |
| | | AGGCCGAGAATCAGGACCCCGATATTAAG | |
| | | GCTCACGTGAACTCACTGGGCGAGAACCT | |
| | | GAAAACCCTGAGACTGAGGCTGAGGCGGT | |
| | | GTCACCGGTTTCTGCCCTGCGAGAACGGC | |
| | | GGAGGTAGCGGCGGTAAATCTAAGGCCGT | |
| | | GGAACAGGTCAAAAACGCCTTTAACAAGC | |
| | | TGCAGGAAAAGGGAATCTATAAGGCTATG | |
| | | AGCGAGTTCGACATCTTTATTAACTATAT | |
| | | CGAGGCCTATATGACTATGAAGATTAGGA | |
| | | ACGTGGGCTATATGCACTGGTATCAGCAG | |
| | | AAGCCCGGTAAAGCCCCTAAGCTGCTGAT | |
| | | CTACGACACCTCTAAGCTGGCTAGTGGCG | |
| | | TGCCCTCTAGGTTTAGCGGTAGCGGTAGT | |
| | | GGCACCGCCTTCACCCTGACTATCTCTAG | |
| | | CCTGCAGCCCGACGACTTCGCTACCTACT | |
| | | ACTGTTTTCAGGGTAGCGGCTACCCCTTC | |
| | | ACCTTCGGCGGAGGCACTAAGCTGGAGAT | |
| | | TAAGCGTACGGTGGCCGCTCCCAGCGTGT | |
| | | TCATCTTCCCCCCCAGCGACGAGCAGCTG | |
| | | AAGAGCGGCACCGCCAGCGTGGTGTGCCT | |
| | | GCTGAACAACTTCTACCCCCGGGAGGCCA | |
| | | AGGTGCAGTGGAAGGTGGACAACGCCCTG | |
| | | CAGAGCGGCAACAGCCAGGAGAGCGTCAC | |
| | | CGAGCAGGACAGCAAGGACTCCACCTACA | |
| | | GCCTGAGCAGCACCCTGACCCTGAGCAAG | |
| | | GCCGACTACGAGAAGCATAAGGTGTACGC | |
| | | CTGCGAGGTGACCCACCAGGGCCTGTCCA | |
| | | GCCCCGTGACCAAGAGCTTCAACAGGGGC | |
| | | GAGTGC | |
| 3966 | VH of | CAGGTCACACTGAGAGAGTCAGGCCCTGC | |
| | IgGIL10M13 | CCTGGTCAAGCCTACTCAGACCCTGACCC | |
| | | TGACCTGCACCTTTAGCGGCTTTAGCCTG | |
| | | AGCACTAGCGGAATGAGCGTGGGCTGGAT | |
| | | TAGACAGCCCCCTGGTAAAGCCCTGGAGT | |
| | | GGCTGGCCGATATTTGGTGGGACGATAAG | |
| | | AAGGACTATAACCCTAGCCTGAAGTCTAG | |
| | | GCTGACTATCTCTAAGGACACTAGCGCTA | |
| | | ATCAGGTGGTGCTGAAAGTGACTAATATG | |
| | | GACCCCGCCGACACCGCTACCTACTACTG | |
| | | CGCTAGATCTATGATCACTAACTGGTACT | |
| | | TCGACGTGTGGGGCGCTGGCACTACCGTG | |
| | | ACCGTGTCTAGC | |
| 3967 | VL of | GATATTCAGATGACTCAGTCACCTAGCAC | |
| | IgGIL10M13 | CCTGAGCGCTAGTGTGGGCGATAGAGTGA | |
| | | CTATCACCTGTAAAGCTCAGCTGTCTAGC | |
| | | CCAGGTCAGGGAACTCAGTCAGAGAATAG | |
| | | CTGCACTCACTTCCCCGGTAACCTGCCTA | |
| | | ATATGCTGAGAGATCTGAGGGACGCCTTC | |
| | | TCTAGGGTCAAGACCTTCTTTCAGATGAA | |
| | | GGATCAGCTGGATAACCTGCTGCTGAAAG | |
| | | AGTCACTGCTGGAGGACTTTAAGGGCTAC | |
| | | CTGGGCTGTCAGGCCCTGAGCGAGATGAT | |
| | | TCAGTTCTACCTGGAAGAAGTGATGCCCC | |
| | | AGGCCGAGAATCAGGACCCCGATATTAAG | |
| | | GCTCACGTGAACTCACTGGGCGAGAACCT | |
| | | GAAAACCCTGAGACTGAGGCTGAGGCGGT | |
| | | GTCACCGGTTTCTGCCCTGCGAGAACGGC | |
| | | GGAGGTAGCGGCGGTAAATCTAAGGCCGT | |
| | | GGAACAGGTCAAAAACGCCTTTAACAAGC | |
| | | TGCAGGAAAAGGGAATCTATAAGGCTATG | |
| | | AGCGAGTTCGACATCTTTATTAACTATAT | |
| | | CGAGGCCTATATGACTATGAAGATTAGGA | |
| | | ACGTGGGCTATATGCACTGGTATCAGCAG | |
| | | AAGCCCGGTAAAGCCCCTAAGCTGCTGAT | |
| | | CTACGACACCTCTAAGCTGGCTAGTGGCG | |
| | | TGCCCTCTAGGTTTAGCGGTAGCGGTAGT | |
| | | GGCACCGCCTTCACCCTGACTATCTCTAG | |
| | | CCTGCAGCCCGACGACTTCGCTACCTACT | |
| | | ACTGTTTTCAGGGTAGCGGCTACCCCTTC | |
| | | ACCTTCGGCGGAGGCACTAAGCTGGAGAT | |
| | | TAAG | |
| 3968 | Heavy Chain | CAGGTCACACTGAGAGAGTCAGGCCCTGC | |
| | of IgGIL10M13 | CCTGGTCAAGCCTACTCAGACCCTGACCC | |
| | | TGACCTGCACCTTTAGCGGCTTTAGCCTG | |
| | | AGCACTAGCGGAATGAGCGTGGGCTGGAT | |
| | | TAGACAGCCCCCTGGTAAAGCCCTGGAGT | |
| | | GGCTGGCCGATATTTGGTGGGACGATAAG | |
| | | AAGGACTATAACCCTAGCCTGAAGTCTAG | |
| | | GCTGACTATCTCTAAGGACACTAGCGCTA | |
| | | ATCAGGTGGTGCTGAAAGTGACTAATATG | |
| | | GACCCCGCCGACACCGCTACCTACTACTG | |
| | | CGCTAGATCTATGATCACTAACTGGTACT | |
| | | TCGACGTGTGGGGCGCTGGCACTACCGTG | |
| | | ACCGTGTCTAGCGCTAGCACTAAGGGCCC | |
| | | CTCCGTGTTCCCTCTGGCCCCTTCCAGCA | |
| | | AGTCTACCTCCGGCGGCACAGCTGCTCTG | |
| | | GGCTGCCTGGTCAAGGACTACTTCCCTGA | |
| | | GCCTGTGACAGTGTCCTGGAACTCTGGCG | |
| | | CCCTGACCTCTGGCGTGCACACCTTCCCT | |
| | | GCCGTGCTGCAGTCCTCCGGCCTGTACTC | |
| | | CCTGTCCTCCGTGGTCACAGTGCCTTCAA | |
| | | GCAGCCTGGGCACCCAGACCTATATCTGC | |
| | | AACGTGAACCACAAGCCTTCCAACACCAA | |
| | | GGTGGACAAGCGGGTGGAGCCTAAGTCCT | |
| | | GCGACAAGACCCACACCTGTCCTCCCTGC | |
| | | CCTGCTCCTGAACTGCTGGGCGGCCCTTC | |
| | | TGTGTTCCTGTTCCCTCCAAAGCCCAAGG | |
| | | ACACCCTGATGATCTCCCGGACCCCTGAA | |
| | | GTGACCTGCGTGGTGGTGGCCGTGTCCCA | |
| | | CGAGGATCCTGAAGTGAAGTTCAATTGGT | |
| | | ACGTGGACGGCGTGGAGGTGCACAACGCC | |
| | | AAGACCAAGCCTCGGGAGGAACAGTACAA | |
| | | CTCCACCTACCGGGTGGTGTCCGTGCTGA | |
| | | CCGTGCTGCACCAGGACTGGCTGAACGGC | |
| | | AAAGAGTACAAGTGCAAAGTCTCCAACAA | |
| | | GGCCCTGGCCGCCCCTATCGAAAAGACAA | |
| | | TCTCCAAGGCCAAGGGCCAGCCTAGGGAA | |
| | | CCCCAGGTGTACACCCTGCCACCCAGCCG | |
| | | GGAGGAAATGACCAAGAACCAGGTGTCCC | |
| | | TGACCTGTCTGGTCAAGGGCTTCTACCCT | |
| | | TCCGATATCGCCGTGGAGTGGGAGTCTAA | |
| | | CGGCCAGCCTGAGAACAACTACAAGACCA | |
| | | CCCCTCCTGTGCTGGACTCCGACGGCTCC | |
| | | TTCTTCCTGTACTCCAAACTGACCGTGGA | |
| | | CAAGTCCCGGTGGCAGCAGGGCAACGTGT | |
| | | TCTCCTGCTCCGTGATGCACGAGGCCCTG | |
| | | CACAACCACTACACCCAGAAGTCCCTGTC | |
| | | CCTGTCTCCCGGCAAG | |
| 3969 | Light Chain | GATATTCAGATGACTCAGTCACCTAGCAC | |
| | of IgGIL10M13 | CCTGAGCGCTAGTGTGGGCGATAGAGTGA | |
| | | CTATCACCTGTAAAGCTCAGCTGTCTAGC | |
| | | CCAGGTCAGGGAACTCAGTCAGAGAATAG | |
| | | CTGCACTCACTTCCCCGGTAACCTGCCTA | |
| | | ATATGCTGAGAGATCTGAGGGACGCCTTC | |
| | | TCTAGGGTCAAGACCTTCTTTCAGATGAA | |
| | | GGATCAGCTGGATAACCTGCTGCTGAAAG | |
| | | AGTCACTGCTGGAGGACTTTAAGGGCTAC | |
| | | CTGGGCTGTCAGGCCCTGAGCGAGATGAT | |
| | | TCAGTTCTACCTGGAAGAAGTGATGCCCC | |
| | | AGGCCGAGAATCAGGACCCCGATATTAAG | |
| | | GCTCACGTGAACTCACTGGGCGAGAACCT | |
| | | GAAAACCCTGAGACTGAGGCTGAGGCGGT | |
| | | GTCACCGGTTTCTGCCCTGCGAGAACGGC | |
| | | GGAGGTAGCGGCGGTAAATCTAAGGCCGT | |
| | | GGAACAGGTCAAAAACGCCTTTAACAAGC | |
| | | TGCAGGAAAAGGGAATCTATAAGGCTATG | |
| | | AGCGAGTTCGACATCTTTATTAACTATAT | |
| | | CGAGGCCTATATGACTATGAAGATTAGGA | |
| | | ACGTGGGCTATATGCACTGGTATCAGCAG | |
| | | AAGCCCGGTAAAGCCCCTAAGCTGCTGAT | |
| | | CTACGACACCTCTAAGCTGGCTAGTGGCG | |
| | | TGCCCTCTAGGTTTAGCGGTAGCGGTAGT | |
| | | GGCACCGCCTTCACCCTGACTATCTCTAG | |
| | | CCTGCAGCCCGACGACTTCGCTACCTACT | |
| | | ACTGTTTTCAGGGTAGCGGCTACCCCTTC | |
| | | ACCTTCGGCGGAGGCACTAAGCTGGAGAT | |
| | | TAAGCGTACGGTGGCCGCTCCCAGCGTGT | |
| | | TCATCTTCCCCCCCAGCGACGAGCAGCTG | |
| | | AAGAGCGGCACCGCCAGCGTGGTGTGCCT | |
| | | GCTGAACAACTTCTACCCCCGGGAGGCCA | |
| | | AGGTGCAGTGGAAGGTGGACAACGCCCTG | |
| | | CAGAGCGGCAACAGCCAGGAGAGCGTCAC | |
| | | CGAGCAGGACAGCAAGGACTCCACCTACA | |
| | | GCCTGAGCAGCACCCTGACCCTGAGCAAG | |
| | | GCCGACTACGAGAAGCATAAGGTGTACGC | |
| | | CTGCGAGGTGACCCACCAGGGCCTGTCCA | |
| | | GCCCCGTGACCAAGAGCTTCAACAGGGGC | |
| | | GAGTGC | |
| 3970 | Monomeric | AGTCCCGGTCAGGGAACTCAGTCAGAGAA | |
| | IL10 | TAGCTGCACTCACTTCCCCGGTAACCTGC | |
| | | CTAATATGCTGAGAGATCTGAGGGACGCC | |
| | | TTCTCTAGGGTCAAGACCTTCTTTCAGAT | |
| | | GAAGGATCAGCTGGATAACCTGCTGCTGA | |
| | | AAGAGTCACTGCTGGAGGACTTTAAGGGC | |
| | | TACCTGGGCTGTCAGGCCCTGAGCGAGAT | |
| | | GATTCAGTTCTACCTGGAAGAAGTGATGC | |
| | | CCCAGGCCGAGAATCAGGACCCCGATATT | |
| | | AAGGCTCACGTCAACTCACTGGGCGAGAA | |
| | | CCTGAAAACCCTGAGACTGAGGCTGAGGC | |
| | | GGTGTCACCGGTTTCTGCCCTGCGAGAAC | |
| | | GGCGGAGGTAGCGGCGGTAAATCTAAGGC | |
| | | CGTGGAACAGGTCAAAAACGCCTTTAACA | |
| | | AGCTGCAGGAAAAGGGAATCTATAAGGCT | |
| | | ATGAGCGAGTTCGACATCTTTATTAACTA | |
| | | TATCGAGGCCTATATGACTATGAAGATTA | |
| | | GGAAC | |
| 3971 | linker | GGGSGG | |
| 3972 | linker | GGGGS | |
| 3973 | linker | GGGGA | |

### Example 39: Antibody Cytokine Engrafted Proteins have Anti-Inflammatory Activity

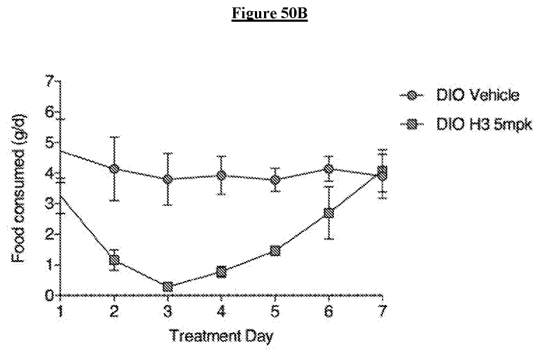

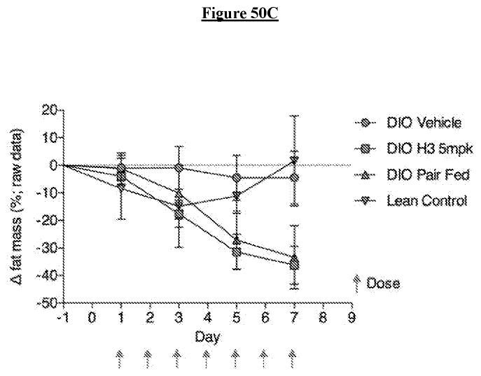

Using an assay developed in support of rhIL10's pro-inflammatory activity in the clinic (Lauw et al., J Immunol. 2000; 165(5):2783-9), the pro-inflammatory activity of IgGIL10M13 in human whole blood was assessed. In order to assess pro-inflammatory activity, antibody cytokine engrafted proteins were profiled for their ability to induce interferon gamma (IFNγ) or granzyme B in activated primary human CD8 T cells. It was found that antibody cytokine engrafted proteins such as IgGIL10M13 demonstrated significantly less pro-inflammatory activity than recombinant human IL10 (rhIL10) as measured by IFNγ production. This data is shown in FIG. 51A. Similar results were found in assays measuring granzyme B (data not shown), as well as with other exemplary antibody cytokine engrafted proteins (IgGIL10M7). The significantly decreased pro-inflammatory activity demonstrated by IgGIL10M13 as compared to rhIL10 indicates it would be superior to rhIL10 for treating immune related disorders, as IgGIL10M13 could be administered over a broader dose range.

To examine anti-inflammatory activity, antibody cytokine engrafted proteins and rhIL10 were tested for their ability to inhibit LPS-induced TNFα in human whole blood. This data is shown in FIG. 51B, wherein increasing concentrations of either rhIL10 or IgGIL10M13 reduced TNFα production. Note that the rhIL10 and IgGIL10M13 curves are similar, indicating that both molecules had potent anti-inflammatory activity.

In summary, these results show that antibody cytokine engrafted proteins have the desired properties of having anti-inflammatory properties similar to IL10, but without the dose limiting, and unwanted pro-inflammatory properties.

### Example 40: IL10 Dependent Signaling

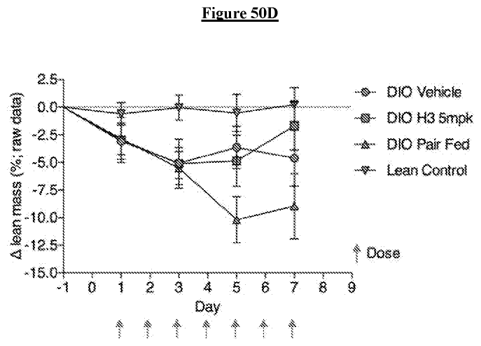

In vitro signaling studies in human PBMCs and whole blood indicate that antibody cytokine engrafted proteins such as IgGIL10M13 had a more specific signaling profile when compared to rhIL10. Using CyTOF, a FACS based method that utilizes mass spectrometry, antibody cytokine engrafted protein signaling in multiple different cell populations in whole blood was assessed by pSTAT3 detection (FIG. 52). Antibody cytokine engrafted proteins such IgGIL10M13 induced a pSTAT3 signal only on monocytes, macrophages and plasmacytoid dendritic cells above μM concentrations (up to 1.8 μM). All of these cell types are known to have increased expression of IL10 receptor. rhIL10 induced a pSTAT3 signal on monocytes, but also on additional cell types such as T cells, B cells, and NK cells. This was seen even at low nM concentrations of rhIL10. In whole blood treated with rhIL10 at a concentration of 100 nM, the strongest pSTAT3 signal was seen on monocytes and myeloid dendritic cells with additional moderate activation of T, NK, B cells, and Granulocytes. The functional consequences of pSTAT3 signaling leads to increased production of IFNγ and Granzyme B from CD8 T cells and NK cells. There is also proliferation of B cells in response to rhIL10 signaling. This pro-inflammatory activity of rhIL10 in human whole blood is observed at exposures less than 5-fold above the anti-inflammatory IC90. The more selective cellular profile of antibody cytokine engrafted proteins such as IgGIL10M13 resulted in reduced pro-inflammatory activity leading to better anti-inflammatory efficacy.

### Example 41: Antibody Cytokine Engrafted Protein Signaling in Various Species

rhIL10 potently inhibits LPS-induced pro-inflammatory cytokine production in human monocytes, PBMCs, and whole blood. The antibody cytokine engrafted protein IgGIL10M13 exhibits pM potency on target cells, although 10-fold less potent than rhIL10. Table 4 is a potency comparison for IL10 or IgGIL10M13 activity in human whole blood as well as whole blood of selected toxicity species.

Potency calculations are based on ex vivo whole blood assays from either mouse, cynomolgus monkey or human. For each species tested, IgGIL10M13 or rhIL10 were titrated and assessed for ability to inhibit LPS-induced TNFα production. IC50s were calculated as the level of molecule that gave rise to 50% inhibition of total TNFα signal. IC90s and IC30s were calculated taking into account Hill slope value for each assay with the following equation: log EC50=log ECF−(1/HillSlope)*lob(F/100-F)), where ECF is the concentration that gives a response of F percent of total TNFα signal.

TABLE 4

| | | IgGIL10M13 (CV %) | | IL10 (CV %) | |
|---|---|---|---|---|---|
| Mouse | IC30 | 2.2 | pM (pooled blood) | 0.57 | pM (pooled blood) |
| | IC50 | 12 | pM | 1.7 | pM |
| | IC90 | 108 | pM | 15 | pM |
| Cyno | IC30 | 4.13 | pM (48% n = 3) | 0.44 | pM (28% n = 3) |
| | IC50 | 6.67 | pM (53%) | 0.65 | pM (31%) |
| | IC90 | 24 | pM (73%) | 1.9 | pM (43%) |
| Human | IC30 | 10.8 | pM (76% n = 48) | 1.3 | pM (96% n = 48) |
| | IC50 | 25.2 | pM (76%) | 2.8 | pM (98%) |
| | IC90 | 262 | pM (94%) | 22.8 | pM (79%) |

### Example 42: Evaluation of Antibody Cytokine Engrafted Protein Pharmacokinetics

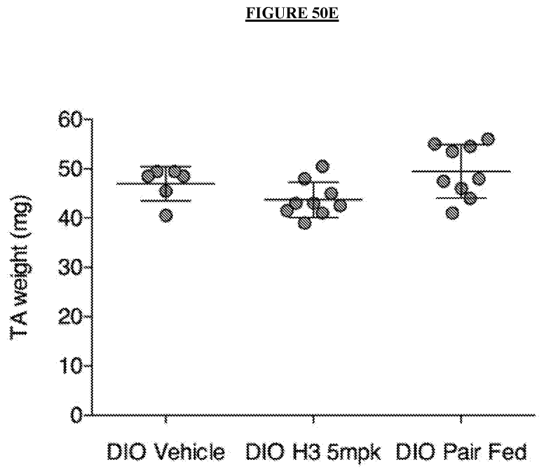

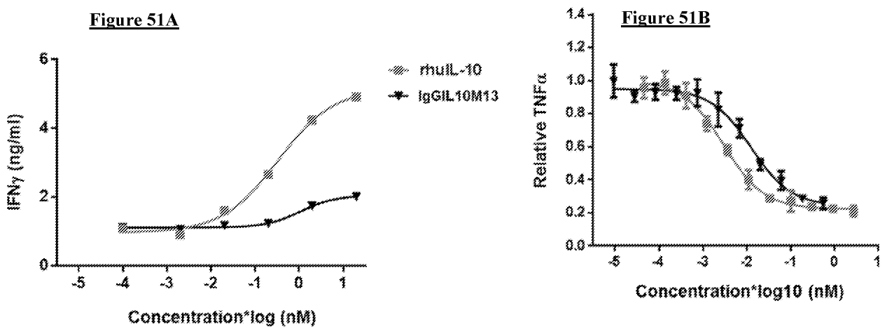

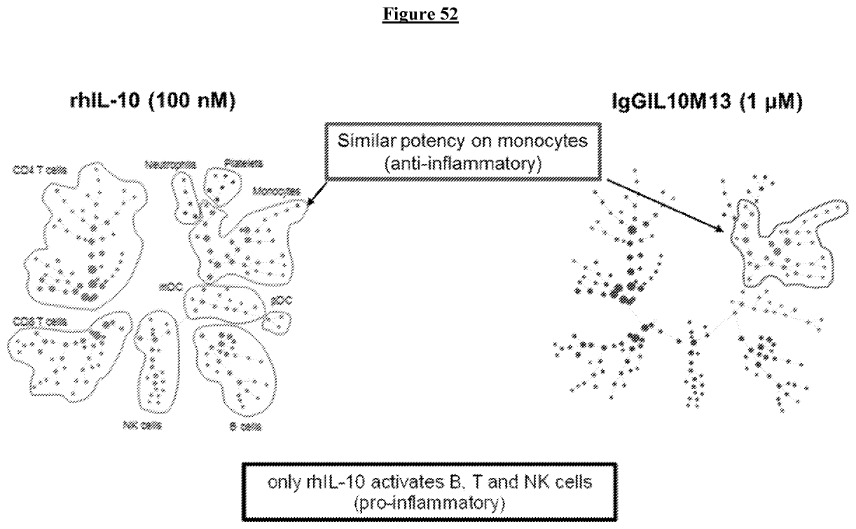

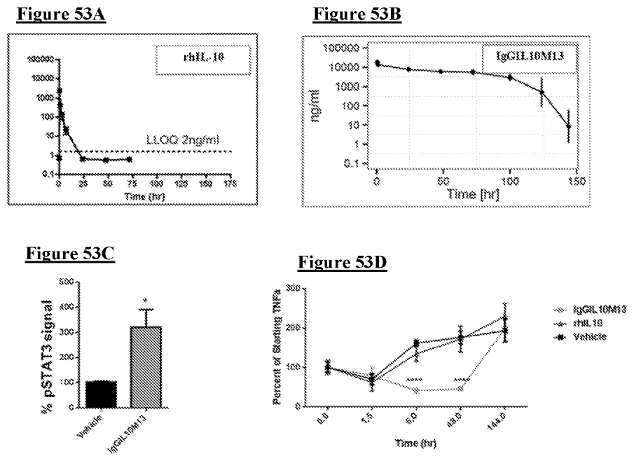

rhIL10 has a short half-life, limiting its target tissue exposure and requiring the patient to undergo multiple dosing. The half-life of antibody cytokine engrafted proteins was assessed in C57Bl/6 mice. Antibody cytokine engrafted proteins (e.g. IgGIL10M13) were injected at 0.2 mg/kg subcutaneously and blood was sampled beginning at 5 minutes post-injection up to 144 hours post-injection. IgGIL10M13 had a significant half-life extension of approximately 4.4 days (FIG. 53B) compared to rhIL10 which had a half-life of approximately 1 hr (FIG. 53A).

### Example 43: Evaluation of Antibody Cytokine Engrafted Protein Pharmacodynamics

Consistent with extended half-life, antibody cytokine engrafted proteins also demonstrated improved pharmacodynamics. Phospho-STAT3 (pSTAT3), a marker of IL10 receptor activation and signaling was monitored in mouse colon after subcutaneous dosing of IgGIL10M13. Enhanced pSTAT3 signal was detected in colon at least up to 72 hours post-dose, and absent by 144 hours post-dose. See FIG. 53C. This profile is a dramatic improvement over rhIL10, whose signal is absent by 24 hours post-dose. FIG. 53D depicts improved duration of in vivo response of IgGIL10M13 as compared to rhIL10 as measured by inhibition of TNFα in blood in response to LPS challenge following antibody cytokine engrafted protein dosing.

### Example 44: Efficacy of Antibody Cytokine Engrafted Proteins in a Mouse Model

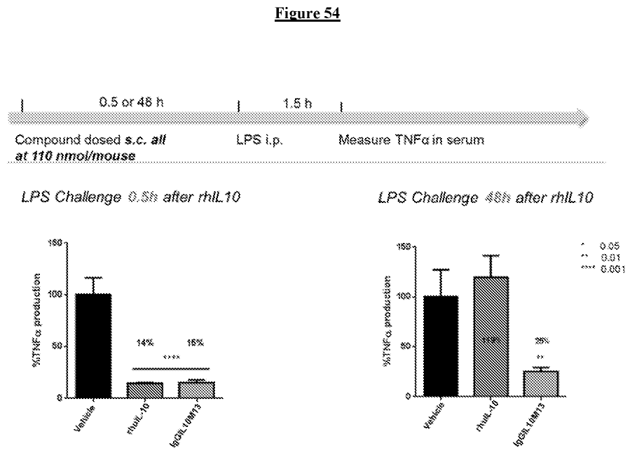

A direct comparison of efficacy for TNFα inhibition after LPS challenge was performed. C57/BL6 mice were dosed subcutaneously with vehicle, or equimolar levels of IL10 at 110 nmol/mouse, calculated for both recombinant IL10 and IgGIL10M13. Mice were then challenged with LPS delivered intraperitoneally to assess IL10 dependent inhibition of TNFα levels. IgGIL10M13 demonstrated comparable efficacy to rhIL10 at the initial assessment time period of 0.5 hour, however, up to at least forty-eight hours post dosing, IgGIL10M13 sustained superior efficacy to rhIL10 as measured by TNFα production. This data is shown in FIG. 54.

### Example 45: Antibody Cytokine Engrafted Proteins have Improved Exposure

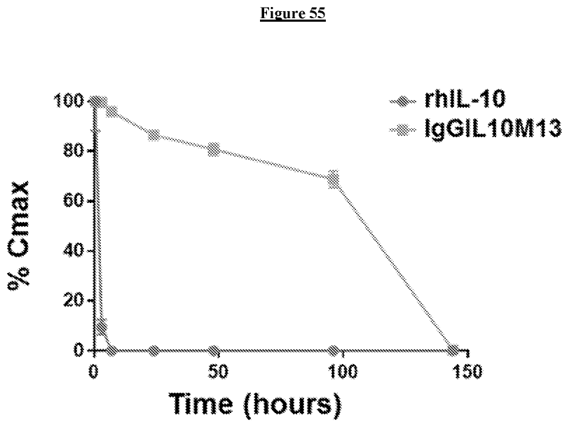

The peak serum concentration (Cmax) of antibody cytokine engrafted proteins was assessed in C57Bl/6 mice. Antibody cytokine engrafted proteins were injected at 0.2 mg/kg (10 ml/kg dose volume) in 0.9% saline subcutaneously and blood was sampled beginning at 1 hour post-injection and up to 144 hours post-injection. Whole blood was collected into heparin-treated tubes at each time point and centrifuged at 12,500 rpm for 10 minutes at 4° C. Plasma supernatant was collected and stored at −80° C. until all time points were collected. Antibody cytokine engrafted proteins levels in plasma were measured using two different immunoassay methods to enable detection of both the IL10 and antibody domains of the antibody cytokine engrafted protein. As shown in FIG. 55, the antibody cytokine engrafted protein (e.g. IgGIL10M13) maintained greater than 60% Cmax past 100 hours. In contrast, rhIL10 levels dropped below 20% Cmax within 3.5 hours.

### Example 46: Antibody Cytokine Engrafted Proteins Act Only on Certain Cell Types in Human Patients

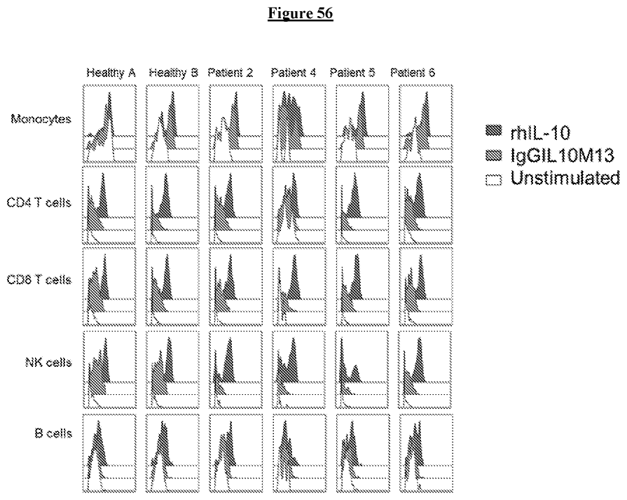

CyTOF was run as previously described on immune cells from human healthy donors and patients with Crohn's disease. As shown in the graphs in FIG. 56, IgGIL10M13 stimulated only monocytes, and the stimulation as measured by pSTAT3 levels is comparable to rhIL10. Monocytes are the target cells for inflammatory related disorders such as Crohn's disease and Ulcerative Colitis and express very high levels of IL10 receptor. However, FIG. 56 also shows the unwanted pro-inflammatory effects of rhIL10, for example, the increased pSTAT3 signaling on CD4 T cells, CD8 T cells and NK cells. It is noteworthy that IgGIL10M13, does not display this unwanted pro-inflammatory effect either on normal human cells or in cells taken Crohn's disease patients. This demonstrates that IgGIL10M13 has a larger, safer therapeutic index as administration of the antibody cytokine engrafted protein will act only on the desired cell type and not on other cell types such as CD8 T cells which would only worsen immune related disorders such as Crohn's disease and Ulcerative Colitis.

### Example 47: IgGIL10M13 has Reduced Pro-Inflammatory Activity in PHA Stimulated Human Whole Blood Compared to rhIL10

Despite extensive clinical data linking genetic IL10 deficiency to IBD susceptibility, rhIL10 showed only mild efficacy in IBD clinical trials (Herfarth et al., Gut 2002: 50(2):146-147). Retrospective analyses of trial data suggest that rhIL10's efficacy was limited by its intrinsic pro-inflammatory activity such as enhanced production of IFNγ. As discussed previously, in human functional cell-based assays, rhIL10 signaling leads to production of IFNγ and Granzyme B from T cells and NK cells.

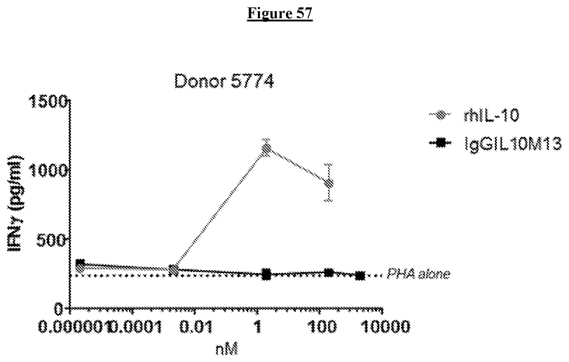

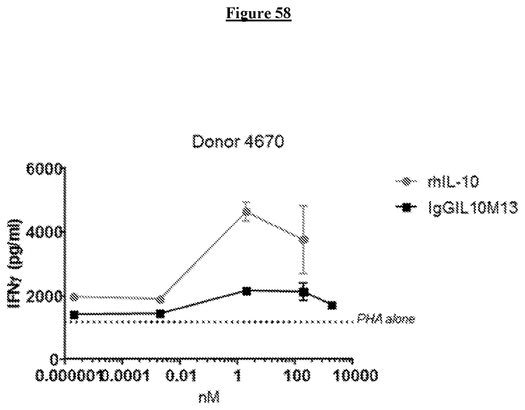

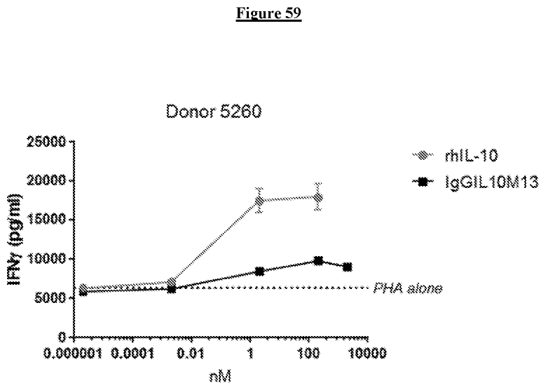

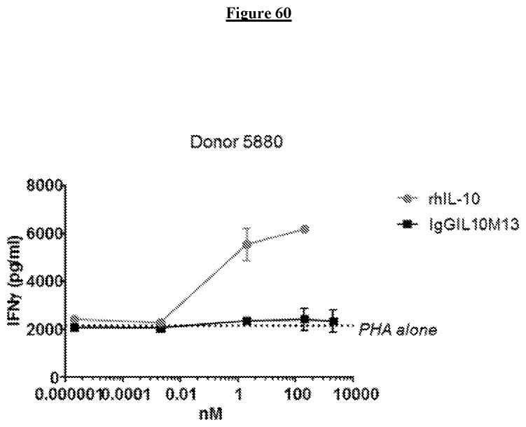

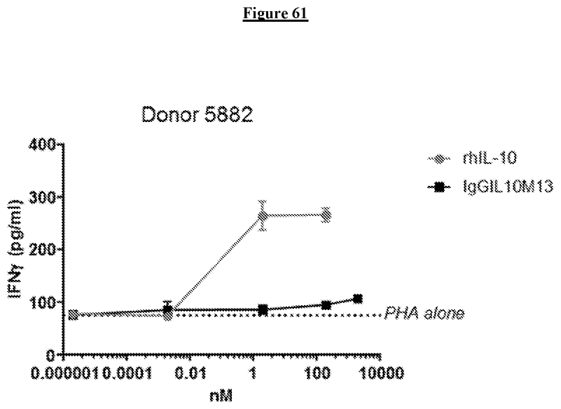

Whole blood was taken from patients with Crohn's Disease and the levels of IFNγ were measured after stimulation with rhIL10, IgGIL10M13 and PHA alone. This data is shown in FIGS. 57-61. Increasing doses of rhIL10 causes a sharp increase in IFNγ production, which then plateaus. In contrast, in treatment of these cells with IgGIL10M13 little to no production of IFNγ was seen, indicating that IgGIL10M13 did not induce, or induced only very low levels of IFNγ production from T cells or NK cells.

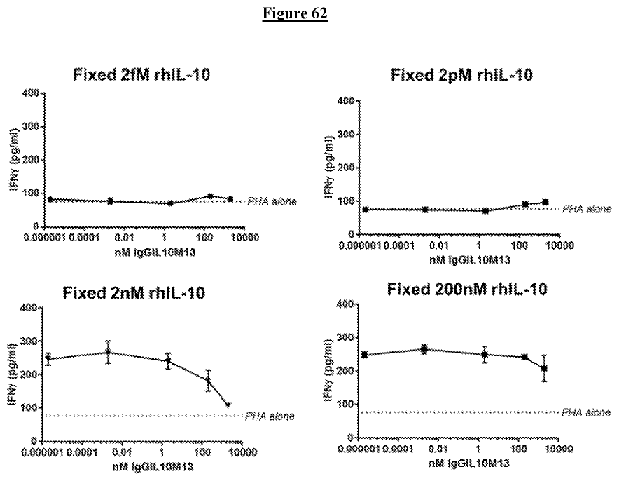

An additional titration experiment was performed with these patient donor samples. In this experiment, IL10 levels from the donor patient sera was measured and found to be in the range of 1.5 to 5 femtomolar (fM), although the scientific literature has reported that patient IL10 levels could be as high as 20 fM (Szkaradkiewicz et al., Arch. Immunol. Ther Exp 2009: 57(4):291-294). rhIL10 was administered to the donor patient cells at the fixed concentrations of 2 femtomolar (fM), 2 pM, 2 nM and 200 nM. To these fixed concentrations of rhIL10, increasing concentrations of the antibody cytokine engrafted protein IgGIL10M13 was administered, and IFNγ production assayed. The data is shown in FIG. 62. At the fixed concentrations of 2 fM and 2 pM, IgGIL10M13 competes with rhIL10 and reduced the production of IFNγ to baseline levels. At the fixed concentration of 2 nM, IFNγ production was reduced by nanomolar concentrations of IgGIL10M13. Finally, at the fixed excess concentration of 200 nM rhIL10, only very little reduction of IFNγ production by IgGIL10M13 was seen. This indicates that at physiological levels of IL10, IgGIL10M13 competed out IL10, reducing the production of IFNγ, and the unwanted pro-inflammatory effects.

### Example 48: Aggregation Properties of Antibody Cytokine Engrafted Proteins

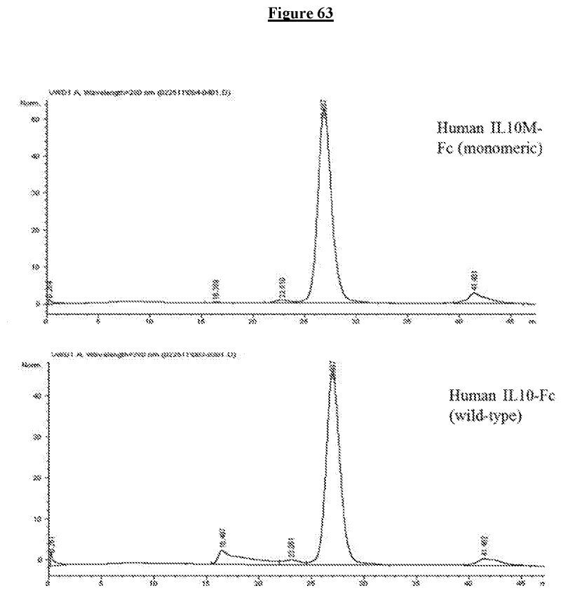

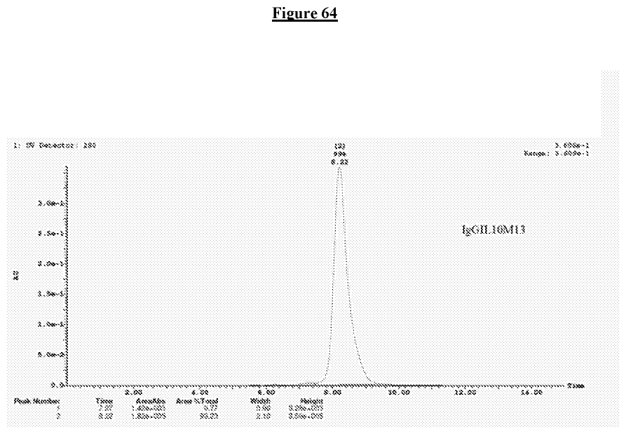

In clinical trials for IBD, rhIL10 was observed to have a very short half-life; however simple Fc fusions to the IL10 dimer to extend half-life were not pursued given aggregation properties of such a molecule. FIG. 63 shows aggregation of both an IL10 wild type linked to an Fc and IL10 monomer linked to an Fc. However, as shown in FIG. 64, the antibody structure of the antibody cytokine engrafted protein prevents IL10 aggregation, thus promoting ease of administration. In addition, reducing aggregation has the benefit of reducing an immune reaction to the therapeutic, and the generation of anti-drug antibodies.

### Example 49: Retained Binding of Antibody Cytokine Engrafted Proteins

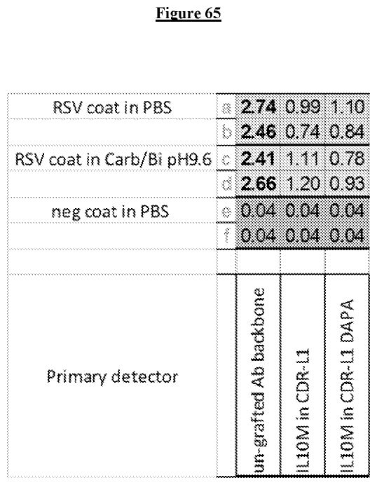

Palivizumab is an anti-RSV antibody, and was chosen as the antibody structure for cytokine engrafting. This antibody had the advantages of a known structure, and as its target was RSV, a non-human target. The choice of a non-human target was to insure that there would be no toxicity associated with the antibody cytokine engrafted protein binding to an off target human antigen. It was uncertain after engrafting IL10M into palivizumab, whether the final IL10 antibody cytokine engrafted protein would still bind the RSV target protein. As assayed by ELISA, the IL10 antibody cytokine engrafted protein still bound to RSV target protein, despite the presence of the IL10M. This data is shown in FIG. 65.

### Example 50: Structural Conformation of the Antibody Cytokine Engrafted Protein Results in Differential Activity Across Cell Types

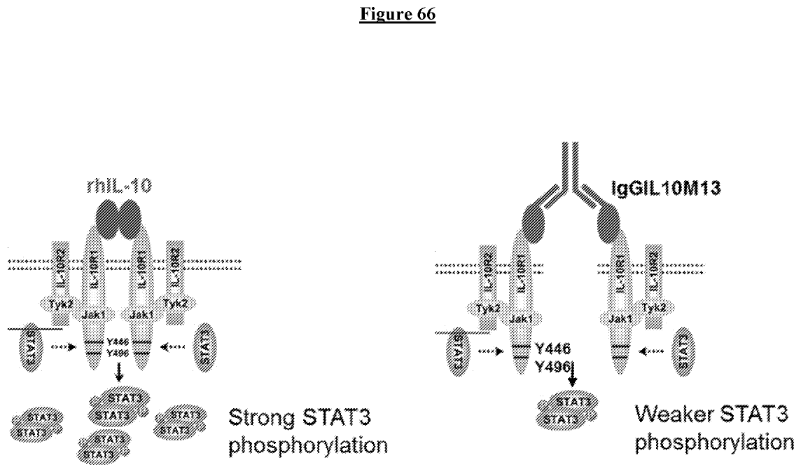

Antibody cytokine engrafted proteins (e.g. IgGIL10M13) incorporates monomeric IL10 into the Light Chain CDR 1 of an antibody. Insertion of a 6 amino acid glycine-serine linker between helices D and E of IL10 renders the normally heterodimeric molecule incapable of domain swapping dimerization. As such, engrafting IL10M into an antibody results in an antibody cytokine engrafted protein with 2 monomeric IL10 molecules. However, due to flexibility of the antibody Fab arms, the angle and distance between the IL10 monomers is not fixed, as in the wild-type IL10 dimer, thus affecting its interaction with the IL10R1/R2 receptor complex. This is shown graphically in FIG. 66. Specifically, due to antibody engraftment, the angle of the engrafted IL10 dimer is larger and variable, rendering signal transduction less efficient on cells with lower expression levels of IL10R1 and R2 as found on the pro-inflammatory cell types such as CD4 and CD8 T cells, B cells and NK cells. In contrast, antibody cytokine engrafted proteins signal more efficiently on cells with high IL-10R1 and R2 expression such as monocytes. A class average negative stain EM study of IgGIL10M13 highlighted the additional flexibility and wider angle between monomers, confirming that the geometry is altered compared to rhIL10. The less restricted geometry of the IL10 dimer in IgGIL10M13 alters its interaction with IL10R complex. As a consequence, the structure of the IgGIL10M13 antibody cytokine engrafted protein results in the biological effect of only producing a productive signal on cell types with high levels of IL10R1 and R2 expression.

### Example 51: Crystal Structure of IgGIL10M13

The IgGIL10M13 Fab was concentrated to 16.2 mg/ml in 20 mM HEPES pH 8.0, 150 mM NaCl and used directly in hanging drop vapor diffusion crystallization trials. Crystallization screens were setup by mixing 0.2 μd of protein solution with 0.2 μd of reservoir solution and equilibrated against 50 μl of the same reservoir solutions. Crystals for data collection appeared after 3-4 weeks at 20° C. from a reservoir solution of 20% PEG3350, 200 mM magnesium acetate, pH 7.9. Prior to data collection, the crystals were soaked in reservoir solution supplemented with 20% ethylene glycol and flash cooled in liquid nitrogen. Diffraction data were collected at the ALS beamline 5.0.3 with an ADSC Quantum 315R detector. Data was indexed and scaled using the HKL2000 software package (Otwinowski and Minor. (1997) Methods in Enzymology, Volume 276: Macromolecular Crystallography, part A, p. 307-326). The data for the IgGIL10M13 Fab was processed to 2.40 Å in space group P2₁ with cell dimensions a=80.6 Å, b=104.7 Å, c=82.8 Å, alpha=90°, beta=115.3°, gamma=90°. The structure was solved by molecular replacement using PHASER (McCoy et al., (2007) J. Appl. Cryst. 40:658-674) with the palivizumab Fab structure (PDB code: 2HWZ) and monomeric IL10 structure (PDB Code: 1LK3 chain A) as search models. The top molecular replacement solution contained 2 molecules of the IgGIL10M13 Fab in the asymmetric unit. The final model was built in COOT (Emsley & Cowtan (2004) Acta Cryst. D60:2126-2132) and refined with PHENIX (Adams et al., (2010) Acta Cryst. D66, 213-221). The R_{work} and R_{free} values are 18.8% and 23.9% respectively with root-mean-square (r.m.s) deviation values from ideal bond lengths and bond angles were 0.005 Å and 0.882° respectively.

### Overall Structure

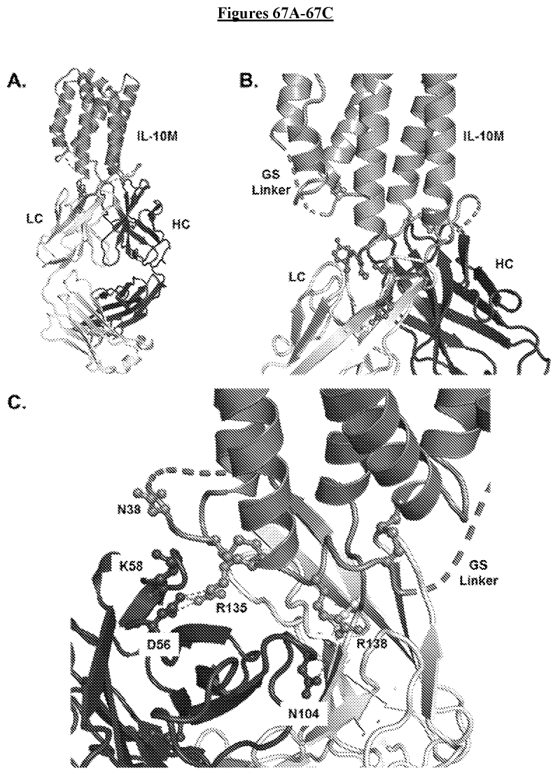

The IgGIL10M13 Fab crystallized with 2 molecules in the asymmetric unit, both with similar conformations. The electron density maps were similar for both molecules. The overall structure (FIG. 67A) shows that the Fab and grafted monomeric IL10 (IL10M) can adopt a collinear arrangement (Fab light chain in white, Fab heavy chain in black, IL10M in dark grey). FIG. 67B shows a closer view of the grafting point in CDR-L1. The three flanking CDR residues are show with dark grey sticks. Dashed lines illustrate portions of the structure which could not be fit in the model due to missing electron density, presumably due to structural flexibility in these areas. The two areas include 6 residues at N-terminus of IL10M just after the grafting point and 8 residues between helices 4 and 5 in IL10M which encompass the inserted 6 residue linker. There are also 3 pairs of hydrogen bonding interactions between the grafted IL10M molecule and portions of the Fab heavy chain (FIG. 67C). These include R138 and N104 (sidechain), R135 and D56 (sidechain), and N38 and K58 (backbone/sidechain).

### Example 52: ACE Proteins Using Alternative Scaffolds

ACE proteins were initially constructed using GFTX3b, an anti-RSV antibody, as the scaffold. However, ACE proteins were also constructed using GFTX, and anti-IgE antibody as an additional scaffold. As native IL10 signals as a homodimer, IL10 ACE proteins were constructed using IL10 in the same antibody “arm.” For example, IL10 was engrafted into the third CDR of the variable heavy chain (CDRH3) of the GFTX scaffold, resulting in an ACE molecule with an IL10 molecule in both CDRH3 “arms” of the antibody. In addition, ACE proteins were constructed with an IL10 molecule in the first CDR of the variable light chain (CDRL1) and an IL10 molecule in to CDRH3. This created an ACE protein with an IL10 cytokine engrafted into two separate and distinct locations within the GFTX scaffold. Both types of GFTX ACE proteins were compared to native IL10 cytokine and to IL10Fc fusion proteins.

Human whole blood was obtained from The Scripps Research Institute Normal Blood Donor Service. Whole blood donors were anonymized but were requested to be free of anti-inflammatory medication. After pick up, whole blood was kept at 37° C. for 1 hr prior to isolation as the assay was prepared. Whole blood was processed to PBMCs using Lymphoprep density gradient (STEMCELL, Cat #07851, Lot #12ISf11) by layering 15 ml of whole blood on 10 ml of gradient and centrifuged at 800×G for 20 minutes, no brake, at room temperature. PBMCs were collected from the density gradient interface and washed two times in medium. This was repeated for 50 ml of blood per donor. PBMCs were prepared at 2.2e6 cells/ml (100,000 cells/well in 384 well plate in 45 ul volume).

GFTX constructs and rhIL-10 (Biolegend) were thawed and diluted to a working solution of 1000 ng/mL [final in assay 100 ng/mL] in lymphocyte culture medium (RPMI 1640, 10% FBS, 50 μM BME, 10 mM Hepes, 0.1 mM NEAA, 1 mM Sodium Pyruvate, 2 mM glutamine, 1× Human Insulin Transferrin Selenium, 60 mg/ml Pen/100 mg/ml Strep). An 11 point dose titration was prepared using the working solution as the starting concentration and performing a 1:3 dilution for each subsequent concentration in medium. LPS (100 μg/ml stock) was prepared and thawed and kept on ice prior to assay.

Titration curves were prepared. For “no LPS” control wells, 45 μl/well of PBMCs was dispensed into respective wells of a 384 well plate and brought up to 50 μl with medium. For LPS stimulation, LPS was added to the 50 ml conical containing human PBMCs to a working concentration of 1.1 ng/ml [final in assay 1 ng/mL]. The PBMCs and the LPS was well mixed and then 45 μl/well was dispensed into designated wells on the plate followed by 5 μl/well of designated IL-10 formulations. Assay plate was well mixed and incubated for 20 hrs in a 37° C., 5% CO₂ incubator.

The following day, the assay plate was mixed centrifuged at 1400 rpm for 5 minutes at room temp. Supernatant (approximately 10 μl) was removed from each well and transferred to a 384 well proxy plate. For the HTRF assay, antibodies directed to TNFa were reconstituted 1:40 in Reconstitution buffer provided in the HTRF kit (Cisbio, Bedford Mass.). HTRF mix was then added to proxy wells (10 μl/well) and the proxy plate was incubated for 3 hours at room temperature in the dark. Samples were then analyzed for FRET towards the wavelength 665 nm. Data was normalized for each donor using the donor's lowest titration results as a baseline. LPS induction was calculated for each donor using the “no LPS” wells. Data was analyzed using nonlinear regression to calculate IC50s for each donor.

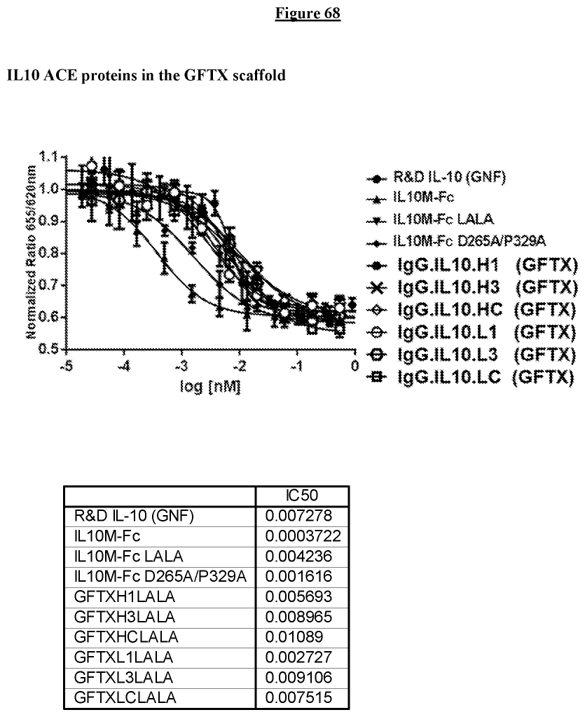

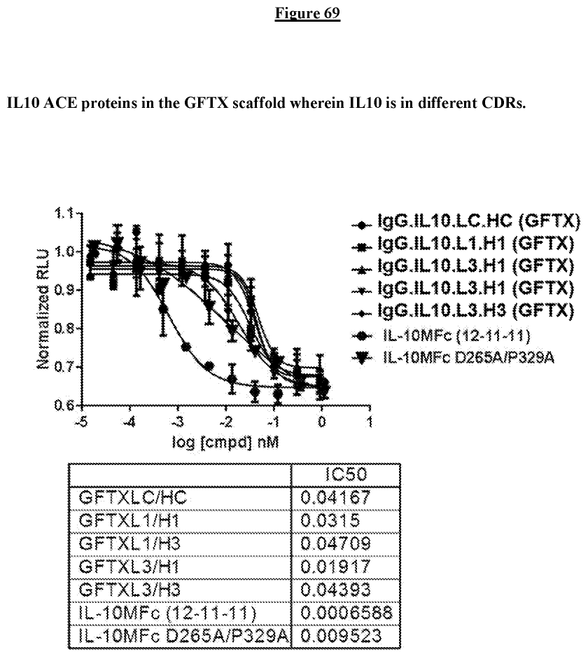

As shown in FIG. 68, IL10 antibody cytokine proteins that have IL10 engrafted into the same CDR (eg., CDRH1) show similar IC50 potencies to recombinant human IL10 (rhIL10) and IL-10 Fc fusions, either Fc wild type fusions or fusions containing an Fc silencing mutation (LALA or DAPA). In contrast, as shown in FIG. 69, where IL10 is engrafted into different CDRs (e.g., CDRL1 and CDRH1) in the same ACE protein, lower IC50 potencies are seen when compared to IL-10M Fc fusions (wild-type Fc or DAPA Fc).

Alternative scaffolds were also constructed for IL2. In contrast to IL10, IL2 can act as a monomer, so IL2 was engrafted into the same CDRs (e.g. CDRL3) and no ACE proteins were made where IL2 was engrafted into different CDRs of the same antibody (e.g., CDRL3 and CDRH1).

Pre-diabetic NOD females were administered low dose equimolar IL2 (5× weekly) or an IL2 ACE protein wherein IL2 was engrafted into CDRL3 (1×/weekly) by intraperitoneal injection. Five mice per group were taken down 7 days after the first dose, spleens processed to obtain single cell suspensions and washed in RPMI (10% FBS). Red blood cells were lysed with Red Blood Cell Lysis Buffer and cells counted for cell number and viability. FACS staining was performed under standard protocols using FACS buffer (1×PBS+0.5% BSA+0.05% sodium azide). Cells were stained with surface antibodies: Rat anti-mouse CD3-BV605 (BD Pharmingen #563004), Rat anti-mouse CD4-Pacific Blue (BD Pharmingen #558107), Rat antimouse CD8-PerCp (BD Pharmingen #553036), CD44 FITC (Pharmingen #553133) Rat anti-mouse CD25-APC (Ebioscience #17-0251), and subsequently fixed/permeabilized and stained for FoxP3 according to the Anti-Mouse/Rat FoxP3 Staining Set PE (Ebioscience #72-5775). Cells were analyzed on the BD LSR Fortessa® or BD FACS LSR II, and data analyzed with FlowJo® software.

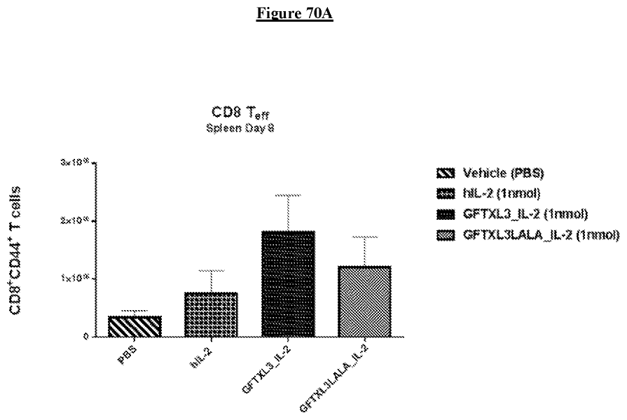

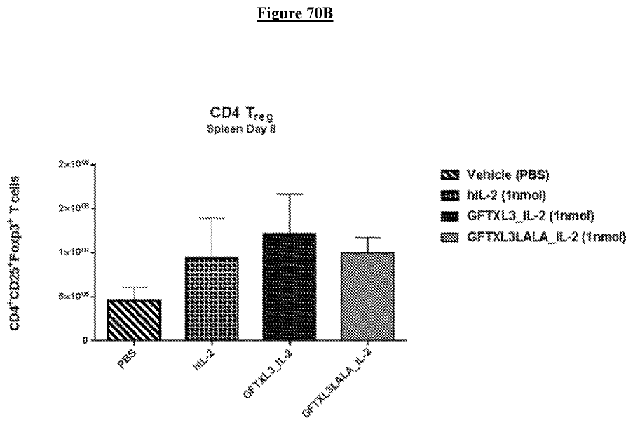

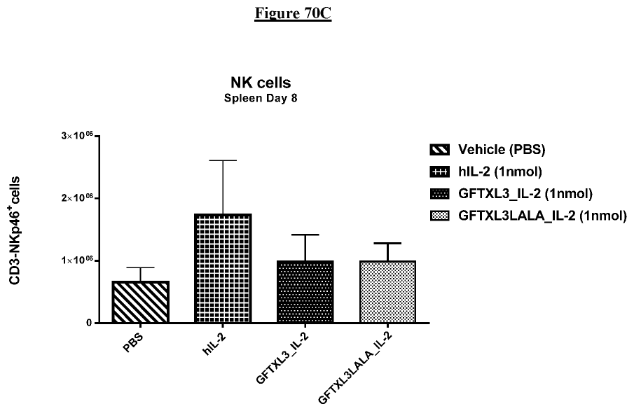

As shown in FIG. 70A, the IL2 ACE protein (GFTXIL3_IL-2) expands CD8+T effectors more effectively than recombinant human IL2 (hIL-2). An IL2 ACE protein with an Fc silent modification (GFTXL3LALA_IL2) also expands expands CD8+T effectors more effectively than recombinant human IL2. FIG. 70B demonstrates that the IL2 ACE protein (GFTXIL3_IL-2) expands CD4+ Treg cells more effectively than recombinant human IL2 (hIL-2). The effect on NK cells is shown in FIG. 70C, where recombinant human IL2 expands NK cells more effectively than IL2 ACE proteins either with or without Fc silencing mutations. In summary, this data shows that IL2 ACE proteins can be effective using a different antibody scaffold.

### Example 53: Cytof Data of ACE Proteins

CyTOF, a FACS based method that combines mass cytometry, incorporates flow cytometry technology with a time-of-flight inductively coupled plasma mass spectrometry (ICP-MS). It allows for the simultaneous detection and quantification of over 40 parameters from a single cell. It utilizes rare-earth metal conjugated monoclonal antibodies to specific cell surface or intracellular molecules. Using CyTOF, in vitro signaling studies were performed on ACE proteins in human PBMCs assessed by pSTAT1, pSTAT3, pSTAT4, and pSTAT5 detection.

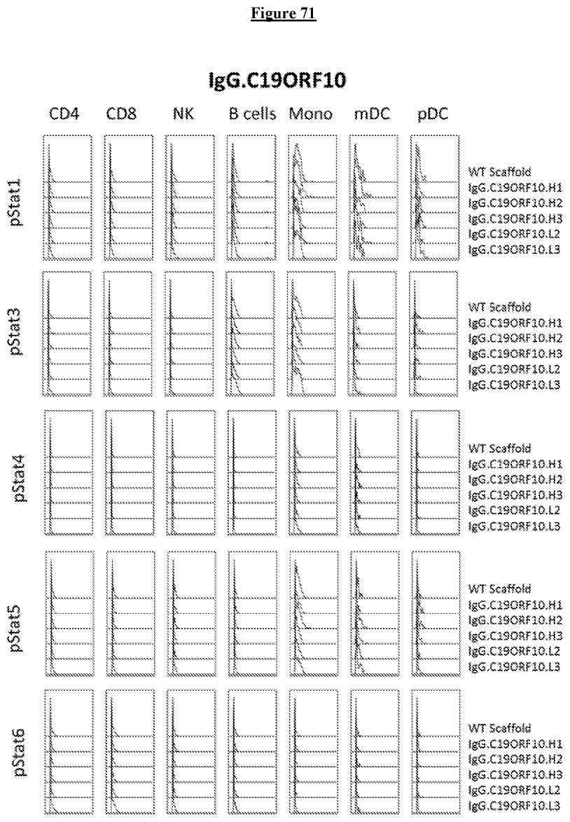

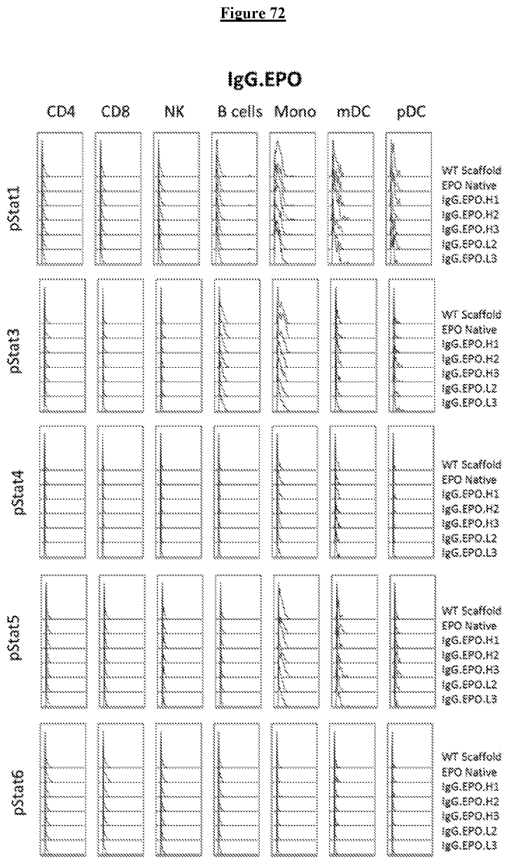

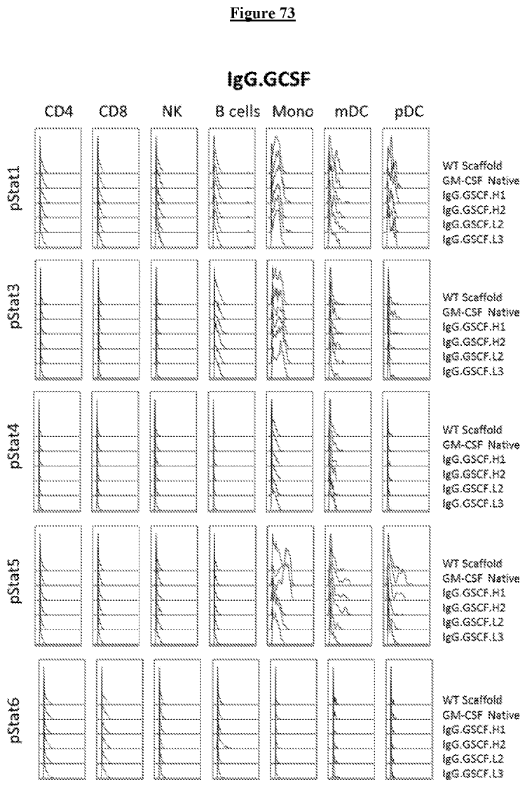

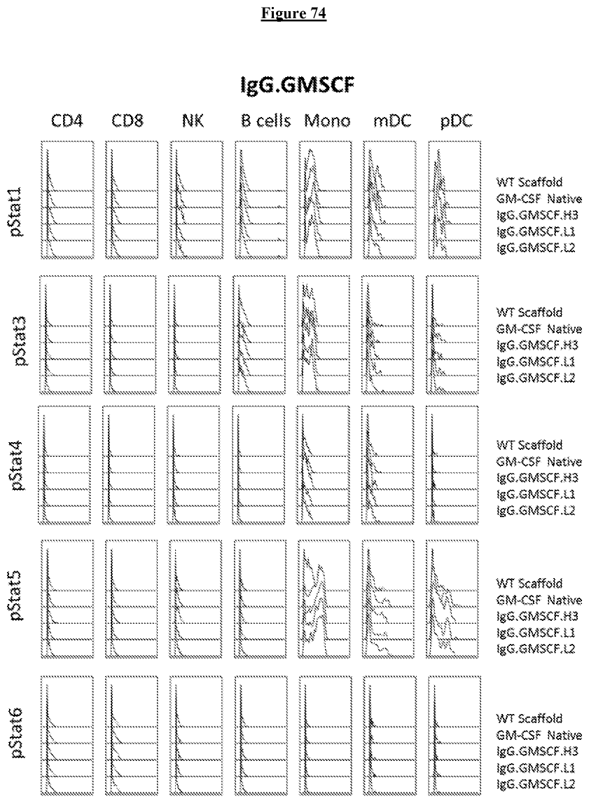

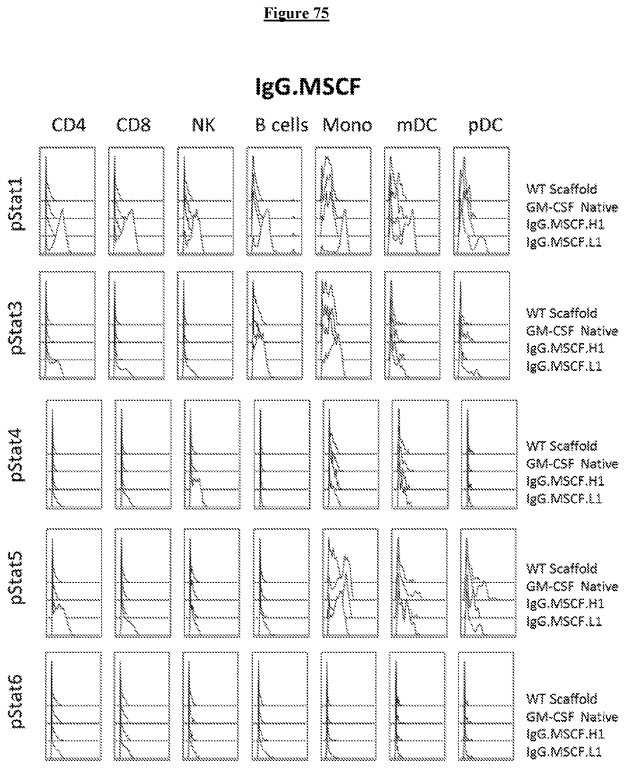

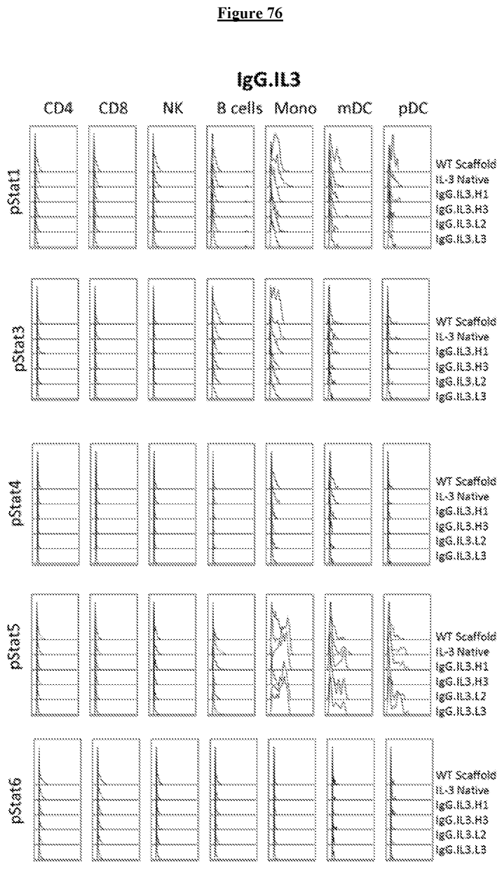

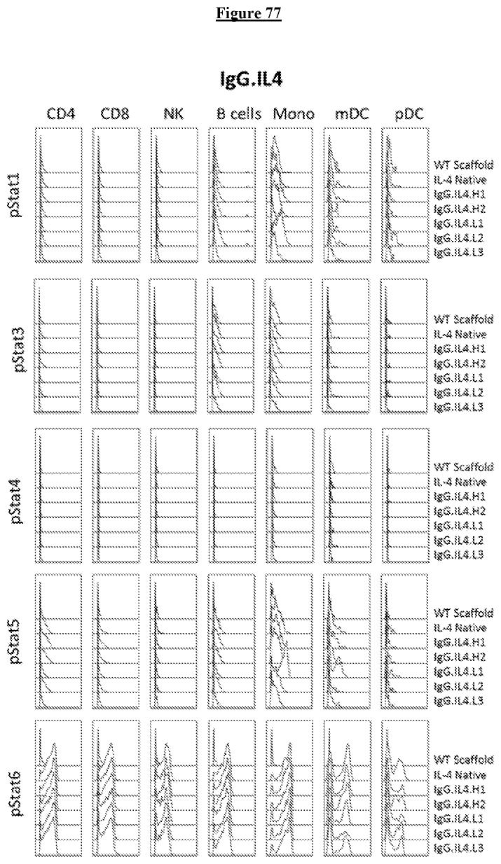

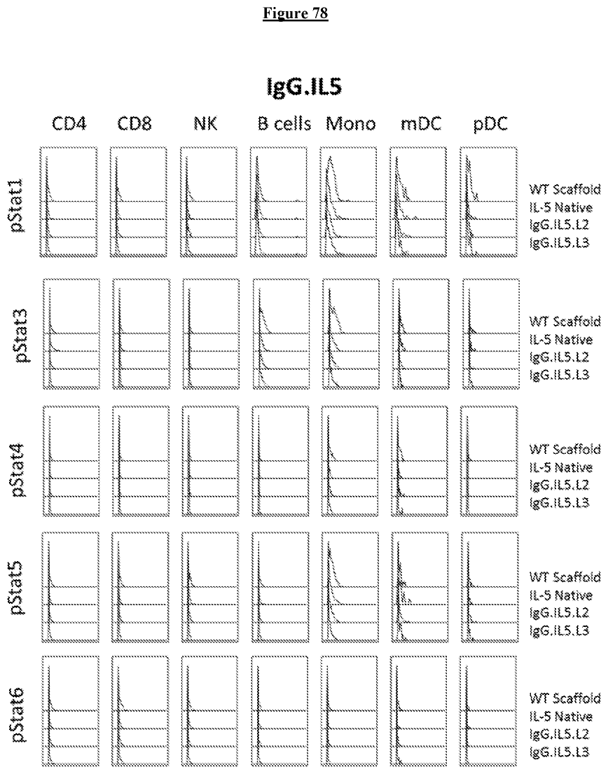

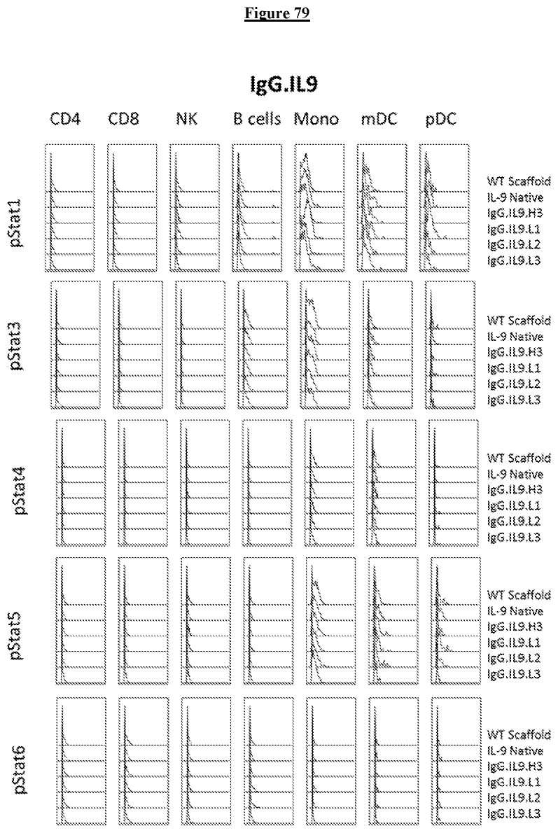

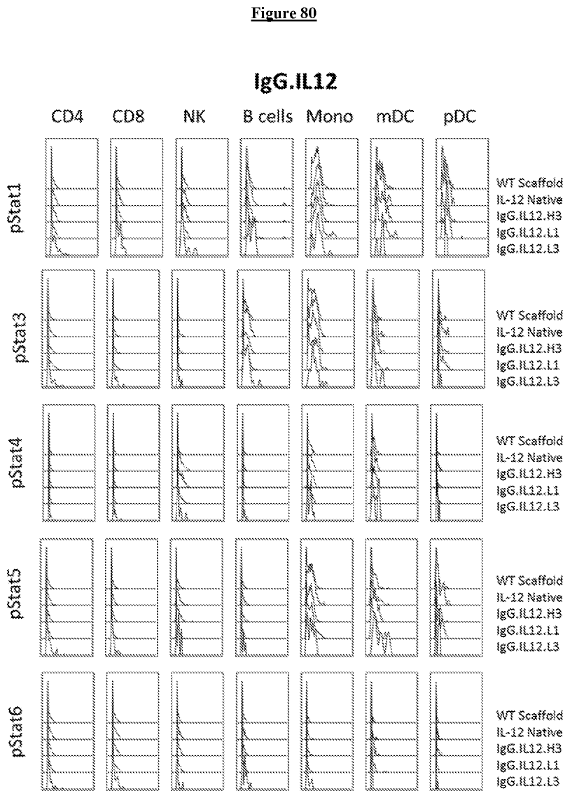

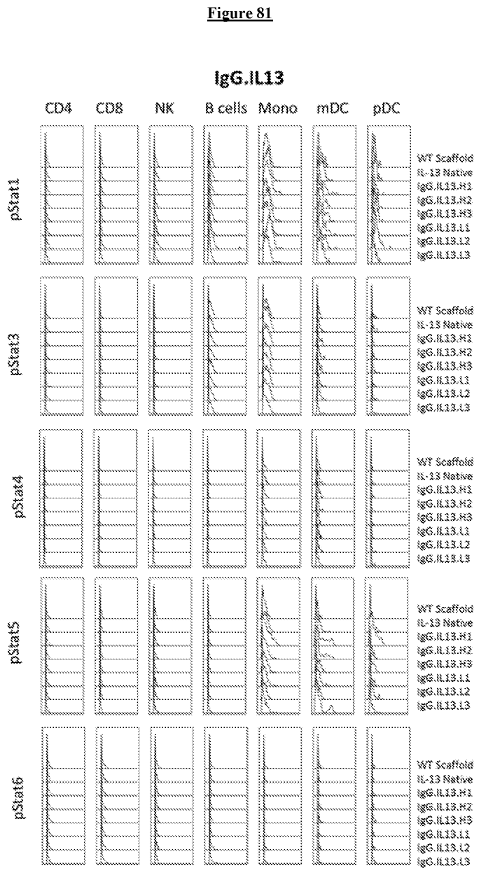

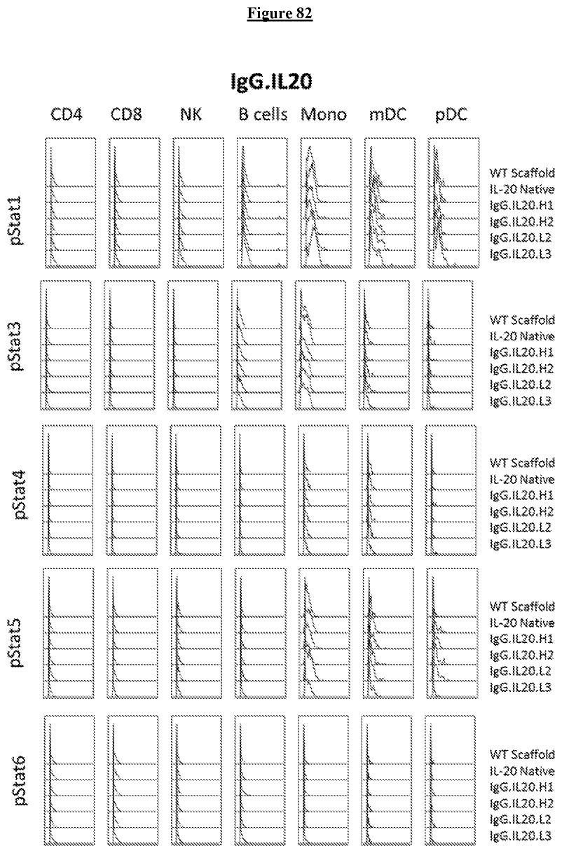

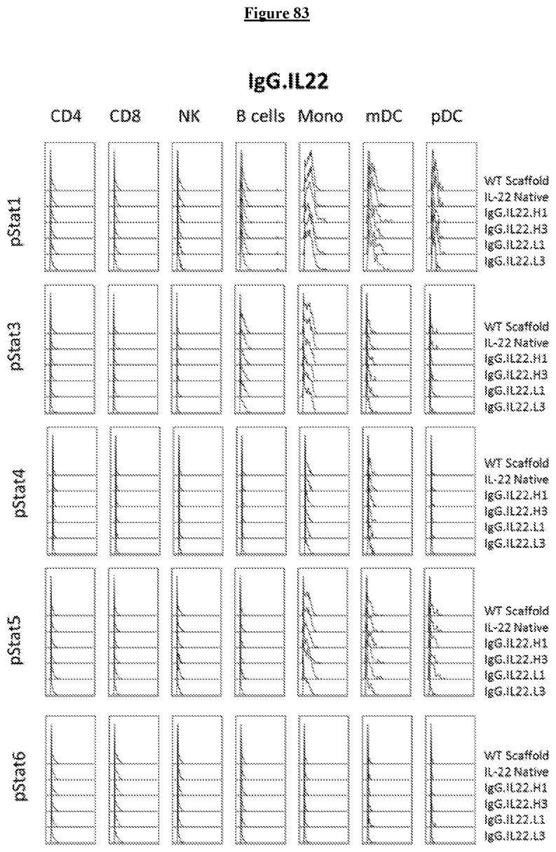

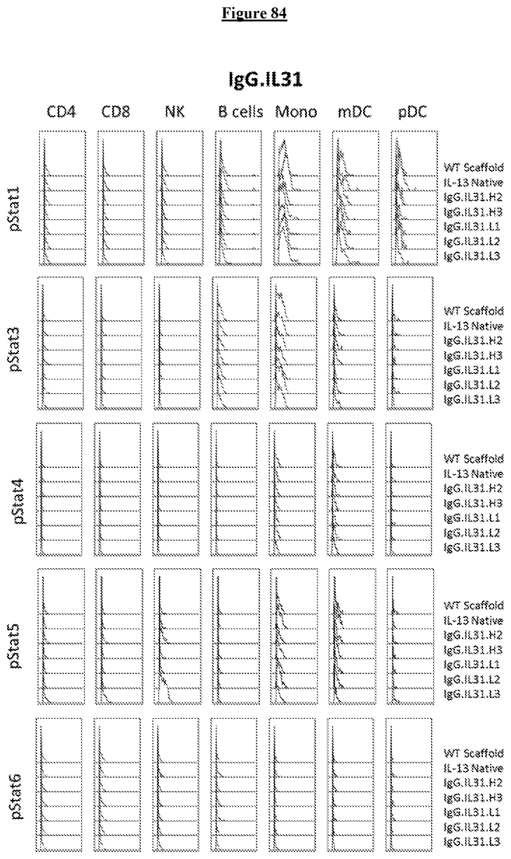

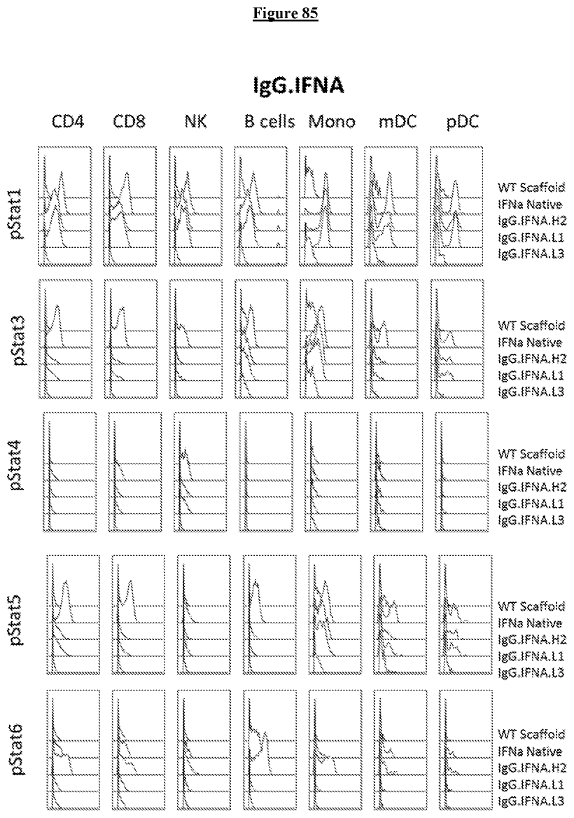

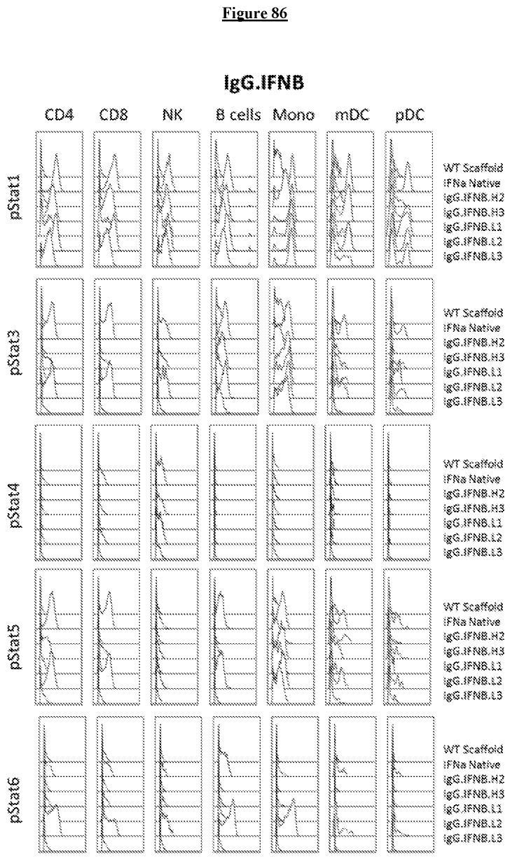

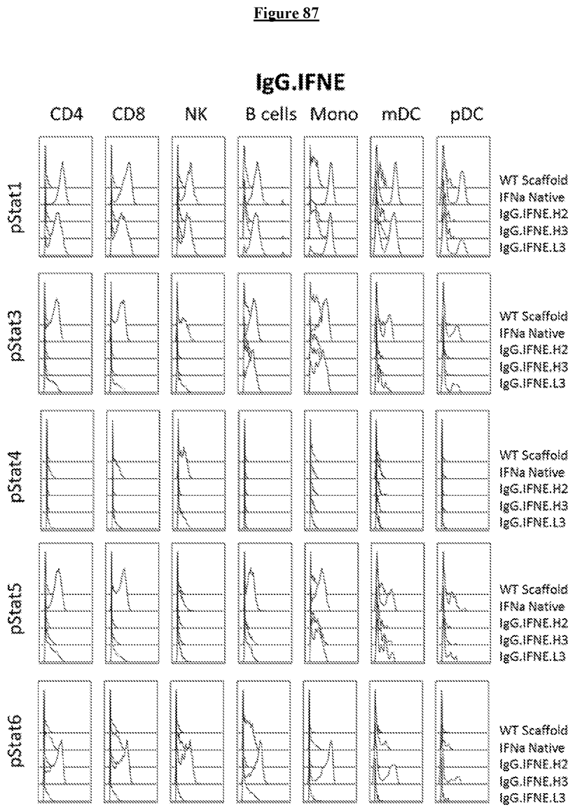

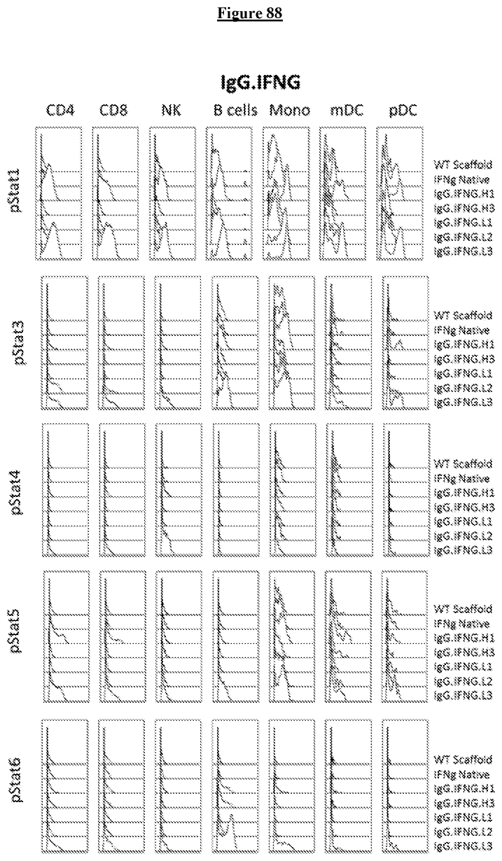

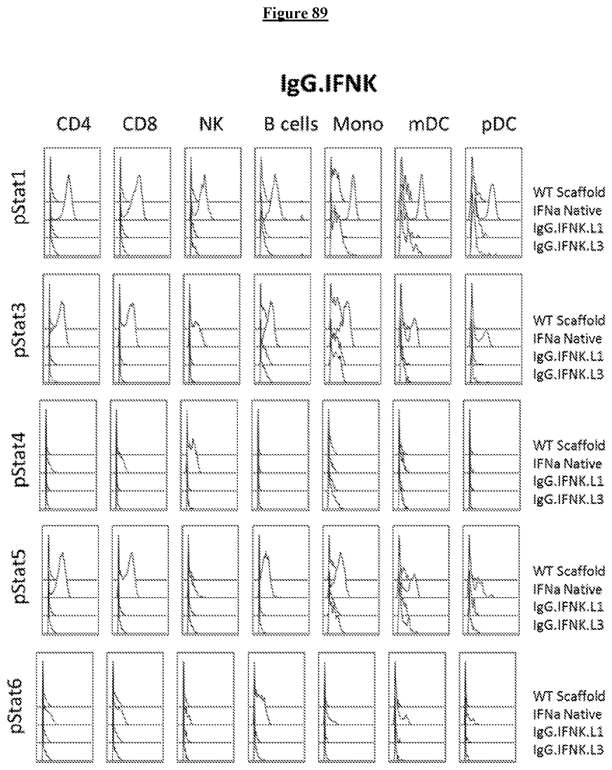

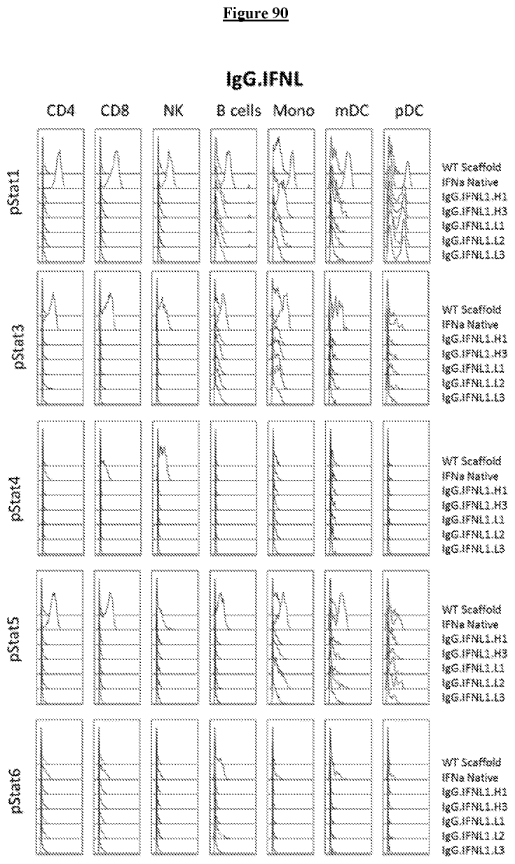

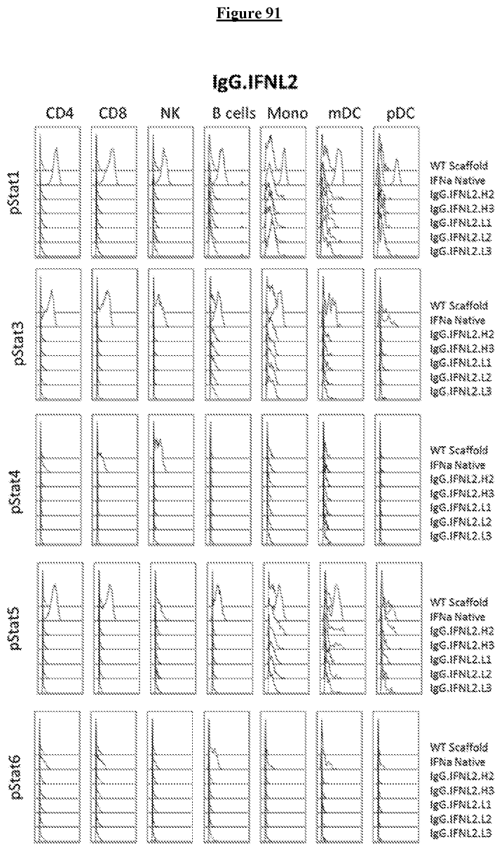

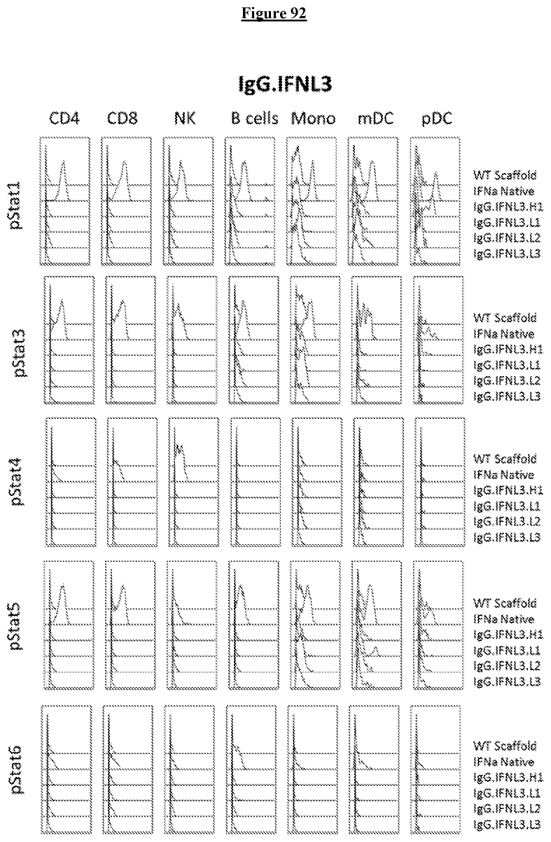

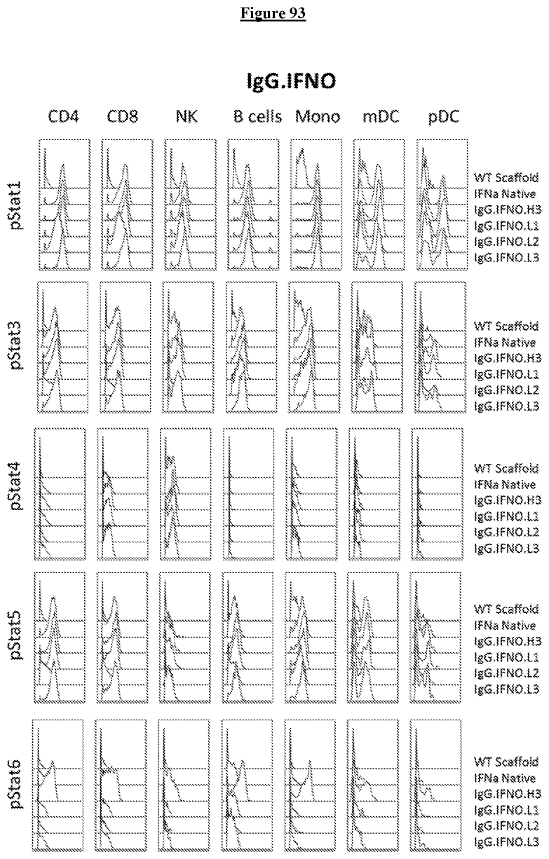

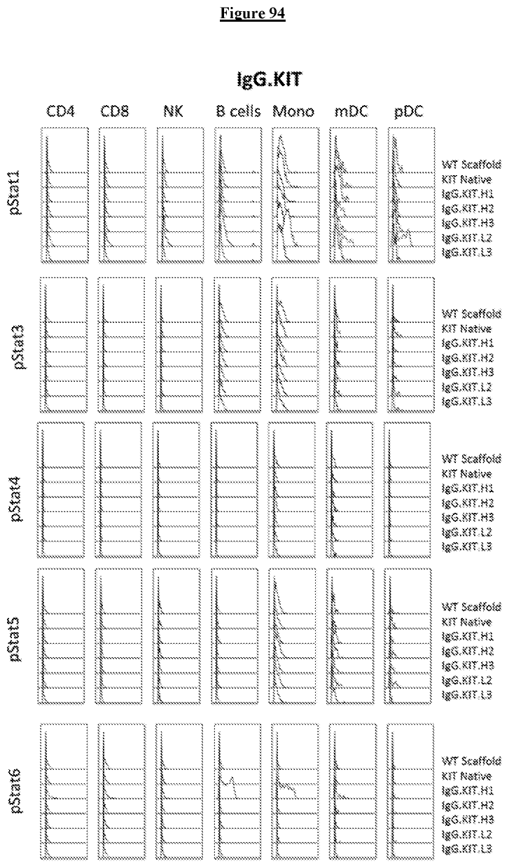

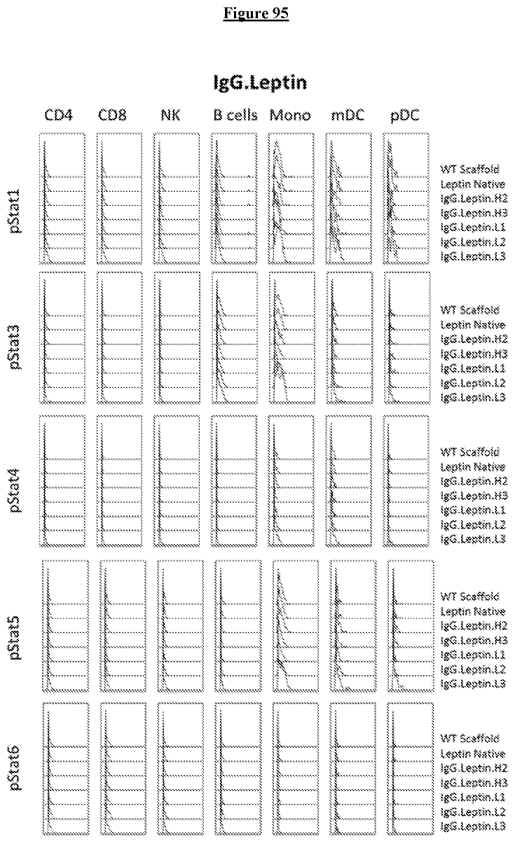

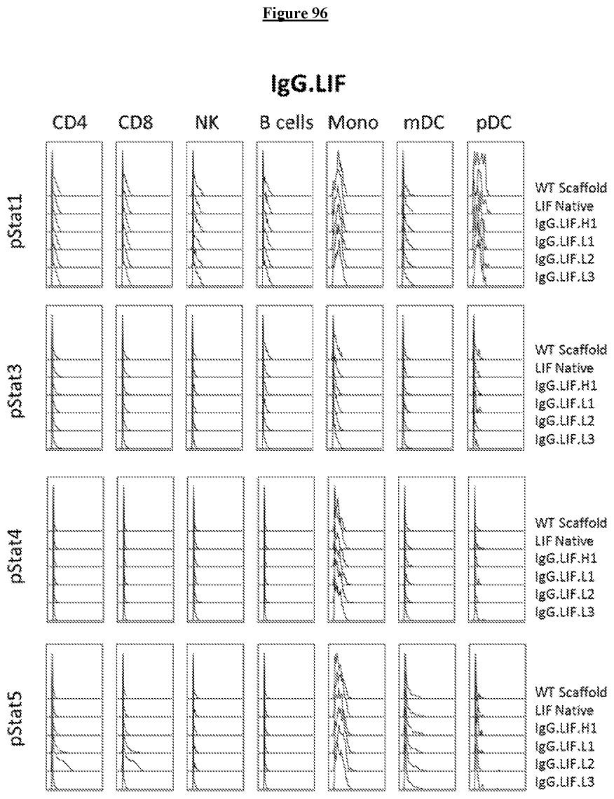

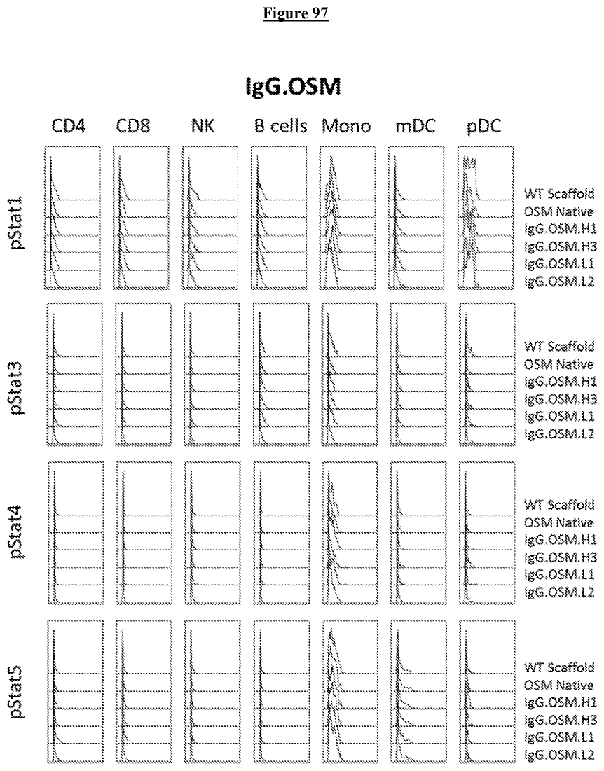

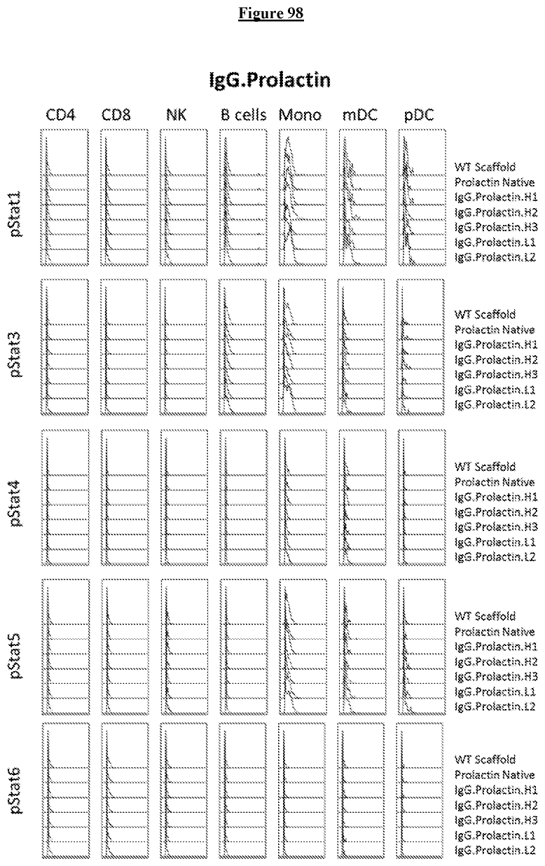

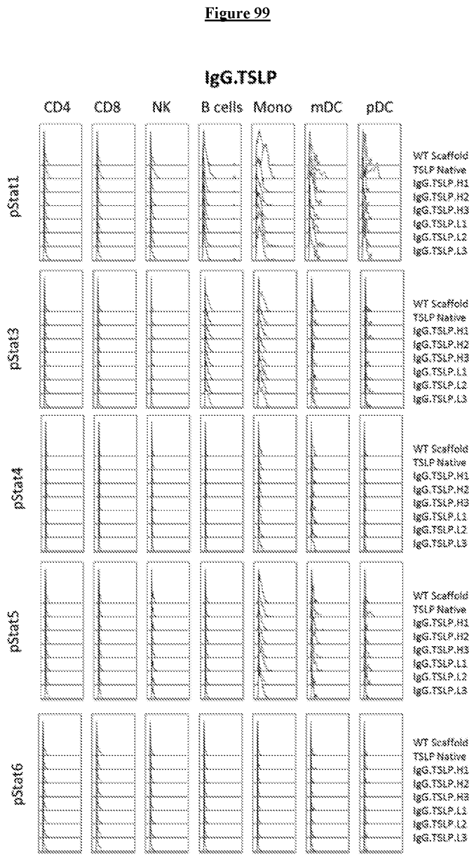

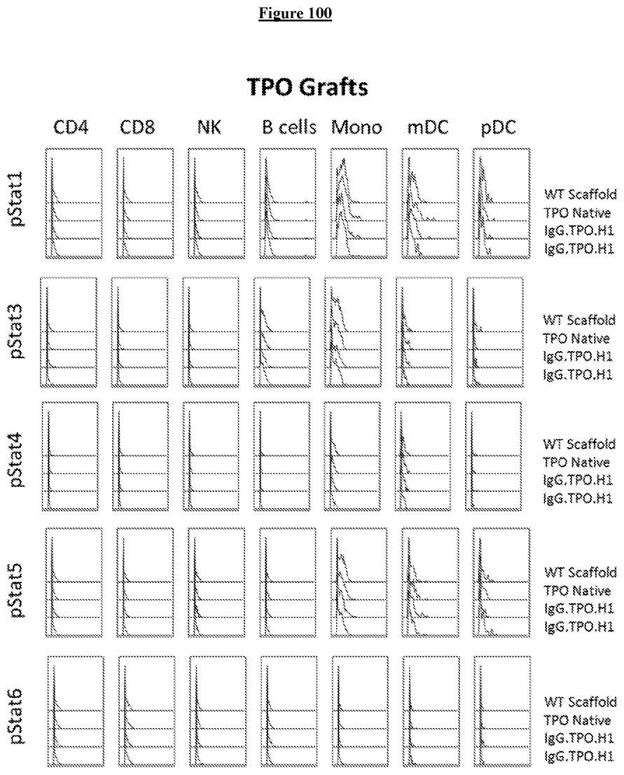

Human PBMCs were treated with the wild type antibody used for the scaffold of the ACE protein or the respective ACE protein. The native cytokine (e.g., IL3) was also included as a control if available. The cells were fixed with 1.6% PFA to preserve phosphorylation status on signaling molecules. The cells were then stained with a combination of cell surfaces receptors for specific lineages and intracellular signaling molecules of the JAK/Stat pathway. The samples were then acquired and analyzed on the CyTOF. Results for each ACE protein are shown in FIGS. 71-100.

### Example 54: Flt3L Grafts Inducing DC Differentiation

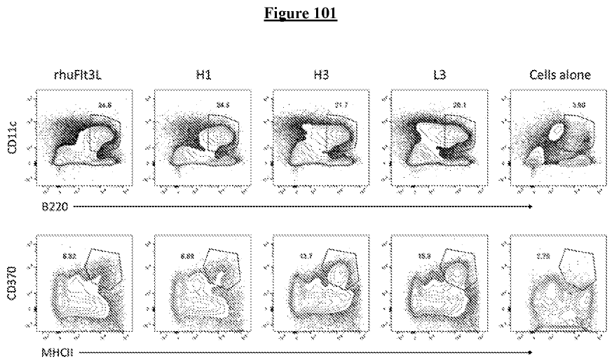

Mouse bone marrow from C57/BL6 mice was isolated by flushing femur and tibia bones with complete RPMI media (10% FBS, Pen/Step, Non-essential amino acids, sodium pyruvate, HEPES and Beta mercapto ethanol). Bone marrow was pelleted by centrifugation and red blood cells were lysed by addition of ACK lysis buffer (ThermoFisher #A1049201). Cells were plated at 2×10⁶ per mL in complete RPMI with recombinant human Flt3L (Peprotech #300-19-50UG) at 10.53 nM or molar equivalent doses of H1, H3 or L3 human Flt3L grafts and cultured for 5 days at 37° C. Cells were harvested by pipetting for flow cytometric analysis and stained with antibodies to CD103 (Biolegend #121422), CD11b (Biolegend #101257), CD11c (Biolegend #117306), MHCII (Biolegend #107628), CD370 (Biolegend #143504) and B220 (BD #552772). FACS staining was performed under standard protocols using FACS buffer (1×PBS+2% FBS+0.5 mM EDTA). FIG. 101 shows that H1, H3 and L3 Flt3L grafts are capable of inducing B220+CD11c+ plasmacytoid DC differentiation (top panels) and CD370+DC1 differentiation (bottom panels) comparable to what is observed with recombinant human Flt3L.

### Example 55: GM-CSF Grafts Inducing DC Differentiation

Human CD14+ monocytes were isolated from a leukapheresis using positive selection (Stem cell Technologies #17858). In order to induce monocyte dendritic cell (DC) differentiation, cells were cultured in duplicate in the presence of 20 ng/mL of recombinant human IL-4 (Peprotech #200-04-100UG) and varying concentrations of recombinant human GM-CSF (Peprotech #300-03-100UG) or GM-CSF grafts in complete RPMI (10% FBS, Pen/Step, Non-essential amino acids, sodium pyruvate, HEPES and Beta mercapto ethanol). Ungrafted palivizumab was used as a control (graft scaffold control).

After 6 days in culture at 37° C., cells were harvested and stained for flow cytometric analysis for CD16 (Biolegend #302032), HLA-DR (Biolegend 307644), CD86 (Biolegend #305414), DC-SIGN (Biolegend #330106), CD24 (Biolegend #311134), CD80 (Biolegend #305218), CD40 (Biolegend 313008), CD11c (eBioscience #56-0116-42) and CD14 (BD #557831). FACS staining was performed under standard protocols using FACS buffer (1×PBS+2% FBS+0.5 mM EDTA)

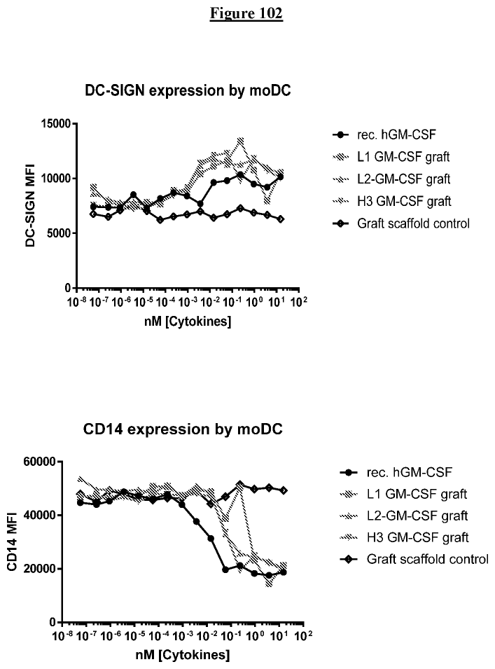

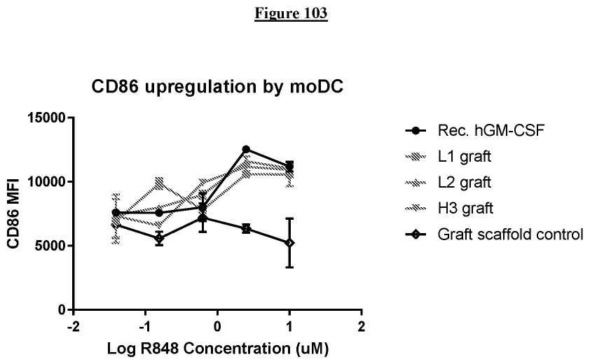

For R848 stimulation, cells were cultured for 6 days as described above (3.9 nM GM-CSF was used for recombinant human GM-CSF and GM-CSF grafts). GM-CSF and IL-4 media was washed off and cells were incubated with R848 (in house generated) in varying concentrations in complete RMPI overnight. The following morning, cells were stained for flow cytometric analysis as described above. FIG. 102 shows that GM-CSF cytokine grafts are capable of inducing monocyte DC differentiation as evidenced by upregulation of DC-SIGN on the cells and downregulation of CD14. FIG. 103 shows that monocyte DCs generated with GM-CSF grafts are capable of responding to TLR7/8 activation.

It is understood that the examples and embodiments described herein are for illustrative purposes and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

LENGTHY TABLES
The patent application contains a lengthy table section. A copy of the table is available in electronic form from the USPTO web site (https://seqdata.uspto.gov/?pageRequest=docDetail&DocID=US20200362058A1). An electronic copy of the table will also be available from the USPTO upon request and payment of the fee set forth in 37 CFR 1.19(b)(3).

1. An antibody cytokine engrafted (ACE) protein comprising:
(a) a heavy chain variable region (VH), comprising Complementarity Determining Regions (CDR) HCDR1, HCDR2, HCDR3; and
(b) a light chain variable region (VL), comprising LCDR1, LCDR2, LCDR3; and
(c) a cytokine molecule engrafted into a CDR of the VH or the VL,
wherein the cytokine molecule is directly engrafted into the CDR, and wherein the cytokine molecule is not interleukin-10 (IL-10).

2. The ACE protein of claim 1, wherein the cytokine molecule is engrafted into a heavy chain CDR.

3. (canceled)

4. The ACE protein of claim 1, wherein the cytokine molecule is engrafted into a light chain CDR.

5. (canceled)

6. (canceled)

7. The ACE protein of claim 1, wherein the cytokine molecule is a molecule selected from Table 1.

8. The ACE protein of claim 1, further comprising an IgG class antibody heavy chain.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The ACE protein of claim 1,
wherein the differential binding affinity or avidity of the engrafted cytokine molecule to two or more receptors is changed in comparison to a free cytokine molecule.

25. The ACE protein of claim 1,
wherein an activity of the engrafted cytokine molecule is increased in comparison to a free cytokine molecule.

26. The ACE protein of claim 1,
wherein an activity of the engrafted cytokine molecule is decreased in comparison to a free cytokine molecule.

27. (canceled)

28. The ACE protein of claim 1 comprising:
a heavy chain variable region that comprises: (a) a HCDR1, (b) a HCDR2, and (c) a HCDR3, wherein each of the HCDR sequences are set forth in TABLE 2, and
a light chain variable region that comprises: (d) a LCDR1, (e) a LCDR2, and (f) a LCDR3, wherein each of the LCDR sequences are set forth in TABLE 2,
wherein a cytokine molecule is engrafted into a CDR.

29. The ACE protein of claim 1 comprising:
a heavy chain variable region (VH) that comprises a VH set forth in TABLE 2, and
a light chain variable region (VL) that comprises a VL set forth in TABLE 2,
wherein a cytokine molecule is engrafted into a VH or VL.

30. The ACE protein of claim 1, further comprising a modified Fc region corresponding with reduced effector function.

31. The ACE protein of claim 30, wherein the modified Fc region comprises a mutation selected from one or more of D265A, P329A, P329G, N297A, L234A, and L235A.

32. (canceled)

33. (canceled)

34. An isolated nucleic acid encoding an ACE protein comprising:
a heavy chain variable region as set forth in TABLE 2, and
a light chain variable region as set forth in TABLE 2,
wherein a cytokine molecule is engrafted into the heavy chain variable region or the light chain variable region.

35. A recombinant host cell suitable for the production of an ACE protein, comprising the isolated nucleic acid of claim 34, and optionally, a secretion signal.

36. (canceled)

37. (canceled)

38. A pharmaceutical composition comprising the ACE protein of claim 1 and a pharmaceutically acceptable carrier.

39. A method of treating a disease in an individual in need thereof, comprising administering to the individual a therapeutically effective amount of the pharmaceutical composition of claim 38.

40. The method of claim 39, wherein the disease is a cancer.

41. The method of claim 40, wherein the cancer is selected from the group consisting of: melanoma, lung cancer, colorectal cancer, prostate cancer, breast cancer and lymphoma.

42. The method of claim 39, wherein the pharmaceutical composition is administered in combination with another therapeutic agent.

43. The method of claim 42, wherein the therapeutic agent is an immune checkpoint inhibitor.

44. The method of claim 43, wherein the immune checkpoint is selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4, and TGFR.

45. (canceled)

46. (canceled)

47. The method of claim 39, wherein the disease is an immune related disorder.

48. The method of claim 47, wherein the immune related disorder is selected from the group consisting of: inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

49. The method of claim 47,
wherein the pharmaceutical composition is administered in combination with another therapeutic agent.

50. The method of claim 49, wherein the therapeutic agent is an anti-TNF agent selected from the group consisting of: infliximab, adalimumab, certolizumab, golimumab, natalizumab, and vedolizumab; an aminosalicylate agent selected from the group consisting of: sulfasalazine, mesalamine, balsalazide, olsalazine and other derivatives of 5-aminosalicylic acid; a corticosteroid selected from the group consisting of: methylprednisolone, hydrocortisone, prednisone, budenisonide, mesalamine, and dexamethasone; or an antibacterial agent.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)
